(19) United States
(12) Patent Application Publication
(10) Pub. No.: US 2025/0144069 A1
(43) Pub. Date: May 8, 2025

(54) BILOBALIDE DERIVATIVE COMPOUNDS FOR TREATING NEUROLOGICAL DISEASES AND CANCERS

(71) Applicant: The Chinese University of Hong Kong, Hong Kong (CN)

(72) Inventors: Wai Lung NG, Hong Kong (CN); Stephan Juergen Wolfgang SCHEEFF, Hong Kong (CN); Josefina XEQUE AMADA, Hong Kong (CN); Xiaoding JIANG, Hong Kong (CN); Xu HE, Hong Kong (CN)

(21) Appl. No.: 18/793,753

(22) Filed: Aug. 3, 2024

Related U.S. Application Data
(60) Provisional application No. 63/517,627, filed on Aug. 4, 2023.
(60) Provisional application No. 63/610,394, filed on Dec. 14, 2023.

(51) Int. Cl. A61K 31/365; A61K 31/407; A61K 31/4178; A61K 31/4196; A61K 31/4439; A61K 31/454; A61K 31/496; A61K 31/497; A61K 31/5377; A61P 35/00; C07D 491/153; C07D 493/20

(52) U.S. Cl. CPC A61K 31/365; A61K 31/407; A61K 31/4178; A61K 31/4196; A61K 31/4439; A61K 31/454; A61K 31/496; A61K 31/497; A61K 31/5377; A61P 35/00; C07D 491/153; C07D 493/20

(57) ABSTRACT

Provided herein are bilobalide derivative compounds, processes for making, methods of using, and uses thereof for preventing or treating neurological disease and cancer.

### CROSS-REFERENCE TO RELATED APPLICATION

This application claims benefit under 35 U.S.C. § 119(e) of U.S. Provisional Application having Ser. No. 63/517,627 filed on Aug. 4, 2023 and U.S. Provisional Application having Ser. No. 63/610,394 filed on Dec. 14, 2023, the entire contents of which are hereby incorporated by reference herein.

### FIELD OF INVENTION

This application relates to bilobalide derivative compounds, processes for making, methods of using, and uses of said compounds for preventing or treating cancers and neurological diseases.

### BACKGROUND

High-throughput screening of chemical libraries is a common starting point in modern drug discovery. Unfortunately, many existing libraries consist of planar molecules with minimal structural or stereochemical complexity, thereby impeding the process of drug discovery.

Terpene trilactones (TTLs), such as ginkgolides and bilobalide, are polyoxygenated diterpenoids isolated from the Ginkgo tree. Bilobalide is not acutely toxic and has been demonstrated to exert a broad range of biological activities. However, the therapeutic potential of bilobalide is limited and its effects on the mammalian central nervous system, neurological diseases or cancers are not fully corroborated because of its instability. Due to the synthetic challenges for structural modification of bilobalide, no synthetic routes exist that enable facile access to bilobalide analogues for systematic structure-activity relationship (SAR) studies. As such, no effective bilobalide compounds have been identified that are useful for treating diseases.

### SUMMARY

Disclosed herein are novel compounds useful against cancers, processes for making, methods of using, and intermediates used in preparing the novel compounds.

In some embodiments, provided is a compound of Formula I:

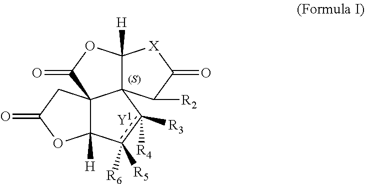

or a stereomer, a tautomer, or a pharmaceutically acceptable salt thereof,
wherein
X is —O—, —NR¹—, —N═CR¹—NH—, or —NR¹—NH—; wherein when X is —O—, R¹ is absent;
bond Y¹ is between R⁴ and R⁵ and is a single bond or a double bond;
R¹ is H, R^{1B}, or -(L¹)ᵤ-(Z¹)ᵥ; wherein
   L¹ is C₁-C₁₀ aliphatic wherein up to three carbon atoms of the C₁-C₁₀ aliphatic are optionally replaced by N, O, or S; wherein L¹ is optionally substituted with 1-3 occurrences of halo, CN, R, OR′, or R^{1C};
      u is 0 or 1;
      v is 0 or 1;
   Z¹ is a 5-16 membered aromatic or nonaromatic monocyclic, bicyclic, or tricyclic ring system having 0-7 heteroatoms selected from O, N, or S; wherein Z¹ is optionally substituted with 1-5 occurrences of R^{1A}, R^{1C} or combinations thereof;
R^{1A} is -(L²)ₘ-(Z²)_{w}; wherein
   L² is C₁-C₁₀ aliphatic wherein up to three carbon atoms of the C₁-C₁₀ aliphatic are optionally replaced by N, NR, O, S, C═O, SO₂, S═O, (C═O)N, N(C═O)N, (C═O)O, or Si; wherein L² is optionally substituted with 1-3 occurrences of halo, CN, R, OR′ or R^{1C};
   m is 0 or 1;
   w is 0 or 1;
Z² is a C₁-C₁₀ aliphatic, or 3-16 membered aromatic or nonaromatic monocyclic, bicyclic or tricylic ring system having 0-7 heteroatoms selected from O, N, or S; wherein Z² is optionally substituted with 1-5 occurrences of R^{1B};
R^{1B} is H, halo, CN, R*, OR*, NRR*; or two R^{1B}, taken together with the atom to which they are attached, form a 3-6 membered ring having 0-4 heteroatoms;
R^{1C} is H, halo, CN, a 5-10 membered aromatic or nonaromatic monocyclic or bicyclic ring system having 0-5 heteroatoms selected from O, N, or S; R*, OR*, NRR*; or two R^{1C}, taken together with the atom or atoms to which they are attached, optionally form a 3-16 membered ring having 0-4 heteroatoms; wherein R^{1C} is optionally substituted with 1-3 occurrences of halo, CN, R′ or OR′;
R* is C₁-C₆ aliphatic wherein up to three methylene units of the C₁-C₆ aliphatic are optionally replaced by N, NR, O, S, C═O, SO, SO₂ or Si and wherein the C₁-C₆ aliphatic is optionally substituted with 1-3 occurrences of halo, CN, R′ or OR′;
R² is R^{2A} or OR^{2A}, wherein R^{2A} is H, a C₁-C₁₆ aliphatic, a 5-10 membered aromatic or nonaromatic monocyclic or bicyclic ring system, or —(C₁-C₁₆ aliphatic)-(5-10 membered aromatic or nonaromatic monocyclic or bicyclic ring system); wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{2A} is optionally substituted with 1-5 occurrences of R^{2B}, wherein R^{2B} is halo, R′ or OR′;
R³ is OH, R^{3A}, or OR^{3A}; wherein R^{3A} is C₁-C₁₀ aliphatic optionally substituted with 1-3 occurrences of halo, R or OR′;
R⁴ is OH, R^{4A}, OR^{4A}; or when bond Y¹ between R⁴ and R⁵ is a double bond, R⁴ is absent; wherein R^{4A} is C₁-C₇ aliphatic and R^{4A} is optionally substituted with 1-3 occurrences of halo, R′ or OR′;
R⁵ is H or OH;
R⁶ is H; or when bond Y¹ between R⁴ and R⁵ is a double bond, R⁶ is absent;
R is H or C₁-C₆ aliphatic optionally substituted by 1-3 occurrences of F; or two R, taken together with the atom(s) to which they are attached, form a 3-6 membered ring having 0-4 heteroatoms; and
R′ is H, a C₁-C₆ aliphatic wherein up to three carbon atoms of the C₁-C₆ aliphatic are optionally replaced with O, NH, N(C₁-C₆alkyl), C(O), or S(O)₂; wherein said C₁-C₆ aliphatic is optionally substituted by 1-3 occurrences of F, OR, NH₂, NHR″, or NR″₂, wherein R″ is C₁-C₆ aliphatic or a 5-10 membered aromatic or nonaromatic monocyclic or bicyclic ring system having 0-5 heteroatoms selected from O, N, or S;
wherein when R² is OH, R³ is tert-butyl, R⁴ is OH, R⁵ is H, and R⁶ is H, X is not —O—.

In some embodiments, provided is a compound of Formula I:

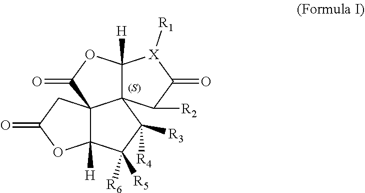

or a stereomer, a tautomer, or a pharmaceutically acceptable salt thereof,
wherein
X is —O—, —NR¹—, —N═CR¹—NH—, or —NR¹—NH—; wherein when X is —O—, R¹ is absent; bond Y¹ is a single bond or a double bond;
R¹ is H, RB, or -(L¹)ᵤ-(Z¹)ᵥ; wherein
   L¹ is C₁-C₁₀ aliphatic wherein up to three carbon atoms of the C₁-C₁₀ aliphatic are optionally replaced by N, O, or S; wherein L¹ is optionally substituted with 1-3 occurrences of halo, CN, R, OR′, or R^{1C};
      u is 0 or 1;
      v is 0 or 1;
   Z¹ is a 5-16 membered aromatic or nonaromatic monocyclic, bicyclic, or tricyclic ring system having 0-7 heteroatoms selected from O, N, or S; wherein Z¹ is optionally substituted with 1-5 occurrences of R^{1A}, R^{1C} or combinations thereof;
R^{1A} is -(L²)ₘ-(Z²)_{w}; wherein
   L² is C₁-C₁₀ aliphatic wherein up to three carbon atoms of the C₁-C₁₀ aliphatic are optionally replaced by N, NR, O, S, C═O, SO₂, S═O, (C═O)N, N(C═O)N, (C═O)O, or Si; wherein L² is optionally substituted with 1-3 occurrences of halo, CN, R, OR′ or; m is 0 or 1;
   w is 0 or 1;
Z² is a C₁-C₁₀ aliphatic, or 3-16 membered aromatic or nonaromatic monocyclic, bicyclic or tricylic ring system having 0-7 heteroatoms selected from O, N, or S; wherein Z² is optionally substituted with 1-5 occurrences of R^{1B};
R^{1B} is H, halo, CN, R*, OR*, NRR*; or two R^{1B}, taken together with the atom to which they are attached, form a 3-6 membered ring having 0-4 heteroatoms;
R^{1C} is H, halo, CN, R*, OR*, NRR*; or two R^{1C}, taken together with the atom or atoms to which they are attached, optionally form a 3-16 membered ring having 0-4 heteroatoms; R* is C₁-C₆ aliphatic wherein up to three methylene units of the C₁-C₆ aliphatic are optionally replaced by N, NR, O, S, C═O, SO, SO₂ or Si and wherein the C₁-C₆ aliphatic is optionally substituted with 1-3 occurrences of halo, CN, R′ or OR′;
R² is R^{2A} or OR^{2A}, wherein R^{2A} is H, a C₁-C₁₆ aliphatic, a 5-10 membered aromatic or nonaromatic monocyclic or bicyclic ring system, or —(C₁-C₁₆ aliphatic)-(5-10 membered aromatic or nonaromatic monocyclic or bicyclic ring system); wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{2A} is optionally substituted with 1-5 occurrences of R^{2B}, wherein R^{2B} is halo, R′ or OR′;
R³ is OH, R^{3A}, or OR^{3A}; wherein R^{3A} is C₁-C₁₀ aliphatic optionally substituted with 1-3 occurrences of halo, R or OR′;
R⁴ is OH, R^{4A}, OR^{4A}; or when bond Y¹ between R⁴ and R⁵ is a double bond, R⁴ is absent; wherein R^{4A} is C₁-C₇ aliphatic and R^{4A} is optionally substituted with 1-3 occurrences of halo, R′ or OR′;
R⁵ is H or OH;
R⁶ is H; or when bond Y¹ between R⁴ and R⁵ is a double bond, R⁶ is absent;
R is H or C₁-C₆ aliphatic optionally substituted by 1-3 occurrences of F; or two R, taken together with the atom(s) to which they are attached, form a 3-6 membered ring having 0-4 heteroatoms; and
R′ is H, a C₁-C₆ aliphatic optionally substituted by 1-3 occurrences of F, OR, NH₂, NHR″, NR″₂, wherein R″ is C₁-C₆ aliphatic, or a 5-10 membered aromatic or nonaromatic monocyclic or bicyclic ring system having 0-5 heteroatoms selected from O, N, or S;
wherein when R² is OH, R³ is tert-butyl, R⁴ is OH, R⁵ is H, and R⁶ is H, X is not —O—.

For the sake of clarity, when u is 0 or v is 0, then the bond before -(L¹))ᵤ or before —(Z¹)ᵥ is also absent, respectively. Likewise, when m and w is 0, the bond before -(L²)ₘ or before —(Z²)_{w} is also absent.

In some embodiments, provided is a compound of Formula II:

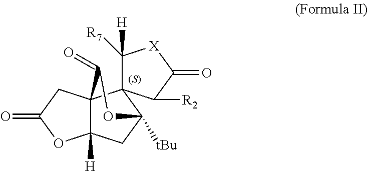

or a stereomer, a tautomer, or a pharmaceutically acceptable salt thereof,
wherein
X is —O—, —NR¹—, —N═CR¹—NH—, or —NR¹—NH—; wherein R¹ is as defined herein;
R² is R^{2A} or OR^{2A}, wherein R^{2A} is H, a C₁-C₁₆ aliphatic or a 5-10 membered aromatic or nonaromatic monocyclic or bicyclic ring system, wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{2A} is optionally substituted with 1-5 occurrences of R^{2B}, wherein R^{2B} is halo, R′ or OR′; and
R⁷ is R^{7A} or OR^{7A}, wherein R^{7A} is H, a C₁-C₁₆ aliphatic or a 5-10 membered aromatic or nonaromatic monocyclic or bicyclic ring system, wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{7A} is optionally substituted with 1-5 occurrences of R^{7B}, wherein R^{7B} is halo, R′ or OR′.

In some embodiments, provided is a process for preparing a compound as described herein, including at least the following steps: (i) treating bilobalide with R^{2A}—X in a suitable solvent to form protected product IIa

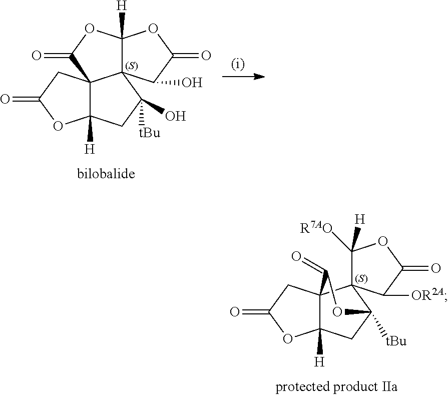

and (ii) treating protected product IIa with at least one base or an acceptable salt thereof to form aminated product IIb

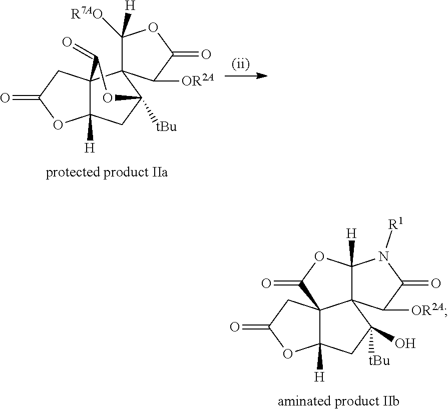

wherein R^{2A} and R^{7A} are as defined in any one of the preceding embodiments or described herein.

In some embodiments, provided is a method of treating or preventing cancer in a subject in need thereof, including administering to the subject a compound described herein.

In some embodiments, provided is a use of a compound described herein for treating or preventing cancer.

In some embodiments, provided is a use of a compound described herein for the manufacture of a medicament for treating or preventing cancer.

In some embodiments, provided is a method of inducing cell death in a cancer cell, including contacting a compound described herein with the cancer cell.

In some embodiments, provided is a method of inhibiting cell growth in a cancer cell, including contacting a compound described herein with the cancer cell.

In some embodiments, provided is a method of treating or preventing neurological related disease in a subject in need thereof, including administering to the subject a compound of any one of the embodiments herein.

In some embodiments, provided is a use of a compound described herein for treating or preventing Alzheimer's disease or Parkinson's disease.

In some embodiments, provided is a use of a compound described herein for inhibiting ferroptosis by restoring glutathione peroxidase 4 (GPX4), thereby mitigating GPX4 degradation induced by ferroptosis inducers.

In some embodiments, provided is a use of a compound described herein for inhibiting ferroptosis by reducing intracellular reactive oxygen species (ROS level).

In some embodiments, provided is a use of a compound described herein for inhibiting ferroptosis by reducing lipid peroxidation.

In some embodiments, provided is a use of a compound described herein for the manufacture of a medicament for treating or preventing Alzheimer's disease or Parkinson's disease.

There are many advantages of the invention. In certain embodiments, the methods and processes disclosed herein enable the synthesis of bilobalide (BB) compounds that can be used for SAR studies. In certain embodiments, the novel compounds are particularly effective at treating or preventing cancer. In certain embodiments, the novel compounds are effective at inducing cell death in a cancer cell. In certain embodiments, the novel compounds are effective at inhibiting cell growth in a cancer cell. In other embodiments, the novel compounds have demonstrated surprising pan-anti-cancer effect against human and mouse cancer cells. In certain embodiments, the novel compositions are particularly effective reducing chemically-induced ferroptosis and/or chemically-induced oxidative stress. In other embodiments, the novel compounds have demonstrated surprising neuroprotective properties in mouse models.

### BRIEF DESCRIPTION OF FIGURES

FIG. 1A is an illustration showing the overall molecular editing schemes of bilobalide as disclosed herein.

FIG. 1B is an illustration of Scheme 12.

FIG. 1C is a plot showing the X-ray crystal structure of bilobalide analogue XBB-002.

FIG. 1D is a plot showing the X-ray crystal structure of bilobalide analogue XBB-003.

FIG. 1E is a plot showing the X-ray crystal structure of bilobalide analogue XBB-018.

FIG. 1F is a plot showing the X-ray crystal structure of bilobalide analogue JW072.

FIG. 1G is a plot showing the X-ray crystal structure of bilobalide analogue XBB-009.

FIG. 1H is a plot showing the X-ray crystal structure of bilobalide analogue XBB-010.

FIG. 2 is a heat map showing cell viability after treatment with 50 uM DW192 for 48 hours in A549 and Jurkat cells.

FIG. 3A is a plot showing the dose-response curve on A549 cells treated with DW192 for 48 hours.

FIG. 3B is a plot showing the dose-response curve on A549 cells treated with P-29 for 48 hours.

FIG. 3C is a plot showing the dose-response curve on A549 cells treated with P-21 for 48 hours.

FIG. 3D is a plot showing the dose-response curve on KP-1 cells treated with SCC506 for 48 hours.

FIG. 3E is a plot showing the dose-response curve on A549 cells treated with SCC363 for 48 hours.

FIG. 3F is a plot showing overlaid dose-dependent curves on Jurkat cells, A549 cells, KP-1 cells, and MCF-7 cells treated with DW192 for 48 hours.

FIG. 4A is a one-dose mean graph of percentage growth of cell lines across the NCI-60 cell line panel when treated DW192 (10 uM, 48 h).

FIG. 4B is a plot showing the dose-response curves of leukemia cell lines treated with DW192.

FIG. 4C is a plot showing the dose-response curves of CNS cancer cell lines treated with DW192.

FIG. 4D is a plot showing the dose-response curves of renal cancer cell lines treated with DW192.

FIG. 4E is a plot showing the dose-response curves of NSCLC cell lines treated with DW192.

FIG. 4F is a plot showing the dose-response curves of melanoma cell lines treated with DW192.

FIG. 4G is a plot showing the dose-response curves of prostate cancer cell lines treated with DW192.

FIG. 4H is a plot showing the dose-response curves of colon cancer cell lines treated with DW192.

FIG. 4I is a plot showing the dose-response curves of ovarian cancer cell lines treated with DW192.

FIG. 4J is a plot showing the dose-response curves of breast cancer cell lines treated with DW192.

FIG. 4K shows the mean graphs of GI50, TGI and LC50 calculated from five-dose screen results.

FIG. 5A, is a chart showing the hydrolytic stabilities of bilobalide versus bilobalide analogue in buffer with pH=6.8.

FIG. 5B, is a chart showing the hydrolytic stabilities of bilobalide versus bilobalide analogue in buffer with pH=7.4.

FIG. 6A is a chart comparing the phenotypic screening of bilobalide and bilobalide analogue against RSL3-induced ferroptosis through 3 cell lines is shown.

FIG. 6B is a chart showing the dose-dependent curves of RSL3 on HT22 cell line treated with or without bilobalide or bilobalide analogue.

FIG. 6C is a chart showing the dose-dependent curves of RSL3 on HMC3 cell line treated with or without bilobalide or bilobalide analogue.

FIG. 6D is a chart showing the dose-dependent curves of RSL3 on BV-2 cell line treated with or without bilobalide or bilobalide analogue.

FIG. 6E is a chart comparing the fluorescent staining on HMC3 cell lines treated with RSL3 treated with or without bilobalide or bilobalide analogue.

FIG. 6F is a plot showing the normalization of ROS level against the Control based on the fluorescent intensity of CellROX.

FIG. 6G is a plot showing the normalization of ROS level in cells treated with various concentrations of XBB-037.

FIG. 6H is a plot showing the lipid peroxidation level (%) in cells treated with various concentrations of XBB-037.

FIG. 6I is a plot showing the lipid peroxidation level (%) in cells treated with various concentrations of bilobalide.

FIG. 7A is a chart showing the killing effect of ferroptosis inducer ML162 on HMC3 cells with or without bilobalide or bilobalide analogue.

FIG. 7B is a chart showing the killing effect of ferroptosis inducer ML210 on HMC3 cells with or without bilobalide or bilobalide analogue.

FIG. 7C is a chart showing the killing effect of ferroptosis inducer erastin on HMC3 cells with or without bilobalide or bilobalide analogue.

FIG. 7D is a chart showing the killing effect of ferroptosis inducer FIN56 on HMC3 cells with or without bilobalide or bilobalide analogue.

FIG. 7E is a chart of the Western-blot images showing the levels of GPX4 and 3-actin at 0, 250, 500 or 750 nM RSL3.

FIG. 7F is a plot showing the normalized plot of GPX4 levels with or without bilobalide analogue XBB-037, according to FIG. 7E.

FIG. 7G is a chart of the Western-blot images showing the levels of GAPDH at 0 or 500 nM RSL3.

FIG. 7H is a chart of the Western-blot images showing the levels of GPX4 and (3-actin at 0, 0.625, 1.25 and 2.5 μM FIN56.

FIG. 7I is a plot showing the normalized plot of GPX4 levels according to FIG. 7H.

FIG. 8A is a chart comparing the phenotypic screening of bilobalide, SXQ087-1 and XBB-037 against RSL3-induced ferroptosis through 3 cell lines.

FIG. 8B is a plot showing the dose-dependent cell viability (%) curves of SXQ087-1 or XBB-037 on HMC3 cell line treated with RSL3.

FIG. 8C is a plot showing the lipid peroxidation level (%) in cells treated with various concentrations of SXQ087-1.

FIG. 9A is a chart of the Western-blot images showing the levels of GPX4 and GAPDH at various concentrations of SXQ087-1 against RSL3.

FIG. 9B is a plot showing the normalized plot of GPX4 levels with various concentrations of SXQ087-1, according to FIG. 9A.

FIG. 9C is a chart of the Western-blot images showing the levels of LC3-II/LC3-I and GAPDH at various concentrations of SXQ087-1 against RSL3.

FIG. 9D is a plot showing the normalized plot of LC3-II/LC3-I level (%) levels according to FIG. 9C.

FIG. 10A is a chart showing the cell viability (%) curves of HMC3 cells treat with FIN56 or FIN56+SXQ087-1.

FIG. 10B is a chart showing the cell viability (%) curves of HMC3 cells treat with ML162 or ML162+SXQ087-1.

FIG. 10C is a chart showing the cell viability (%) curves of HMC3 cells treat with ML210 or ML210+SXQ087-1.

FIG. 10D is a chart showing the cell viability (%) curves of HMC3 cells treat with Erastin or Erastin+SXQ087-1.

FIG. 11A is a chart showing the cell viability (%) curves of HMC3 cells treated with SXQ087-1, XBB-037 or bilobalide.

FIG. 11B is a chart showing the radical scavenging activity of SXQ087-1.

FIG. 12A is a plot showing the tumor volume measurements of C57BL/6 mice with B16 melanoma allograft treated with DW192 or vehicle.

FIG. 12B is a photograph of tumors after DW192 or vehicle treatment.

### DETAILED DESCRIPTION

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

For example, alternate embodiments of “a composition comprising A, B, and C” would be “a composition consisting of A, B, and C” and “a composition consisting essentially of A, B, and C.” Even if the latter two embodiments are not explicitly written out, this disclosure/application includes those embodiments. Furthermore, it shall be understood that the scopes of the three embodiments listed above are different.

For the sake of clarity, “comprising”, including, and “containing”, and any related forms are open-ended terms which allows for additional elements or features beyond the named essential elements, whereas “consisting of” is a closed end term that is limited to the elements recited in the claim and excludes any element, step, or ingredient not specified in the claim.

For the sake of clarity, “characterized by” or “characterized in” (together with their related forms as described above), does not limit or change the nature of whether the list of terms following it are open or closed. For example, in a claim directed towards “a composition comprising A, B, C, and characterized in D, E, and F”, the elements D, E, and F are still open-ended terms and the claim is meant to include other elements due to the use of the word “comprising” earlier in the claim.

“Consisting essentially of” limits the scope of a claim to the specified materials, components, or steps (“essential elements”) that do not materially affect the essential characteristic(s) of the claimed invention. In some embodiments, the essential characteristics are the basic and novel characteristic(s) of the claimed invention. For example, in some embodiments, the essential elements of a composition of the disclosure can be “Xmg to Ymg” of compound A. Even if the composition includes additional excipients, as long as the additional excipients do not materially affect the essential characteristics of the compound, e.g., in compound A's cytotoxic effect against cancer cell lines, then such embodiment that “consists essentially of compound A” still includes compositions with the aforementioned additional excipients.

As used herein, the singular forms “a”, “an” and “the” are intended to include the plural forms as well, unless the context clearly indicates otherwise. Where a range is referred in the specification, the range is understood to include each discrete point within the range. For example, 1-7 means 1, 2, 3, 4, 5, 6, and 7.

As used herein, the term “about” is understood as within a range of normal tolerance in the art and not more than ±10% of a stated value. By way of example only, about 50 means from 45 to 55 including all values in between. As used herein, the phrase “about” a specific value also includes the specific value, for example, about 50 includes 50.

As used herein and in the claims, an “effective amount”, is an amount that is effective to achieve at least a measurable amount of a desired effect. For example, the amount may be effective to cause cell death.

As used herein, the term “cancer” refers to a group of diseases characterized by the uncontrolled growth and spread of abnormal cells that tend to invade surrounding tissue or organs and to metastasize to other parts of the body through the bloodstream or lymphatic system. Examples of cancer include, but are not limited to bladder cancer, brain cancer, breast cancer, CNS cancer, colon cancer, colorectal cancer, hematopoietic cancer, kidney cancer, leukemia (such as lymphocytic leukemia), lung cancer (such as non-small cell lung cancer (NSCLC)), melanoma, multiple myeloma, ovarian cancer, pancreatic cancer, prostate cancer, renal cancer and other cancers. In some embodiments, NSCLC are associated with KRAS and/or P53 mutations.

As used herein and in the claims, a “subject” refers to animals such as mammals, including, but not limited to, primates (e.g., humans), cows, sheep, goats, horses, dogs, cats, rabbits, rats, mice and the like.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

In various aspects, the compounds disclosed herein further comprise their isotopically-labelled or isotopically-substituted variants, i.e., compounds identical to those described, but for the fact that one or more atoms are replaced by an atom having an atomic mass or mass number different from the atomic mass or mass number typically found in nature. For example, the isotopically-labelled or isotopically-substituted atom has one or more neutrons in the nucleus compared to the natural atom. In some embodiments, the disclosed compounds comprise a mixture of natural atoms and their isotopically labeled variants. Examples of isotopes that can be incorporated into the disclosed compounds include isotopes of hydrogen, carbon, nitrogen, oxygen, sulfur, phosphorous, fluorine and chlorine, such as ²H, ³H, ¹³C, ¹⁴C, ¹⁵N, ¹⁷O, ¹⁸O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this disclosure.

As used herein, the terms “halogen” or “halo” mean F, Cl, Br, or I (fluoro, chloro, bromo, or iodo).

As used herein, the term “aliphatic”, “aliphatic group”, “alkyl” or “alkyl group”, means a straight-chain (i.e., unbranched), branched, or cyclic, substituted or unsubstituted hydrocarbon chain that is completely saturated or that contains one or more units of unsaturation that has a single point of attachment to the rest of the molecule.

In some embodiments, an aliphatic group contains 1-20 carbon atoms. In some embodiments, an aliphatic group contains 1-10 carbon atoms. In some embodiments, an aliphatic group contains 1-6 carbon atoms. In some embodiments, an aliphatic group contains 1-4 carbon atoms. In some embodiments, aliphatic groups are linear or branched, substituted or unsubstituted alkyl, alkenyl, or alkynyl groups. Specific examples include, but are not limited to, methyl, ethyl, isopropyl, isopropenyl, n-propyl, sec-butyl, vinyl. N-butenyl, ethynyl, and tert-butyl. In some embodiments, aliphatic groups are cyclic, or have a combination of linear or branched and cyclic groups, which are known as “cyloalkyls”. Specific examples of such types of aliphatic groups or cycloalkyls include, but are not limited to cyclopropyl, cyclobutyl, cyclopentyl, cyclohexyl, cyclohexenyl, cyclooctyl, cyclobutylmethyl, cyclopropylmethyl, and —CHCH—CH(CH)-cyclohexyl. Cyclic groups, (e.g. cycloaliphatic and heterocycles), can be linearly fused, bridged, or spirocyclic, for example, 1-(bicyclo[2.2.1]hept-5-en-2-yl)methyl, bicyclo[1.1.1]pentyl, 1-adamantanemethyl, norbornane and decahydronaphthalene.

In some embodiments, carbon atom(s) of an aliphatic group are replaced by any number of non-carbon atoms such as N, O, S, B, P, Al, and Si. For example, a C2 aliphatic where two carbon atoms are replaced by two nitrogens forms —N═NH or —N═NR. In some embodiments, cycloalkyl is a “heterocyclic ring” which is a 5-10 membered nonaromatic monocyclic, bicyclic or tricyclic ring having 0-5 heteroatoms selected from N, O and/or S. In some embodiments, a heterocyclic ring include a spirocyclic ring.

As used herein, the term “heteroatom” means one or more of N, O, S, B, P, Al, Si, or any oxidized forms thereof, such as SO and SO₂.

In some embodiments, aliphatic groups are “haloalkyls” which contain halogens. Specific examples of such types of aliphatic groups or haloalkyls include, but are not limited to, trifluoromethyl, flouromethyl, 1,2,3,4,5-pentafluoro-phenyl. In some embodiments, haloalkyls include “perfluoro” compounds where at least two available hydrogens are substituted with fluorine. Examples of “perfluoro” compounds include, but are not limited to, perfluorophenyl (i.e., 1,2,3,4,5-pentafluorophenyl), perfluoromethane (i.e., 1,1,1-trifluoromethyl), and perfluoromethoxy (i.e., 1,1,1-trifluoromethoxy).

As used herein, the terms “aryl”, “aryl group”, or “aromatic” or “aromatic ring” refer to mono cyclic, bicyclic, and tricyclic ring systems having a total of 5 to 20 ring members, wherein at least one ring in the system is fully unsaturated (i.e., aromatic), wherein the ring may be substituted or unsubstituted, wherein each ring in the system contains 3 to 7 ring members, and wherein the ring members may be heteroatoms. In some embodiments, the terms refer to a 5-10 membered aromatic monocyclic or bicyclic ring having 0-5 heteroatoms selected from oxygen, nitrogen, or sulfur. In some embodiments, the aryl is a “heteroaryl”, “heteroaryl group”, or “heterocyclic aromatic ring”. These refer to an “aryl”, “aryl group”, or “aromatic ring” wherein at least one ring in the mono cyclic, bicyclic, and tricyclic ring system includes a ring member that is a heteroatom. Examples of heteroaryl groups include, but are not limited to, optionally substituted phenyl, naphthyl, furanyl (e.g., 2-furanyl, 3-furanyl), imidazolyl (e.g., N-imidazolyl, 2-imidazolyl, 4-imidazolyl, 5-imidazolyl), benzimidazolyl, isoxazolyl (e.g., 3-isoxazolyl, 4-isoxazolyl, 5-isoxazolyl), oxazolyl (e.g., 2-oxazolyl, 4-oxazolyl, 5-oxazolyl), pyrrolyl (e.g., N-pyrrolyl, 2-pyrrolyl, 3-pyrrolyl), pyridyl (e.g., pyridinyl, 2-pyridyl, 3-pyridyl, 4-pyridyl), pyrimidinyl (e.g., 2-pyrimidinyl, 4-pyrimidinyl, 5-pyrimidinyl), pyrazinyl (e.g., 4-pyrazinyl), pyridazinyl (e.g., 3-pyridazinyl), thiazolyl (e.g., 2-thiazolyl, 4-thiazolyl, 5-thiazolyl), tetrazolyl (e.g., 5-tetrazolyl), triazolyl (e.g., 2-triazolyl and 5-triazolyl), thienyl (e.g., 2-thienyl, 3-thienyl), benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, 1,3,5-triazinyl, quinolinyl (e.g., 2-quinolinyl, 3-quinolinyl, 4-quinolinyl), isoquinolinyl (e.g., 1-isoquinolinyl, 3-isoquinolinyl, or 4-isoquinolinyl), tetrahydrothiophenyl, morpholino, quinuclidinyl, and 1,4-dioxa-8-azaspiro[4.5]dec-8-yl. In some embodiments, aryl groups are optionally substituted by one or more groups such as alkyl, alkoxy, aryl, hydroxy, halogen, cyano, amino, amino-alkyl, trifluoromethyl, alkylenedioxy (i.e., methylenedioxy or ethylenedioxy) and oxy-Cg-C3-alkylene (i.e., 2,3-dihydrobenzofuran-5-yl).

As used herein, the term “alkoxy” or “OR” refers to a group where the attaching oxygen is bound to an R group. In some embodiments, the R group is an alkyl group defined in the preceding paragraphs. Examples of an alkoxy group include, but are not limited to, methoxy, ethoxy, propoxy, iso-propoxy, butoxy, 2-butoxy, iso-butoxy, sec-butoxy, tert-butoxy, pentoxy, and hexoxy.

As used herein, the term “nonaromatic” refers to aliphatics that are fully saturated or partially saturated.

As used herein, the terms “carbonyl” or “C═O” refers to a group where a carbon is forms a double bond with an oxygen. Examples include, but are not limited to, aldehydes, ketones, carboxylic acids, esters, and amides.

Unless otherwise indicated, structures depicted herein are also meant to include all isomeric (e.g., enantiomeric, diastereomeric, geometric, conformational, and rotational) forms of the structure.

The terms “stereomer” or “stereoisomer” include the R and S configurations for each asymmetric center.

The term “tautomer” includes all the structural isomers resulted from interconversion. Examples of tautomers include, but are not limited to, keto-enol tautomers, amine-imine tautomers, amide-imidic acid tautomers, and lactam-lactim tautomers. Unless otherwise indicated, all tautomeric forms of the compounds of the disclosure are within the scope of the invention.

The term “acceptable salt” refers to a compound which has a net charge of zero. In some embodiments, an acceptable salt may be a salt of an acid or a salt of a base. Examples of acceptable salts include, but not limited to, fluorides (e.g., F⁻), chlorides (e.g., Cl⁻), bromides (e.g., Br⁻), iodides, carbonates, triflates (e.g., OTf⁻), hydroxides (e.g., OH⁻), formates, acetates (e.g., OAc⁻), oxides (e.g., Q²⁻), sulfates, pyrosulfates, bisulfates, sulfites, bisulfites, phosphates (e.g., monohydrophosphates, dihydrophosphates, metaphosphates, pyrophosphates), fluoromethoxides (e.g., [OCF₃]⁻, [OCF₂R]⁻, [OCFR₂]⁻), propionates, butyrates (e.g., isobutyrates), oxalates, malonates, succinates, fumarates, maleates, benzoates (e.g., chlorobenzoates, methyl benzoates, nitrobenzoates, hydroxybenzoates, methoxybenzoates), phthalates, sulfonates, citrates, lactates, glycolates, tartrates, ascorbate, acrylates, sulfides, phosphides, cyanides, azides, oxochlorides (ClO⁻, ClO₂⁻, ClO₃⁻ ClO₄⁻), tetrafluoroborates, tetraphenylborates, hexafluorophosphates, and the like.

As used herein, the term “amine” refers to a compound having at least one or more nitrogen groups. In some embodiments, the nitrogen groups are primary, secondary or tertiary. In some embodiments, an amine is NH₃, NH₂R, NHR₂, or NR₃, and its acceptable salt is referred as “ammounium” in the form of NH₄⁺, [H₃NR]⁺, [H₂NR₂]⁺, [HNR₃]⁺, or [NR₄]⁺, respectively. In some embodiments, an amine is a carbamate, an amino acid, an amide, a hydrazine, or acceptable salts thereof. In some embodiments, an amine is a combination of the amine and its acceptable salt. In some embodiments, an amine is ammonia, trimethylamine, triethylamine, diisopropylethylamine, tetramethylethylenediamine, ethyl amine, propyl amine, isopropyl amine, ethylenediamine, ethanolamine, other NH₂R¹, or combination thereof. In some embodiments, an amine is NH₂R¹, and its acceptable salt is [H₃NR¹]Cl. In some embodiments, an amine is a heterocyclic ring or a hetero aromatic ring as defined in the preceding paragraphs. Examples of a heterocyclic ring or a hetero aromatic ring include, but are not limited to, quinuclidine, N,N-methylphenylamine, aniline, 4-methylaniline, pyridine, pyrrolidine, piperdine, imidazole, pyridazine, pyrimidine, pyrazine 1,5-Diazabicyclo[4.3.0]non-5-ene (DBN), and 1,8-Diazabicyclo[5.4.0]undec-7-ene (DBU).

The term “base” refers to an electron donor which can share electrons with electron acceptors. In some embodiments, a base reacts with an electrophile to form a covalent bond. In some embodiments, a base reacts with acids to form salts. In some embodiments, the term “base” refers to an amine defined in the preceding paragraph or an acceptable salt thereof.

As used herein, the terms “bilobalide” or “BB” refers to a terpenic trilactone substance.

As used herein, the terms “solvent” or “suitable solvent” refer to a substance which is capable of dissolving or dispersing other substances. In some embodiments, a solvent is liquids, ionic liquids, gases, and supercritical fluids. In some embodiments, a solvent is water, alcohols, ethers, hydrocarbons, and other organic and inorganic solvents. In some embodiments, the solvent or suitable solvent is dichloromethane (DCM), dichloroethane (DCE), chloroform, tetrahydrofuran (THF), acetonitrile, toluene, chlorobenzene, benzene, pyridine, 2,6-lutidine, dioxane (e.g., 1,4-dioxane), dimethyl sulfoxide (DMSO), dimethylformamide (DMF), ammonia, triethylamine, diisopropylethylamine, tetramethylethylenediamine, hexanes, pentane, dimethyl ether, diethyl ether, petroleum ether, ethyl acetate, carbon tetrachloride, benzenethiol, cyclohexanethiol, 1-diethylaminoethanol, ethylene glycol, xylene, 1,1,2,2-tetrachloroethane, phenol, 2-butanone, diglyme, N-methyl-Z-pyrrolidinone (NMP), heptane, glycerin, hexamethylphosphorus triamide (HMPA), methyl t-butyl ether (MTBE), water, acetone, methanol, ethanol, n-propanol, isopropanol, n-butanol (i.e., 1-butanol, 2-butanol), acetic acid, formic acid, nitromethane, or combination thereof. In some embodiments, the solvent is anhydrous. In some embodiments, the solvent is degassed. In some embodiments, a solvent or a suitable solvent can be polar, aprotic, protic or non-polar. In some embodiments, a “protic solvent” is water, methanol, ethanol, isopropanol, n-butanol, acetic acid, formic acid, nitromethane, or combination thereof.

As used herein, the term “protecting group” refers to a substituent that can be installed and removed selectively under certain reaction conditions. Examples of a protecting group include, but are not limited to, acetate (OAc), mesylate (OMs), tosylate (OTs), triflate (OTf), substituted or non-substituted benzyl ether (OBn), tert-butyldimethylsilyl ether (OTBS), triethylsilyl ether (OTES), trimethylsilyl ether (OTMS), methoxymethyl ether (OMOM), ethoxyl methoxyl ether (OEOM), and t-butyl carbamate (—Nboc). The term “R—X” refers to a substance which is used in a chemical reaction to introduce a protecting group, wherein R is a suitable protecting group selected from the examples above and other protecting groups known to one of skill in the art, and X is a leaving group such as halo (fluoro, chloro, iodo), or OCF₃. In some embodiments, R² is a protecting group. In some embodiments, R² is OBn, substituted OBn (e.g., p-bromobenzoate), or OAc. In some embodiments, R²—X used in the preparation is benzoyl chloride.

As used herein, the term “ligand” refers to an ion or neutral compound which bonds with a catalyst. In some embodiments, the ligand includes, but is not limited to, 1,10-phenanthroline, 2,2′-bipyridine, ammonia, triethylamine, diisopropylethylamine, tetramethylethylenediamine, or combinations thereof.

As used herein, the term “catalyst” refers to a substance which is used to increase the rate of a chemical reaction. In some embodiments, the catalyst includes is a metal catalyst. In some embodiments, the catalyst includes, but is not limited to, CuOTf, Cu(OAc)₂, CuI, Cu₂O, CuBr, or [CuOTf]₂-toluene complex, or combinations thereof.

As used herein, the term “oxidizing agent” refers to a substance that gains an electron in a redox chemical reaction. In some embodiments, the oxidizing agent is osmium (VIII) oxide.

As used herein, the term “alkali salt” refers to a substance which increases pH or neutralizes acidity. In some embodiments, the alkali salt is potassium carbonate, potassium bicarbonate, sodium acetate, sodium carbonate, sodium bicarbonate, cesium carbonate, cesium fluoride, potassium phosphate, potassium dihydrogen phosphate, potassium hydrogen phosphate, lithium hydroxide, sodium hydroxide, potassium hydroxide, lithium hexamethyldisilazide, potassium hexamethyldisilazide, sodium hexamethyldisilazide, lithium hydride, sodium hydride, potassium hydride, or combinations thereof.

As used herein, the term “acid” refers to a substance which can donate a hydrogen proton (H⁺), form a covalent bond with an electron pair, or decrease pH. In some embodiments, the acid is hydrochloric acid, sulfuric acid, acetic acid, formic acid, or combinations thereof. In some embodiments, an acid is an organic acid such as acetyl chloride or oxalyl chloride.

One skilled in the art will understand that the types listed or illustrated above are not exhaustive and that additional types within these specific terms can also be selected.

As used herein, the term “substituent” or “substituted” is used to describe an R group attached to a second group via at least one bond between the group and a portion of the second group. In some embodiments, when “a compound is substituted by an R group”, it means that at least one atom of the compound forms a single, double or triple bond with the R group. In some embodiments, a compound can be substituted in two different positions by two R groups, wherein the two R groups, taken together with a portion of the compound, form a ring. For example, the compound bicyclo[2.2.1]hept-2-ene can be understood as a cyclopentane substituted with two R groups, wherein the two R groups, taken together, is a diene, and taken together with the cyclopentane, from bicyclo[2.2.1]hept-2-ene.

As used herein, the terms “benzene” and “phenyl” are used interchangeably to refer to an optionally substituted 6-membered aromatic (fully unsaturated) carbocyclic ring. One of skill in the art would understand, for example, that the phenyl group in the R¹ group 3,5-difluorophenyl can also be described as having a formula of C₆H₃.

As used herein, compound names with “-yl” suffix are used to denote that the compound is a substituent or a group attached to another compound via at least one atom.

In some embodiments, the formula of an amine is written as NH₃, RNH₂, R₂NH, or R₃N, and acceptable salts thereof include the cations NH₄⁺, [RH₃N]⁺, [R₂H₂N]⁺, [R₃HN]⁺, or [R₄N]⁺, respectively, together with one or more anions to form a salt. Examples of anions include, but are not limited to halides (F, Cl, I), hydroxides, carbonates, phosphates, sulfates, sulfites, cyanides, azides, oxochlorides (ClO⁻, ClO₂⁻, ClO₃⁻ ClO₄⁻), tetrafluoroborates, tetraphenylborates, hexafluorophosphates, and the like.

As used herein, the term “hindered base” refers to a subgroup of base as defined herein which are sterically hindered. Examples of hindered base include, but are not limited to, triethylamine, diisopropylethylamine, tributylamine and tetramethylethylenediamine.

Although the description referred to particular embodiments, the disclosure should not be construed as limited to the embodiments set forth herein.

### Embodiments I

In some embodiments, provided is a compound of Formula I:

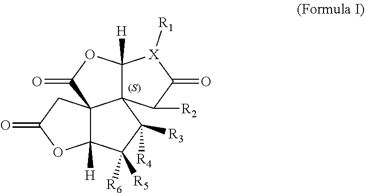

or a stereomer, a tautomer, or a pharmaceutically acceptable salt thereof,
wherein
X is —O—, —NR¹—, —N═CR¹—NH—, or —NR¹—NH—; wherein when X is —O—, R¹ is absent; bond Y¹ is a single bond or a double bond;
R¹ is H, R^{1B}, or -(L¹)ᵤ-(Z¹)ᵥ; wherein
   L¹ is C₁-C₁₀ aliphatic wherein up to three carbon atoms of the C₁-C₁₀ aliphatic are optionally replaced by N, O, or S; wherein L¹ is optionally substituted with 1-3 occurrences of halo, CN, R, OR′, or R^{1C};
      u is 0 or 1;
      v is 0 or 1;
   Z¹ is a 5-16 membered aromatic or nonaromatic monocyclic or bicyclic ring system having 0-5 heteroatoms selected from O, N, or S; wherein Z¹ is optionally substituted with 1-5 occurrences of R^{1A}, R^{1C} or combinations thereof;
R^{1A} is -(L²)ₘ-(Z²)_{w}; wherein
   L² is C₁-C₁₀ aliphatic wherein up to three carbon atoms of the C₁-C₁₀ aliphatic are optionally replaced by N, NR, O, S, C═O, SO₂, S═O, (C═O)N, N(C═O)N, (C═O)O, or Si; wherein L² is optionally substituted with 1-3 occurrences of halo, CN, R, OR′ or; m is 0 or 1;
   w is 0 or 1;
Z² is a C₁-C₁₀ aliphatic, or 3-16 membered aromatic or nonaromatic monocyclic or bicyclic ring system having 0-5 heteroatoms selected from O, N, or S; wherein Z² is optionally substituted with 1-5 occurrences of R^{1B};
R^{1B} is H, halo, CN, R*, OR*, NRR*; or two R^{1B}, taken together with the atom to which they are attached, form a 3-6 membered ring having 0-4 heteroatoms;
R^{1C} is H, halo, CN, R*, OR*, NRR*; or two R^{1C}, taken together with the atom to which they are attached, form a 3-6 membered ring having 0-4 heteroatoms; R* is C₁-C₆ aliphatic wherein up to three methylene units of the C₁-C₆ aliphatic are optionally replaced by N, NR, O, S, C═O, SO, SO₂ or Si and wherein the C₁-C₆ aliphatic is optionally substituted with 1-3 occurrences of halo, CN, R′ or OR′;
R² is R^{2A} or OR^{2A}, wherein R^{2A} is H, a C₁-C₁₆ aliphatic, a 5-10 membered aromatic or nonaromatic monocyclic or bicyclic ring system, or —(C₁-C₁₆ aliphatic)-(5-10 membered aromatic or nonaromatic monocyclic or bicyclic ring system); wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{2A} is optionally substituted with 1-5 occurrences of R^{2B}, wherein R^{2B} is halo, R′ or OR′;
R³ is OH, R^{3A}, or OR^{3A}; wherein R^{3A} is C₁-C₁₀ aliphatic optionally substituted with 1-3 occurrences of halo, R or OR′;
R⁴ is OH, R^{4A}, OR^{4A}; or when bond Y¹ between R⁴ and R⁵ is a double bond, R⁴ is absent; wherein R^{4A} is C₁-C₇ aliphatic and R^{4A} is optionally substituted with 1-3 occurrences of halo, R′ or OR′;
R⁵ is H or OH;
R⁶ is H; or when bond Y¹ between R⁴ and R⁵ is a double bond, R⁶ is absent;
R is H or C₁-C₆ aliphatic optionally substituted by 1-3 occurrences of F; or two R, taken together with the atom(s) to which they are attached, form a 3-6 membered ring having 0-4 heteroatoms; and
R′ is H, a C₁-C₆ aliphatic optionally substituted by 1-3 occurrences of F, or a 5-10 membered aromatic or nonaromatic monocyclic or bicyclic ring system having 0-5 heteroatoms selected from O, N, or S;
wherein when R² is OH, R³ is tert-butyl, R⁴ is OH, R⁵ is H, and R⁶ is H, X is not —O—.

In some embodiments, provided is a compound of Formula I:

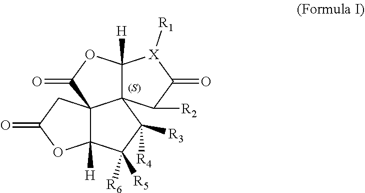

or a stereomer, a tautomer, or a pharmaceutically acceptable salt thereof,
wherein
X is —O—, —NR¹—, —N═CR¹—NH—, or —NR¹—NH—; wherein when X is —O—, R¹ is absent;
bond Y¹ is a single bond or a double bond;
R¹ is H, R^{1B}, or -(L¹)ᵤ-(Z¹)ᵥ; wherein
   L¹ is C₁-C₁₀ aliphatic wherein up to three carbon atoms of the C₁-C₁₀ aliphatic are optionally replaced by N, O, or S; wherein L¹ is optionally substituted with 1-3 occurrences of halo, CN, R, OR′, or R^{1C};
      u is 0 or 1;
      v is 0 or 1;
   Z¹ is a 5-16 membered aromatic or nonaromatic monocyclic, bicyclic, or tricyclic ring system having 0-7 heteroatoms selected from O, N, or S; wherein Z¹ is optionally substituted with 1-5 occurrences of R^{1A}, R^{1C} or combinations thereof;
R^{1A} is -(L²)ₘ-(Z²)_{w}; wherein
   L² is C₁-C₁₀ aliphatic wherein up to three carbon atoms of the C₁-C₁₀ aliphatic are optionally replaced by N, NR, O, S, C═O, SO₂, S═O, (C═O)N, N(C═O)N, (C═O)O, or Si; wherein L² is optionally substituted with 1-3 occurrences of halo, CN, R, OR′ or; m is 0 or 1;
   w is 0 or 1;
Z² is a C₁-C₁₀ aliphatic, or 3-16 membered aromatic or nonaromatic monocyclic, bicyclic or tricylic ring system having 0-7 heteroatoms selected from O, N, or S; wherein Z² is optionally substituted with 1-5 occurrences of R^{1B};
R^{1B} is H, halo, CN, R*, OR*, NRR*; or two R^{1B}, taken together with the atom to which they are attached, form a 3-6 membered ring having 0-4 heteroatoms;
R^{1C} is H, halo, CN, R*, OR*, NRR*; or two R^{1C}, taken together with the atom or atoms to which they are attached, optionally form a 3-16 membered ring having 0-4 heteroatoms;
R* is C₁-C₆ aliphatic wherein up to three methylene units of the C₁-C₆ aliphatic are optionally replaced by N, NR, O, S, C═O, SO, SO₂ or Si and wherein the C₁-C₆ aliphatic is optionally substituted with 1-3 occurrences of halo, CN, R′ or OR′;
R² is R^{2A} or OR^{2A}, wherein R^{2A} is H, a C₁-C₁₆ aliphatic, a 5-10 membered aromatic or nonaromatic monocyclic or bicyclic ring system, or —(C₁-C₁₆ aliphatic)-(5-10 membered aromatic or nonaromatic monocyclic or bicyclic ring system); wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{2A} is optionally substituted with 1-5 occurrences of R^{2B}, wherein R^{2B} is halo, R′ or OR′;
R³ is OH, R^{3A}, or OR^{3A}; wherein R^{3A} is C₁-C₁₀ aliphatic optionally substituted with 1-3 occurrences of halo, R or OR′;
R⁴ is OH, R^{4A}, OR^{4A}; or when bond Y¹ between R⁴ and R⁵ is a double bond, R⁴ is absent; wherein R^{4A} is C₁-C₇ aliphatic and R^{4A} is optionally substituted with 1-3 occurrences of halo, R′ or OR′;
R⁵ is H or OH;
R⁶ is H; or when bond Y¹ between R⁴ and R⁵ is a double bond, R⁶ is absent;
R is H or C₁-C₆ aliphatic optionally substituted by 1-3 occurrences of F; or two R, taken together with the atom(s) to which they are attached, form a 3-6 membered ring having 0-4 heteroatoms; and
R′ is H, a C₁-C₆ aliphatic optionally substituted by 1-3 occurrences of F, OR, NH₂, NHR″, NR″₂, wherein R″ is C₁-C₆ aliphatic, or a 5-10 membered aromatic or nonaromatic monocyclic or bicyclic ring system having 0-5 heteroatoms selected from O, N, or S;
wherein when R² is OH, R³ is tert-butyl, R⁴ is OH, R⁵ is H, and R⁶ is H, X is not —O—.

For the sake of clarity, when u is 0 or v is 0, then the bond before -(L¹))ᵤ or before —(Z¹)ᵥ is also absent, respectively. Likewise, when m and w is 0, the bond before -(L²)ₘ or before —(Z²)_{w} is also absent.

In some embodiments, X is —NR¹—, —N═CR¹—NH—, or —NR¹—NH—.

In some embodiments, the compound has the structure of Formula Ia:

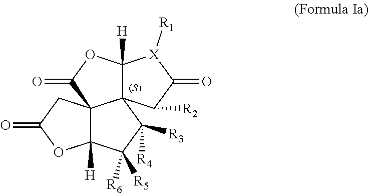

and R¹, R², R³, R⁴, R⁵, and R⁶ are as described herein. It shall be understood that superscripts and subscripts are interchangeable when referring to functional groups herein. For example R¹ is the same as R₁.

In some embodiments, the compound has the structure of Formula Ib:

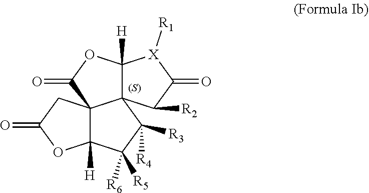

and R¹, R², R³, R⁴, R⁵, and R⁶ are as described herein.

In some embodiments, R² is R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅, (C═O)C₆H₄CH₂NH₂, or NH(C═O)OC(CH₃)₃; wherein phenyl is optionally substituted with 1-5 occurrences of R^{2B}, wherein R^{2B} is halo, R′ or OR′; R³ is methyl, ethyl, propyl, isopropyl, butyl, isobutyl, or tert-butyl; the bond Y¹ between R⁴ and R⁵ is a single bond; R⁴ is OH or OR^{4A}; and R⁵ is H or OH. In some embodiments, R^{2A} is

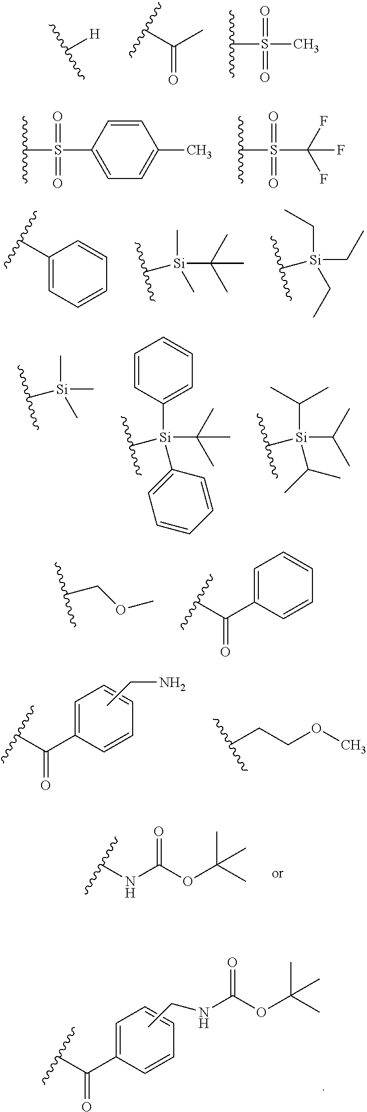

In some embodiments, R¹ is H.

In some embodiments, R¹ is -(L¹)ᵤ-(Z¹)ᵥ; wherein L¹ is C₁-C₁₀ aliphatic wherein up to three carbon atoms of the C₁-C₁₀ aliphatic are optionally replaced by N, O, or S; Z¹ is phenyl, 1-methyl-1,2,3,4-tetrahydronaphthalen-2-y, 1-methyl-2H-isoindol-2-yl, imidazol, indolyl, napthalenyl, adamantanyl, azetidinyl, bicyclo[1.1.1]pentyl, 1-oxa-8-azaspiro[4.5]decan-3-yl, cyclobutanyl, cyclohexanyl, cyclopentanyl, cyclopropanyl, norbornenyl, oxetanyl, piperazinyl, piperidinyl, pyridinyl, pyrrolidinyl, tetrahydrofuranyl, tetrahydropyranyl, tetrahydrothiopyranyl, or C₃-C₁₂ cycloaliphatic having 0-5 heteroatoms selected from O, N, or S; u is 0 or 1; and v is 0 or 1; wherein Z¹ is optionally substituted with 1-5 occurrences of R^{1C} morpholinyl, —OCH₂O—, —(C═O)-(pyrazinyl)-R^{1B}, —(C═O)(phenyl)-R^{1B}, or —(SO₂)-(phenyl)-R^{1B}; wherein each independent occurrence of R^{1B} is H, halo, R*, OR*, or NRR*; wherein each independent occurrence of R^{1C} is H, halo, R*, OR*, or NRR*; and wherein each independent occurrence of R* is H, ═N, —C≡CH, —N═N—, —CH₃, —CH₂F, —CHF₂, —CF₃, —CN, —CH₂O—, —CF₂O—, —CH₂CH₂O—, or -Boc (—(C═O)OC(CH₃)₃).

In some embodiments, when Z¹ is phenyl, Z¹ is optionally substituted with 1-5 occurrences of morpholinyl or R^{1C}, wherein R^{1C} is halo, CH₃, —CH₂F, —CHF₂, —CF₃, —CN—OCH₃, —OCH₂O—, —OCF₂O—, —OCH₂CH₂O—, —NH₂, —NH(C═O)CH₃, or —N(Boc) (NH(C═O)OC(CH₃)₃).

In some embodiments, when Z¹ is piperidinyl, Z¹ is optionally substituted with 1-2 occurrences of R^{1C}, wherein R^{1C} is tert-butoxylcarbonyl, 5-(difluoromethyl)pyrazine-2-carbonyl, 2,2-difluoro-2H-1,3-benzodioxole-5-carbonyl, 2,3-dihydro-1,4-benzodioxine-6-carbonyl, 2,3-dihydro-1-benzofuran-5-sulfonyl, 4-chlorobenzoyl, 2,3-dihydro-1-benzofuran-5-carbonyl, or prop-2-enoyl.

In some embodiments, when Z¹ is pyrrolidinyl, Z¹ is optionally substituted with 1-2 occurrences of R^{1C}, wherein R^{1C} is tert-butoxylcarbonyl.

In some embodiments, R¹ is H, 2,4-dimethoxybenzyl, [1-(tert-butoxycarbonyl)piperidin-4-yl]methyl, piperidin-4-ylmethyl, 2-[1-(tert-butoxycarbonyl)piperidin-4-yl]ethyl, 2-(piperidin-4-yl)ethyl, 3-[1-(tert-butoxycarbonyl)piperidin-4-yl]propyl, 3-(piperidin-4-yl)propyl, 2-[4-(tert-butoxycarbonyl)piperazin-1-yl]ethyl, 2-(piperazin-1-yl)ethyl, 2-(3-methyl-1H-indol-2-yl)ethyl, 3-(1H-imidazol-1-yl)propyl, (R)-[1-(tertbutoxycarbonyl)pyrrolidin-3-yl]methyl, (S)-pyrrolidin-3-yl)methyl), ((2R)-bicyclo[2.2.1]hept-5-en-2-yl)methyl, phenyl, 4-acetamidophenyl, 4-[(tert-butoxycarbonyl)amino]phenyl, 4-aminophenyl, 4-(morpholin-4-yl)phenyl, benzo[d][1,3]dioxol-5-yl, pyridin-3-yl, benzyl, methyl, bicyclo[1.1.1]pentyl, oxetan-3-yl, cyclobutyl methyl, cyclopropyl methyl, (oxetan-3-yl)methyl, adamantan-2-yl methyl, NH₂, cyclopropyl, 3-methoxy phenyl, 4-methoxy phenyl, naphthalen-2-yl, 3-(trifluoromethyl) phenyl, 4-cyano phenyl, 2-[3-(but-3-yn-1-yl)-3H-diazirin-3-yl]ethyl, cyclohexyl, 4-fluoro phenyl, 4-(trifluoromethyl) phenyl, 4-toluyl, 3-toluyl, 2-toluyl, (oxolan-2-yl)methyl, 2-methoxy-2-oxoethyl, (1-(5-(difluoromethyl)pyrazine-2-carbonyl)piperidin-4-yl)methyl, [1-(2,3-dihydro-1-benzofuran-5-sulfonyl)piperidin-4-yl]methyl, (1-(2,2-difluorobenzo[d][1,3]dioxole-5-carbonyl)piperidin-4-yl)methyl, (1-(2,3-dihydrobenzo[b][1,4]dioxine-6-carbonyl)piperidin-4-yl)methyl, (1-(4-chlorobenzoyl)piperidin-4-yl)methyl, (1-(2,3-dihydrobenzofuran-5-carbonyl)piperidin-4-yl)methyl, (1-acryloylpiperidin-4-yl)methyl, (1-(quinoxaline-6-carbonyl)piperidin-4-yl)methyl, (tetrahydro-2H-pyran-4-yl)methyl, (tetrahydro-2H-thiopyran-4-yl)methyl, 2-(1-methyl-1,2,3,4-tetrahydronaphthalen-2-yl)ethyl, 2-(1-methyl-2H-isoindol-2-yl)ethyl, 2-(azetidin-1-yl)ethyl, 2-(trifluoromethyl) phenyl, 2-fluoro phenyl, 2-methoxy phenyl, 3,4-difluoro phenyl, 3,5-difluoro phenyl, 3-fluoro phenyl, 4-hydroxy phenyl, 8-(tert-butoxycarbonyl)-1-oxa-8-azaspiro[4.5]decan-3-yl, anilinyl, benzo[d][1,3]dioxol-4-yl, cyclobutyl, cyclohexyl methyl, naphthalen-1-yl, piperidin-4-yl methyl, pyridin-2-yl, or pyridin-4-yl. In some embodiments, R¹ is H, 2,4-dimethoxybenzyl, [1-(tert-butoxycarbonyl)piperidin-4-yl]methyl, piperidin-4-ylmethyl, 2-[1-(tert-butoxycarbonyl)piperidin-4-yl]ethyl, 2-(piperidin-4-yl)ethyl, 3-[1-(tert-butoxycarbonyl)piperidin-4-yl]propyl, 3-(piperidin-4-yl)propyl, 2-[4-(tert-butoxycarbonyl)piperazin-1-yl]ethyl, 2-(piperazin-1-yl)ethyl, 2-(3-methyl-1H-indol-2-yl)ethyl, 3-(1H-imidazol-1-yl)propyl, [1-(tertbutoxycarbonyl)pyrrolidin-3-yl]methyl, (pyrrolidin-3-yl)methyl), (bicyclo[2.2.1]hept-5-en-2-yl)methyl, phenyl, 4-acetamidophenyl, 4-[(tert-butoxycarbonyl)amino]phenyl, 4-aminophenyl, 4-(morpholin-4-yl)phenyl, benzo[d][1,3]dioxol-5-yl, pyridin-3-yl, benzyl, methyl, bicyclo[1.1.1]pentyl, oxetan-3-yl, cyclobutyl methyl, cyclopropyl methyl, (oxetan-3-yl)methyl, adamantan-2-yl methyl, NH₂, cyclopropyl, 3-methoxy phenyl, 4-methoxy phenyl, naphthalen-2-yl, 3-(trifluoromethyl) phenyl, 4-cyano phenyl, 2-[3-(but-3-yn-1-yl)-3H-diazirin-3-yl]ethyl, cyclohexyl, 4-fluoro phenyl, 4-(trifluoromethyl) phenyl, 4-toluyl, 3-toluyl, 2-toluyl, (oxolan-2-yl)methyl, 2-methoxy-2-oxoethyl, (1-(5-(difluoromethyl)pyrazine-2-carbonyl)piperidin-4-yl)methyl, [1-(2,3-dihydro-1-benzofuran-5-sulfonyl)piperidin-4-yl]methyl, (1-(2,2-difluorobenzo[d][1,3]dioxole-5-carbonyl)piperidin-4-yl)methyl, (1-(2,3-dihydrobenzo[b][1,4]dioxine-6-carbonyl)piperidin-4-yl)methyl, (1-(4-chlorobenzoyl)piperidin-4-yl)methyl, (1-(2,3-dihydrobenzofuran-5-carbonyl)piperidin-4-yl)methyl, (1-acryloylpiperidin-4-yl)methyl, (1-(quinoxaline-6-carbonyl)piperidin-4-yl)methyl, (tetrahydro-2H-pyran-4-yl)methyl, (tetrahydro-2H-thiopyran-4-yl)methyl, 2-(1-methyl-1,2,3,4-tetrahydronaphthalen-2-yl)ethyl, 2-(1-methyl-2H-isoindol-2-yl)ethyl, 2-(azetidin-1-yl)ethyl, 2-(trifluoromethyl) phenyl, 2-fluoro phenyl, 2-methoxy phenyl, 3,4-difluoro phenyl, 3,4-dichloro phenyl, 3,5-difluoro phenyl, 3-fluoro phenyl, 4-hydroxy phenyl, 8-(tert-butoxycarbonyl)-1-oxa-8-azaspiro[4.5]decan-3-yl, anilinyl, benzo[d][1,3]dioxol-4-yl, cyclobutyl, cyclohexyl methyl, naphthalen-1-yl, pyridin-2-yl, pyridin-4-yl, adamantan-1-yl methyl, 1-(tert-butoxycarbonyl)-1H-indol-5-yl, 1H-indol-5-yl, 3-[(tert-butoxycarbonyl)amino]phenyl, 4-Hydroxyphenyl ethyl, 1H-indole-3-ethyl, ((1R,4aS,10aR)-7-isopropyl-1,4a-dimethyl-1,2,3,4,4a,9,10,10a-octahydrophenanthren-1-yl) methyl, [((tert-butoxycarbonyl)aminomethyl) adamantan-1-yl]methyl, (aminomethyl)adamantan-1-yl) methyl, 3,5-di-tert butyl phenyl, 3,4-dihydroxyphenyl ethyl, 3-methoxy, 4-hydroxyphenyl ethyl, 1H-indole-5-hydroxy-3-ethyl, 1H-indole-5-methoxy-3-ethyl, or 1H-indole-4-hydroxy-3-ethyl.

In some embodiments, R² is R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅, (C═O)C₆H₄CH₂NH₂, or NH(C═O)OC(CH₃)₃; wherein phenyl is optionally substituted with 1-5 occurrences of halo, R′ or OR′; R³ is tert-butyl; the bond Y¹ between R⁴ and R⁵ is a single bond; R⁴ is OH; R⁵ is H; X is —NR¹—, —N═CR¹—NH—, or —NR¹—NH—; and R¹ is selected from

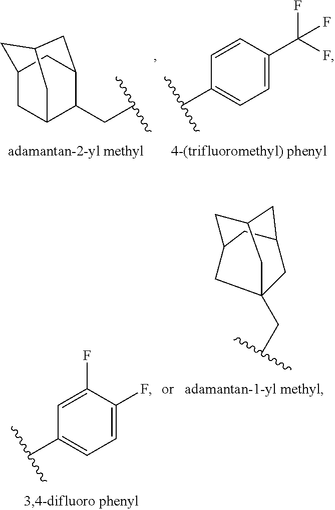 [SMILES: *CC1C2CC3CC(C2)CC1C3 ; *c1ccc(C(F)(F)F)cc1 ; *CC12CC3CC(CC(C3)C1)C2 ; *c1ccc(F)c(F)c1]

In some embodiments, R^{2A} is

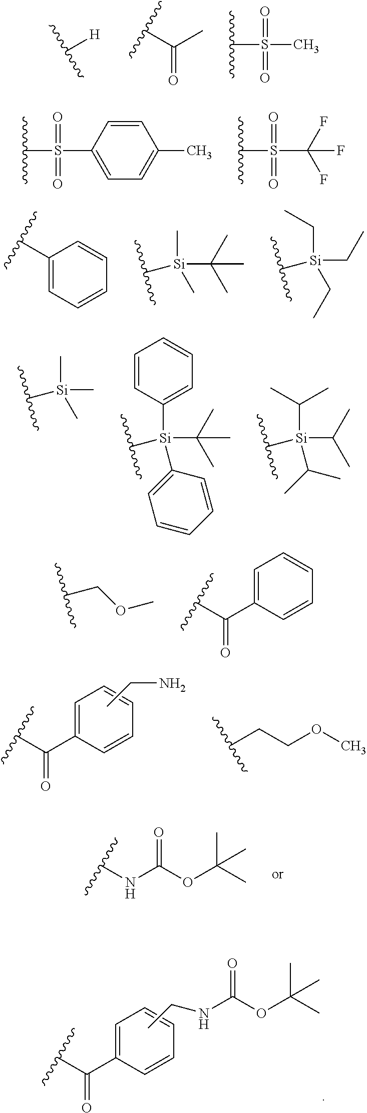

In some embodiments, R¹ is

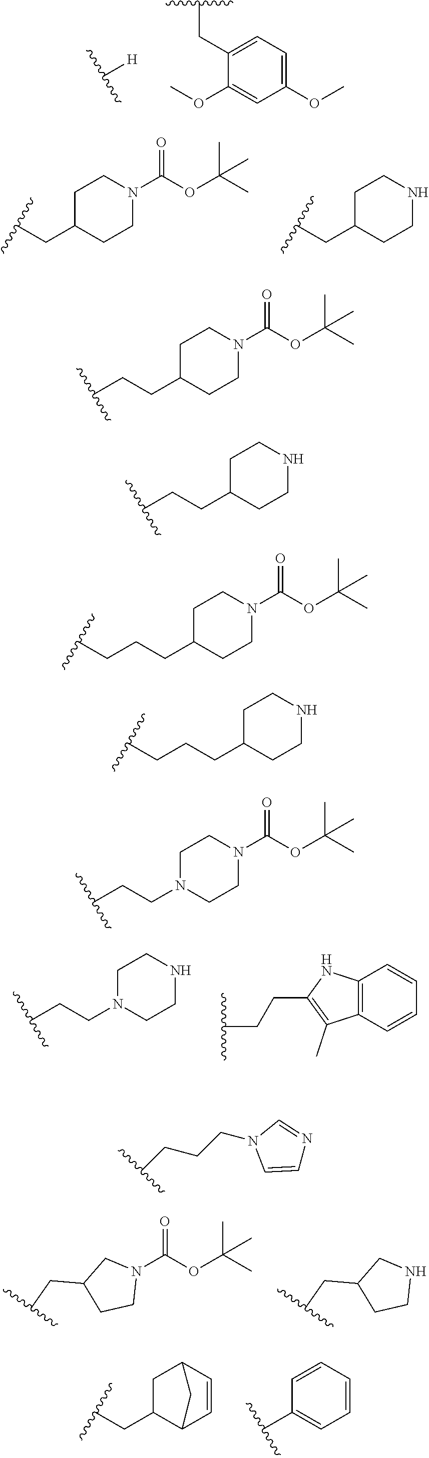

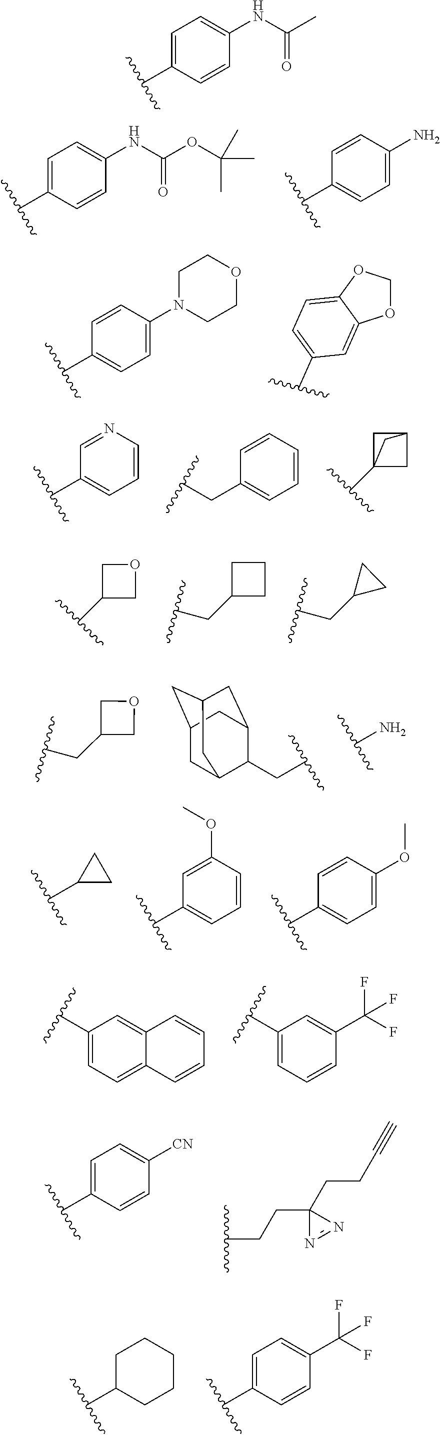

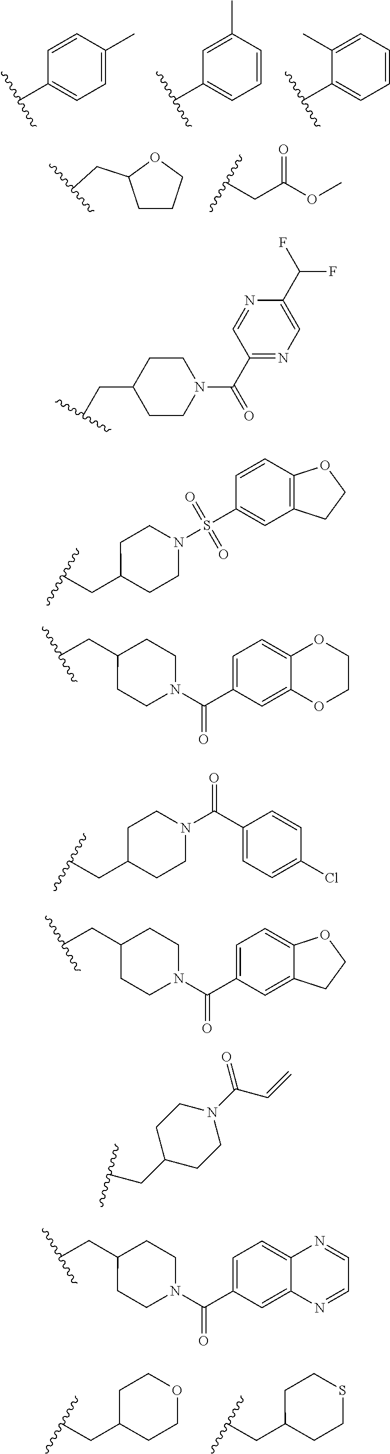

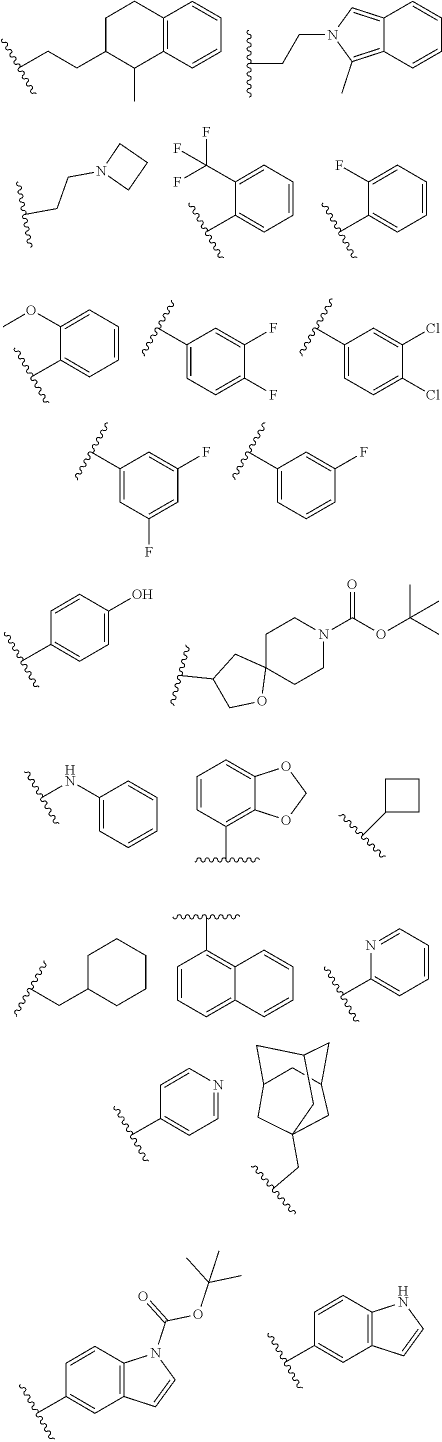

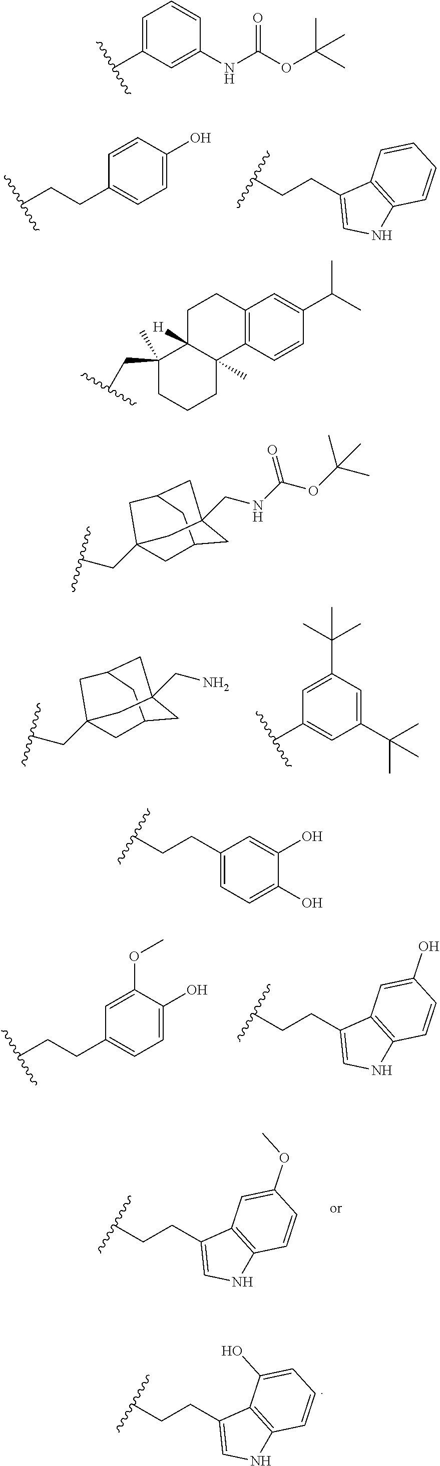

In some embodiments, the compound is selected from the group consisting of the compounds as described in Table 1d.

In some embodiments, the compound is DW192, P-29, P-21, P-30, P-33, JW093, XBB-023, P-28, JW107, XBB-039, JW094, P-34, XBB-045, JW081, XBB-028, XBB-038, XBB-037, XBB-054, XBB-025, XBB-029, XBB-024, DW172, XBB-004, XBB-042, XBB-068, XBB-040, XBB-006, JW072, DW189, P-8, DW191, DW168, XBB-013, XBB-037′, XBB-009, XBB-060, XBB-016, DW182, XBB-010, SCC506, or SCC363.

In some embodiments, the bond Y¹ is a double bond, having Formula I′:

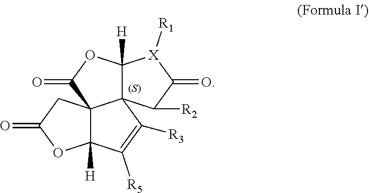

In some embodiments, X is —O— and R¹ is absent; R² is R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅, (C═O)C₆H₄CH₂NH₂, or NH(C═O)OC(CH₃)₃; wherein phenyl is optionally substituted with 1-5 occurrences of halo, R′ or OR′; R³ is tert-butyl; R⁴ is absent; R⁵ is H; and R⁶ is absent.

In some embodiments, R^{2A} is

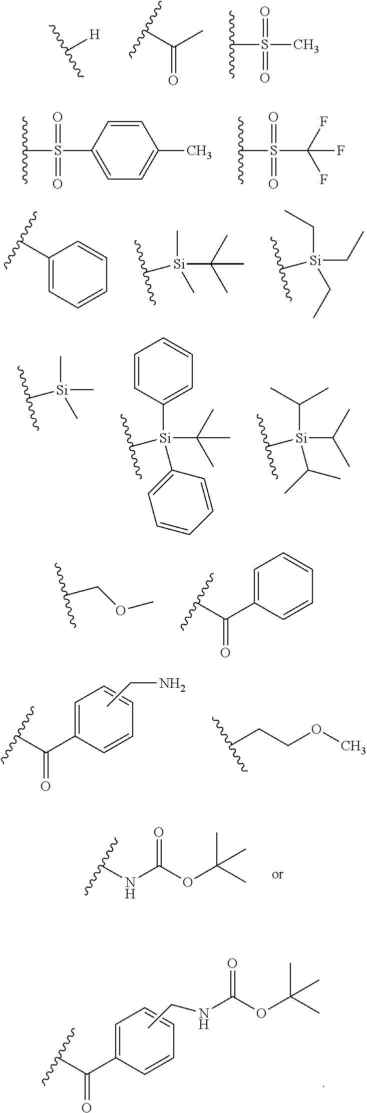

In some embodiments, the compound has a structure of Formula I′a:

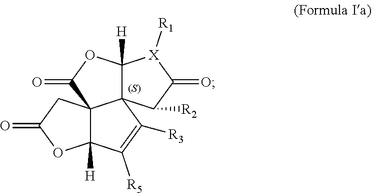

wherein R¹, R², R³, and R⁴ are as described herein.

In some embodiments, the compound has a structure of Formula I′b:

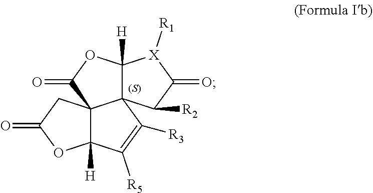

wherein R¹, R², R³, and R⁴ are as described herein.

In some embodiments, X is —O— and R¹ is absent; bond Y¹ is a single bond; R² is OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅, (C═O)C₆H₄CH₂NH₂, or NH(C═O)OC(CH₃)₃; wherein phenyl is optionally substituted with 1-5 occurrences of halo, R′ or OR′; R³ is isopropenyl; R⁴ is CH₃; R⁵ is H; and R⁶ is H.

In some embodiments, R^{2A} is

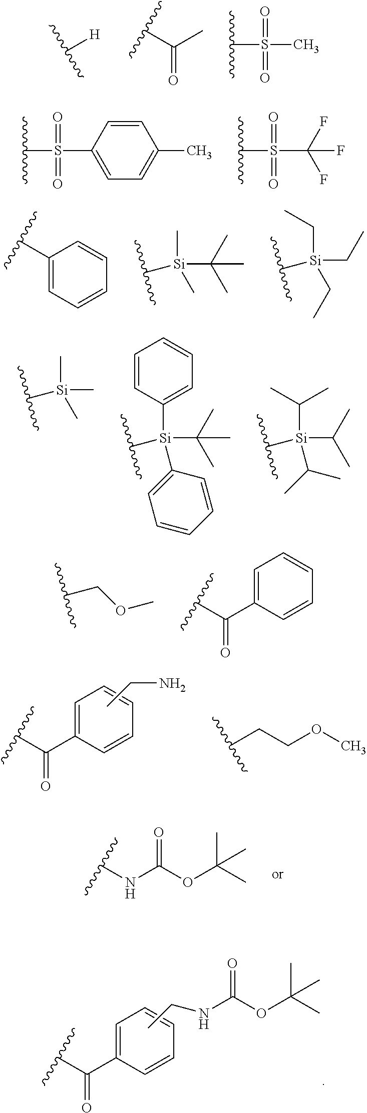

In some embodiments, provided is a compound of Formula II:

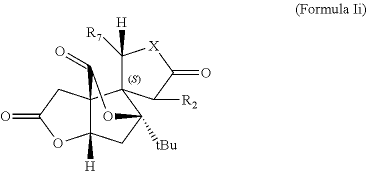

or a stereomer, a tautomer, or a pharmaceutically acceptable salt thereof, wherein X is —O—, —NR¹—, —N═CR¹—NH—, or —NR¹—NH—; wherein R¹ is as defined in any one of the preceding embodiments or described herein; R² is R^{2A} or OR^{2A}, wherein R^{2A} is H, a C₁-C₁₆ aliphatic or a 5-10 membered aromatic or nonaromatic monocyclic or bicyclic ring system, wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{2A} is optionally substituted with 1-5 occurrences of R²¹, wherein R²¹ is halo, R′ or OR′; and R⁷ is R^{7A} or OR^{7A}, wherein R^{7A} is H, a C₁-C₁₆ aliphatic or a 5-10 membered aromatic or nonaromatic monocyclic or bicyclic ring system, wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{7A} is optionally substituted with 1-5 occurrences of R^{7B}, wherein R^{7B} is halo, R′ or OR′.

In some embodiments, the compound has a structure of Formula IIa:

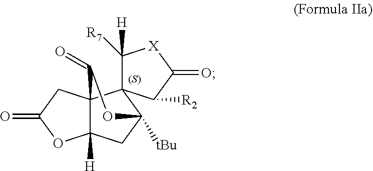

wherein R² and R⁷ are as described herein.

In some embodiments, the compound has a structure of Formula IIb:

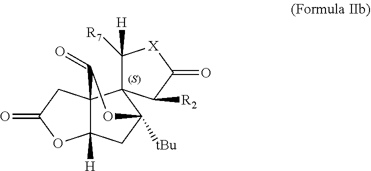

wherein R² and R⁷ are as described herein.

In some embodiments, X is —O—; R² is R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅, (C═O)C₆H₄CH₂NH₂ or NH(C═O)OC(CH₃)₃; wherein phenyl is optionally substituted with 1-5 occurrences of halo, R′ or OR′; and R⁷ is R^{7A} or OR^{7A}, wherein R^{7A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅, (C═O)C₆H₄CH₂NH₂ or NH(C═O)OC(CH₃)₃; wherein phenyl is optionally substituted with 1-5 occurrences of halo, R′ or OR′.

In some embodiments, R^{2A} and/or R^{7A} is

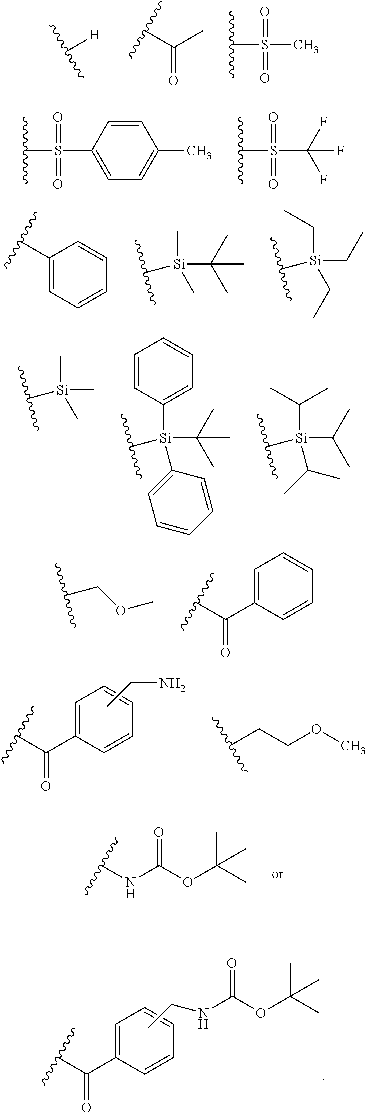

In some embodiments, the compound is

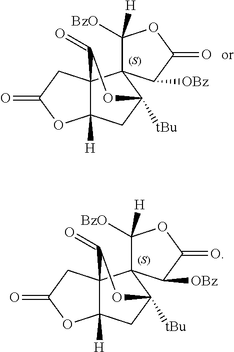

In some embodiments, provided is a process for preparing a compound as described herein, including at least the following steps:
(i) treating bilobalide with R^{2A}—X in a suitable solvent to form protected product IIa

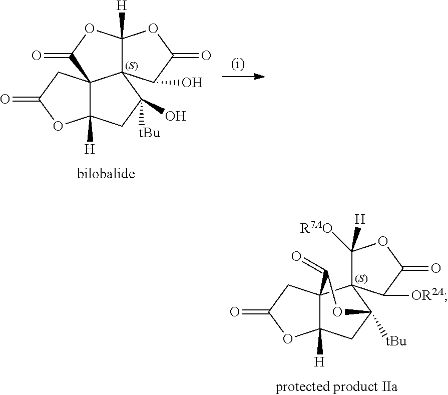

and
(ii) treating protected product IIa with at least one base or an acceptable salt thereof to form aminated product IIb

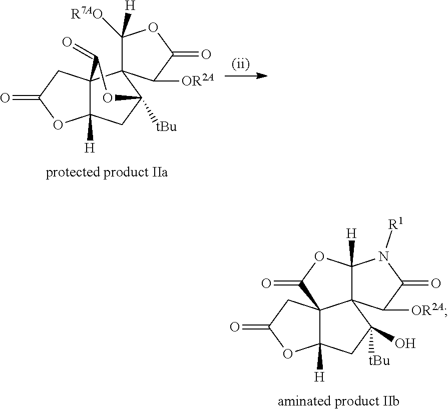

wherein R^{2A} and R^{7A} are as defined in any one of the preceding embodiments or described herein.

In some embodiments, wherein the aminated product IIb has the formula of aminated product IIb′, further comprising the step of: (iii) treating aminated product IIb′ with an R′—B(OH)₂ in the presence of a catalyst to form a N-arylated product IIc

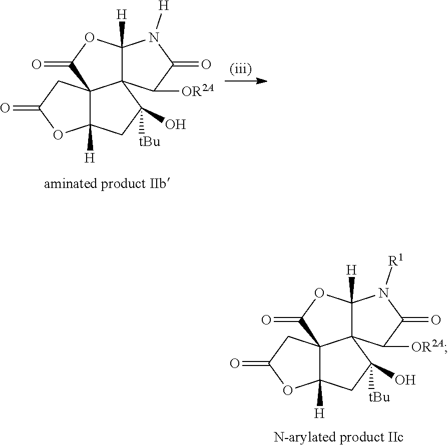

wherein R¹ and R^{2A} are as defined in any one of the preceding embodiments or described herein. In some embodiments, a ligand is optionally added to the catalyst. In some embodiments, the catalyst is a Cu catalyst. In some embodiments, Cu:ligand molar ratio is such that there is an excess of either Cu or the ligand. In some embodiments, the Cu:ligand ratio is 5:1, 4:1, 3:1, 2:1, 1:2, 1:3, 1:4, or 1:5. In some embodiments, the Cu:ligand ratio is A:B, wherein A is 1.1 to 10 and B is 1 A is 1 and B is 1.1 to 10. In some embodiments, the Cu:ligand ratio is not 1:1.

In some embodiments, the process further includes the step of:
(iv) treating the aminated product IIb of any one of the preceding embodiments or the N-arylated product IIc of any one of the preceding embodiments with an alkali salt or an acid in a protic solvent to form a deprotected product. In some embodiments, the deprotected product is deprotected product IId and the alkali salt is K₂CO₃.

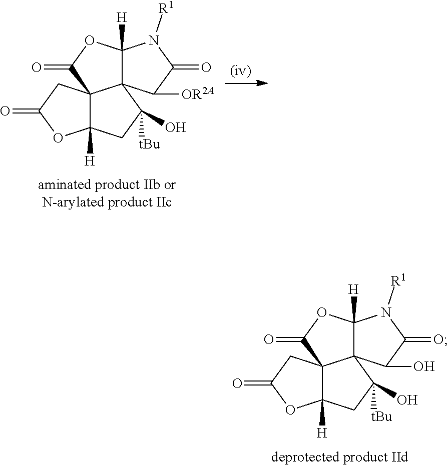

wherein R¹ and R^{2A} are as defined in any one of the preceding embodiments or described herein. In some embodiments, the deprotected product is deprotected product IIe and the acid is an organic acid such as acetyl chloride or oxalyl chloride, provided that R¹ contains other protecting groups such as tert-butox carbonyl (boc); for example:

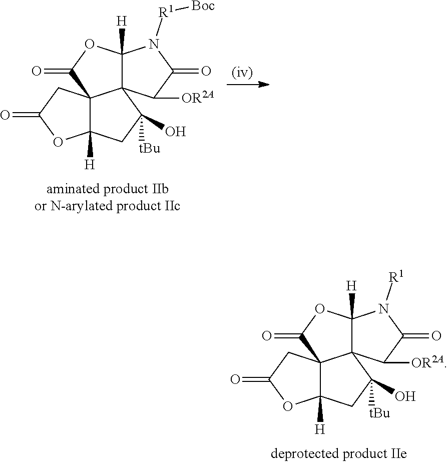

In some embodiments, R^{2A} and R^{7A} of the protected product IIa is as defined in any one of the preceding embodiments or described herein.

In some embodiments, R^{2A}—X is benzoyl chloride, and the suitable solvent is pyridine. In some embodiments, R^{2A}—X is 4-(Boc-aminomethyl) benzoic acid and the suitable solvent is DCM (dichloromethane). In some embodiments, R^{2A}—X is benzoyl chloride or 4-(Boc-aminomethyl) benzoic acid and the suitable solvent is pyridine or DCM.

In some embodiments, R¹ and R^{2A} of aminated product IIb are as defined in any one of the preceding embodiments or described herein.

In some embodiments, R¹ and R^{2A} of the N-arylated product IIc are as defined in any one of the preceding embodiments or described herein.

In some embodiments, the at least one base is ammonia, and the aminated product IIb is

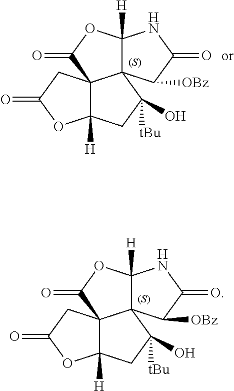

In some embodiments, the deprotected product, such as deprotected product IId or deprotected product IIe, is as defined in any one of the preceding embodiments or described herein.

In some embodiments, the at least one base is NH₂R¹. In some embodiments, the at least one base in step (ii) is [H₃NR¹]⁺.

In some embodiments, the process further includes a second base in step (ii), wherein in the second base is a hindered base such as triethylamine, diisopropylethylamine, tributylamine or tetramethylethylenediamine.

In some embodiments, the protected product IIa is as defined herein.

In some embodiments, the [H₃NR¹]⁺ is provided as XYa prepared by the steps of: (a) treating R—COOH with 1-hydroxybenzotriazole, N-(3-dimethylaminopropyl)-N′-ethylcarbodiimide hydrochloride, and tert-butyl (piperidin-4-ylmethyl)carbamate, to form a boc-protected product SXa, wherein R═(Z²)_{w}—R^{1B}, wherein Z², w and R^{1B} are as defined in any one of the preceding embodiments or as described herein; and (b) treating the boc-protected product SXa with an acid in a solvent to form XYa

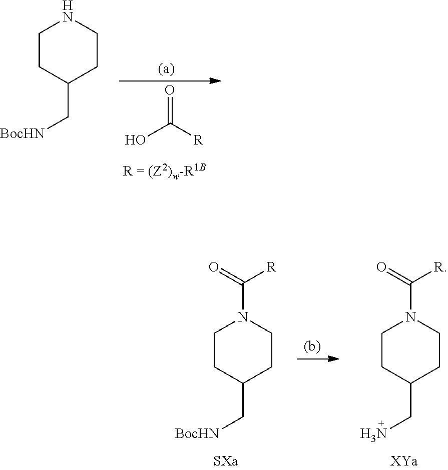

In some embodiments, the [H₃NR¹]⁺ is provided as XYb prepared by the steps of: (a) treating R—SO₂ with tert-butyl (piperidin-4-ylmethyl)carbamate and triethylamine, to form a boc-protected product SXb, wherein R═(Z²)_{w}—R^{1B}, wherein Z², w and R^{1B} are as defined in any one of the preceding embodiments or as described herein; and (b) treating the boc-protected product SXb with an acid in a solvent to form XYb

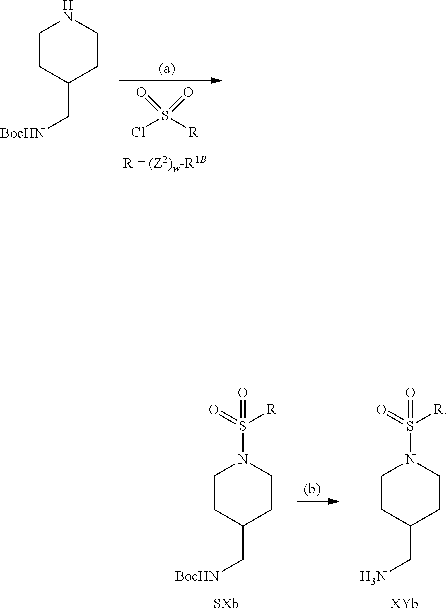

In some embodiments, provided is a process of preparing a compound as defined herein, which include the steps of:
(i) treating bilobalide with Ac₂O and an acid to form a protected product IVa and/or protected product Va

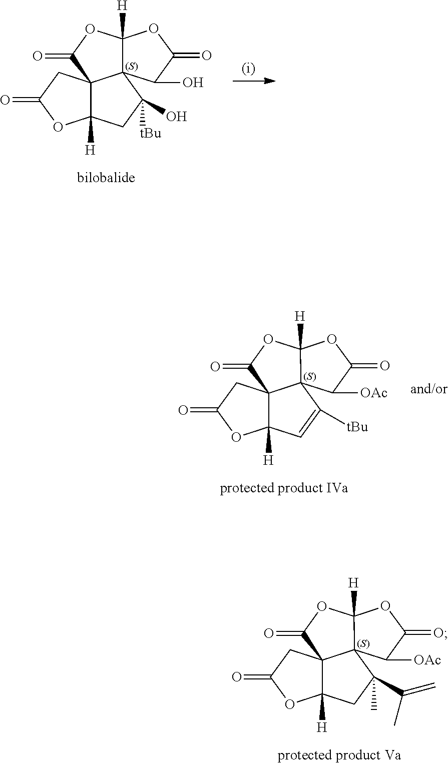

and
(ii) treating protected product IVa and/or protected product Va with at least one base or an acceptable salt thereof to form aminated product IVb or aminated product Vb

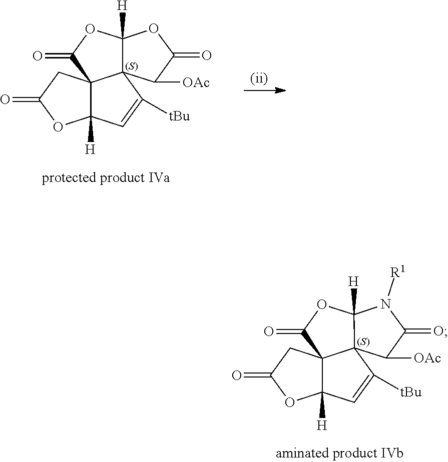

and/or

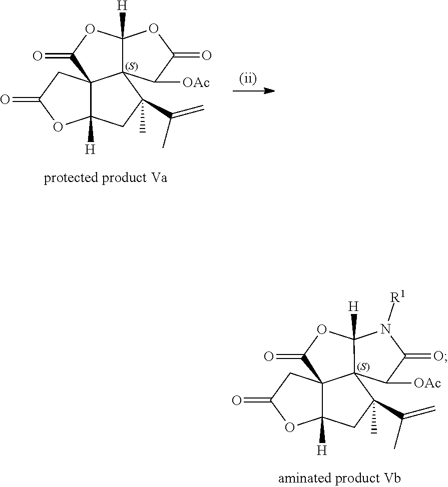

wherein R¹ is as defined in any one of the preceding embodiments or as described herein. In some embodiments, the step (i) forms a mixture of protected product IVa and protected product Va which account for the total weight of the reaction product in a ratio (protected product IVa:protected product Va) of about 0:100, 10:90, 20:80, 30:70, 40:60, 50:50, 60:40, 70:30, 80:20, 90:10, or 100:0. In some embodiments, the total weight of the reaction product in step (i) is 100%. In some embodiments, the step (i) forms protected product IVa or protected product Va.

In some embodiments, the process further includes the step of: (iii) treating the aminated product IVb with an oxidizing agent and a solvent to form oxidized product VIc

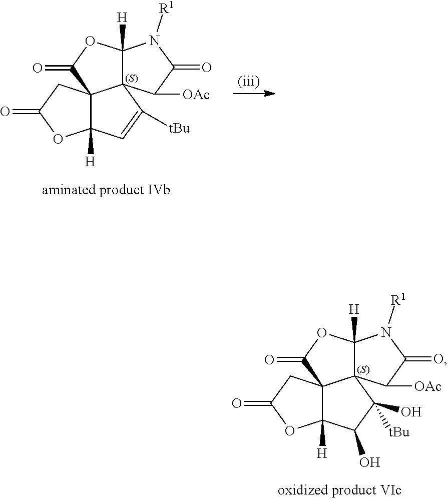

wherein R¹ is as defined in any one of the preceding embodiments or as described herein.

In some embodiments, the process further includes at least one of the steps of: (iv) treating the aminated product IVb or the aminated product Vb with an acid to form deprotected product IVd or deprotected product Vd,

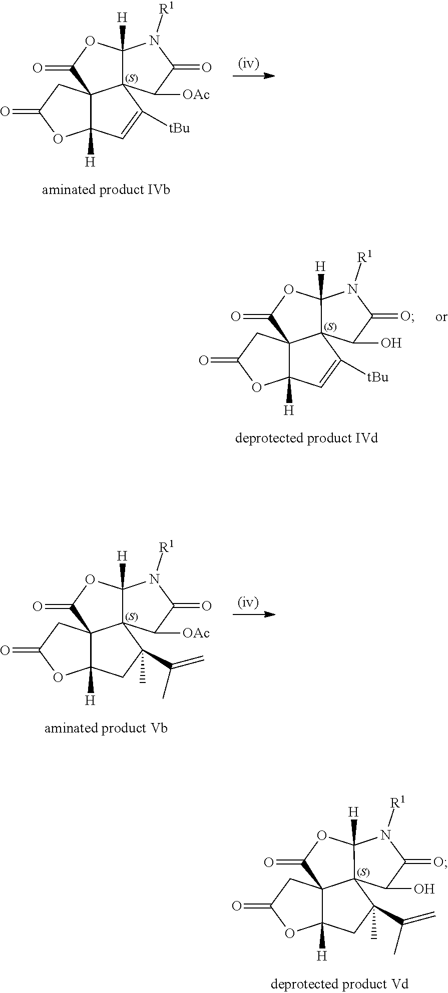

wherein R¹ is as defined in any one of the preceding embodiments or as described herein.

In some embodiments, the process further includes the step of: (v) treating the oxidized product VIc with an acid to form deprotected product VId,

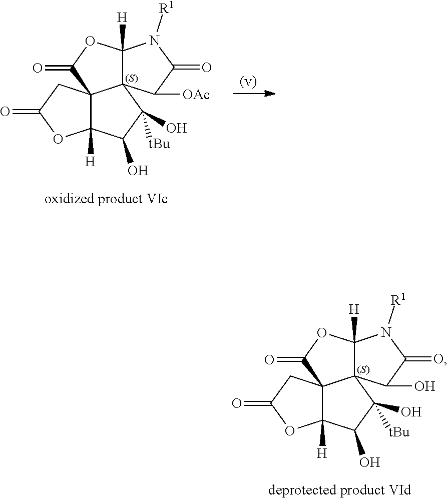

wherein R¹ is as defined in any one of the preceding embodiments or as described herein.

In some embodiments, provided is a method of treating or preventing cancer in a subject in need thereof, including administering to the subject a compound described herein.

In some embodiments, the cancer is bladder cancer, brain cancer, breast cancer, CNS cancer, colon cancer, hematopoietic cancer, kidney cancer, leukemia, lung cancer, melanoma, ovarian cancer, pancreatic cancer, prostate cancer, or renal cancer.

In some embodiments, the cancer is leukemia, colon cancer, lung cancer, melanoma or renal cancer.

In some embodiments, the lung cancer is non-small cell lung cancer (NSCLC).

In some embodiments, the leukemia is lymphocytic leukemia.

In some embodiments, provided is a use of a compound described herein for treating or preventing cancer.

In some embodiments, provided is a use of a compound described herein for the manufacture of a medicament for treating or preventing cancer.

In some embodiments, provided is a method of inducing cell death in a cancer cell, comprising contacting a compound of a compound described herein with the cancer cell.

In some embodiments, provided is a method of inhibiting cell growth in a cancer cell, comprising contacting a compound of a compound described herein with the cancer cell.

In some embodiments, the method is an in vitro method.

In some embodiments, the compound is DW192, P-29, P-21, P-30, P-33, JW093, XBB-023, P-28, JW107, XBB-039, JW094, P-34, XBB-045, JW081, XBB-028, XBB-038, XBB-037, XBB-054, XBB-025, XBB-029, XBB-024, DW172, XBB-004, XBB-042, XBB-068, XBB-040, XBB-006, JW072, DW189, P-8, DW191, DW168, XBB-013, XBB-‘37’, XBB-009, XBB-060, XBB-016, DW182, XBB-010, SCC506, or SCC363.

In some embodiments, the compound is DW192, P-29, P-21, SCC506, or SCC363.

### Embodiments II

In some embodiments, provided is a compound of Formula I:

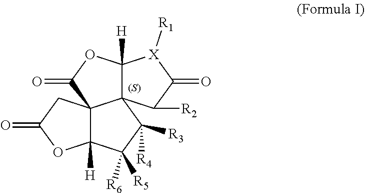

or a stereomer, a tautomer, or a pharmaceutically acceptable salt thereof, wherein
X is —O—, —NR¹—, —N═CR¹—NH—, or —NR¹—NH—; wherein when X is —O—, R¹ is absent; bond
Y¹ is a single bond or a double bond; R¹ is H, RB, or -(L¹)ᵤ-(Z¹)ᵥ; wherein
   L¹ is C₁-C₁₀ aliphatic wherein up to three carbon atoms of the C₁-C₁₀ aliphatic are optionally replaced by N, O, or S; wherein L¹ is optionally substituted with 1-3 occurrences of halo, CN, R, OR′, or R^{1C};
      u is 0 or 1;
      v is 0 or 1;
   Z¹ is a 5-16 membered aromatic or nonaromatic monocyclic or bicyclic ring system having 0-5 heteroatoms selected from O, N, or S; wherein Z¹ is optionally substituted with 1-5 occurrences of R^{1A}, R^{1C} or combinations thereof;
   R^{1A} is (L²)ₘ-(Z²)_{w}; wherein
      L² is C₁-C₁₀ aliphatic wherein up to three carbon atoms of the C₁-C₁₀ aliphatic are optionally replaced by N, NR, O, S, C═O, SO₂, S═O, (C═O)N, N(C═O)N, (C═O)O, or Si; wherein L² is optionally substituted with 1-3 occurrences of halo, CN, R, OR′ or; m is 0 or 1;
      w is 0 or 1;
Z² is a C₁-C₁₀ aliphatic, or 3-16 membered aromatic or nonaromatic monocyclic or bicyclic ring system having 0-5 heteroatoms selected from O, N, or S; wherein Z² is optionally substituted with 1-5 occurrences of R^{1B};
R^{1B} is H, halo, CN, R*, OR*, NRR*; or two R^{1B}, taken together with the atom to which they are attached, form a 3-6 membered ring having 0-4 heteroatoms;
R^{1C} is H, halo, CN, R*, OR*, NRR*; or two R^{1C}, taken together with the atom to which they are attached, form a 3-6 membered ring having 0-4 heteroatoms;
R* is C₁-C₆ aliphatic wherein up to three methylene units of the C₁-C₆ aliphatic are optionally replaced by N, NR, O, S, C═O, SO, SO₂ or Si and wherein the C₁-C₆ aliphatic is optionally substituted with 1-3 occurrences of halo, CN, R′ or OR′;
R² is R^{2A} or OR^{2A}, wherein R^{2A} is H, a C₁-C₁₆ aliphatic, a 5-10 membered aromatic or nonaromatic monocyclic or bicyclic ring system, or —(C₁-C₁₆ aliphatic)-(5-10 membered aromatic or nonaromatic monocyclic or bicyclic ring system); wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{2A} is optionally substituted with 1-5 occurrences of R^{2B}, wherein R^{2B} is halo, R′ or OR′;
R³ is OH, R^{3A}, or OR^{3A}; wherein R^{3A} is C₁-C₁₀ aliphatic optionally substituted with 1-3 occurrences of halo, R or OR′;
R⁴ is OH, R^{4A}, OR^{4A}; or when bond Y¹ between R⁴ and R⁵ is a double bond, R⁴ is absent; wherein R^{4A} is C₁-C₇ aliphatic and R^{4A} is optionally substituted with 1-3 occurrences of halo, R′ or OR′;
R⁵ is H or OH;
R⁶ is H; or when bond Y¹ between R⁴ and R⁵ is a double bond, R⁶ is absent;
R is H or C₁-C₆ aliphatic optionally substituted by 1-3 occurrences of F; or two R, taken together with the atom(s) to which they are attached, form a 3-6 membered ring having 0-4 heteroatoms; and
R′ is H, a C₁-C₆ aliphatic optionally substituted by 1-3 occurrences of F, or a 5-10 membered aromatic or nonaromatic monocyclic or bicyclic ring system having 0-5 heteroatoms selected from O, N, or S;
wherein when R² is OH, R³ is tert-butyl, R⁴ is OH, R⁵ is H, and R⁶ is H, X is not —O—.

For the sake of clarity, when u is 0 or v is 0, then the bond before -(L¹))ᵤ or before —(Z¹)ᵥ is also absent, respectively. Likewise, when m and w is 0, the bond before -(L²)ₘ or before —(Z²)_{w} is also absent.

In some embodiments, X is —NR¹—, —N═CR¹—NH—, or —NR¹—NH—.

In some embodiments, the compound described in any one of the preceding embodiments has the structure of Formula Ia:

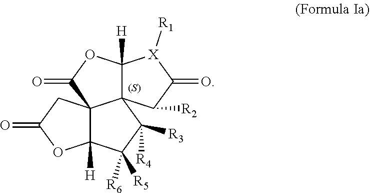

In some embodiments, the compound described in any one of the preceding embodiments has the structure of Formula Ib:

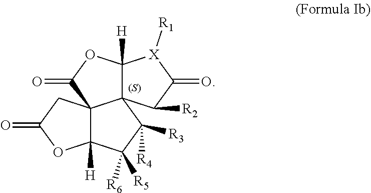

In some embodiments, R² is R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, or N(C═O)OC(CH₃)₃; wherein phenyl is optionally substituted with 1-5 occurrences of R^{2B}, wherein R^{2B} is halo, R′ or OR′; R³ is methyl, ethyl, propyl, isopropyl, butyl, isobutyl, or tert-butyl; the bond Y¹ between R⁴ and R⁵ is a single bond; R⁴ is OH or OR^{4A}; and R⁵ is H or OH.

In some embodiments, R¹ is H.

In some embodiments, R¹ is -(L¹)ᵤ-(Z¹)ᵥ; wherein L¹ is C₁-C₁₀ aliphatic wherein up to three carbon atoms of the C₁-C₁₀ aliphatic are optionally replaced by N, O, or S; Z¹ is phenyl, 1-methyl-1,2,3,4-tetrahydronaphthalen-2-y, 1-methyl-2H-isoindol-2-yl, imidazol, indolyl, napthalenyl, adamantanyl, azetidinyl, bicyclo[1.1.1]pentyl, 1-oxa-8-azaspiro[4.5]decan-3-yl, cyclobutanyl, cyclohexanyl, cyclopentanyl, cyclopropanyl, norbornenyl, oxetanyl, piperazinyl, piperidinyl, pyridinyl, pyrrolidinyl, tetrahydrofuranyl, [0196]tetrahydropyranyl, tetrahydrothiopyranyl, or C₃-C₁₂ cycloaliphatic having 0-5 heteroatoms selected from O, N, or S; u is 0 or 1; and v is 0 or 1; wherein Z¹ is optionally substituted with 1-5 occurrences of R^{1C}, morpholinyl, —OCH₂O—, —(C═O)-(pyrazinyl)-R^{1B}, —(C═O)-(phenyl)-R^{1B}, or —(SO₂)-(phenyl)-R^{1B}; wherein each independent occurrence of R^{1B} is H, halo, R*, OR*, or NRR*; wherein each independent occurrence of R^{1C} is H, halo, R*, OR*, or NRR*; and wherein each independent occurrence of R* is H, ═N, —C≡CH, —N═N—, —CH₃, —CH₂F, —CHF₂, —CF₃, —CN, —CH₂O—, —CF₂O—, —CH₂CH₂O—, or -Boc (—(C═O)OC(CH₃)₃).

In some embodiments, when Z¹ is phenyl, Z¹ is optionally substituted with 1-5 occurrences of morpholinyl or R^{1C}, wherein R^{1C} is halo, CH₃, —CH₂F, —CHF₂, —CF₃, —CN—OCH₃, —OCH₂O—, —OCF₂O—, —OCH₂CH₂O—, —NH₂, —NH(C═O)CH₃, or —N(Boc)-(N(C═O)OC(CH₃)₃).

In some embodiments, when Z¹ is piperidinyl, Z¹ is optionally substituted with 1-2 occurrences of R^{1C}, wherein R^{1C} is tert-butoxylcarbonyl, 5-(difluoromethyl)pyrazine-2-carbonyl, 2,2-difluoro-2H-1,3-benzodioxole-5-carbonyl, 2,3-dihydro-1,4-benzodioxine-6-carbonyl, 2,3-dihydro-1-benzofuran-5-sulfonyl, 4-chlorobenzoyl, 2,3-dihydro-1-benzofuran-5-carbonyl, or prop-2-enoyl.

In some embodiments, when Z¹ is pyrrolidinyl, Z¹ is optionally substituted with 1-2 occurrences of R^{1C}, wherein R^{1C} is tert-butoxylcarbonyl.

In some embodiments, R¹ is H, 2,4-dimethoxybenzyl, [1-(tert-butoxycarbonyl)piperidin-4-yl]methyl, piperidin-4-ylmethyl, 2-[1-(tert-butoxycarbonyl)piperidin-4-yl]ethyl, 2-(piperidin-4-yl)ethyl, 3-[1-(tert-butoxycarbonyl)piperidin-4-yl]propyl, 3-(piperidin-4-yl)propyl, 2-[4-(tert-butoxycarbonyl)piperazin-1-yl]ethyl, 2-(piperazin-1-yl)ethyl, 2-(3-methyl-1H-indol-2-yl)ethyl, 3-(1H-imidazol-1-yl)propyl, (R)-[1-(tertbutoxycarbonyl)pyrrolidin-3-yl]methyl, (S)-pyrrolidin-3-yl)methyl), ((2R)-bicyclo[2.2.1]hept-5-en-2-yl)methyl, phenyl, 4-acetamidophenyl, 4-[(tert-butoxycarbonyl)amino]phenyl, 4-aminophenyl, 4-(morpholin-4-yl)phenyl, benzo[d][1,3]dioxol-5-yl, pyridin-3-yl, benzyl, methyl, bicyclo[1.1.1]pentyl, oxetan-3-yl, cyclobutyl methyl, cyclopropyl methyl, (oxetan-3-yl)methyl, adamantan-2-yl methyl, NH₂, cyclopropyl, 3-methoxy phenyl, 4-methoxy phenyl, naphthalen-2-yl, 3-(trifluoromethyl) phenyl, 4-cyano phenyl, 2-[3-(but-3-yn-1-yl)-3H-diazirin-3-yl]ethyl, cyclohexyl, 4-fluoro phenyl, 4-(trifluoromethyl) phenyl, 4-toluyl, 3-toluyl, 2-toluyl, (oxolan-2-yl)methyl, 2-methoxy-2-oxoethyl, (1-(5-(difluoromethyl)pyrazine-2-carbonyl)piperidin-4-yl)methyl, [1-(2,3-dihydro-1-benzofuran-5-sulfonyl)piperidin-4-yl]methyl, (1-(2,2-difluorobenzo[d][1,3]dioxole-5-carbonyl)piperidin-4-yl)methyl, (1-(2,3-dihydrobenzo[b][1,4]dioxine-6-carbonyl)piperidin-4-yl)methyl, (1-(4-chlorobenzoyl)piperidin-4-yl)methyl, (1-(2,3-dihydrobenzofuran-5-carbonyl)piperidin-4-yl)methyl, (1-acryloylpiperidin-4-yl)methyl, (1-(quinoxaline-6-carbonyl)piperidin-4-yl)methyl, (tetrahydro-2H-pyran-4-yl)methyl, (tetrahydro-2H-thiopyran-4-yl)methyl, 2-(1-methyl-1,2,3,4-tetrahydronaphthalen-2-yl)ethyl, 2-(1-methyl-2H-isoindol-2-yl)ethyl, 2-(azetidin-1-yl)ethyl, 2-(trifluoromethyl) phenyl, 2-fluoro phenyl, 2-methoxy phenyl, 3,4-difluoro phenyl, 3,5-difluoro phenyl, 3-fluoro phenyl, 4-hydroxy phenyl, 8-(tert-butoxycarbonyl)-1-oxa-8-azaspiro[4.5]decan-3-yl, anilinyl, benzo[d][1,3]dioxol-4-yl, cyclobutyl, cyclohexyl methyl, naphthalen-1-yl, piperidin-4-yl methyl, pyridin-2-yl, or pyridin-4-yl.

In some embodiments, R² is R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, or N(C═O)OC(CH₃)₃; wherein phenyl is optionally substituted with 1-5 occurrences of halo, R′ or OR′; R³ is tert-butyl;
the bond Y¹ between R⁴ and R⁵ is a single bond; R⁴ is OH; R⁵ is H; X is —NR¹—, —N═CR¹—NH—, or —NR¹—NH—; and R¹ is selected from a group in Table 1a or Table 1b.

In some embodiments, R¹ is

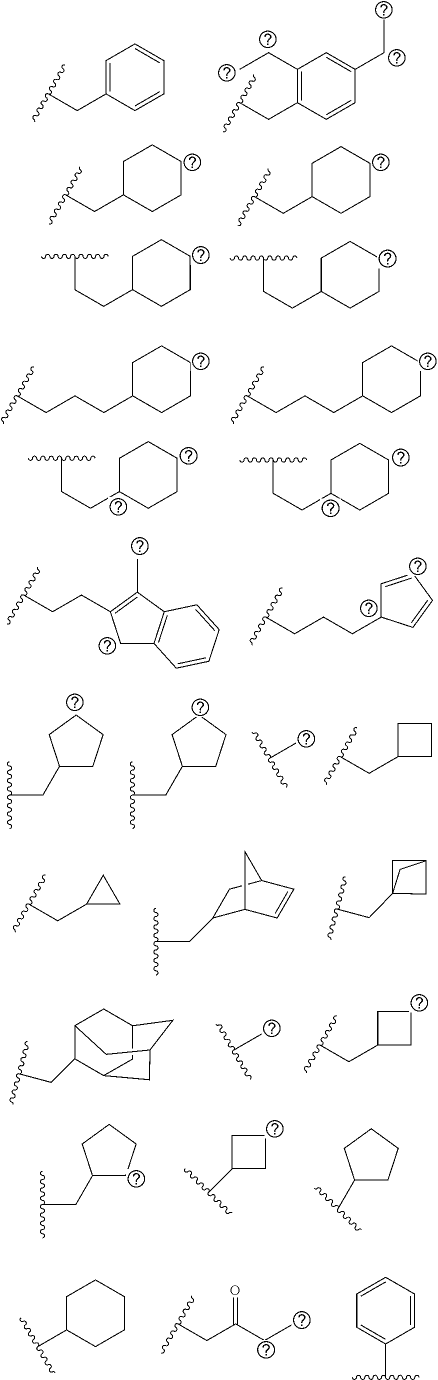

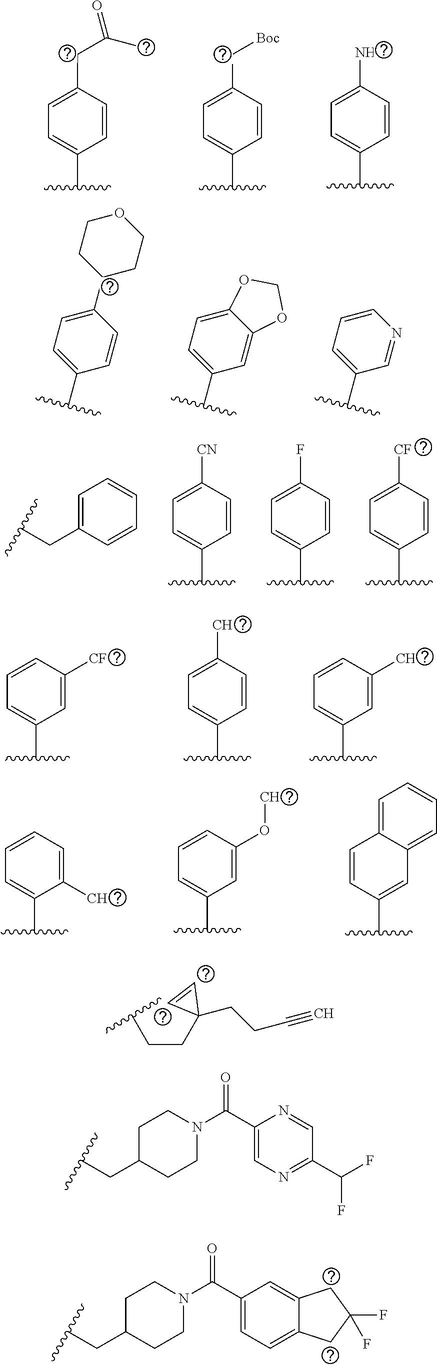

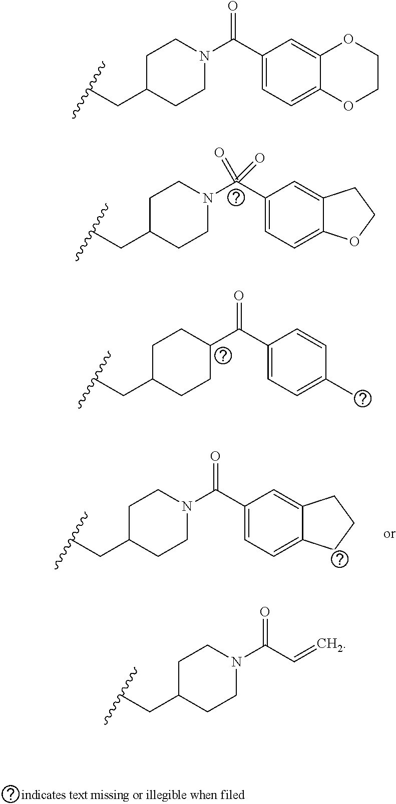

In some embodiments, the compound is selected from the group consisting of the compounds as described in Table 1d.

In some embodiments, the compound is XBB-037 or XBB-037′.

In some embodiments, the bond Y¹ is a double bond, having Formula I′.

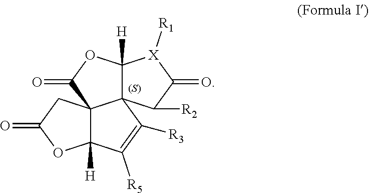

In some embodiments, X is —O— and R¹ is absent; R² is R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, or N(C═O)OC(CH₃)₃; wherein phenyl is optionally substituted with 1-5 occurrences of halo, R′ or OR′; R³ is tert-butyl; R⁴ is absent; R⁵ is H; and R⁶ is absent.

In some embodiments, the compound described in any one of the preceding embodiments has a structure of Formula I′a:

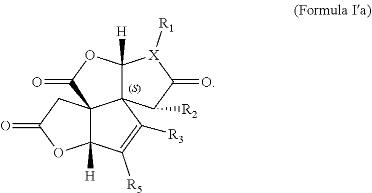

In some embodiments, the compound described in any one of the preceding embodiments has a structure of Formula I′b:

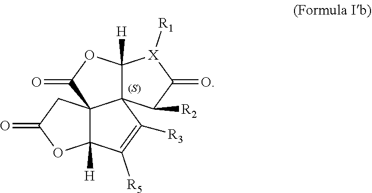

In some embodiments, X is —O— and R¹ is absent; bond Y¹ is a single bond; R² is OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, or CH₂CH₂OCH₃; wherein phenyl is optionally substituted with 1-5 occurrences of halo, R′ or OR′; R³ is isopropenyl; R⁴ is CH₃; R⁵ is H; and R⁶ is H.

In some embodiments, provided is a compound of Formula II:

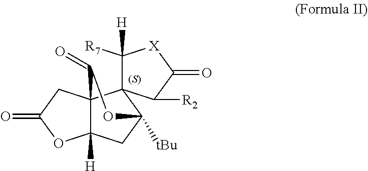

or a stereomer, a tautomer, or a pharmaceutically acceptable salt thereof, wherein X is —O—, —NR¹—, —N═CR¹—NH—, or —NR¹—NH—; wherein R¹ is as defined in claim 1; R² is R^{2A} or OR^{2A}, wherein R^{2A} is H, a C₁-C₁₆ aliphatic or a 5-10 membered aromatic or nonaromatic monocyclic or bicyclic ring system, wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{2A} is optionally substituted with 1-5 occurrences of R^{2B}, wherein R^{2B} is halo, R′ or OR′; and R⁷ is R^{7A} or OR^{7A}, wherein R^{7A} is H, a C₁-C₁₆ aliphatic or a 5-10 membered aromatic or nonaromatic monocyclic or bicyclic ring system, wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{7A} is optionally substituted with 1-5 occurrences of R^{7B}, wherein R^{7B} is halo, R′ or OR′.

In some embodiments, the compound described in any one of the preceeding embodiments has a structure of Formula IIa:

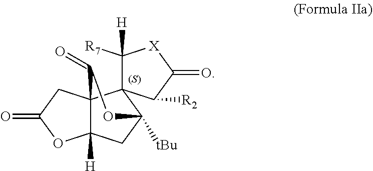

In some embodiments, the compound described in any one of the preceding embodiments has a structure of Formula IIb:

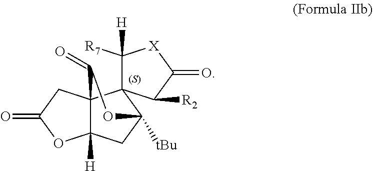

In some embodiments, X is —O—; R² is R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, or N(C═O)OC(CH₃)₃; wherein phenyl is optionally substituted with 1-5 occurrences of halo, R′ or OR′; and R⁷ is R^{7A} or OR^{7A}, wherein R^{7A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, or N(C═O)OC(CH₃)₃; wherein phenyl is optionally substituted with 1-5 occurrences of halo, R′ or OR′.

In some embodiments, the compound is

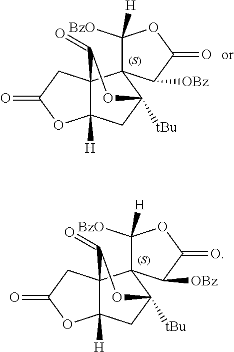

In some embodiments, provided is a process for preparing a compound of any one of the preceding embodiments, including at least the following steps: (i) treating bilobalide with R^{2A}—X in a suitable solvent to form protected product IIa

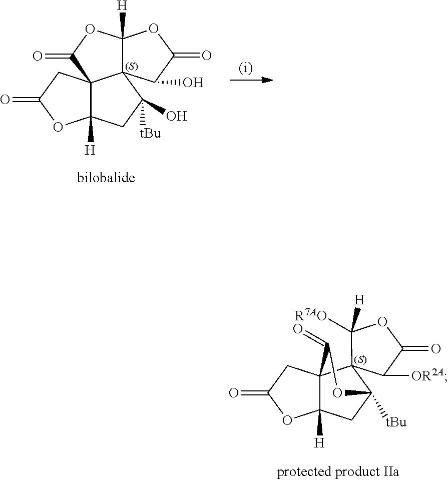

and (ii) treating protected product IIa with at least one base or an acceptable salt thereof to form aminated product IIb

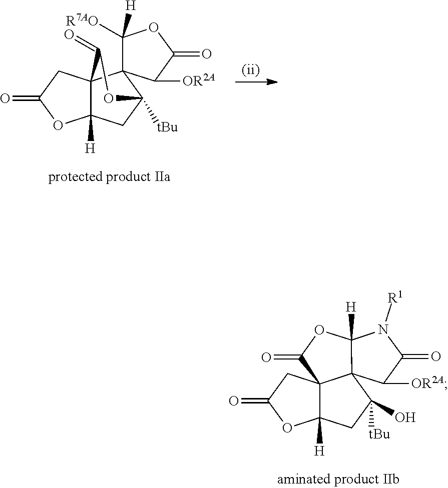

wherein R^{2A} and R^{7A} are as defined in any one of the preceding embodiments.

The process of claim 26, wherein the aminated product IIb has the formula of aminated product IIb′, further comprising the step of: (iii) treating aminated product IIb′ with an R^{1′}—B(OH)₂ in the presence of a catalyst to form a N-arylated product IIc

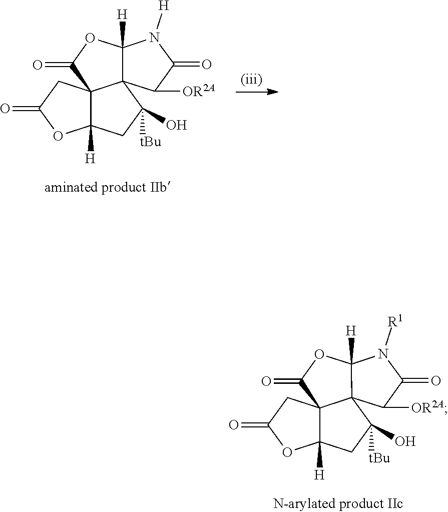

wherein R¹ and R^{2A} are as defined in any one of the preceding embodiments. In some embodiments, a ligand is optionally added to the catalyst. In some embodiments, the catalyst is a Cu catalyst. In some embodiments, Cu:ligand molar ratio is such that there is an excess of either Cu or the ligand. In some embodiments, the Cu:ligand ratio is 5:1, 4:1, 3:1, 2:1, 1:2, 1:3, 1:4, or 1:5. In some embodiments, the Cu:ligand ratio is A:B, wherein A is 1.1 to 10 and B is 1 A is 1 and B is 1.1 to 10. In some embodiments, the Cu:ligand ratio is not 1:1.

In some embodiments, the process further include the step of: (iv) treating the aminated product IIb of any one of the preceding embodiments or the N-arylated product IIc of any one of the preceding embodiments with an alkali salt or an acid in a protic solvent to form a deprotected product. In some embodiments, the deprotected product is deprotected product IId and the alkali salt is K₂CO₃

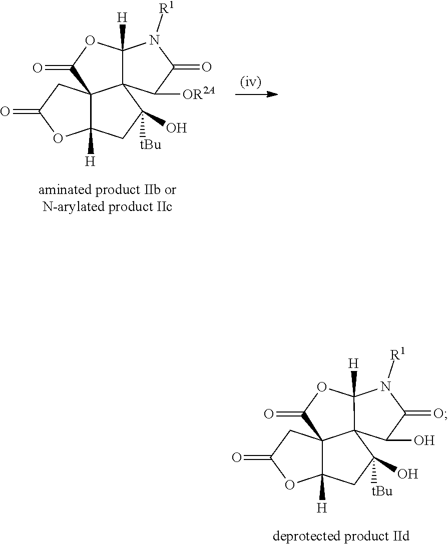

wherein R¹ and R^{2A} are as defined in any one of the preceding embodiments. In some embodiments, the deprotected product is deprotected product IIe and the acid is an organic acid such as acetyl chloride or oxalyl chloride, provided that R¹ contains other protecting groups such as tert-butoxycarbonyl (boc); for example:

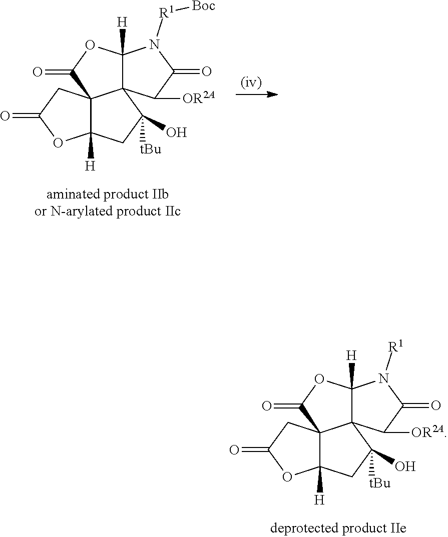

In some embodiments, R^{2A} and R^{7A} of the protected product IIa is as defined in any one of the preceding embodiments.

In some embodiments, R^{2A}—X is benzoyl chloride, and the suitable solvent is pyridine.

In some embodiments, R¹ and R^{2A} of aminated product IIb are as defined in any one of the preceding embodiments.

In some embodiments, R¹ and R^{2A} of the N-arylated product IIc are as defined in any one of the preceding embodiments.

In some embodiments, the at least one base is ammonia, and the aminated product IIb is

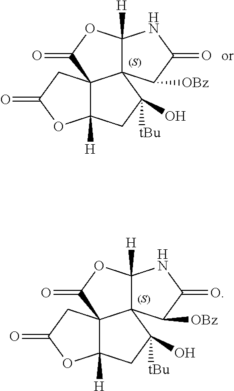

In some embodiments, the deprotected product, such as deprotected product IId or deprotected product IIe, is as defined in any one of the preceding embodiments.

In some embodiments, the at least one base is NH₂R¹.

In some embodiments, the at least one base is [H₃NR¹]⁺, triethylamine or diisopropylethylamine.

In some embodiments, the protected product IIa is as defined in any one of the preceding embodiments.

The process of claim 33, wherein the [H₃NR¹]⁺ is provided as XYa prepared by the steps of: (a) treating R—COOH with 1-hydroxybenzotriazole, N-(3-dimethylaminopropyl)-N′-ethylcarbodiimide hydrochloride, and tert-butyl (piperidin-4-ylmethyl)carbamate, to form a boc-protected product SXa, wherein R═(Z²)_{w}—R^{1B}, wherein Z², w and R^{1B} are as defined in claim 1; and (b) treating the boc-protected product SXa with an acid in a solvent to form XYa

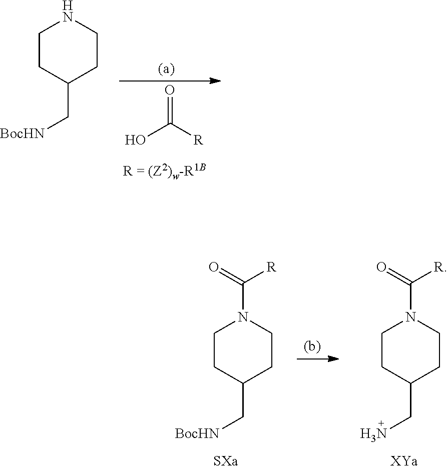

In some embodiments, the [H₃NR¹]⁺ is provided as XYb prepared by the steps of: (a) treating R—SO₂ with tert-butyl (piperidin-4-ylmethyl)carbamate and triethylamine, to form a boc-protected product SXb, wherein R═(Z²)_{w}—R^{1B}, wherein Z², w and R^{1B} are as defined in claim 1; and (b) treating the boc-protected product SXb with an acid in a solvent to form XYb

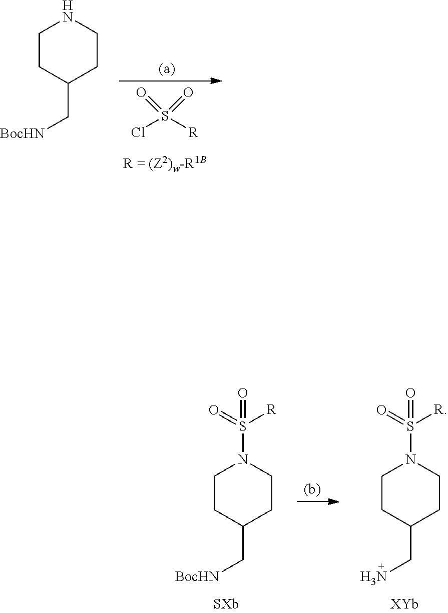

In some embodiments, provided is a process of preparing a compound of claim 1, which include the steps of: (i) treating bilobalide with Ac₂O and an acid to form a protected product IVa

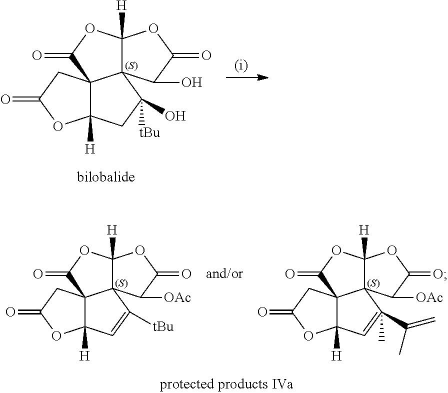

and
(ii) treating protected product IVa with at least one base or an acceptable salt thereof to form aminated product IVb

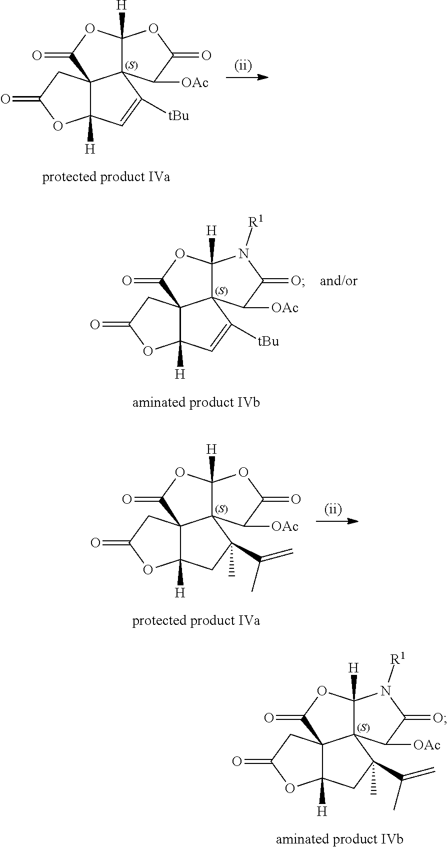

wherein R¹ is as defined in claim 1.

In some embodiments, the process further includes at least one of the steps of: (iii) treating the aminated product IVb with an oxidizing agent and a solvent to form oxidized product IVc

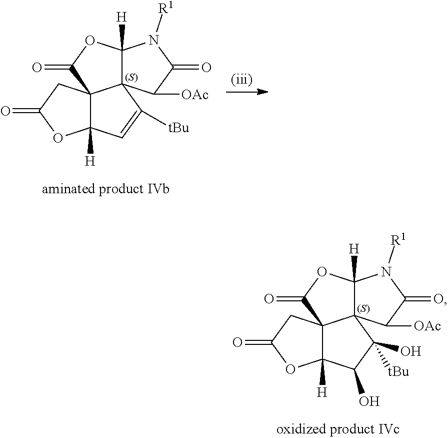

In some embodiments, the process further includes at least one of the steps of: (iv) treating the aminated product IVb or the oxidized product IVc with an acid to form deprotected product IVd

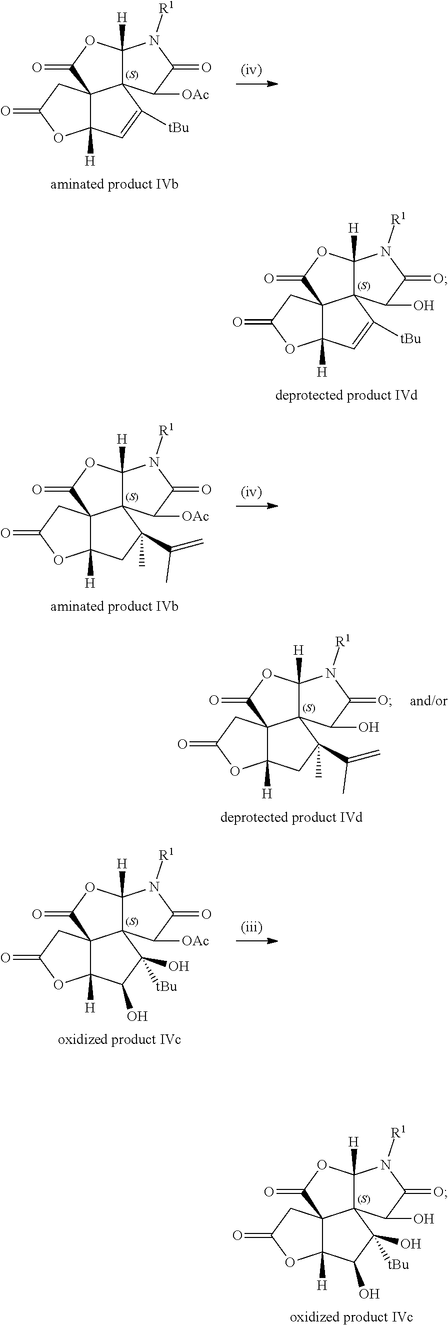

wherein R¹ is as defined in claim 1.

In some embodiments, provided is a method of treating or preventing neurological related disease in a subject in need thereof, comprising administering to the subject a compound of any one of the preceding embodiments.

In some embodiments, the neurological related disease is a neurodegenerative disease.

In some embodiments, the neurodegenerative disease is caused by ferroptosis.

In some embodiments, the neurodegenerative disease is Alzheimer's disease or Parkinson's disease.

In some embodiments, provided is a use of a compound of any one of the preceding embodiments for treating or preventing Alzheimer's disease or Parkinson's disease.

In some embodiments, provided is a use of a compound of any one of the preceding embodiments for inhibiting ferroptosis by restoring glutathione peroxidase 4 (GPX4), thereby mitigating GPX4 degradation induced by ferroptosis inducers.

In some embodiments, the ferroptosis inducers are RSL3, FIN56, ML162, ML210, or erastin.

In some embodiments, provided is a use of a compound of any one of the preceding embodiments for inhibiting ferroptosis by reducing intracellular reactive oxygen species (ROS level).

In some embodiments, provided is a use of a compound of any one of the preceding embodiments for inhibiting ferroptosis by reducing lipid peroxidation.

In some embodiments, provided is a use of a compound of any one of the preceding embodiments for the manufacture of a medicament for treating or preventing Alzheimer's disease or Parkinson's disease.

### Embodiments III

In some embodiments, provided is a compound of Formula I:

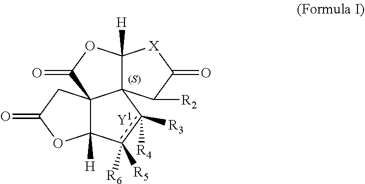

or a stereomer, a tautomer, or a pharmaceutically acceptable salt thereof,
wherein
X is —O—, —NR¹—, —N═CR¹—NH—, or —NR¹—NH—; wherein when X is —O—, R¹ is absent;
bond Y¹ is between R⁴ and R⁵ and is a single bond or a double bond;
R¹ is H, R^{1B}, or -(L¹)ᵤ-(Z¹)ᵥ; wherein
   L¹ is C₁-C₁₀ aliphatic wherein up to three carbon atoms of the C₁-C₁₀ aliphatic are optionally replaced by N, O, or S; wherein L¹ is optionally substituted with 1-3 occurrences of halo, CN, R, OR′, or R^{1C};
      u is 0 or 1;
      v is 0 or 1;
   Z¹ is a 5-16 membered aromatic or nonaromatic monocyclic, bicyclic, or tricyclic ring system having 0-7 heteroatoms selected from O, N, or S; wherein Z¹ is optionally substituted with 1-5 occurrences of R^{1A}, R^{1C} or combinations thereof;
R^{1A} is -(L²)ₘ-(Z²)_{w}; wherein
   L² is C₁-C₁₀ aliphatic wherein up to three carbon atoms of the C₁-C₁₀ aliphatic are optionally replaced by N, NR, O, S, C═O, SO₂, S═O, (C═O)N, N(C═O)N, (C═O)O, or Si; wherein L² is optionally substituted with 1-3 occurrences of halo, CN, R, OR′ or R^{1C};
   m is 0 or 1;
   w is 0 or 1;
Z² is a C₁-C₁₀ aliphatic, or 3-16 membered aromatic or nonaromatic monocyclic, bicyclic or tricylic ring system having 0-7 heteroatoms selected from O, N, or S; wherein Z² is optionally substituted with 1-5 occurrences of R^{1B};
R^{1B} is H, halo, CN, R*, OR*, NRR*; or two R^{1B}, taken together with the atom to which they are attached, form a 3-6 membered ring having 0-4 heteroatoms;
R^{1C} is H, halo, CN, a 5-10 membered aromatic or nonaromatic monocyclic or bicyclic ring system having 0-5 heteroatoms selected from O, N, or S; R*, OR*, NRR*; or two R^{1C}, taken together with the atom or atoms to which they are attached, optionally form a 3-16 membered ring having 0-4 heteroatoms; wherein R^{1C} is optionally substituted with 1-3 occurrences of halo, CN, R′ or OR′;
R* is C₁-C₆ aliphatic wherein up to three methylene units of the C₁-C₆ aliphatic are optionally replaced by N, NR, O, S, C═O, SO, SO₂ or Si and wherein the C₁-C₆ aliphatic is optionally substituted with 1-3 occurrences of halo, CN, R′ or OR′;
R² is R^{2A} or OR^{2A}, wherein R^{2A} is H, a C₁-C₁₆ aliphatic, a 5-10 membered aromatic or nonaromatic monocyclic or bicyclic ring system, or —(C₁-C₁₆ aliphatic)-(5-10 membered aromatic or nonaromatic monocyclic or bicyclic ring system); wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{2A} is optionally substituted with 1-5 occurrences of R^{2B}, wherein R^{2B} is halo, R′ or OR′;
R³ is OH, R^{3A}, or OR^{3A}; wherein R^{3A} is C₁-C₁₀ aliphatic optionally substituted with 1-3 occurrences of halo, R or OR′;
R⁴ is OH, R^{4A}, OR^{4A}; or when bond Y¹ between R⁴ and R⁵ is a double bond, R⁴ is absent; wherein R^{4A} is C₁-C₇ aliphatic and R^{4A} is optionally substituted with 1-3 occurrences of halo, R′ or OR′;
R⁵ is H or OH;
R⁶ is H; or when bond Y¹ between R⁴ and R⁵ is a double bond, R⁶ is absent;
R is H or C₁-C₆ aliphatic optionally substituted by 1-3 occurrences of F; or two R, taken together with the atom(s) to which they are attached, form a 3-6 membered ring having 0-4 heteroatoms; and
R′ is H, a C₁-C₆ aliphatic wherein up to three carbon atoms of the C₁-C₆ aliphatic are optionally replaced with O, NH, N(C₁-C₆ alkyl), C(O), or S(O)₂; wherein said C₁-C₆ aliphatic is optionally substituted by 1-3 occurrences of F, OR, NH₂, NHR″, or NR″₂, wherein R″ is C₁-C₆ aliphatic or a 5-10 membered aromatic or nonaromatic monocyclic or bicyclic ring system having 0-5 heteroatoms selected from O, N, or S;
wherein when R² is OH, R³ is tert-butyl, R⁴ is OH, R⁵ is H, and R⁶ is H, X is not —O—.

In some embodiments, R² is
R^{2A} or OR^{2A}, wherein R^{2A} is H, C═O(C₁₋₁₀ aliphatic), SO₂(C₁₋₁₀ aliphatic), SO₂(phenyl), phenyl, Si(C₁₋₁₀ aliphatic)₁₋₂, Si(phenyl)₁₋₂, —(C₁₋₁₀ aliphatic)O(C₁₋₁₀ aliphatic)-, (C═O)(phenyl), NH(C═O) (C₁₋₁₀ aliphatic)

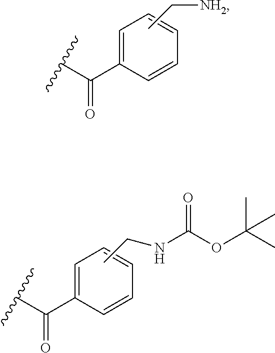

or NH(C═O)O(C₁₋₁₀ aliphatic); wherein each R^{2A} is independently and optionally substituted with 1-5 occurrences of R^{2B}, wherein R^{2B} is halo, R′ or OR′;
R³ is C₁₋₁₀ aliphatic;
the bond Y¹ between R⁴ and R⁵ is a single bond;
R⁴ is OH or OR^{4A};
and R⁵ is H or OH.

In some embodiments, R² is
R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅,

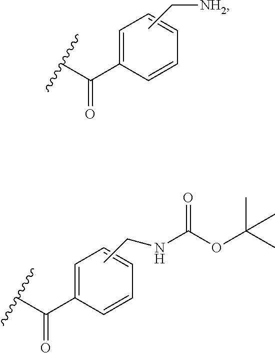

or NH(C═O)OC(CH₃)₃;
wherein phenyl, C₆H₄, and C₆H₅ are each independently and optionally substituted with 1-5 occurrences of R^{2B}, wherein R^{2B} is halo, R′ or OR′;
R³ is methyl, ethyl, propyl, isopropyl, butyl, isobutyl, or tert-butyl;
the bond Y¹ between R⁴ and R⁵ is a single bond;
R⁴ is OH or OR^{4A};
and R⁵ is H or OH.

In some embodiments, R¹ is
H,
2,4-dimethoxybenzyl,
[1-(tert-butoxycarbonyl)piperidin-4-yl]methyl,
piperidin-4-yl methyl,
2-[1-(tert-butoxycarbonyl)piperidin-4-yl]ethyl,
2-(piperidin-4-yl)ethyl,
3-[1-(tert-butoxycarbonyl)piperidin-4-yl]propyl,
3-(piperidin-4-yl)propyl,
2-[4-(tert-butoxycarbonyl)piperazin-1-yl]ethyl,
2-(piperazin-1-yl)ethyl,
2-(3-methyl-1H-indol-2-yl)ethyl,
3-(1H-imidazol-1-yl)propyl,
[1-(tertbutoxycarbonyl)pyrrolidin-3-yl]methyl,
(pyrrolidin-3-yl)methyl,
(bicyclo[2.2.1]hept-5-en-2-yl)methyl,
phenyl,
4-acetamidophenyl,
4-[(tert-butoxycarbonyl)amino]phenyl,
4-aminophenyl,
4-(morpholin-4-yl)phenyl,
benzo[d][1,3]dioxol-5-yl,
pyridin-3-yl,
benzyl,
methyl,
bicyclo[1.1.1]pentyl,
oxetan-3-yl,
cyclobutyl methyl,
cyclopropyl methyl,
(oxetan-3-yl)methyl,
adamantan-2-yl methyl,
NH₂,
cyclopropyl,
3-methoxy phenyl,
4-methoxy phenyl,
naphthalen-2-yl,
3-(trifluoromethyl) phenyl,
4-cyano phenyl,
2-[3-(but-3-yn-1-yl)-3H-diazirin-3-yl]ethyl,
cyclohexyl,
4-fluoro phenyl,
4-(trifluoromethyl) phenyl,
4-toluyl,
3-toluyl,
2-toluyl,
(oxolan-2-yl)methyl,
2-methoxy-2-oxoethyl,
(1-(5-(difluoromethyl)pyrazine-2-carbonyl)piperidin-4-yl)methyl,
[1-(2,3-dihydro-1-benzofuran-5-sulfonyl)piperidin-4-yl]methyl,
(1-(2,2-difluorobenzo[d][1,3]dioxole-5-carbonyl)piperidin-4-yl)methyl,
(1-(2,3-dihydrobenzo[b][1,4]dioxine-6-carbonyl)piperidin-4-yl)methyl,
(1-(4-chlorobenzoyl)piperidin-4-yl)methyl,
(1-(2,3-dihydrobenzofuran-5-carbonyl)piperidin-4-yl)methyl,
(1-acryloylpiperidin-4-yl)methyl,
(1-(quinoxaline-6-carbonyl)piperidin-4-yl)methyl,
(tetrahydro-2H-pyran-4-yl)methyl,
(tetrahydro-2H-thiopyran-4-yl)methyl,
2-(1-methyl-1,2,3,4-tetrahydronaphthalen-2-yl)ethyl,
2-(1-methyl-2H-isoindol-2-yl)ethyl,
2-(azetidin-1-yl)ethyl,
2-(trifluoromethyl) phenyl,
2-fluoro phenyl,
2-methoxy phenyl,
3,4-difluoro phenyl,
3,4-dichloro phenyl,
3,5-difluoro phenyl,
3-fluoro phenyl,
4-hydroxy phenyl,
8-(tert-butoxycarbonyl)-1-oxa-8-azaspiro[4.5]decan-3-yl,
anilinyl,
benzo[d][1,3]dioxol-4-yl,
cyclobutyl,
cyclohexyl methyl,
naphthalen-1-yl,
pyridin-2-yl,
pyridin-4-yl,
adamantan-1-yl methyl,
1-(tert-butoxycarbonyl)-1H-indol-5-yl,
1H-indol-5-yl,
3-[(tert-butoxycarbonyl)amino]phenyl,
4-Hydroxyphenyl ethyl,
1H-indole-3-ethyl,
((1R,4aS,10aR)-7-isopropyl-1,4a-dimethyl-1,2,3,4,4a,9,10,10a-octahydrophenanthren-1-yl) methyl,
[((tert-butoxycarbonyl)aminomethyl) adamantan-1-yl]methyl,
(aminomethyl)adamantan-1-yl) methyl,
3,5-di-tert butyl phenyl,
3,4-dihydroxyphenyl,
3-methoxy-4-hydroxyphenyl ethyl,
1H-indole-5-hydroxy-3-ethyl,
1H-indole-5-methoxy-3-ethyl,
1H-indole-4-hydroxy-3-ethyl,
piperonyl,
2-(4-Imidazolyl)ethyl (histamine),
2,2-diphenylethyl,
3-hydroxy-4-methoxyphenyl ethyl,
3,4-methylenedioxyphenyl ethyl,
1H-indole-5-hydroxy-3-ethyl (serotonin),
3,4-dihydroxyphenyl ethyl (dopamine),
1H-indole-3-ethyl (tryptamine),
3-methoxy-4-hydroxyphenyl ethyl (3-O-methyldopamine), or methylenedioxyphenyl.

In some embodiments, R² is
R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅,

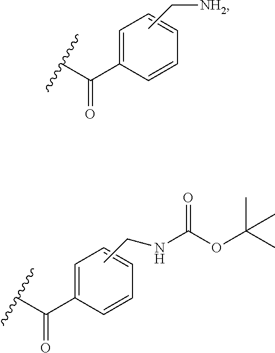

or NH(C═O)OC(CH₃)₃;
   wherein phenyl is optionally substituted with 1-5 occurrences of halo, R′ or OR′;
R³ is tert-butyl;
the bond Y¹ between R⁴ and R⁵ is a single bond;
R⁴ is OH;
R⁵ is H;
X is —NR¹—, —N═CR¹—NH—, or —NR¹—NH—; and.

R¹ is selected from

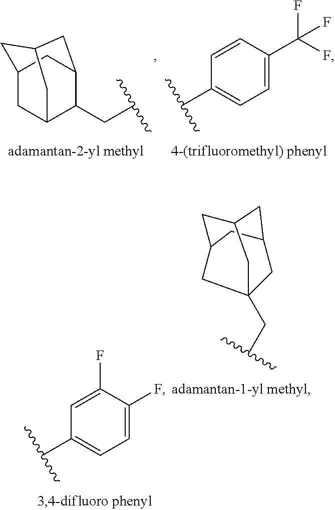 [SMILES: *CC1C2CC3CC(C2)CC1C3 ; *c1ccc(C(F)(F)F)cc1 ; *CC12CC3CC(CC(C3)C1)C2 ; *c1ccc(F)c(F)c1]

2-(4-imidazolyl)ethyl (histamine),
1H-indole-5-hydroxy-3-ethyl (serotonin),
3,4-dihydroxyphenyl ethyl (dopamine),
1H-indole-3-ethyl (tryptamine), or
3-methoxy-4-hydroxyphenyl ethyl (3-O-methyldopamine).

In some embodiments,

In some embodiments, provided is a method of treating or preventing cancer in a subject in need thereof, including administering to the subject a compound described herein.

In some embodiments, the cancer is bladder cancer, brain cancer, breast cancer, CNS cancer, colon cancer, hematopoietic cancer, kidney cancer, leukemia, lung cancer, melanoma, ovarian cancer, pancreatic cancer, prostate cancer, or renal cancer.

In some embodiments, the cancer is leukemia, colon cancer, lung cancer, melanoma or renal cancer.

In some embodiments, the lung cancer is non-small cell lung cancer (NSCLC).

In some embodiments, the leukemia is lymphocytic leukemia.

In some embodiments, provided is a use of a compound described herein for treating or preventing cancer.

In some embodiments, provided is a use of a compound described herein for the manufacture of a medicament for treating or preventing cancer.

In some embodiments, provided is a method of inducing cell death in a cancer cell, including contacting a compound described herein with the cancer cell.

In some embodiments, a method of inhibiting cell growth in a cancer cell, including contacting a compound described herein with the cancer cell.

In some embodiments, the method is an in vitro method.

In some embodiments, the compound is DW192, P-29, P-21, P-30, P-33, JW093, XBB-023, P-28, JW107, XBB-039, JW094, P-34, XBB-045, JW081, XBB-028, XBB-038, XBB-037, XBB-054, XBB-025, XBB-029, XBB-024, DW172, XBB-004, XBB-042, XBB-068, XBB-040, XBB-006, JW072, DW189, P-8, DW191, DW168, XBB-013, XBB-037′, XBB-009, XBB-060, XBB-016, DW182, XBB-010, SCC506, SCC363, or SXQ087-1.

In some embodiments, the compound is DW192, P-29, P-21, SCC506, SCC363, or SXQ087-1.

In some embodiments, provided is a method of treating or preventing neurological related disease in a subject in need thereof, including administering to the subject a compound of any one of the embodiments here.

In some embodiments, the neurological related disease is caused by ferroptosis.

In some embodiments, the neurological related disease is Alzheimer's disease or Parkinson's disease.

In some embodiments, provided is a use of a compound described herein for treating or preventing Alzheimer's disease or Parkinson's disease.

In some embodiments, provided is a use of a compound described herein for inhibiting ferroptosis by restoring glutathione peroxidase 4 (GPX4), thereby mitigating GPX4 degradation induced by ferroptosis inducers.

In some embodiments, provided is a use of a compound described herein for inhibiting ferroptosis by reducing intracellular reactive oxygen species (ROS level).

In some embodiments, provided is a use of a compound described herein for inhibiting ferroptosis by reducing lipid peroxidation.

In some embodiments, provided is a use of a compound described herein for the manufacture of a medicament for treating or preventing Alzheimer's disease or Parkinson's disease.

In some embodiments, the compound is DW192, P-29, P-21, P-30, P-33, JW093, XBB-023, P-28, JW107, XBB-039, JW094, P-34, XBB-045, JW081, XBB-028, XBB-038, XBB-037, XBB-054, XBB-025, XBB-029, XBB-024, DW172, XBB-004, XBB-042, XBB-068, XBB-040, XBB-006, JW072, DW189, P-8, DW191, DW168, XBB-013, XBB-037′, XBB-009, XBB-060, XBB-016, DW182, XBB-010, SCC506, SCC363, or SXQ087-1.

In some embodiments, the compound is DW192, P-29, P-21, SCC506, SCC363, or SXQ087-1.

In some embodiments, the compounds in the current disclosure include those wherein R¹ is derived from a neurotransmitter or derivatives thereof. For example, the compounds SXQ087-1, SXQ090-1, SXQ092-1, SXQ091-1, SXQ125-2, and SXQ128-1 are compounds wherein R¹ is derived from a neurotransmitter or derivatives thereof. Being “derived from a neurotransmitter or derivatives thereof” means that the structure of R¹ includes the structure of a neurotransmitter or at least a portion of the structure of a neurotransmitter. In some example embodiments, the neurotransmitter is a monoamine neurotransmitter, and R¹ is the portion of the molecule without the amine (see, for example, SXQ091-1 and serotonin),
In some embodiments, R¹ is derived from dopamine, 3-O-methyldopamine, serotonin, 5-hydroxytryptamin, tryptamine or histamine.

The compounds of the disclosure may be prepared in light of the specification using steps generally known to those of ordinary skill in the art. Those compounds may be analyzed by known methods, including but not limited to HRMS-ESI (high resolution mass spectrometry with electrospray ionization) and NMR (nuclear magnetic resonance).

### Generic Schemes

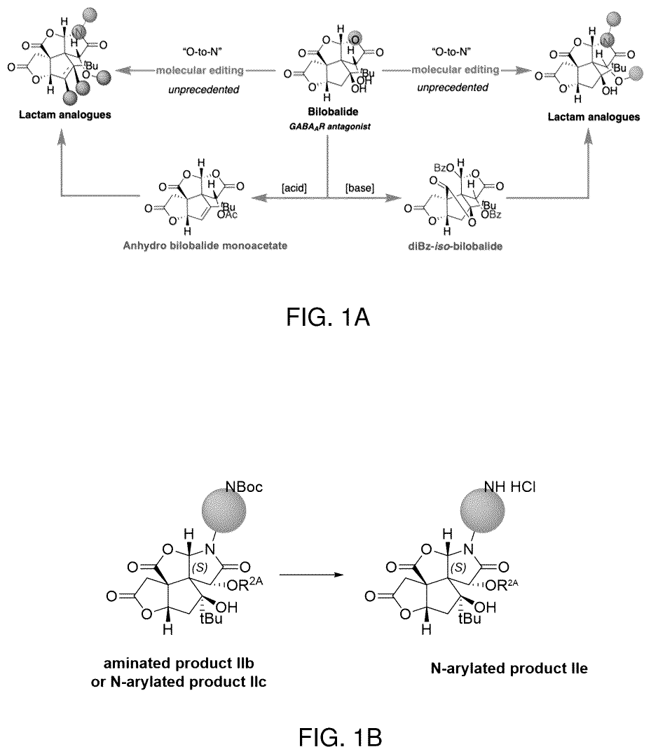

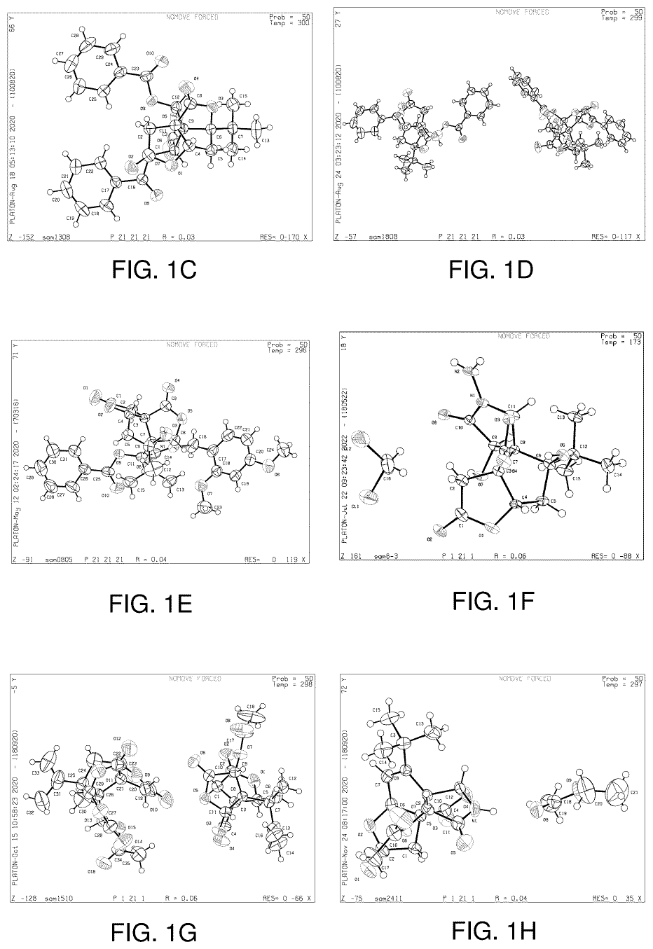

The following generic schemes and examples illustrate how to prepare the compounds of the present disclosure. The examples are for the purpose of illustration only and are not to be construed as limiting the scope of the invention in any way. FIG. 1A is an illustration showing the overall molecular editing schemes of bilobalide as disclosed herein.

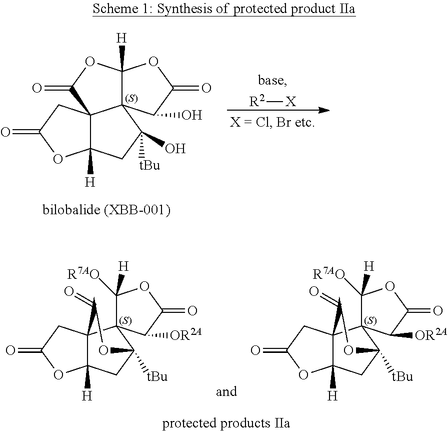

To a solution of bilobalide (1 equiv.), a suitable anhydrous solvent, R^{2A}—X (3-15 equiv.) and a base (0-6 equiv.) are added. In some examples, the base is a sterically hindered base. After stirring at a temperature of 20-80° C. for 16-72 h, the reaction solution is treated with the steps generally known to those of ordinary skill in the art. Purification using silica gel column chromatography afforded di-protected iso-bilobalides, wherein R² ═R⁷ (protected product IIa).

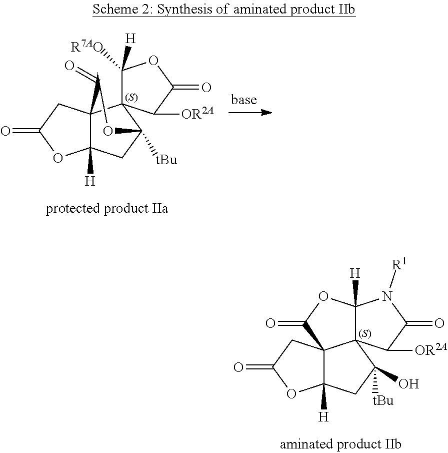

To a solution of protected product IIa (1 equiv) in a suitable anhydrous solvent is added a base (2 equiv). In some embodiments, the base is NH₂R¹. The resulting solution is then stirred for 15-60 minutes at room temperature. The reaction is diluted with dichloromethane and treated with the steps generally known to those of ordinary skill in the art to afford aminated product IIb.

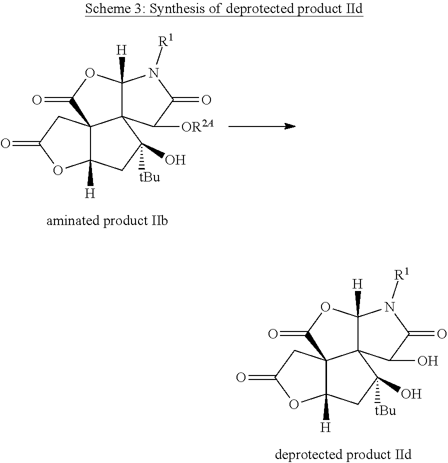

To a solution of aminated product IIb (1.0 equiv) in a protic solvent, a basic alkali salt (2 equiv.) is added. The resulting mixture is stirred at room temperature for 2 hours. The reaction mixture is then treated with the steps generally known to those of ordinary skill in the art to afford deprotected product IId.

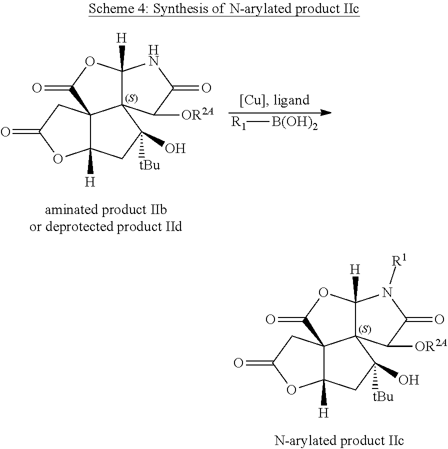

An oven-dried round-bottom flask is charged with aminated product IIb or deprotected product IId (1 equiv), R¹—B(OH)₂ (1.5-2 equiv), a catalyst (10-100 mol %), optionally a ligand (30-50 mol %), and a suitable solvent. The reaction mixture is stirred at room temperature under open air for 12-72 hours. The crude reaction mixture is then treated with the steps generally known to those of ordinary skill in the art to provide N-arylated product IIc. The N-arylated product IIc can be treated with the same procedure described in Scheme 3 to afford deprotected product IId.

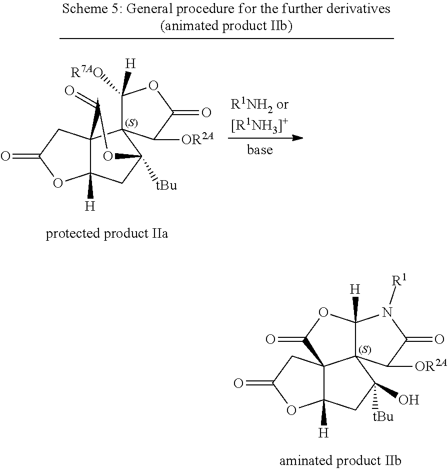

To a solution of protected product IIa (1 equiv) in a suitable anhydrous solvent is added R¹NH₂ or [R¹NH₃]⁺ (1-2 equiv) together with a suitable hindered base (0-3 equiv). The resulting solution is then stirred for 15 minutes up until 24 h at room temperature. The crude reaction mixture is then treated with the steps generally known to those of ordinary skill in the art to provide aminated product IIb.

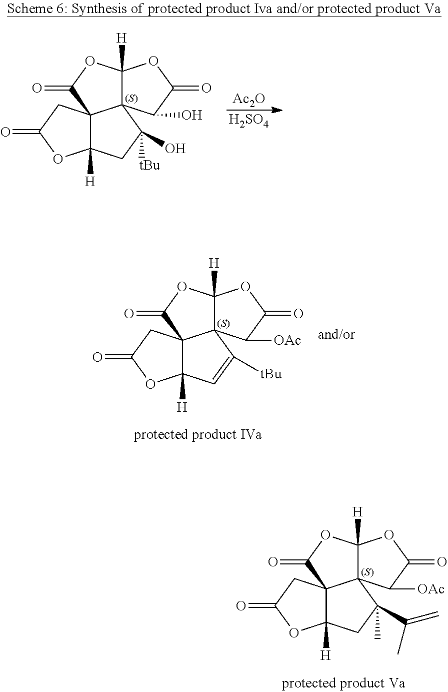

To a solution of Bilobalide in acetic anhydride is added a trace of concentrated sulfuric acid. The resulting solution is stirred at 50° C. for 3 h. The reaction solution is then treated with the steps generally known to those of ordinary skill in the art to yield protected product IVa and/or protected product Va.

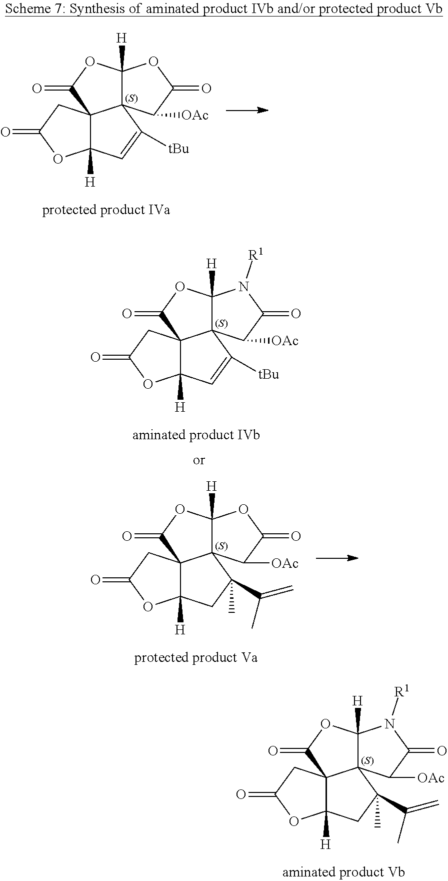

To a solution of protected product IVa (1.0 equiv) or protected product Va (1.0 equiv) in an anhydrous solvent is added a base (2.0 equiv). The resulting solution is then allowed to be stirred for 30 min at room temperature. The reaction is diluted with dichloromethane and treated with the steps generally known to those of ordinary skill in the art to afford aminated product IVb and/or aminated product Vb.

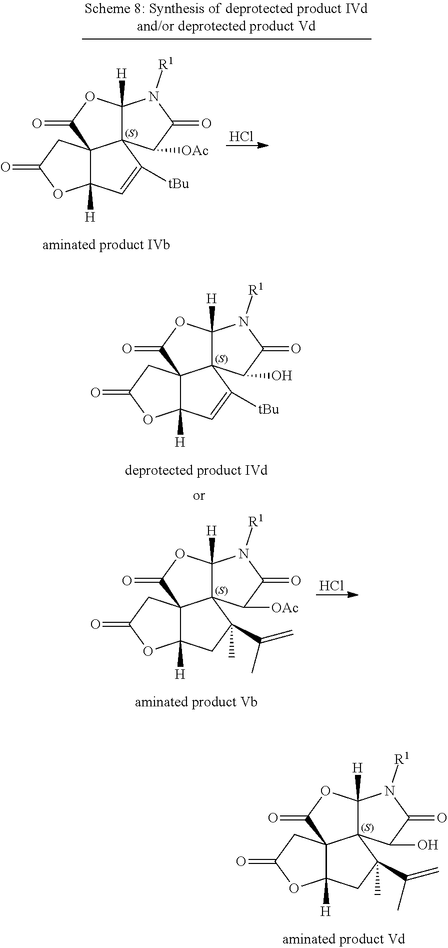

To a round-bottom flask is added aminated product IVb and/or aminated product Vb and a strong acid in H₂O. The resulting solution is allowed to be stirred under reflux condition for 12 h. the reaction solution is then treated with the steps generally known to those of ordinary skill in the art to give deprotected product IVd and/or deprotected product Vd.

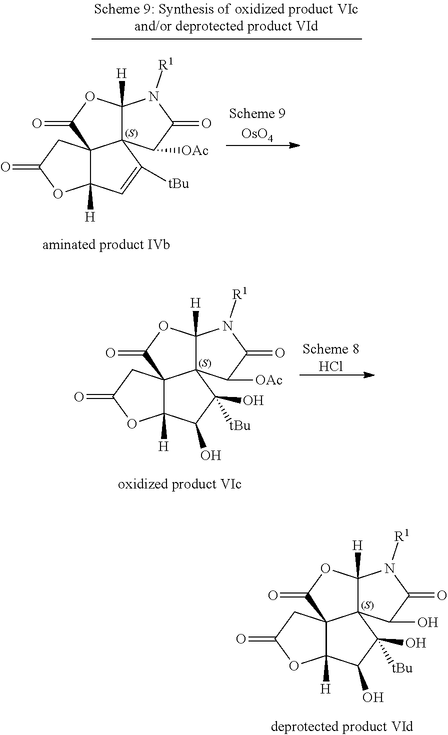

To a round-bottom flask is added aminated product IVb (1.0 equiv.) and a suitable solvent, followed by the addition of pyridine and osmium (VIII) oxide. The resulting solution is allowed to be stirred at room temperature for 18 h. The resulting solution is treated with the steps generally known to those of ordinary skill in the art to give oxidized product VIc. The oxidized product VIc can be treated with the same procedure described in Scheme 8 to afford deprotected product VId.

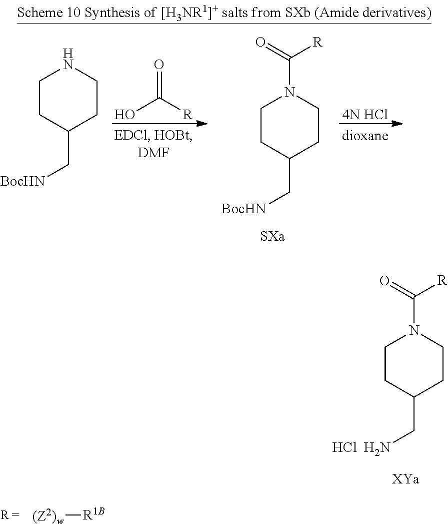

Syntheses of other [H₃NR¹]⁺ salts for use in Scheme 5 are described herein.

A solution of R-substituted carboxylic acid, wherein R═(Z²)_{w}—R^{1B} (1.0 equiv.), in a suitable solvent is treated with 1-hydroxybenzotriazole (HOBt) (1.1 equiv.), and N-(3-dimethylaminopropyl)-N′-ethylcarbodiimide hydrochloride (EDCI) (1.1 equiv.). This mixture was stirred for 30 minutes at room temperature, then tert-butyl (piperidin-4-ylmethyl)carbamate (1.0 equiv.) is added. The crude reaction mixture is then treated with the steps generally known to those of ordinary skill in the art to afford a SXa. Then, SXa is treated with 4 N HCl in dioxane. The resulting solution is stirred at room temperature for 1 h. The reaction solution is then treated with the steps generally known to those of ordinary skill in the art to afford [H₃NR¹]⁺ XYa.

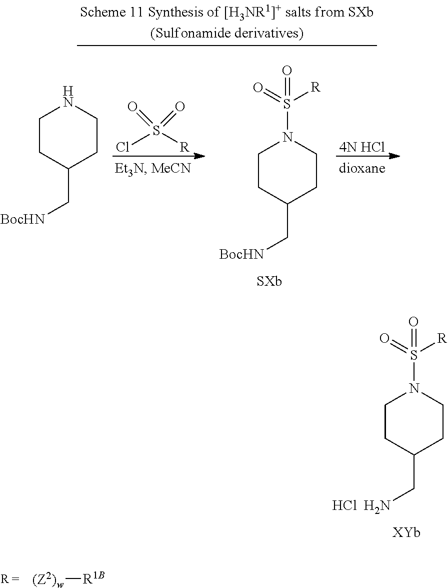

To a solution of tert-butyl (piperidin-4-ylmethyl)carbamate (1.0 equiv.) in a suitable solvent is added R-substituted sulfonyl chloride, wherein R═(Z²)_{w}—R^{1B} (1.1 equiv.), and triethylamine. The resulting mixture is stirred at room temperature for 4 h. The reaction solution treated with the steps generally known to those of ordinary skill in the art to afford SXb. Then, SXb is treated with 4 N HCl in dioxane. The resulting solution is stirred at room temperature for 1 h. The reaction solution is then treated with the steps generally known to those of ordinary skill in the art to afford [H₃NR¹]⁺ XYb.

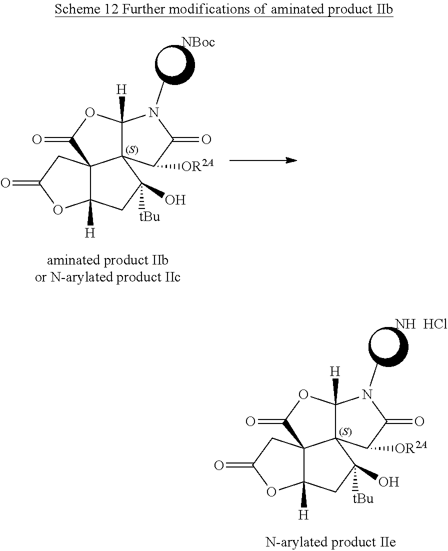

Aminated product IIb or N-arylated product IIc having the indicated structure (i.e., when R¹ is Boc-protected, 1 equiv.) is dissolved in a solution of HCl (2-4 N) in an appropriate solvent (MeOH or dioxane). The reaction is stirred at room temperature until complete conversion (1-24 h). The volatiles are removed, and the resulting N-arylated product IIe can be crystallized as HCl salt. FIG. 1B is an illustration of Scheme 12.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Example 1.0: Example Bilobalide Analogues

Provided herein are bilobalide analogues synthesized according to the methods in the present disclosure. The example bilobalide analogues and side chains (i.e., X, R¹, R², R³, R⁴, R⁵, R⁶ and R⁷) thereof below are provided for illustration purposes only and should not be construed as an exhaustive list of all possible bilobalide analogues. It shall be understood that terms such as “benzene” and pyrazine”, when used in a chart or table herein, are interchangeable with the terms such as “phenyl” and “pyrazinyl”, and refer to functional groups that may be optionally substituted.

In some embodiments, R² is OH or O(C═O)R^{2B}, wherein R^{2B} is —C₆H₅ or —CH₃, R₃ is tert-butyl, bond Y1 between R⁴ and R⁵ is a single bond, R⁴ is OH, R⁵ is H, X is —NR¹—, and R¹ is -(L¹)ᵤ-(Z¹)ᵥ; wherein L¹, Z¹, u, and v correspond as shown in Table 1a and 1b:

TABLE 1a
List of example R¹ groups where R¹ is —(L¹)ᵤ—(Z¹)ᵥ and Z¹ is substituted with R^{1C}
Two dashed lines “--” denotes that the group is absent.

| | | | | | | | R/ |
|---|---|---|---|---|---|---|---|
| R¹ | L¹ | u | Z¹ | v | R^{1C} | R* | R′ |
| 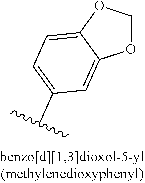 [SMILES: *c1ccc2c(c1)OCO2] | -- | 0 | piperonyl | 1 | -- | -- | -- |
| 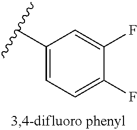 [SMILES: *c1ccc(F)c(F)c1] | -- | 0 | phenyl | 1 | —F | -- | -- |
| 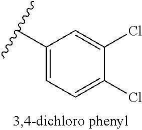 [SMILES: *c1ccc(Cl)c(Cl)c1] | -- | 0 | phenyl | 1 | —Cl | -- | -- |
| 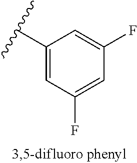 [SMILES: *c1cc(F)cc(F)c1] | -- | 0 | phenyl | 1 | —F | -- | -- |
| 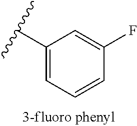 [SMILES: *c1cccc(F)c1] | -- | 0 | phenyl | 1 | —F | -- | -- |
| 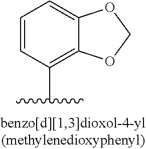 [SMILES: *c1cccc2c1OCO2] | -- | 0 | piperonyl | 1 | -- | -- | -- |
| 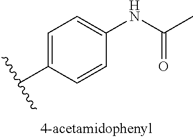 [SMILES: *c1ccc(NC(C)=O)cc1] | -- | 0 | phenyl | 1 | NRR* | —(C═O) CH₃ | H |
| 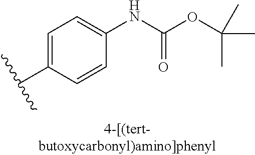 [SMILES: *c1ccc(NC(=O)OC(C)(C)C)cc1] | -- | 0 | phenyl | 1 | NRR* | —(C═O) OC(CH₃)₃ | H |
| 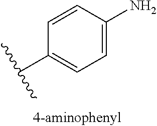 [SMILES: *c1ccc(N)cc1] | -- | 0 | phenyl | 1 | NRR* | H | H |
| 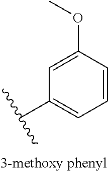 [SMILES: *c1cccc(OC)c1] | -- | 0 | phenyl | 1 | OR* | —CH₃ | -- |
| 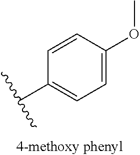 [SMILES: *c1ccc(OC)cc1] | -- | 0 | phenyl | 1 | OR* | —CH₃ | -- |
| 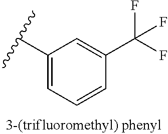 [SMILES: *c1cccc(C(F)(F)F)c1] | -- | 0 | phenyl | 1 | —CF3 | -- | -- |
| 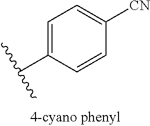 [SMILES: *c1ccc(C#N)cc1] | -- | 0 | phenyl | 1 | R* | —CN | -- |
| 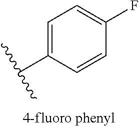 [SMILES: *c1ccc(F)cc1] | -- | 0 | phenyl | 1 | R* | F | -- |
| 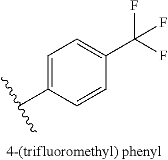 [SMILES: *c1ccc(C(F)(F)F)cc1] | -- | 0 | phenyl | 1 | R* | CF₃ | -- |
| 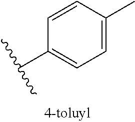 [SMILES: *c1ccc(C)cc1] | -- | 0 | phenyl | 1 | R* | CH₃ | -- |
| 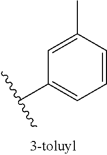 [SMILES: *c1cccc(C)c1] | -- | 0 | phenyl | 1 | R* | CH₃ | -- |
| 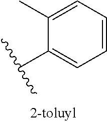 [SMILES: *c1ccccc1C] | -- | 0 | phenyl | 1 | R* | CH₃ | -- |
| 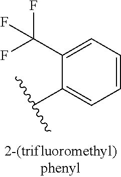 [SMILES: *c1ccccc1C(F)(F)F] | -- | 0 | phenyl | 1 | R* | CF₃ | -- |
| 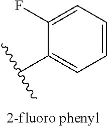 [SMILES: *c1ccccc1F] | -- | 0 | phenyl | 1 | —F | -- | -- |
| 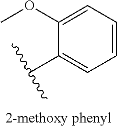 [SMILES: *c1ccccc1OC] | -- | 0 | phenyl | 1 | R* | OCH₃ | -- |
| 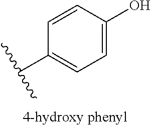 [SMILES: *c1ccc(O)cc1] | -- | 0 | phenyl | 1 | R* | OH | -- |
| 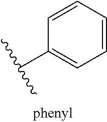 [SMILES: *c1ccccc1] | -- | 0 | phenyl | 1 | -- | -- | -- |
| 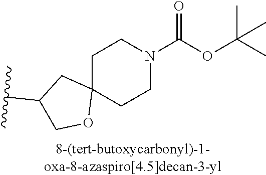 [SMILES: *C1COC2(CCN(C(=O)OC(C)(C)C)CC2)C1] | -- | 0 | 1-oxa-8- azaspiro[4.5] decan-3-yl | 1 | R* | (C═O)OC (CH₃)₃ | -- |
| 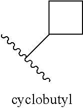 [SMILES: *C1CCC1] | -- | 0 | cyclobutanyl | 1 | -- | -- | -- |
| 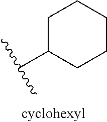 [SMILES: *C1CCCCC1] | -- | 0 | cyclohexanyl | 1 | -- | -- | -- |
| 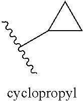 [SMILES: *C1CC1] | -- | 0 | cyclopentanyl | 1 | -- | -- | -- |
| 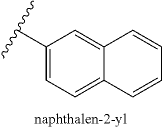 [SMILES: *c1ccc2ccccc2c1] | -- | 0 | napthalenyl | 1 | OR* | CH₃ | -- |
| 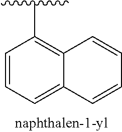 [SMILES: *c1cccc2ccccc12] | -- | 0 | napthalenyl | 1 | OR* | CH₃ | -- |
| 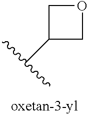 [SMILES: *C1COC1] | -- | 0 | oxetanyl | 1 | -- | -- | -- |
| 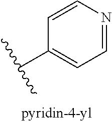 [SMILES: *c1ccncc1] | -- | 0 | pyridinyl | 1 | -- | -- | -- |
| 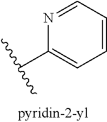 [SMILES: *c1ccccn1] | -- | 0 | pyridinyl | 1 | -- | -- | -- |
| 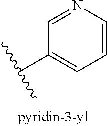 [SMILES: *c1cccnc1] | -- | 0 | pyridinyl | 1 | -- | -- | -- |
| 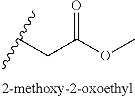 [SMILES: *CC(=O)OC] | CH₂(C═O) OCH₃ | 1 | H | 0 | -- | -- | -- |
| 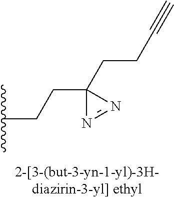 [SMILES: *CCC1(CCC#C)N=N1] | CH₂CH₂C (N═N) H₂CH₂CH₂CCH | 1 | -- | 0 | -- | -- | -- |
| methyl | CH₃ | 1 | -- | 0 | -- | -- | -- |
| NH₂ | NH₂ | 1 | -- | 0 | -- | -- | -- |
| 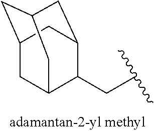 [SMILES: *CC1C2CC3CC(C2)CC1C3] | CH₂ | 1 | adamantanyl | 1 | -- | -- | -- |
| 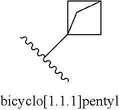 [SMILES: *C12CC(C1)C2] | CH₂ | 1 | bicyclo[1.1.1] pentyl | 1 | -- | -- | -- |
| 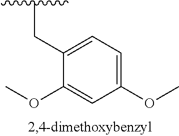 | CH₂ | 1 | phenyl | 1 | OR* | CH₃ | -- |
| 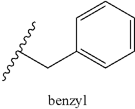 [SMILES: *Cc1ccccc1] | CH₂ | 1 | phenyl | 1 | -- | -- | -- |
| 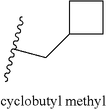 [SMILES: *CC1CCC1] | CH₂ | 1 | cyclobutanyl | 1 | -- | -- | -- |
| 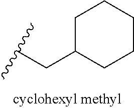 [SMILES: *CC1CCCCC1] | CH₂ | 1 | cyclohexanyl | 1 | -- | -- | -- |
| 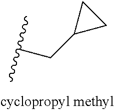 [SMILES: *CC1CC1] | CH₂ | 1 | cyclopropanyl | 1 | -- | -- | -- |
| 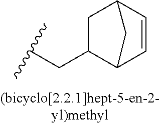 [SMILES: *CC1CC2C=CC1C2] | CH₂ | 1 | norbornenyl | 1 | -- | -- | -- |
| 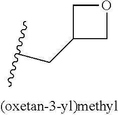 [SMILES: *CC1COC1] | CH₂ | 1 | oxetanyl | 1 | -- | -- | -- |
| 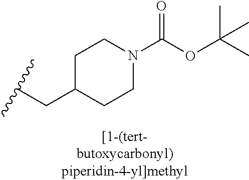 [SMILES: *CC1CCN(C(=O)OC(C)(C)C)CC1] | CH₂ | 1 | piperidinyl | 1 | R* | (C═O)OC (CH₃)₃ | -- |
| 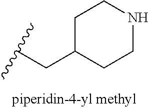 [SMILES: *CC1CCNCC1] | CH₂ | 1 | piperidinyl | 1 | R* | H | -- |
| 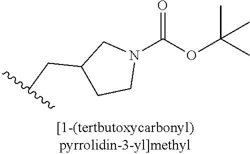 [SMILES: *CC1CCN(C(=O)OC(C)(C)C)C1] | CH₂ | 1 | pyrrolidinyl | 1 | R* | (C═O)OC (CH₃)₃ | -- |
| 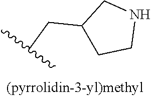 [SMILES: *CC1CCNC1] | CH₂ | 1 | pyrrolidinyl | 1 | R* | H | -- |
| 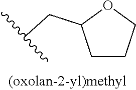 [SMILES: *CC1CCCO1] | CH₂ | 1 | tetrahydrofuranyl | 1 | -- | -- | -- |
| 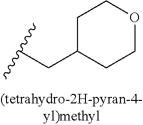 [SMILES: *CC1CCOCC1] | CH₂ | 1 | tetrahydropyranyl | 1 | -- | -- | -- |
| 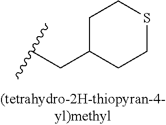 [SMILES: *CC1CCSCC1] | CH₂ | 1 | tetrahydrothio- pyranyl | 1 | -- | -- | -- |
| 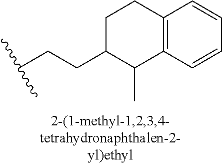 [SMILES: *CCC1CCc2ccccc2C1C] | CH₂CH₂ | 1 | 1-methyl-1,2,3,4- tetrahydro- naphthalen-2-y | 1 | -- | -- | -- |
| 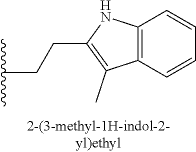 [SMILES: *CCc1[nH]c2ccccc2c1C] | CH₂CH₂ | 1 | indolyl | 1 | R* | CH₃ | -- |
| 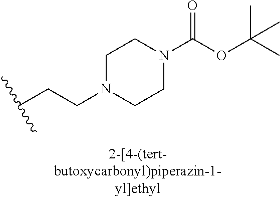 [SMILES: *CCN1CCN(C(=O)OC(C)(C)C)CC1] | CH₂CH₂ | 1 | piperazinyl | 1 | R* | (C═O)OC (CH₃)₃ | -- |
| 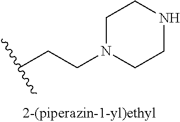 [SMILES: *CCN1CCNCC1] | CH₂CH₂ | 1 | piperazinyl | 1 | R* | H | -- |
| 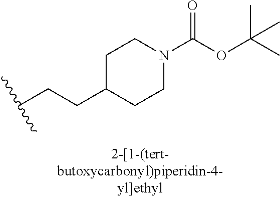 [SMILES: *CCC1CCN(C(=O)OC(C)(C)C)CC1] | CH₂CH₂ | 1 | piperidinyl | 1 | R* | (C═O)OC (CH₃)₃ | -- |
| 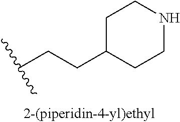 [SMILES: *CCC1CCNCC1] | CH₂CH₂ | 1 | piperidinyl | 1 | R* | H | -- |
| 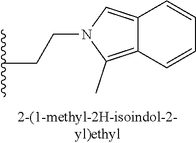 [SMILES: *CCn1cc2ccccc2c1C] | CH₂CH₂ | 1 | 1-methyl-2H- isoindol-2-yl | 1 | -- | -- | -- |
| 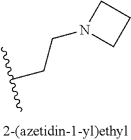 [SMILES: *CCN1CCC1] | CH₂CH₂ | 1 | azetidinyl | 1 | -- | -- | -- |
| 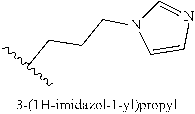 [SMILES: *CCCn1ccnc1] | CH₂CH₂CH₂ | 1 | imidazol | 1 | -- | -- | -- |
| 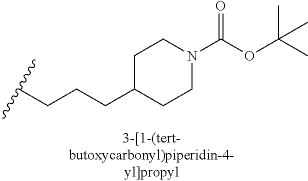 [SMILES: *CCCC1CCN(C(=O)OC(C)(C)C)CC1] | CH₂CH₂CH₂ | 1 | piperidinyl | 1 | R* | (C═O)OC (CH₃)₃ | -- |
| 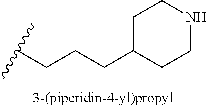 [SMILES: *CCCC1CCNCC1] | CH₂CH₂CH₂ | 1 | piperidinyl | 1 | R* | H | -- |
| 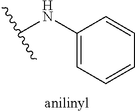 [SMILES: *Nc1ccccc1] | NH | 1 | phenyl | 1 | -- | -- | -- |
| 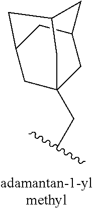 [SMILES: *CC12CC3CC(CC(C3)C1)C2] | CH₂ | 1 | adamantanyl | 1 | | -- | -- |
| 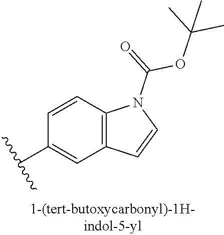 [SMILES: *c1ccc2c(ccn2C(=O)OC(C)(C)C)c1] | -- | 1 | indolyl | 1 | R* | (C═O)OC (CH₃)₃ | |
| 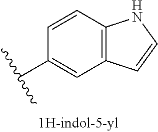 [SMILES: *c1ccc2[nH]ccc2c1] | -- | 0 | indolyl | 1 | R* | H | -- |
| 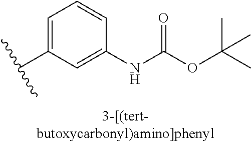 [SMILES: *c1cccc(NC(=O)OC(C)(C)C)c1] | -- | 0 | phenyl | 1 | NRR* | (C═O)OC (CH₃)₃ | H |
| 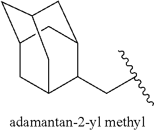 [SMILES: *CC1C2CC3CC(C2)CC1C3] | CH₂ | 1 | adamantanyl | 1 | -- | -- | -- |
| 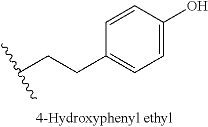 [SMILES: *CCc1ccc(O)cc1] | CH₂CH₂ | 1 | phenyl | 1 | OR* | H | -- |
| 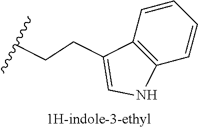 [SMILES: *CCc1c[nH]c2ccccc12] | CH₂CH₂ | 1 | indolyl | 1 | R* | H | -- |
| 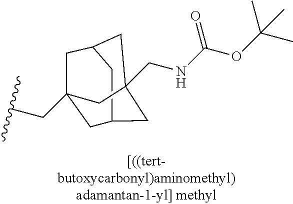 [SMILES: *CC12CC3CC(C1)CC(CNC(=O)OC(C)(C)C)(C3)C2] | CH₂ | 1 | adamantanyl | 1 | R* | NH(C═O) OC(CH₃)₃ | -- |
| 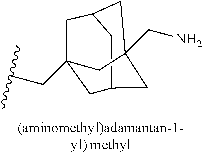 [SMILES: *CC12CC3CC(C1)CC(CN)(C3)C2] | CH₂ | 1 | adamantanyl | 1 | NRR* | H | -- |
| 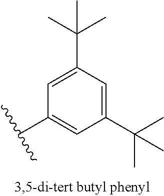 [SMILES: *c1cc(C(C)(C)C)cc(C(C)(C)C)c1] | -- | 0 | phenyl | 1 | R* | C(CH₃)₃ | -- |
| 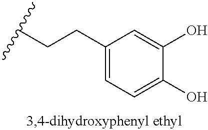 [SMILES: *CCc1ccc(O)c(O)c1] | -- | 0 | phenyl | 1 | OR* | H | -- |
| 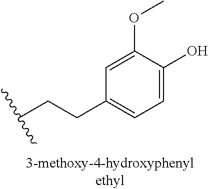 [SMILES: *CCc1ccc(O)c(OC)c1] | CH₂CH₂ | 1 | phenyl | 1 | OR* | H or CH₃ | -- |
| 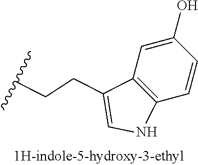 [SMILES: *CCc1c[nH]c2ccc(O)cc12] | CH₂CH₂ | 1 | indolyl | 1 | OR* | H | -- |
| 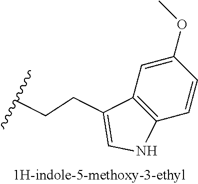 [SMILES: *CCc1c[nH]c2ccc(OC)cc12] | CH₂CH₂ | 1 | indolyl | 1 | OR* | CH₃ | -- |
| 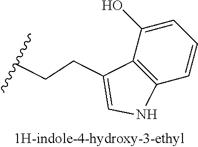 [SMILES: *CCc1c[nH]c2cccc(O)c12] | CH₂CH₂ | 1 | indolyl | 1 | OR* | H | -- |
| 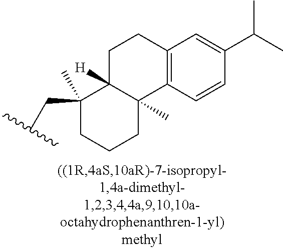 [SMILES: *C[C@]1(C)CCC[C@]2(C)c3ccc(C(C)C)cc3CC[C@@H]12] | CH₂ | 1 | 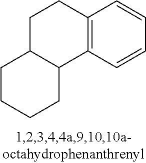 | 1 | R* | CH₃ or isopropyl | -- |
| 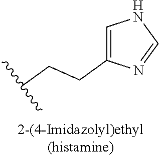 [SMILES: *CCc1c[nH]cn1] | CH₂CH₂ | 1 | imidazol | 1 | -- | -- | -- |
| 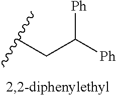 [SMILES: *CC(c1ccccc1)c1ccccc1] | CH₂CH₂ | 1 | Phenyl (C6H5) | 1 | phenyl | -- | -- |
| 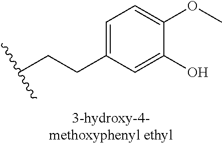 [SMILES: *CCc1ccc(OC)c(O)c1] | CH₂CH₂ | 1 | Phenyl (C6H3) | 1 | OR* | H or CH₃ | -- |
| 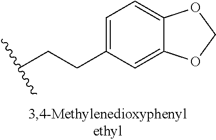 [SMILES: *CCc1ccc2c(c1)OCO2] | CH₂CH₂ | 1 | piperonyl | 1 | -- | -- | -- |
| 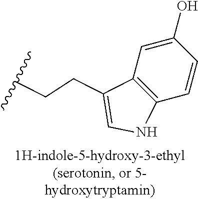 [SMILES: *CCc1c[nH]c2ccc(O)cc12] | CH₂CH₂ | 1 | indolyl | 1 | OR* | H | -- |
| 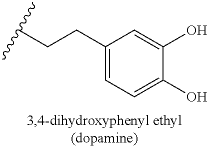 [SMILES: *CCc1ccc(O)c(O)c1] | CH₂CH₂ | 1 | phenyl | 1 | OR* | H | -- |
| 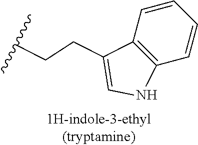 [SMILES: *CCc1c[nH]c2ccccc12] | CH₂CH₂ | 1 | indolyl | 1 | R* | H | -- |
| 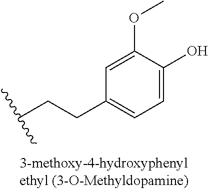 [SMILES: *CCc1ccc(O)c(OC)c1] | CH₂CH₂ | 1 | phenyl | 1 | OR* | H or CH₃ | -- |

TABLE 1b
List of example R¹ groups where R¹ is —(L¹)ᵤ—(Z¹)ᵥ and Z¹ is substituted with R^{1A},
wherein R^{1A} is —(L²)ₘ—(Z²)_{w}—R^{1B}
Two dashed lines “--” denotes that the group is absent.

| | | | | | | | | | | | R/ |
|---|---|---|---|---|---|---|---|---|---|---|---|
| R1 | L¹ | u | Z¹ | v | L² | m | Z² | w | R^{1B} | R* | R′ |
| 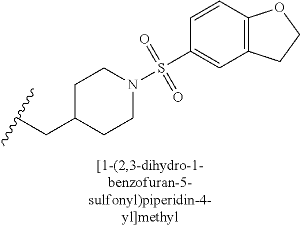 [SMILES: *CC1CCN(S(=O)(=O)c2ccc3c(c2)CCO3)CC1] | CH₂ | 1 | pi- per- idin- yl | 1 | SO₂ | 1 | dihydro- benzo- furan | 1 | -- | -- | -- |
| 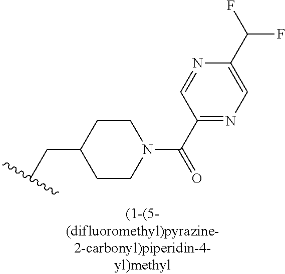 [SMILES: *CC1CCN(C(=O)c2cnc(C(F)F)cn2)CC1] | CH₂ | 1 | pi- per- idin- yl | 1 | C═O | 1 | 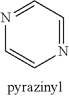 | 1 | R* | —CF₂ | -- |
| 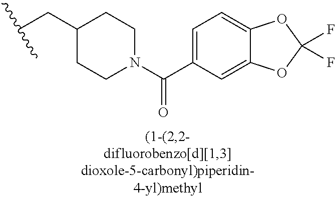 [SMILES: *CC1CCN(C(=O)c2ccc3c(c2)OC(F)(F)O3)CC1] | CH₂ | 1 | pi- per- idin- yl | 1 | C═O | 1 | piperonyl | 1 | F | -- | -- |
| 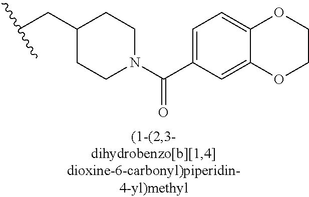 [SMILES: *CC1CCN(C(=O)c2ccc3c(c2)OCCO3)CC1] | CH₂ | 1 | pi- per- idin- yl | 1 | C═O | 1 | dihydro- benzo- dioxine | 1 | -- | -- | -- |
| 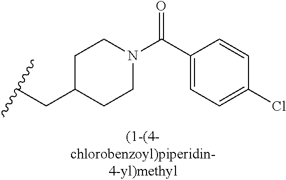 [SMILES: *CC1CCN(C(=O)c2ccc(Cl)cc2)CC1] | CH₂ | 1 | pi- per- idin- yl | 1 | C═O | 1 | phenyl | 1 | Cl | -- | -- |
| 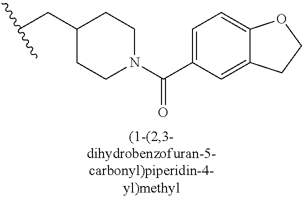 [SMILES: *CC1CCN(C(=O)c2ccc3c(c2)CCO3)CC1] | CH₂ | 1 | pi- per- idin- yl | 1 | C═O | 1 | dihydro- benzo- furan | 1 | -- | -- | -- |
| 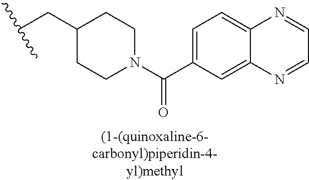 [SMILES: *CC1CCN(C(=O)c2ccc3nccnc3c2)CC1] | CH₂ | 1 | pi- per- idin- yl | 1 | C═O | 1 | 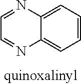 | 1 | -- | -- | -- |
| 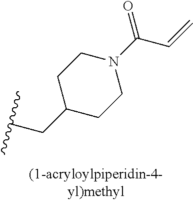 [SMILES: *CC1CCN(C(=O)C=C)CC1] | CH₂ | 1 | pi- per- idin- yl | 1 | C═O | 1 | —CH═CH₂ | 1 | -- | -- | -- |
| 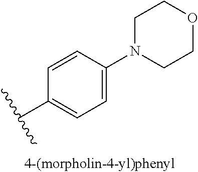 [SMILES: *c1ccc(N2CCOCC2)cc1] | -- | 0 | phe- nyl | 1 | -- | 0 | morpho- linyl | 1 | -- | -- | -- |

According to the methods in the present disclosure, the X group on a bilobalide analogue can be modified to yield lactam rings of various sizes (5-7 membered). Examples are provided in Table 1c for illustration purposes only and should not be construed as an exhaustive list of all possible bilobalide analogues.

TABLE 1c
Examples of X groups

| X | Example structure | R¹ |
|---|---|---|
| —O— | 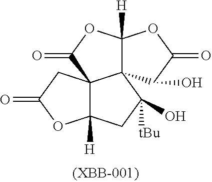 [SMILES: CC(C)(C)[C@]1(O)C[C@@H]2OC(=O)C[C@@]23C(=O)O[C@@H]2OC(=O)[C@H](O)[C@]213] | absent |
| —NR¹— | 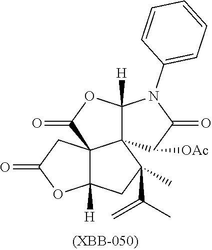 [SMILES: C=C(C)[C@@]1(C)C[C@@H]2OC(=O)C[C@@]23C(=O)O[C@@H]2N(c4ccccc4)C(=O)[C@H](OC(C)=O)[C@]213] | Phenyl |
| —NH—NH— | 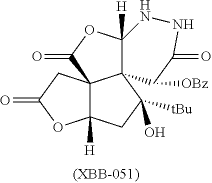 | H |
| —N═CR¹—NH— | 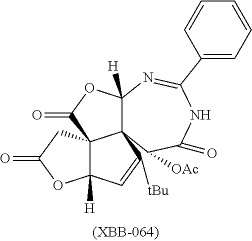 [SMILES: CC(=O)O[C@H]1C(=O)NC(c2ccccc2)=N[C@H]2OC(=O)[C@]34CC(=O)O[C@H]3C=C(C(C)(C)C)[C@@]214] | phenyl |
| —NR¹—NH— | 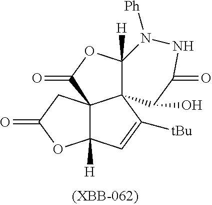 [SMILES: CC(C)(C)C1=C[C@@H]2OC(=O)C[C@@]23C(=O)O[C@@H]2N(c4ccccc4)NC(=O)[C@H](O)[C@@]123] | Phenyl |

Table 1d provides a list of example bilobalide analogs prepared according to the methods of the present disclosure. The examples below are provided for illustration purposes only and should not be construed as an exhaustive list of all possible bilobalide analogues. In some embodiments, the compounds of Table 1d that are described as salts, e.g., HCl or chloride salts, are in their free base form. In some embodiments, the compounds of Table 1d that are described as free bases or neutral, e.g., compounds with —NH₂ groups, are in their salt forms, e.g., —NH₂·HCl.

TABLE 1d
Structures of Compounds

| # | Structure | X | R¹ |
|---|---|---|---|
| XBB-001 (Bilobalide) | 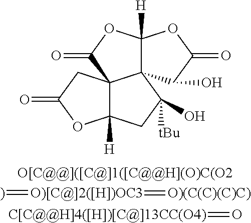 | O | — |
| XBB-002 | 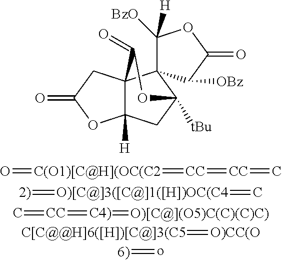 | O | — |
| XBB-003 | 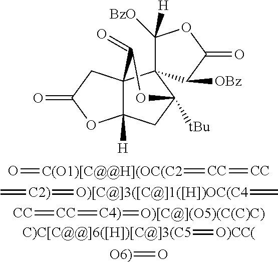 | O | — |
| XBB-004 | 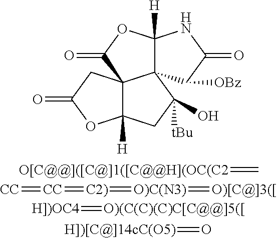 | N | H |
| XBB-005 | 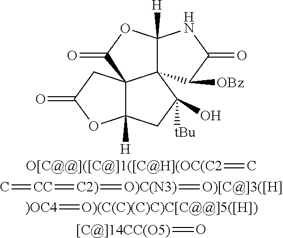 | N | H |
| XBB-006 | 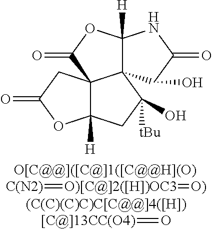 | N | H |
| XBB-007 | 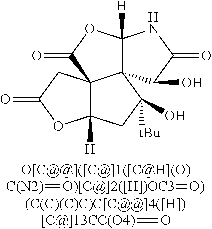 | N | H |
| XBB-008 | 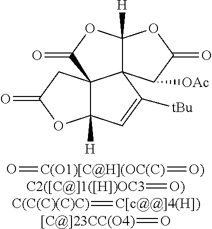 | O | — |
| XBB-009 | 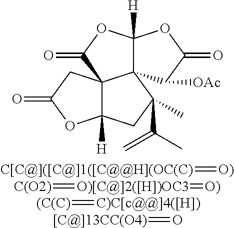 | O | — |
| XBB-010 | 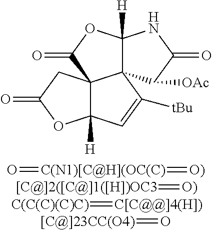 | N | H |
| XBB-011 | 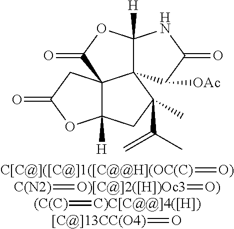 | N | H |
| XBB-012 | 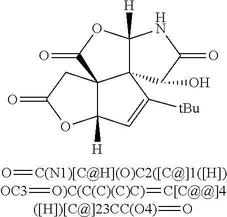 | N | H |
| XBB-013 | 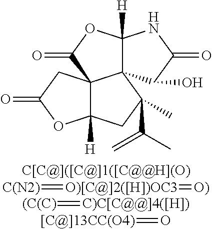 | N | H |
| XBB-014 | 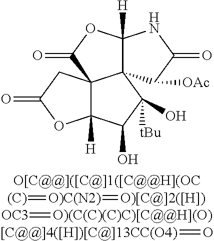 | N | H |
| XBB-015 | 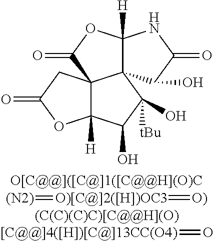 | N | H |
| XBB-016 | 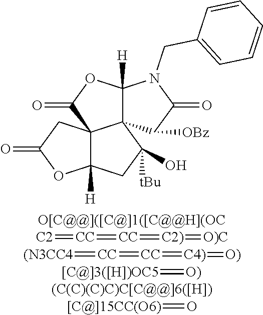 | N | 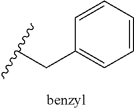 [SMILES: *Cc1ccccc1] |
| XBB-017 | 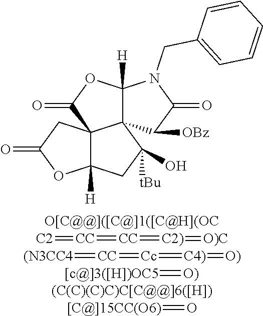 | N | 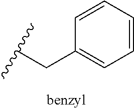 [SMILES: *Cc1ccccc1] |
| XBB-018 | 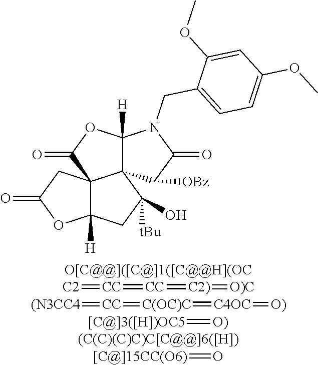 | N | 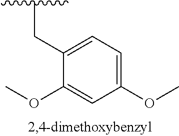 |
| XBB-019 | 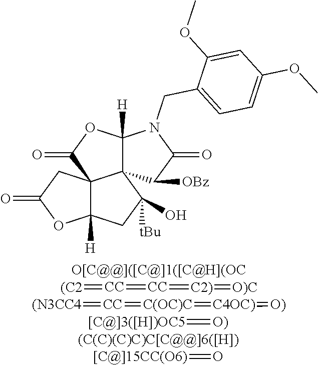 | N | 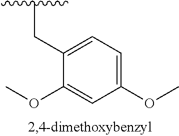 |
| XBB-020 | 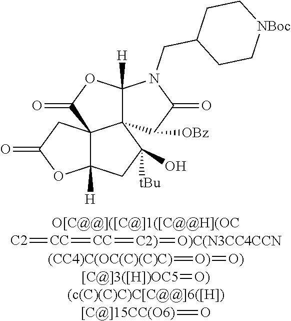 | N | 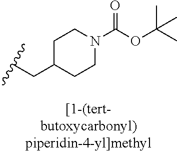 [SMILES: *CC1CCN(C(=O)OC(C)(C)C)CC1] |
| XBB-021 | 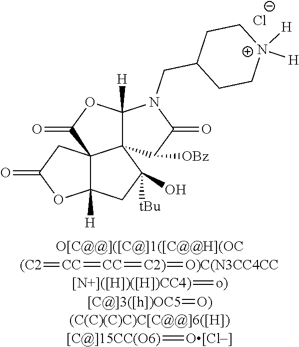 | N | 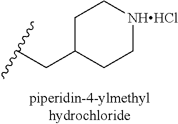 |
| XBB-022 | 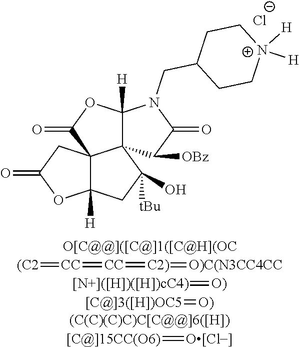 | N | 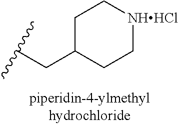 |
| XBB-023 | 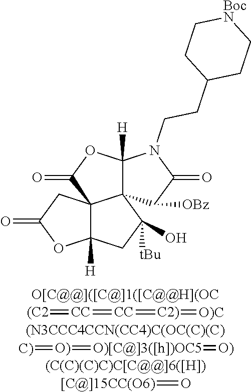 | N | 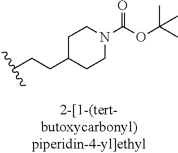 [SMILES: *CCC1CCN(C(=O)OC(C)(C)C)CC1] |
| XBB-024 | 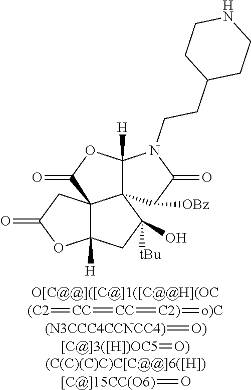 | N | 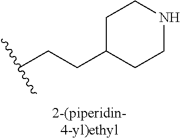 [SMILES: *CCC1CCNCC1] |
| XBB-025 | 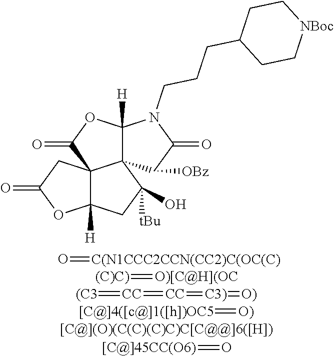 | N | 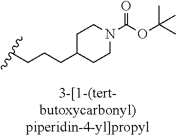 [SMILES: *CCCC1CCN(C(=O)OC(C)(C)C)CC1] |
| XBB-026 | 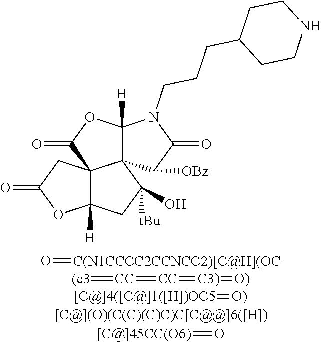 | N | 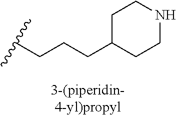 [SMILES: *CCCC1CCNCC1] |
| XBB-027 | 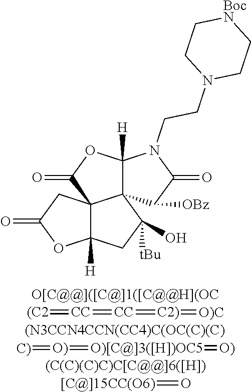 | N | 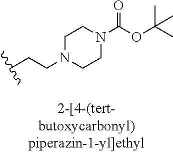 [SMILES: *CCN1CCN(C(=O)OC(C)(C)C)CC1] |
| XBB-028 | 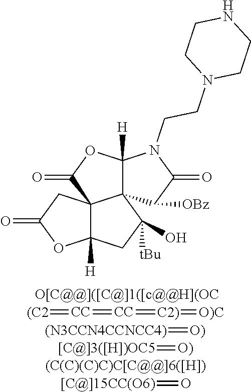 | N | 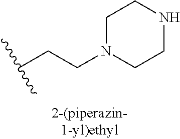 [SMILES: *CCN1CCNCC1] |
| XBB-029 | 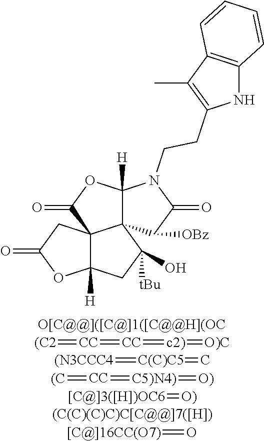 | N | 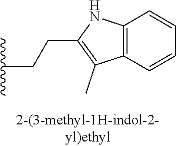 [SMILES: *CCc1[nH]c2ccccc2c1C] |
| XBB-030 | 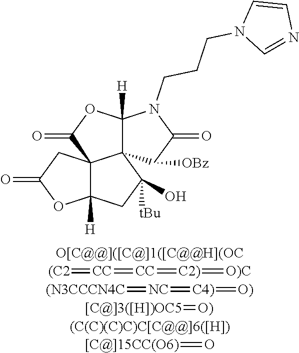 | N | 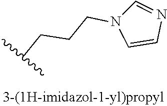 [SMILES: *CCCn1ccnc1] |
| XBB-031 | 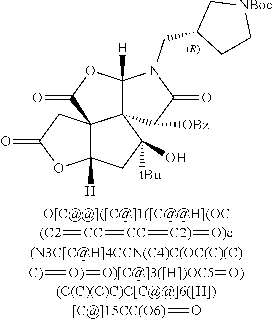 | N | 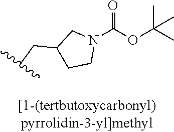 [SMILES: *CC1CCN(C(=O)OC(C)(C)C)C1] |
| XBB-032 | 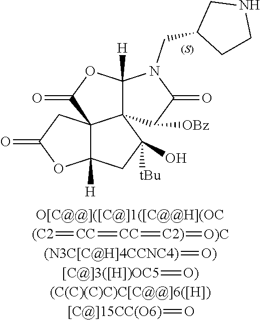 | N | 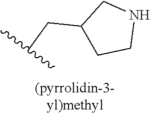 [SMILES: *CC1CCNC1] |
| XBB-033 | 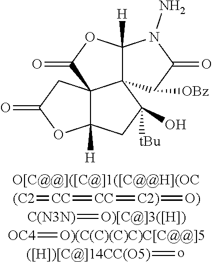 | N | 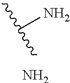 |
| XBB-034 | 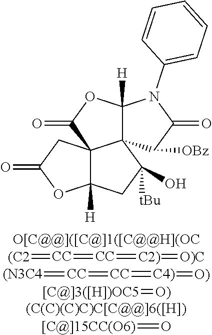 | N | 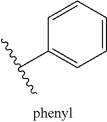 [SMILES: *c1ccccc1] |
| XBB-035 | 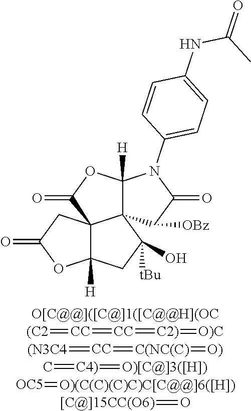 | N | 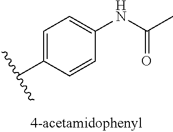 [SMILES: *c1ccc(NC(C)=O)cc1] |
| XBB-036 | 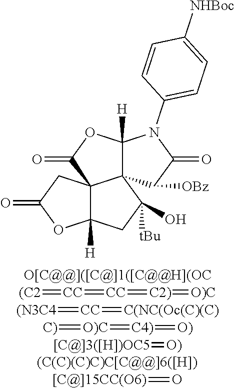 | N | 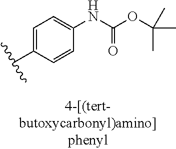 [SMILES: *c1ccc(NC(=O)OC(C)(C)C)cc1] |
| XBB-037 | 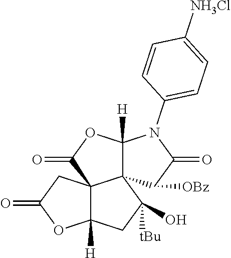 | N | 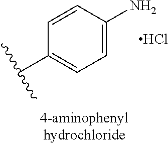 [SMILES: *c1ccc(N)cc1 ; Cl] |
| XBB-037′ | 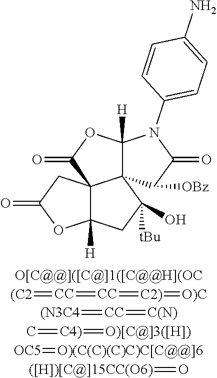 | N | 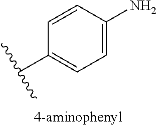 [SMILES: *c1ccc(N)cc1] |
| XBB-038 | 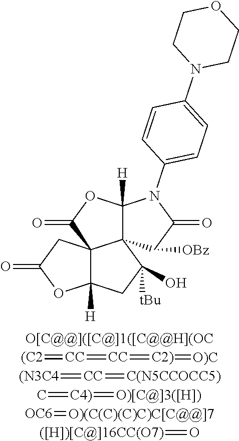 | N | 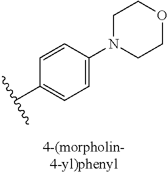 [SMILES: *c1ccc(N2CCOCC2)cc1] |
| XBB-039 | 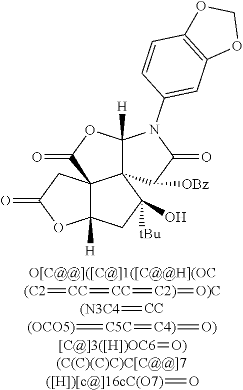 | N | 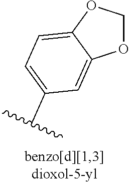 [SMILES: *c1ccc2c(c1)OCO2] |
| XBB-040 | 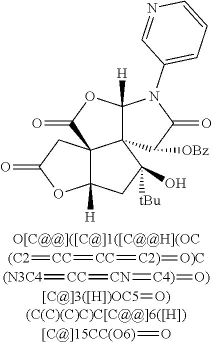 | N | 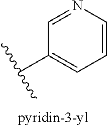 [SMILES: *c1cccnc1] |
| XBB-041 | 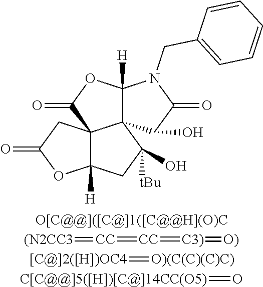 | N | 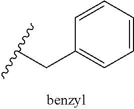 [SMILES: *Cc1ccccc1] |
| XBB-042 | 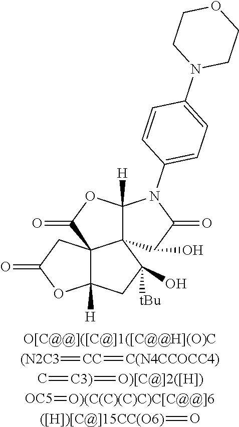 | N | 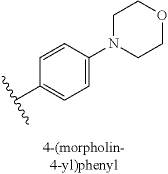 [SMILES: *c1ccc(N2CCOCC2)cc1] |
| XBB-043 | 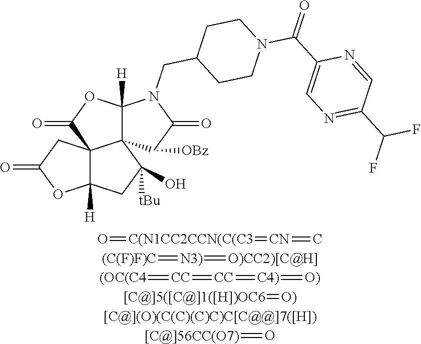 | N | 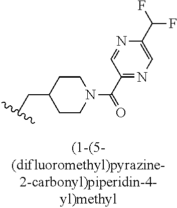 [SMILES: *CC1CCN(C(=O)c2cnc(C(F)F)cn2)CC1] |
| XBB-044 | 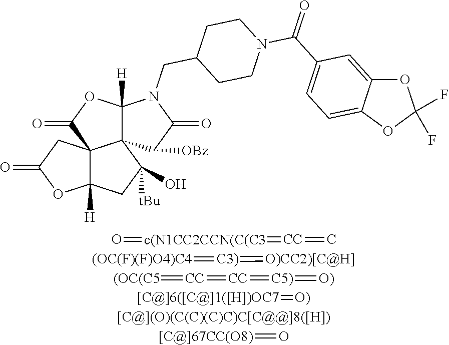 | N | 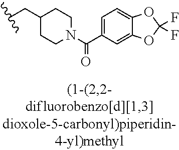 [SMILES: *CC1CCN(C(=O)c2ccc3c(c2)OC(F)(F)O3)CC1] |
| XBB-045 | 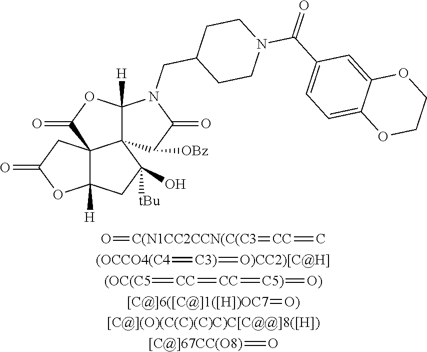 | N | 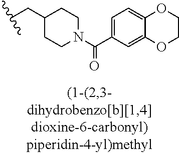 [SMILES: *CC1CCN(C(=O)c2ccc3c(c2)OCCO3)CC1] |
| XBB-046 | 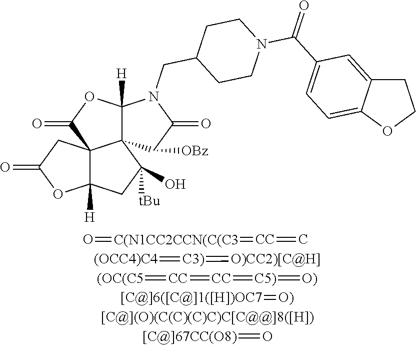 | N | 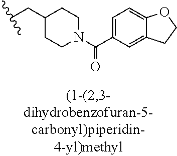 [SMILES: *CC1CCN(C(=O)c2ccc3c(c2)CCO3)CC1] |
| XBB-047 | 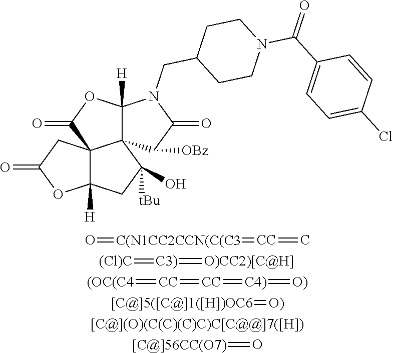 | N | 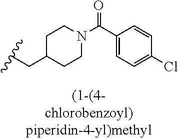 [SMILES: *CC1CCN(C(=O)c2ccc(Cl)cc2)CC1] |
| XBB-048 | 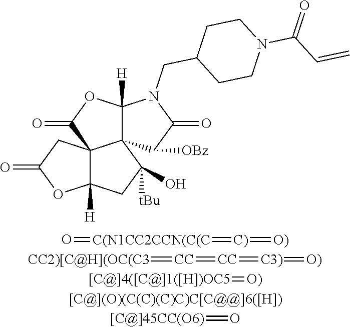 | N | 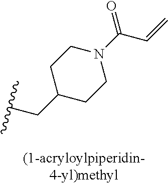 [SMILES: *CC1CCN(C(=O)C=C)CC1] |
| XBB-049 | 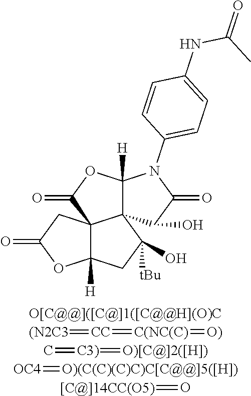 | N | 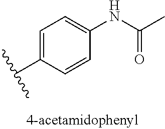 [SMILES: *c1ccc(NC(C)=O)cc1] |
| XBB-050 | 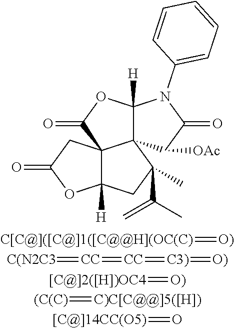 | N | phenyl |
| XBB-051 | 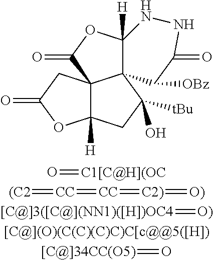 | NH—NH | — |
| XBB-052 | 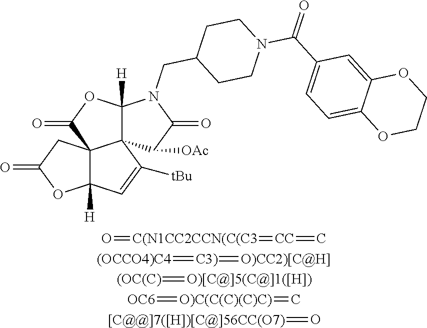 | N | 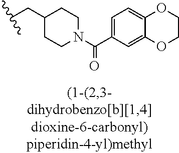 [SMILES: *CC1CCN(C(=O)c2ccc3c(c2)OCCO3)CC1] |
| XBB-053 | 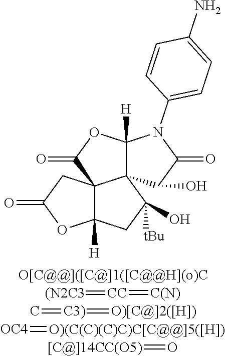 | N | 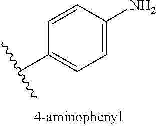 [SMILES: *c1ccc(N)cc1] |
| XBB-054 | 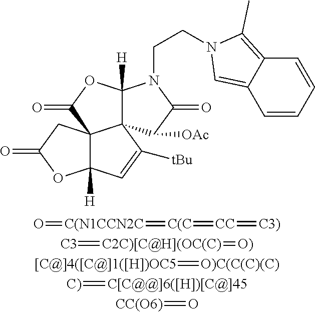 | N | 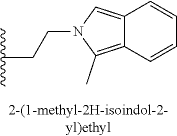 [SMILES: *CCn1cc2ccccc2c1C] |
| XBB-055 | [deleted | | |
| XBB-056 | 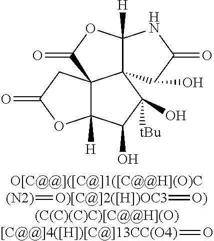 | N | H |
| XBB-057 | 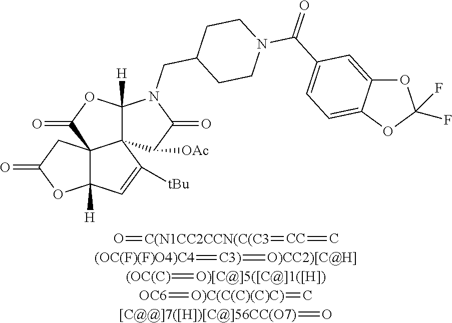 | N | 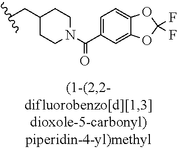 [SMILES: *CC1CCN(C(=O)c2ccc3c(c2)OC(F)(F)O3)CC1] |
| XBB-058 | 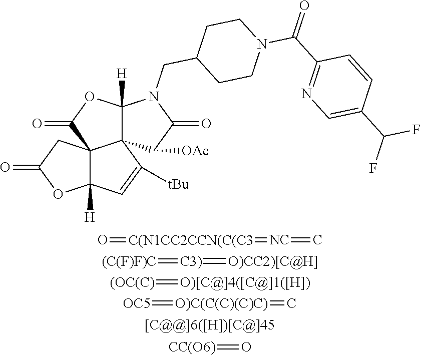 | N | 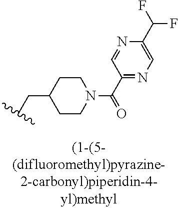 [SMILES: *CC1CCN(C(=O)c2cnc(C(F)F)cn2)CC1] |
| XBB-059 | 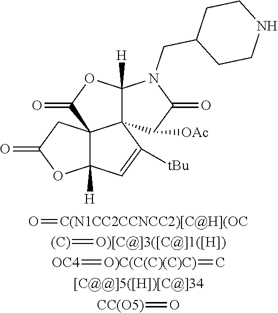 | N | 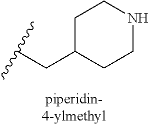 [SMILES: *CC1CCNCC1] |
| XBB-060 | 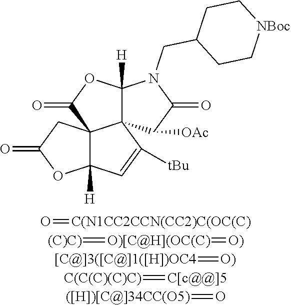 | N | 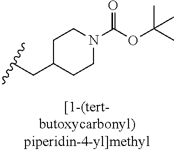 [SMILES: *CC1CCN(C(=O)OC(C)(C)C)CC1] |
| XBB-061 | 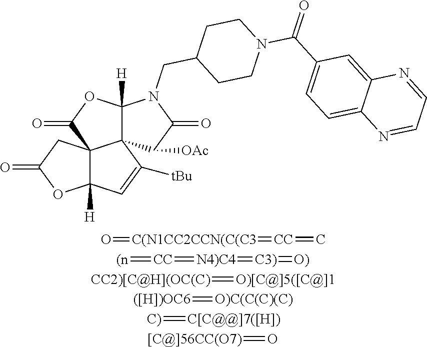 | N | 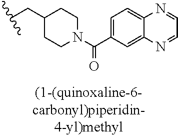 [SMILES: *CC1CCN(C(=O)c2ccc3nccnc3c2)CC1] |
| XBB-062 | 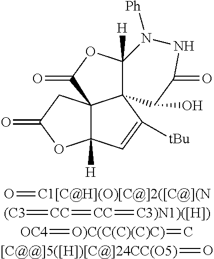 | NR1-—NH | 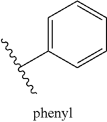 [SMILES: *c1ccccc1] |
| XBB-063 | 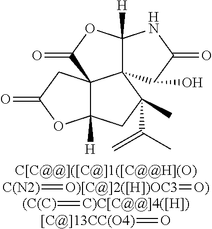 | N | H |
| XBB-064 | 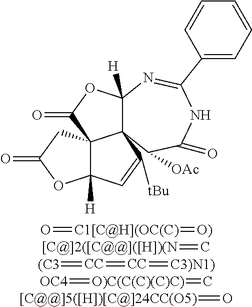 | N═CR1—NH | 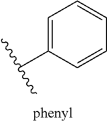 [SMILES: *c1ccccc1] |
| XBB-065 | 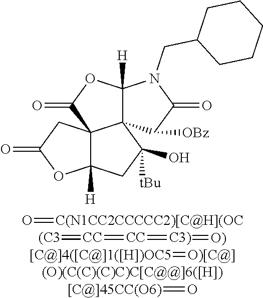 | N | 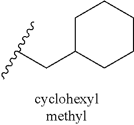 [SMILES: *CC1CCCCC1] |
| XBB-066 | 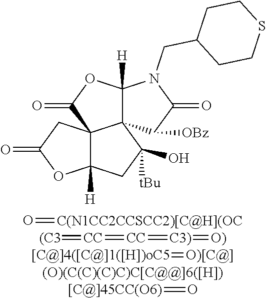 | N | 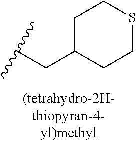 [SMILES: *CC1CCSCC1] |
| XBB-067 | 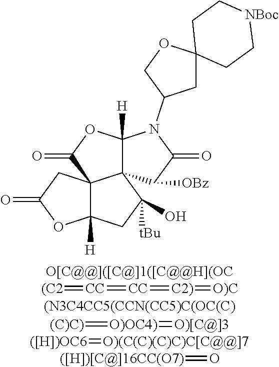 | N | 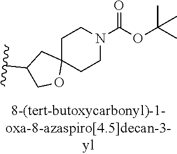 [SMILES: *C1COC2(CCN(C(=O)OC(C)(C)C)CC2)C1] |
| XBB-068 | 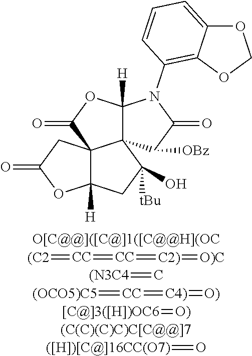 | N | 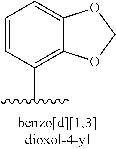 [SMILES: *c1cccc2c1OCO2] |
| XBB-069 | 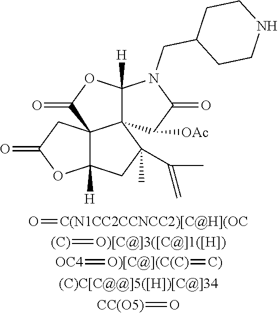 | N | 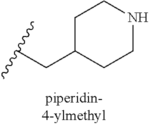 [SMILES: *CC1CCNCC1] |
| XBB-070 | 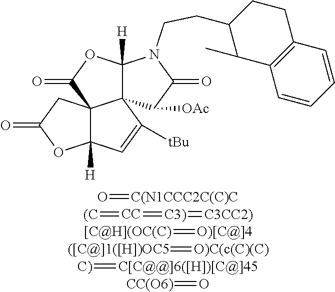 | N | 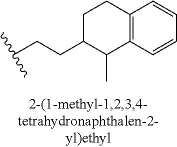 [SMILES: *CCC1CCc2ccccc2C1C] |
| XBB-071 | 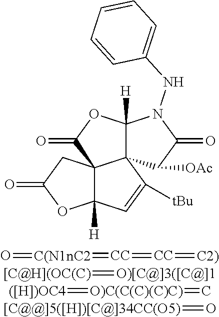 | N | 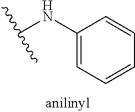 [SMILES: *Nc1ccccc1] |
| XBB-072 | 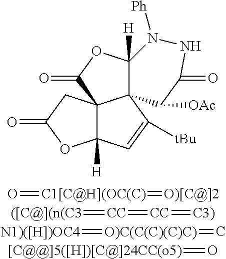 | NR1—NH | 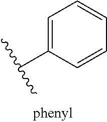 [SMILES: *c1ccccc1] |
| XBB-073 | 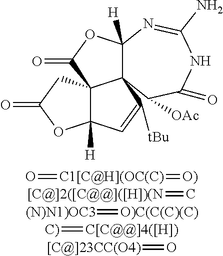 | N═CR1—NH | NH₂ |
| XBB-074 | 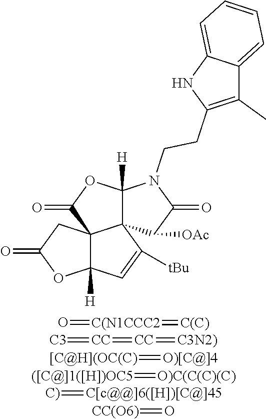 | N | 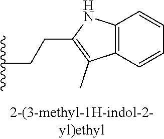 [SMILES: *CCc1[nH]c2ccccc2c1C] |
| XBB-075 | 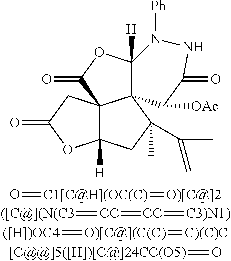 | NR1—NH | 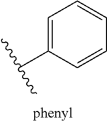 [SMILES: *c1ccccc1] |
| XBB-076 | 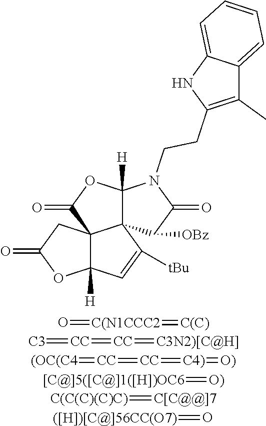 | N | 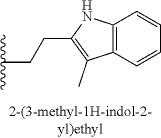 [SMILES: *CCc1[nH]c2ccccc2c1C] |
| XBB-077 | 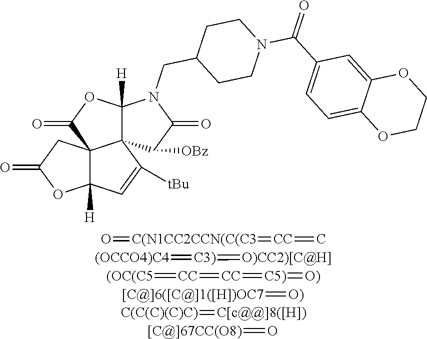 | N | 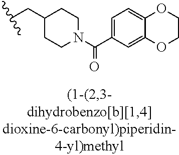 [SMILES: *CC1CCN(C(=O)c2ccc3c(c2)OCCO3)CC1] |
| DW168 | 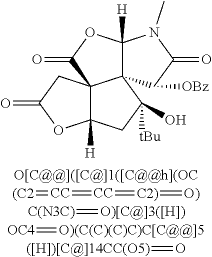 | N | methyl (CH₃) |
| DW189 | 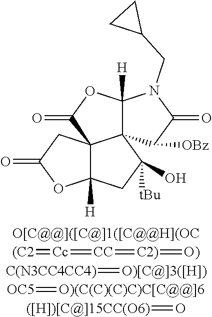 | N | 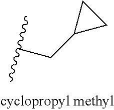 [SMILES: *CC1CC1] |
| DW184 | 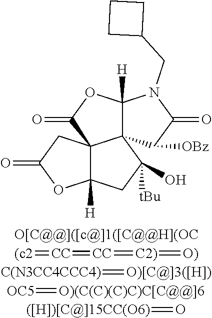 | N | 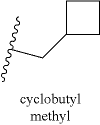 [SMILES: *CC1CCC1] |
| DW191 | 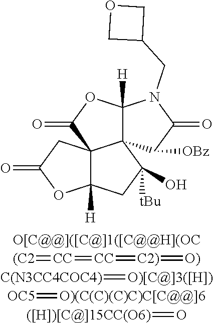 | N | 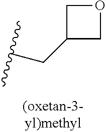 [SMILES: *CC1COC1] |
| P-5 | 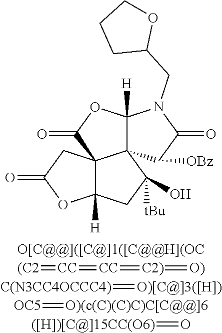 | N | 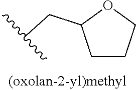 [SMILES: *CC1CCCO1] |
| P-3 | 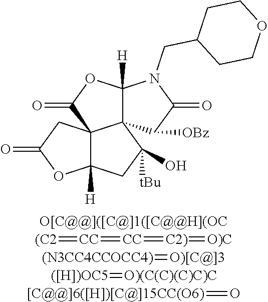 | N | 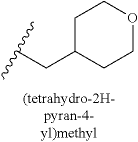 [SMILES: *CC1CCOCC1] |
| DW190 | 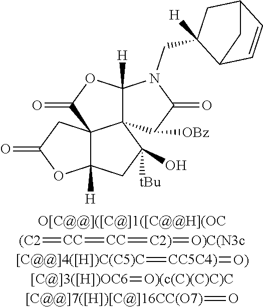 | N | 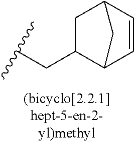 [SMILES: *CC1CC2C=CC1C2] |
| P-10 | 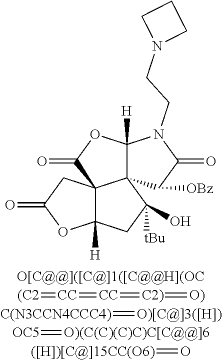 | N | 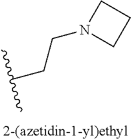 [SMILES: *CCN1CCC1] |
| DW163 | 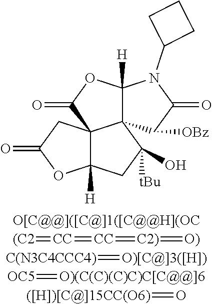 | N | 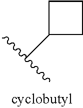 [SMILES: *C1CCC1] |
| DW182 | 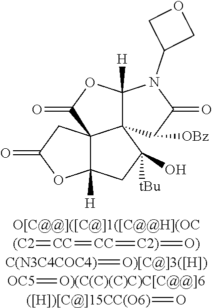 | N | 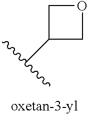 [SMILES: *C1COC1] |
| DW172 | 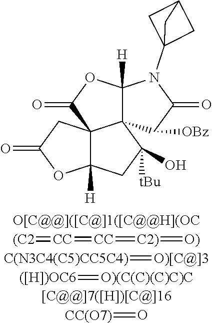 | N | 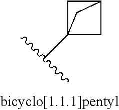 [SMILES: *C12CC(C1)C2] |
| JW081 | 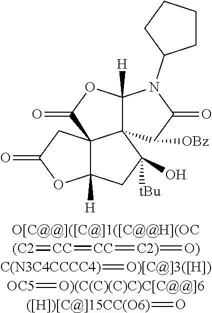 | N | 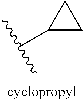 [SMILES: *C1CC1] |
| P-12 | 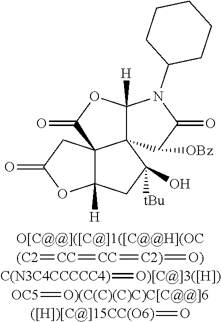 | N | 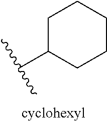 [SMILES: *C1CCCCC1] |
| DW192 | 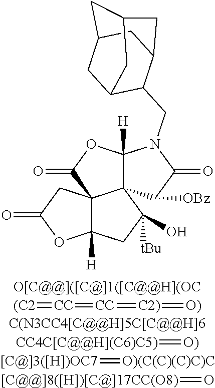 | N | 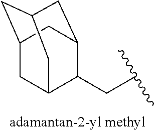 [SMILES: *CC1C2CC3CC(C2)CC1C3] |
| P-8 | 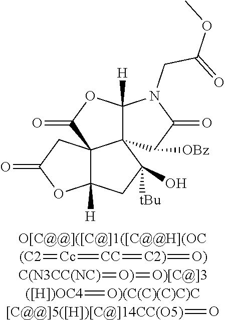 | N | 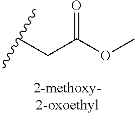 [SMILES: *CC(=O)OC] |
| JW107 | 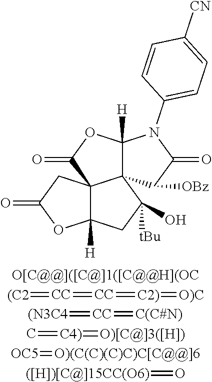 | N | 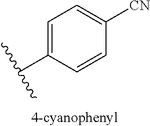 [SMILES: *c1ccc(C#N)cc1] |
| P-28 | 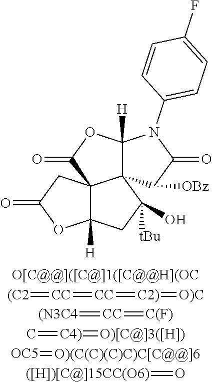 | N | 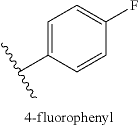 [SMILES: *c1ccc(F)cc1] |
| JW098 | 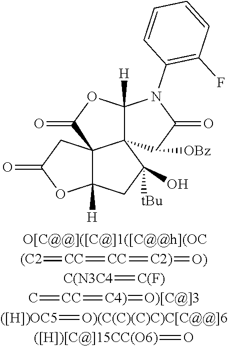 | N | 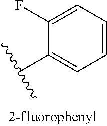 [SMILES: *c1ccccc1F] |
| JW099 | 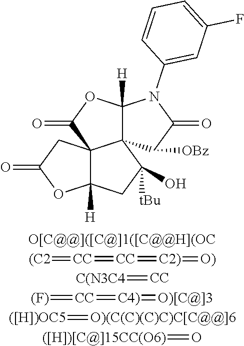 | N | 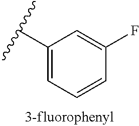 [SMILES: *c1cccc(F)c1] |
| P-19 | 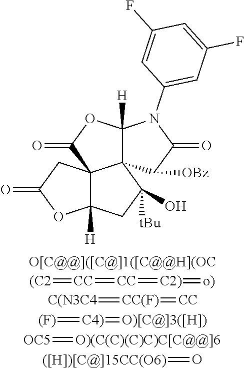 | N | 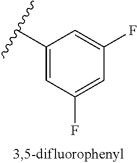 [SMILES: *c1cc(F)cc(F)c1] |
| P-21 | 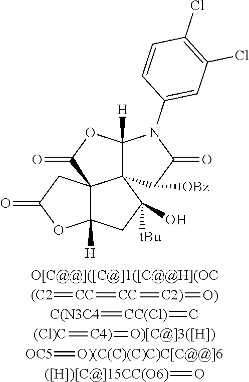 | N | 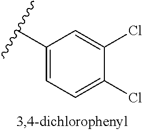 [SMILES: *c1ccc(Cl)c(Cl)c1] |
| P-29 | 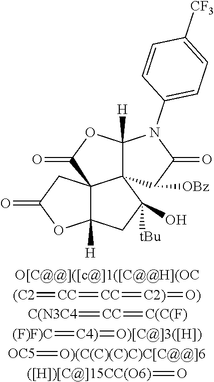 | N | 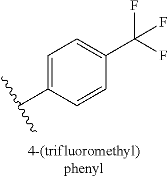 [SMILES: *c1ccc(C(F)(F)F)cc1] |
| JW100 | 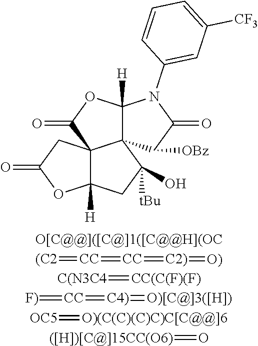 | N | 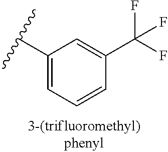 [SMILES: *c1cccc(C(F)(F)F)c1] |
| JW104 | 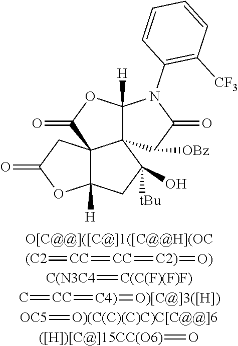 | N | 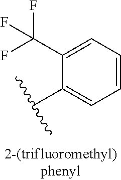 [SMILES: *c1ccccc1C(F)(F)F] |
| P-30 | 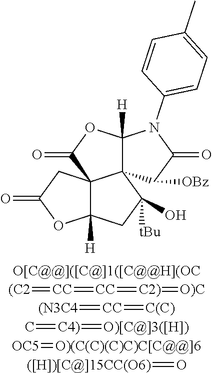 | N | 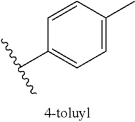 [SMILES: *c1ccc(C)cc1] |
| P-33 | 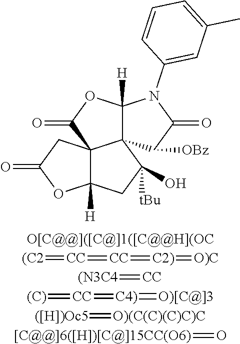 | N | 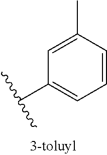 [SMILES: *c1cccc(C)c1] |
| P-34 | 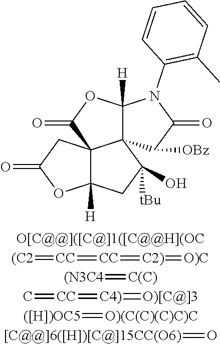 | N | 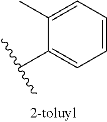 [SMILES: *c1ccccc1C] |
| JW103 | 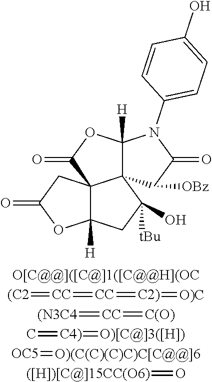 | N | 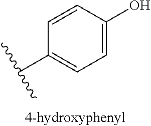 [SMILES: *c1ccc(O)cc1] |
| JW093 | 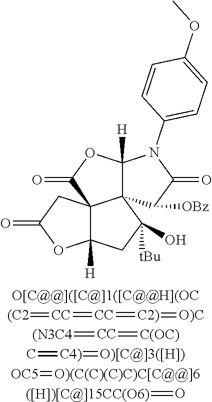 | N | 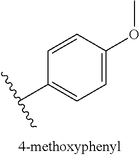 [SMILES: *c1ccc(OC)cc1] |
| JW092 | 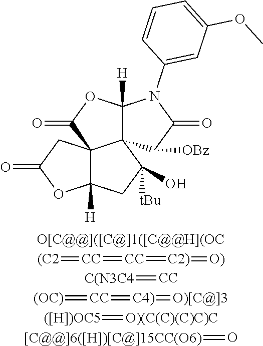 | N | 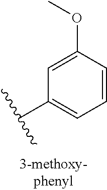 [SMILES: *c1cccc(OC)c1] |
| JW095 | 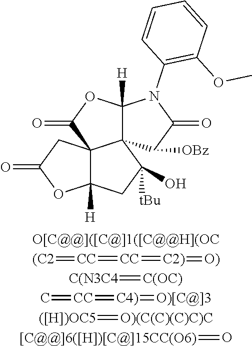 | N | 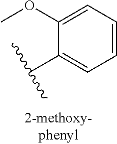 [SMILES: *c1ccccc1OC] |
| JW094 | 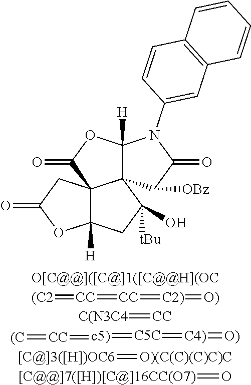 | N | 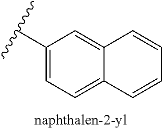 [SMILES: *c1ccc2ccccc2c1] |
| JW097 | 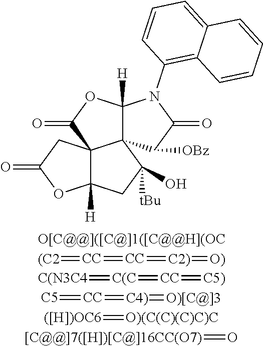 | N | 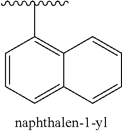 [SMILES: *c1cccc2ccccc12] |
| JW105 | 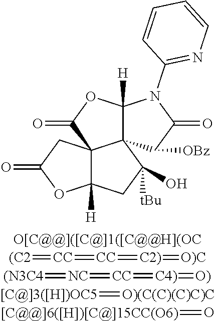 | N | pyridin-2-yl |
| JW096 | 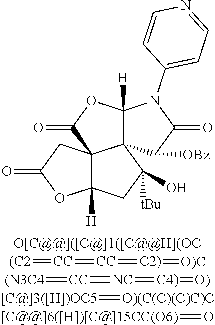 | N | pyridine-4-yl |
| JW120 | 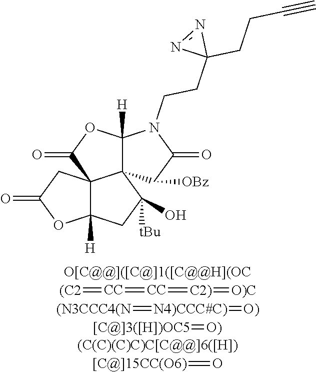 | N | 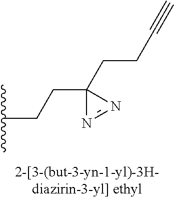 [SMILES: *CCC1(CCC#C)N=N1] |
| JW116 | 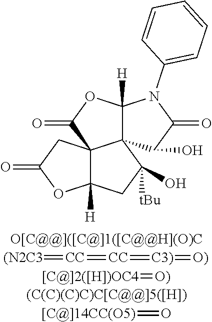 | N | phenyl |
| JW072 | 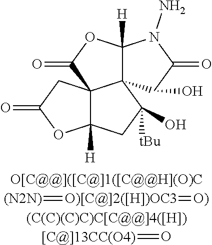 | N | NH₂ |
| SCC363 | 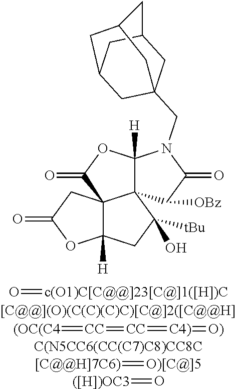 | N | 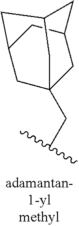 [SMILES: *CC12CC3CC(CC(C3)C1)C2] |
| SCC376 | 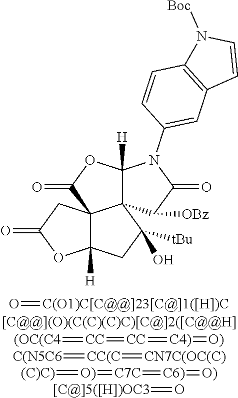 | N | 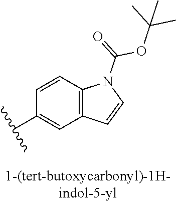 [SMILES: *c1ccc2c(ccn2C(=O)OC(C)(C)C)c1] |
| SCC382 | 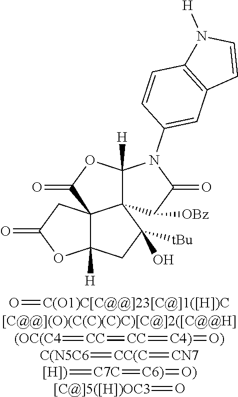 | N | 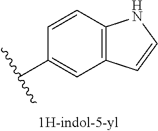 [SMILES: *c1ccc2[nH]ccc2c1] |
| SCC385 | 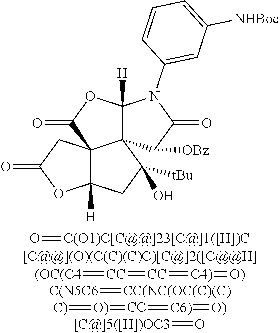 | N | 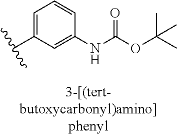 [SMILES: *c1cccc(NC(=O)OC(C)(C)C)c1] |
| SCC501 | 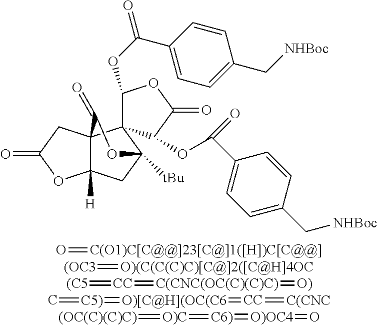 | O | — |
| SCC505 | 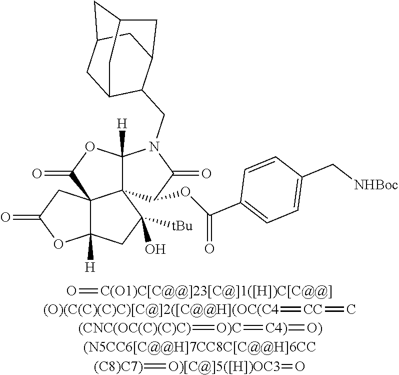 | N | 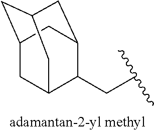 [SMILES: *CC1C2CC3CC(C2)CC1C3] |
| SCC506 | 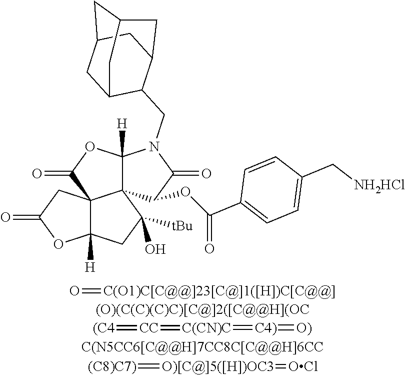 | N | 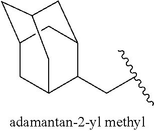 [SMILES: *CC1C2CC3CC(C2)CC1C3] |
| SCC545 | 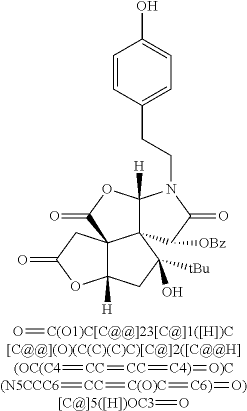 | N | 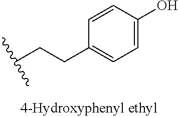 [SMILES: *CCc1ccc(O)cc1] |
| SCC555 | 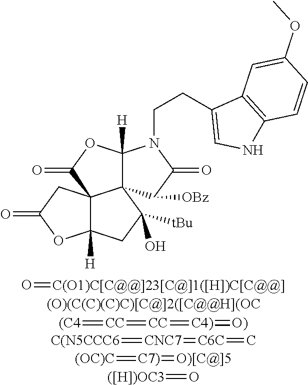 | N | 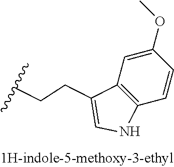 [SMILES: *CCc1c[nH]c2ccc(OC)cc12] |
| SCC558 | 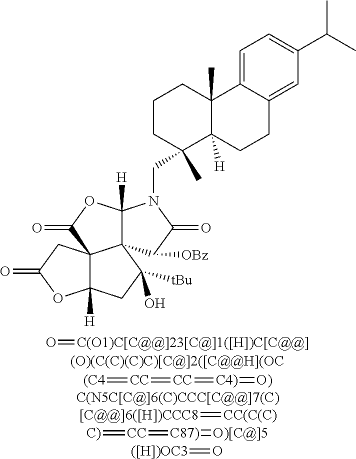 | N | 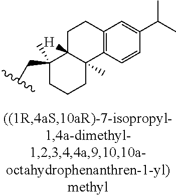 [SMILES: *C[C@]1(C)CCC[C@]2(C)c3ccc(C(C)C)cc3CC[C@@H]12] |
| SCC564 | 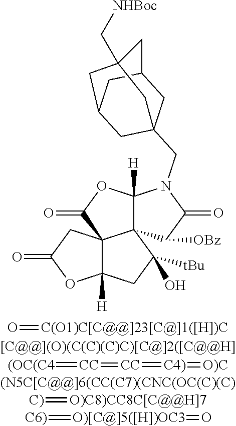 | N | 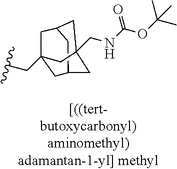 [SMILES: *CC12CC3CC(C1)CC(CNC(=O)OC(C)(C)C)(C3)C2] |
| SCC567 | 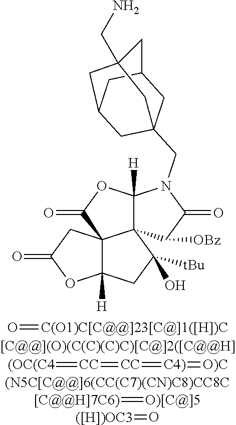 | N | 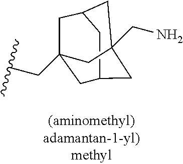 [SMILES: *CC12CC3CC(C1)CC(CN)(C3)C2] |
| SCB001 | 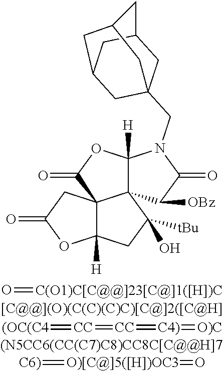 | N | 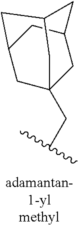 [SMILES: *CC12CC3CC(CC(C3)C1)C2] |
| SCB002 | 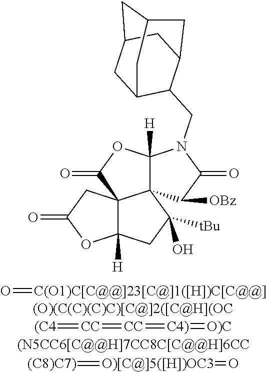 | N | 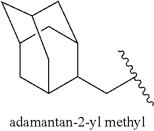 [SMILES: *CC1C2CC3CC(C2)CC1C3] |
| SCB008 | 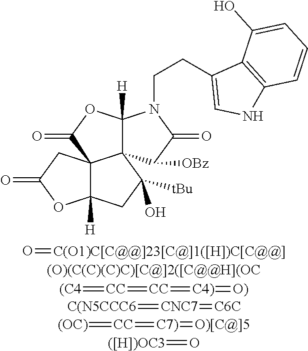 | N | 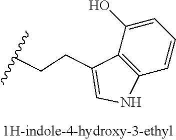 [SMILES: *CCc1c[nH]c2cccc(O)c12] |
| SXQ087-1 | 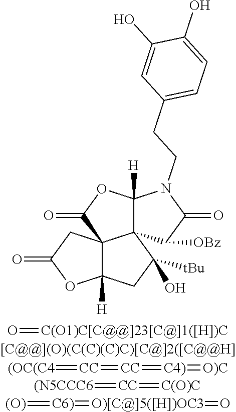 | N | 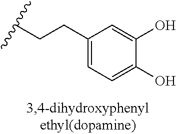 [SMILES: *CCc1ccc(O)c(O)c1] |
| SXQ090-1 | 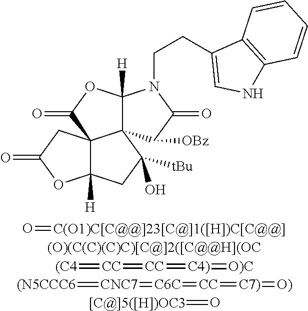 | N | 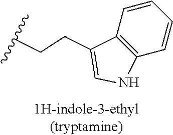 [SMILES: *CCc1c[nH]c2ccccc12] |
| SXQ092-1 | 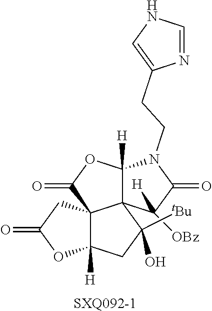 | N | 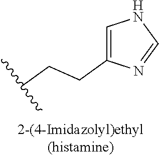 [SMILES: *CCc1c[nH]cn1] |
| SXQ094-1 | 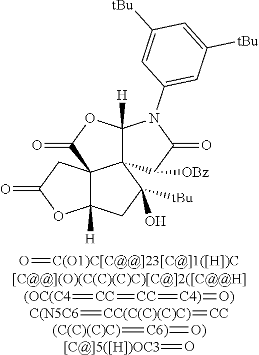 | N | 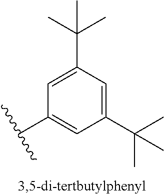 [SMILES: *c1cc(C(C)(C)C)cc(C(C)(C)C)c1] |
| SXQ102-1 | 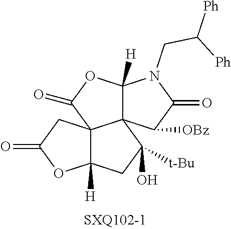 | N | 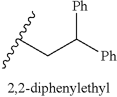 [SMILES: *CC(c1ccccc1)c1ccccc1] |
| SXQ091-1 | 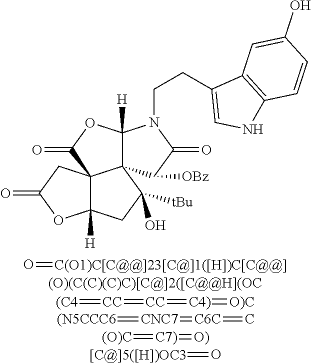 | N | 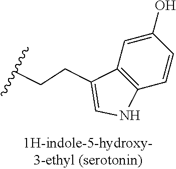 [SMILES: *CCc1c[nH]c2ccc(O)cc12] |
| SXQ125-2 | 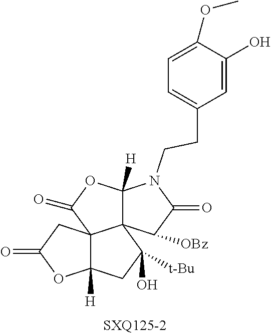 | N | 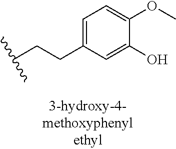 [SMILES: *CCc1ccc(OC)c(O)c1] |
| SXQ126-1 | 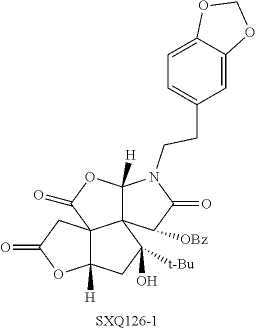 | N | 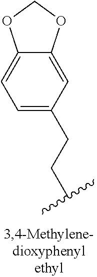 [SMILES: *CCc1ccc2c(c1)OCO2] |
| SXQ128-1 | 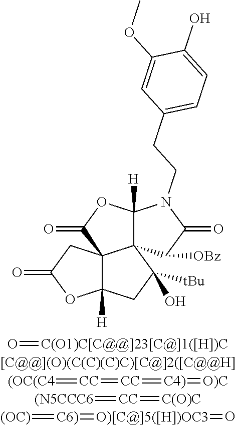 | N | 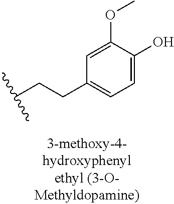 [SMILES: *CCc1ccc(O)c(OC)c1] |

| # | R² | R³ | R⁴ |
|---|---|---|---|
| XBB-001 (Bilobalide) | OH | OH | 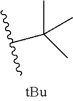 [SMILES: *C(C)(C)C] |
| XBB-002 | 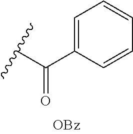 [SMILES: *C(=O)c1ccccc1] | — | 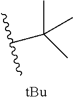 [SMILES: *C(C)(C)C] |
| XBB-003 | 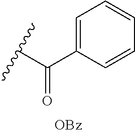 [SMILES: *C(=O)c1ccccc1] | — | 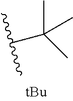 [SMILES: *C(C)(C)C] |
| XBB-004 | 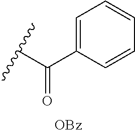 [SMILES: *C(=O)c1ccccc1] | OH | 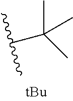 [SMILES: *C(C)(C)C] |
| XBB-005 | 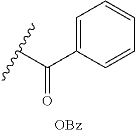 [SMILES: *C(=O)c1ccccc1] | OH | 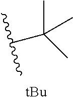 [SMILES: *C(C)(C)C] |
| XBB-006 | OH | OH | tBu |
| XBB-007 | OH | OH | tBu |
| XBB-008 | 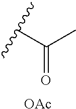 [SMILES: *C(C)=O ; "OAc"] | tBu | — |
| XBB-009 | OAc | isopropenyl | Me |
| XBB-010 | OAc | tBu | — |
| XBB-011 | OAc | isopropenyl | — |
| XBB-012 | OH | tBu | — |
| XBB-013 | OH | isopropenyl | Me |
| XBB-014 | OAc | OH | tBu |
| XBB-015 | OH | OH | tBu |
| XBB-016 | OBz | OH | tBu |
| XBB-017 | OBz | OH | tBu |
| XBB-018 | OBz | OH | tBu |
| XBB-019 | OBz | OH | tBu |
| XBB-020 | OBz | OH | tBu |
| XBB-021 | OBz | OH | tBu |
| XBB-022 | OBz | OH | tBu |
| XBB-023 | OBz | OH | tBu |
| XBB-024 | OBz | OH | tBu |
| XBB-025 | OBz | OH | tBu |
| XBB-026 | OBz | OH | tBu |
| XBB-027 | OBz | OH | tBu |
| XBB-028 | OBz | OH | tBu |
| XBB-029 | OBz | OH | tBu |
| XBB-030 | OBz | OH | tBu |
| XBB-031 | OBz | OH | tBu |
| XBB-032 | OBz | OH | tBu |
| XBB-033 | OBz | OH | tBu |
| XBB-034 | OBz | oH | tBu |
| XBB-035 | OBz | OH | tBu |
| XBB-036 | OBz | OH | tBu |
| XBB-037 | OBz | OH | tBu |
| XBB-037′ | OBz | OH | tBu |
| XBB-038 | OBz | OH | tBu |
| XBB-039 | OBz | OH | tBu |
| XBB-040 | OBz | OH | tBu |
| XBB-041 | OH | OH | tBu |
| XBB-042 | OH | OH | tBu |
| XBB-043 | OBz | OH | tBu |
| XBB-044 | OBz | OH | tBu |
| XBB-045 | OBz | Oh | tBu |
| XBB-046 | OBz | Oh | tBu |
| XBB-047 | OBz | OH | tBu |
| XBB-048 | OBz | OH | tBu |
| XBB-049 | OH | OH | tBu |
| XBB-050 | OAc | isopropenyl | Me |
| XBB-051 | OBz | OH | tBu |
| XBB-052 | OAc | tBu | — |
| XBB-053 | OH | OH | tBu |
| XBB-054 | OAc | tBu | — |
| XBB-056 | OH | OH | tBu |
| XBB-057 | OAc | tBu | — |
| XBB-058 | OAc | tBu | — |
| XBB-059 | OAc | tBu | — |
| XBB-060 | OAc | tBu | — |
| XBB-061 | OAc | tBu | — |
| XBB-062 | OAc | tBu | — |
| XBB-063 | OH | Me | isopropenyl |
| XBB-064 | OAc | tBu | — |
| XBB-065 | OBz | OH | tBu |
| XBB-066 | OBz | OH | tBu |
| XBB-067 | OBz | OH | tBu |
| XBB-068 | OBz | OH | tBu |
| XBB-069 | OAc | isopropenyl | Me |
| XBB-070 | OAc | tBu | — |
| XBB-071 | OAc | tBu | — |
| XBB-072 | OAc | tBu | — |
| XBB-073 | OAc | tBu | — |
| XBB-074 | OAc | tBu | — |
| XBB-075 | OAc | isopropenyl | Me |
| XBB-076 | OBz | tBu | — |
| XBB-077 | OBz | tBu | — |
| DW168 | OBz | OH | tBu |
| DW189 | OBz | OH | tBu |
| DW184 | OBz | OH | tBu |
| DW191 | OBz | OH | tBu |
| P-5 | OBz | OH | tBu |
| P-3 | OBz | OH | tBu |
| DW190 | OBz | OH | tBu |
| P-10 | OBz | OH | tBu |
| DW163 | OBz | OH | tBu |
| DW182 | OBz | OH | tBu |
| DW172 | OBz | OH | tBu |
| JW081 | OBz | OH | tBu |
| P-12 | OBz | OH | tBu |
| DW192 | OBz | OH | tBu |
| P-8 | OBz | OH | tBu |
| JW107 | OBz | OH | tBu |
| P-28 | OBz | oH | tBu |
| JW098 | OBz | OH | tBu |
| JW099 | OBz | OH | tBu |
| P-19 | OBz | OH | tBu |
| P-21 | OBz | OH | tBu |
| P-29 | OBz | OH | tBu |
| JW100 | OBz | OH | tBu |
| JW104 | OBz | OH | tBu |
| P-30 | OBz | OH | tBu |
| P-33 | OBz | OH | tBu |
| P-34 | OBz | OH | tBu |
| JW103 | OBz | OH | tBu |
| JW093 | OBz | OH | tBu |
| JW092 | OBz | OH | tBu |
| JW095 | OBz | OH | tBu |
| JW094 | OBz | OH | tBu |
| JW097 | OBz | OH | tBu |
| JW105 | OBz | OH | tBu |
| JW096 | OBz | OH | tBu |
| JW120 | OBz | OH | tBu |
| JW116 | OH | OH | tBu |
| JW072 | OH | OH | tBu |
| SCC363 | OBz | OH | tBu |
| SCC376 | OBz | OH | tBu |
| SCC382 | OBz | OH | tBu |
| SCC385 | OBz | OH | tBu |
| SCC501 | 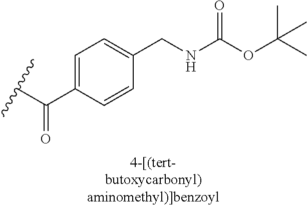 [SMILES: *C(=O)c1ccc(CNC(=O)OC(C)(C)C)cc1] | — | tBu |
| SCC505 | 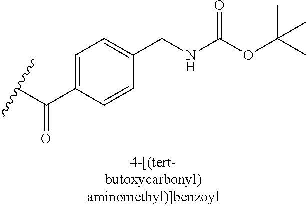 [SMILES: *C(=O)c1ccc(CNC(=O)OC(C)(C)C)cc1] | OH | tBu |
| SCC506 | 4-(aminomethyl)benzoyl | OH | tBu |
| | Hydrochloride | | |
| SCC545 | OBz | OH | tBu |
| SCC555 | OBz | OH | tBu |
| SCC558 | OBz | OH | tBu |
| SCC564 | OBz | OH | tBu |
| SCC567 | OBz | OH | tBu |
| SCB001 | OBz | OH | tBu |
| SCB002 | OBz | OH | tBu |
| SCB008 | OBz | OH | tBu |
| SXQ087-1 | OBz | OH | tBu |
| SXQ090-1 | OBz | OH | tBu |
| SXQ092-1 | OBz | OH | tBu |
| SXQ094-1 | OBz | OH | tBu |
| SXQ102-1 | OBz | OH | tBu |
| SXQ091-1 | OBz | OH | tBu |
| SXQ125-2 | OBz | OH | tBu |
| SXQ126-1 | OBz | OH | tBu |
| SXQ128-1 | OBz | OH | tBu |

| # | R⁵ | R⁶ | R⁷ |
|---|---|---|---|
| XBB-001 | H | H | — |
| (Bilobalide) | | | |
| XBB-002 | H | H | 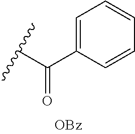 [SMILES: *C(=O)c1ccccc1] |
| XBB-003 | H | H | 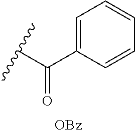 [SMILES: *C(=O)c1ccccc1] |
| XBB-004 | H | H | — |
| XBB-005 | H | H | — |
| XBB-006 | H | H | — |
| XBB-007 | H | H | — |
| XBB-008 | H | — | — |
| XBB-009 | H | H | — |
| XBB-010 | H | — | — |
| XBB-011 | H | — | — |
| XBB-012 | H | — | — |
| XBB-013 | H | H | — |
| XBB-014 | OH | H | — |
| XBB-015 | OH | H | — |
| XBB-016 | H | H | — |
| XBB-017 | H | H | — |
| XBB-018 | H | H | — |
| XBB-019 | H | H | — |
| XBB-020 | H | H | — |
| XBB-021 | H | H | — |
| XBB-022 | H | H | — |
| XBB-023 | H | H | — |
| XBB-024 | H | H | — |
| XBB-025 | H | H | — |
| XBB-026 | H | H | — |
| XBB-027 | H | H | — |
| XBB-028 | H | H | — |
| XBB-029 | H | H | — |
| XBB-030 | H | H | — |
| XBB-031 | H | H | — |
| XBB-032 | H | H | — |
| XBB-033 | H | H | — |
| XBB-034 | H | H | — |
| XBB-035 | H | H | — |
| XBB-036 | H | H | — |
| XBB-037 | h | H | — |
| XBB-037′ | H | H | — |
| XBB-038 | H | H | — |
| XBB-039 | H | H | — |
| XBB-040 | H | H | — |
| XBB-041 | H | H | — |
| XBB-042 | H | H | — |
| XBB-043 | H | H | — |
| XBB-044 | H | H | — |
| XBB-045 | H | H | — |
| XBB-046 | H | H | — |
| XBB-047 | H | H | — |
| XBB-048 | H | H | — |
| XBB-049 | H | H | — |
| XBB-050 | H | H | — |
| XBB-051 | H | H | — |
| XBB-052 | H | — | — |
| XBB-053 | H | H | — |
| XBB-054 | H | — | — |
| XBB-056 | OH | H | — |
| XBB-057 | H | — | — |
| XBB-058 | H | — | — |
| XBB-059 | H | — | — |
| XBB-060 | H | — | — |
| XBB-061 | H | — | — |
| XBB-062 | H | — | — |
| XBB-063 | H | H | — |
| XBB-064 | H | — | — |
| XBB-065 | H | H | — |
| XBB-066 | H | H | — |
| XBB-067 | H | H | — |
| XBB-068 | H | H | — |
| XBB-069 | H | H | — |
| XBB-070 | H | — | — |
| XBB-071 | H | — | — |
| XBB-072 | H | — | — |
| XBB-073 | H | — | — |
| XBB-074 | H | — | — |
| XBB-075 | H | h | — |
| XBB-076 | H | — | — |
| XBB-077 | H | — | — |
| DW168 | H | H | — |
| DW189 | H | H | — |
| DW184 | H | H | — |
| DW191 | H | H | — |
| P-5 | H | H | — |
| P-3 | H | H | — |
| DW190 | H | H | — |
| P-10 | H | H | — |
| DW163 | H | H | — |
| DW182 | h | H | — |
| DW172 | H | H | — |
| JW081 | H | H | — |
| P-12 | H | H | — |
| DW192 | H | H | — |
| P-8 | H | H | — |
| JW107 | H | H | — |
| P-28 | H | H | — |
| JW098 | H | H | — |
| JW099 | H | H | — |
| P-19 | H | H | — |
| P-21 | H | H | — |
| P-29 | H | H | — |
| JW100 | H | H | — |
| JW104 | H | H | — |
| P-30 | H | H | — |
| P-33 | H | H | — |
| P-34 | H | H | — |
| JW103 | H | H | — |
| JW093 | H | H | — |
| JW092 | H | H | — |
| JW095 | H | H | — |
| JW094 | H | H | — |
| JW097 | H | H | — |
| JW105 | H | H | — |
| JW096 | H | H | — |
| JW120 | H | H | — |
| JW116 | H | H | — |
| JW072 | H | H | — |
| SCC363 | H | H | — |
| SCC376 | H | H | — |
| SCC382 | H | H | — |
| SCC385 | H | H | — |
| SCC501 | H | H | 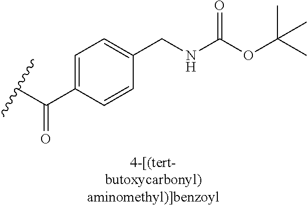 [SMILES: *C(=O)c1ccc(CNC(=O)OC(C)(C)C)cc1] |
| SCC505 | H | H | — |
| SCC506 | H | H | — |
| SCC545 | H | H | — |
| SCC555 | H | H | — |
| SCC558 | H | H | — |
| SCC564 | H | H | — |
| SCC567 | H | H | — |
| SCB001 | H | H | — |
| SCB002 | H | H | — |
| SCB008 | H | H | — |
| SXQ087-1 | H | H | — |
| SXQ090-1 | H | H | — |
| SXQ092-1 | H | H | — |
| SXQ094-1 | H | H | — |
| SXQ102-1 | H | H | — |
| SXQ091-1 | H | H | — |
| SXQ125-2 | H | H | — |
| SXQ126-1 | H | H | — |
| SXQ128-1 | H | H | — |

Example modification conditions are summarized in Table 1e.

### Initial Investigation of the Conditions for Bilobalide Benzoylation.

TABLE 1e
Example conditions for bilobalide benzoylation

| | | | | | Isolated yields | |
|---|---|---|---|---|---|---|
| Entry | Base | Solvent | Temperature | Reaction time | XBB-002 | XBB-003 |
| 1 | pyridine (6.0 equiv) | dioxane | 60° C. | 24 h | 53% | 38% |
| 2 | EDCl (10 equiv.) | DCM | rt | 24 h | 60% | 35% |
| | DMAP (10 equiv.) | | | | | |
| 3 | pyridine | pyridine | 50° C. | 26 h | 80% | 18% |
| 4 | pyridine | pyridine | 60° C. | 26 h | 35% | 65% |
| 5 | pyridine | pyridine | 80° C. | 26 h | 25% | 75% |
| 6 | 2,6-lutidine (6.0 equiv) | DCM | rt | 26 h | 76% | trace |

When conducting the reaction at 50° C., the major product XBB-002 was isolated in 80% yield (Entry 3, Table 1e) and the absolute structure was confirmed by X-ray crystallography. Unexpectedly, the absolute configuration of the minor product XBB-003 was the epimer of XBB-002 since only the configuration at C10 differs between XBB-002 and XBB-003. At lower temperature XBB-002 is the main product while at higher temperature XBB-003 is more favored (Entries 4-5, Table 1e). Only a trace amount of XBB-003 was observed with 2,6-lutidine as the base (Entry 6, Table 1e).

### Example 1.1: Synthesis of Di-Benzoylated Iso-Bilobalides XBB-002 and XBB-003 According to Scheme 1

Unless otherwise stated, all syntheses and manipulations of air- and moisture-sensitive materials were carried out under nitrogen atmosphere using standard Schlenk techniques. All glassware was oven-dried immediately prior to use. For reaction setup dry solvents stored over molecular sieve (MS) were purchased from Meryer (China). Reactions were magnetically stirred and monitored by analytical thin-layer chromatography (TLC). TLC was performed on Merck Kieselgel 60 F₂₅₄ with 0.2 mm thickness and visualized by exposure to ultraviolet light and appropriate staining. Organic solutions were concentrated by rotary evaporation at 20-45° C.

All chemicals and reagents available from commercial sources were directly used without further purification unless otherwise stated. Chromatographic purification of products was accomplished using forced-flow chromatography on silica gel (200-300 mesh). Melting points were measured on an OptiMelt using open glass capillaries, and the data is uncorrected. ¹H and ¹³C{¹H} NMR spectra were recorded on an Bruker instruments at 400, 500 or 600 MHz frequency. ¹H NMR spectra are referred to the residual solvent signal. The data for ¹H NMR is represented as follows: chemical shift (δ, ppm), multiplicity (s=singlet, d=doublet, t=triplet, m=multiplet, br=broad singlet, coupling constant (s) in Hz, integration). ¹³C{¹H} spectra are internally referenced to residual solvent signals. Data for ¹⁹F and ¹³C{¹H} are expressed in terms of chemical shift (6, ppm). High-resolution mass spectra (HRMS) were obtained on a Thermo Q Exactive™ Focus Hybrid Quadrupole-Orbitrap™ Mass Spectrometer. X-ray crystallographic analysis was performed on Bruker D8ventur e Diffractometer. Optical rotations were measured on Digital Polarimeter Jiao DIP-1010. Crystal structural data were collected by the single-crystal X-ray diffraction method with a Bruker D8-Venture system.

Synthesis of (2R,3S,3a'S,4R,6′R,7a'S)-6′-(tert-butyl)-2′,4′,5-trioxohexahydro-4′H,6′H-spiro[furan-3,8′-[3a,6]methanofuro[3,2-c]pyran]-2,4-diyl dibenzoate (XBB-002) and (2R,3S,3a'S,4S,6′R,7a'S)-6′-(tert-butyl)-2′,4′,5-trioxohexahydro-4′H,6′H-spiro[furan-3,8′-[3a,6]methanofuro[3,2-c]pyran]-2,4-diyl dibenzoate (XBB-003) are discussed herein.

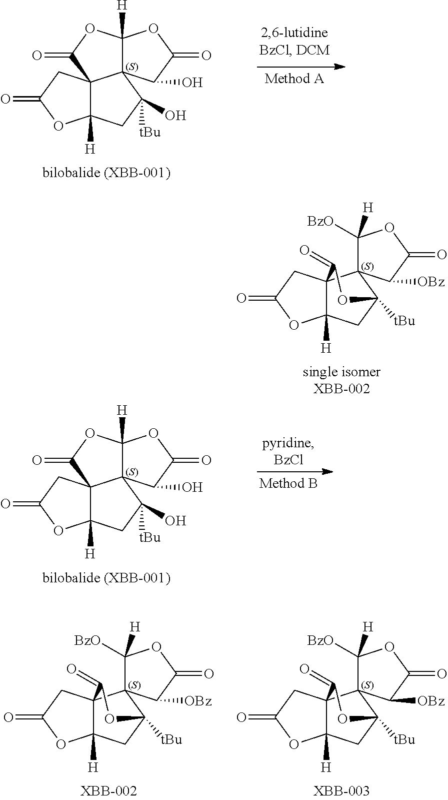

Method I: To a solution of bilobalide (6.00 g, 18.4 mmol, 1 equiv.) in anhydrous dichloromethane (40 mL, 0.46 M), benzoyl chloride (19.2 mL, 165.6 mmol, 9 equiv.), and 2,6-lutidine (12.9 mL, 110.4 mmol, 6 equiv.) was added. The reaction mixture was stirred at room temperature for 72 hours, then quenched with a saturated aqueous solution of sodium bicarbonate (40 mL). The resultant mixture was extracted with ethyl acetate (3×20 mL), dried with anhydrous sodium sulfate, filtered, and concentrated under reduced pressure. The crude mixture is then eluted through silica gel using a hexane/ethyl acetate/dichloromethane (3:1:1) mixture to remove any residual benzoyl chloride. The product mixture is then purified using silica gel column chromatography with dichloromethane to give the desired product XBB-002 (2.7 g, 28%) as a white solid.

Method II: To a solution of bilobalide (0.30 g, 0.920 mmol, 1.0 equiv.) and benzoyl chloride (1.940 g, 13.800 mmol, 15 equiv.) was added anhydrous pyridine (5.0 mL). After stirring at 50° C. or 80° C. for 16 h, the reaction solution was concentrated under reduced pressure to remove pyridine. The residue was diluted with ethyl acetate and quenched with saturated sodium bicarbonate solution. The combined organic layer was then washed with brine, dried over anhydrous Na₂SO₄, and concentrated in vacuo. The crude product was then purified by silica gel column chromatography (hexane/acetone=6:1) to afforded XBB-002 (50° C.: 0.42 g, 85%; 80° C.: 98 mg, 20%) and XBB-003 (50° C.: 49 mg, 10%; 80° C.: 0.38 g, 78%) both as white powder.

Method III: Bilobalide (1.0 eq., 1.53 mmol, 500 mg) was dried using MeCN co-evaporation and high vacuum. Then, dry DCM (20 mL) was added followed by BzCl (3.0 eq., 4.60 mmol, 0.64 g, 0.53 mL) and TMEDA (3.0 eq., 4.60 mmol, 0.53 g, 0.69 mL). The reaction was stirred until completion (18 h, o.n.). Then NH₄Cl sat. solution was added (30 mL) and after phase separation, the aqueous phase was extracted with DCM (2×30 mL). The combined organic phases were washed with brine (40 mL) and dried over MgSO₄. Colum chromatography (SiO2, Hex/EA/DCM 3:1:1 to Hex/EA 1:1) yielding XBB-002 in around 85-95% yield.

XBB-002: R_{f}=0.41 (hexane:EtOAc, 3:1); ¹H-NMR (500 MHz, CDCl₃): δ [ppm]=8.12-8.06 (m, 2H), 8.05-8.01 (m, 2H), 7.76-7.71 (m, 1H), 7.70-7.64 (m, 1H), 7.61-7.55 (m, 2H), 7.54-7.48 (m, 2H), 7.36 (s, 1H), 6.23 (s, 1H), 4.46 (dd, J=7.6, 4.5 Hz, 1H), 3.37 (d, J=18.0 Hz, 1H), 3.09 (d, J=18.0 Hz, 1H), 2.94-2.74 (m, 2H), 1.28 (s, 9H); ¹³C{¹H}-NMR (125 MHz, CDCl₃): δ [ppm]=169.60, 165.74, 165.52, 160.63, 160.59, 133.20, 132.87, 128.72, 128.62, 127.76, 127.30, 126.71, 126.31, 100.79, 94.46, 79.55, 69.00, 65.56, 64.44, 38.75, 37.85, 35.49, 34.27, 29.92; HRMS (ESI) m/z: [M+Na]⁺ Calcd for C₂₉H₂₆O₁₀Na⁺ 557.14182, found 557.14142. The X-ray crystal structure of XBB-002 is shown in FIG. 1A.

XBB-003: R_{f}=0.43 (hexane:EtOAc, 3:1); mp=172.4-173.0° C.; ¹H-NMR (500 MHz, CDCl₃): δ [ppm]=8.06 (d, J=7.7 Hz, 2H), 8.01 (d, J=7.8 Hz, 2H), 7.75-7.65 (m, 2H), 7.61-7.50 (m, 4H), 7.35 (s, 1H), 6.38 (s, 1H), 4.46 (dd, J=8.2, 3.6 Hz, 1H), 3.27 (s, 2H), 3.05 (dd, J=14.9, 3.6 Hz, 1H), 2.64 (dd, J=14.9, 8.2 Hz, 1H), 1.34 (s, 9H); ¹³C{¹H}-NMR (125 MHz, CDCl₃): δ [ppm]=172.16, 169.07, 168.72, 163.91, 163.59, 134.84, 134.41, 130.17, 129.97, 129.16, 128.99, 127.68, 127.15, 101.45, 92.86, 79.03, 63.93, 62.75, 61.32, 35.34, 35.03, 30.16, 26.87; HRMS (ESI) m/z: [M+Na]⁺ Calcd for C₂₉H₂₆O₁₀Na⁺ 557.14182, found 557.14182. The X-ray crystal structure of XBB-003 is shown in FIG. 1D.

Synthesis of (2R,3S,3a'S,4R,6′R,7a'S)-6′-(tert-butyl)-2′,4′,5-trioxohexahydro-4′H,6′H-spiro [furan-3,8′-[3a,6]methanofuro[3,2-c]pyran]-2,4-diyl bis(4-(((tert-butoxycarbonyl) amino) methyl) benzoate) (SCC501).

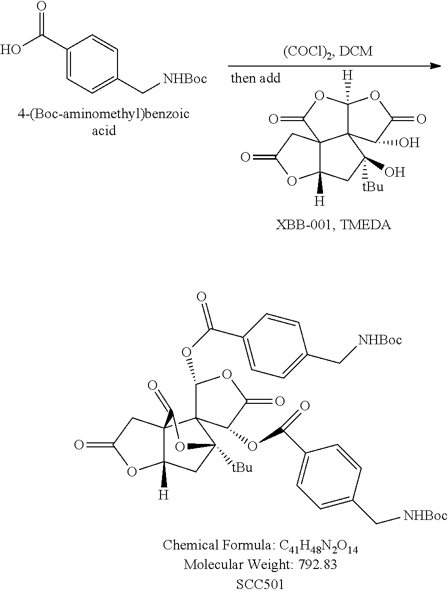

To a solution of 4-(Boc-aminomethyl) benzoic acid (4.0 equiv., 2.00 mmol, 502.1 mg) in DCM (25 mL) was added (COCl)₂ (4.0 equiv., 2.00 mmol, 253 mg, 171 μL) and one drop of DMF at 0° C. The reaction was stirred for 30 min. Then, at room temperature, tetramethylethylenediamine (TMEDA) (4.0 equiv., 2.00 mmol, 232 mg, 300 μL) and bilobalide (1.0 equiv. 0.50 mmol, 163 mg) were added. The reaction was stirred for 19 h. Then sat. NaHCO₃ solution was added (20 mL) and the phases were separated. The aqueous phase was extracted with DCM (3×20 mL) and the combined organic phases were washed with brine, dried over MgSO₄ and concentrated in vacuo. Column chromatography (SiO₂, hex/EtOAc/DCM 3:1:1) yielded in SCC501 as a white solid (215.6 mg, 54%).

### Example 1.2: Synthesis of Bilobalide Lactam Analogues XBB-004 and XBB-005 According to Scheme 2 or Scheme 5

Synthesis of (3aS,5aS,8R,8aS,9R,10aS)-9-(tert-butyl)-9-hydroxy-2,4,7-trioxooctahydro-4H,9H-furo[3″,2″:2′,3′]cyclopenta[1′,2′:3,4]furo[2,3-b]pyrrol-8-yl benzoate (XBB-004) is discussed herein.

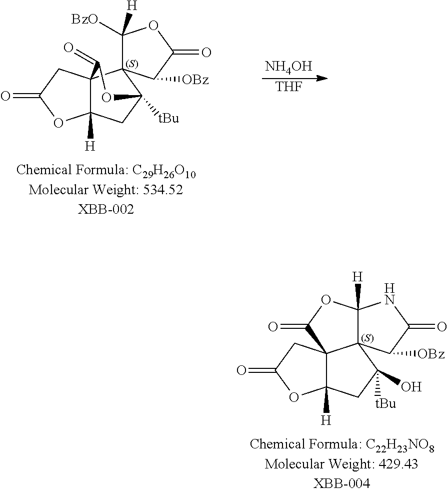

To a solution of XBB-002 (50 mg, 93.607 mmol, 1.0 equiv) in anhydrous THF (2 mL) was added 25% ammonia solution (13 mg, 0.187 mmol, 2.0 equiv) at 0° C. The resulting solution was then allowed to be stirred for 30 min at room temperature. The reaction was monitored by TLC and upon completion the reaction solution was diluted with ethyl acetate. The organic layer was washed with washed with saturated NaHCO₃ solution, and the combined organic layers were dried over Na₂SO₄, filtered and concentrated in vacuo. The crude product was purified via column chromatography (hexane:EtOAc=1:1) to provide XBB-004 (76% yield, 31 mg) as a white powder. R_{f}=0.34 (hexane:EtOAc, 1:1); mp=237.3-238.5° C.; ¹H-NMR (500 MHz, MeOD): δ [ppm]=8.04-7.89 (m, 2H), 7.73-7.61 (m, 1H), 7.56-7.40 (m, 2H), 6.36 (s, 1H), 6.08 (s, 1H), 5.18 (t, J=7.1 Hz, 1H), 3.18-2.94 (m, 2H), 2.72 (dd, J=13.7, 7.2 Hz, 1H), 2.12 (dd, J=13.7, 7.2 Hz, 1H), 1.06 (s, 9H); ¹³C{¹H}-NMR (125 MHz, MeOD): δ [ppm]=179.93, 175.90, 171.03, 166.48, 135.23, 130.93, 130.82, 129.85, 129.45, 128.61, 87.45, 86.39, 85.49, 72.66, 65.87, 60.89, 43.14, 38.39, 37.44, 26.95; HRMS (ESI) m/z: [M+Na]⁺ Calcd for C₂₂H₂₃NO₈Na⁺ 452.13159, found 452.13155.

### Synthesis of (3aS,5aS,8S,8aS,9R,10aS)-9-(tert-butyl)-9-hydroxy-2,4,7-trioxooctahydro-4H,9H-furo[3″,2″:2′,3′]cyclopenta[1′,2′:3,4]furo[2,3-b]pyrrol-8-yl benzoate (XBB-005)

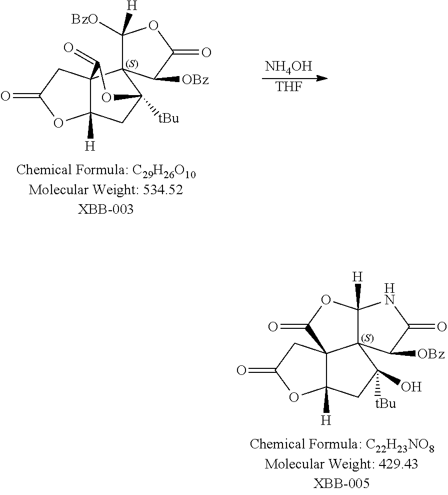

To a solution of XBB-003 (50 mg, 93.6 μmol, 1.0 equiv) in anhydrous THF (2 mL) was added 25% ammonia solution (13 mg, 0.187 mmol, 2.0 equiv) at 0° C. The resulting solution was then allowed to be stirred for 30 min at room temperature. The reaction was monitored by TLC and upon completion the reaction solution was diluted with ethyl acetate. The organic layer was washed with washed with saturated NaHCO₃ solution, and the combined organic layers were dried over Na₂SO₄, filtered and concentrated in vacuo. The crude product was purified via column chromatography (hexane:EtOAc=1:1) to provide XBB-005 (72% yield, 29 mg) as a white powder. R_{f}=0.35 (hexane:EtOAc, 1:1); mp=228.0-228.6° C.; ¹H-NMR (400 MHz, MeOD): δ [ppm]=8.20-8.06 (m, 2H), 7.73-7.64 (m, 1H), 7.60-7.50 (m, 2H), 6.74 (s, 1H), 5.76 (s, 1H), 5.53 (t, J=7.8 Hz, 1H), 2.77 (d, J=17.2 Hz, 1H), 2.63 (dd, J=15.3, 8.0 Hz, 1H), 2.32 (d, J=17.1 Hz, 1H), 2.11 (dd, J=15.3, 7.5 Hz, 1H), 1.17 (s, 8H); ¹³C{¹H}-NMR (100 MHz, MeOD): δ [ppm]182.53, 178.49, 173.63, 169.07, 137.82, 133.51, 132.43, 132.05, 90.04, 88.98, 88.10, 75.25, 68.47, 63.48, 57.38, 45.73, 40.99, 40.02, 29.52; HRMS (ESI) m/z: [M+Na]⁺ Calcd for C₂₂H₂₃NO₈Na⁺ 452.13159, found 452.13165.

### Example 1.3: Synthesis of Bilobalide Lactam Analogues XBB-006 and XBB-007 According to Scheme 3

Synthesis of (3aS,5aS,8R,8aS,9R,10aS)-9-(tert-butyl)-8,9-dihydroxytetrahydro-4H,9Hfuro[3″,2″:2′,3′]cyclopenta[1′,2′:3,4]furo[2,3-b]pyrrole-2,4,7(3H,8H)-trione (XBB-006) is discussed herein.

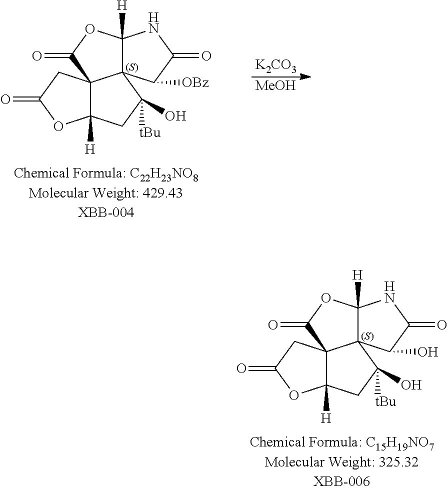

To a solution of XBB-004 (100 mg, 0.23 mmol, 1.0 equiv) in methanol was added potassium carbonate (64 mg, 0.46 mmol, 2.0 equiv). The resulting mixture was allowed to be stirred at room temperature for 2 h. Once the phenomenon of TLC plate indicated the completion of the reaction, the methanol was removed under reduced pressure. The residue was then diluted with water and the pH value of the solution was adjusted to 7.0. The mixture was extracted with ethyl acetate and the organic layer was washed with brine. The combined organic layers were dried over anhydrous sodium sulfate and concentrated under reduced pressure. The crude product was purified by column chromatography with elution system (hexane:EtOAc=1:1) to give XBB-006 (62 mg, 82%) as a white powder. R_{f}=0.10 (hexane:EtOAc, 1:1); mp=128.4-128.9° C.; ¹H-NMR (500 MHz, DMSO-d6): δ [ppm]=9.19 (s, 1H), 6.67 (d, J=4.7 Hz, 1H), 5.75 (s, 1H), 5.18 (s, 1H), 4.89 (t, J=6.9 Hz, 1H), 4.65 (d, J=4.7 Hz, 1H), 2.84 (d, J=17.9 Hz, 1H), 2.70 (d, J=18.0 Hz, 1H), 2.56-2.34 (m, 2H), 2.09 (dd, J=13.2, 6.8 Hz, 1H), 1.01 (s, 9H); ¹³C{¹H}-NMR (125 MHz, DMSO-d6): δ [ppm]=178.44, 174.14, 173.59, 85.34, 84.43, 82.96, 69.00, 64.88, 58.96, 41.38, 36.98, 36.40, 26.64; HRMS (ESI) m/z: [M+Na]⁺ Calcd for C₁₅H₁₉NO₇Na⁺ 348.10537, found 348.10522.

Synthesis of (3aS,5aS,8R,8aS,9R,10aS)-9-(tert-butyl)-8,9-dihydroxytetrahydro-4H,9Hfuro[3″,2″:2′,3′]cyclopenta[1′,2′:3,4]furo[2,3-b]pyrrole-2,4,7(3H,8H)-trione (XBB-007)

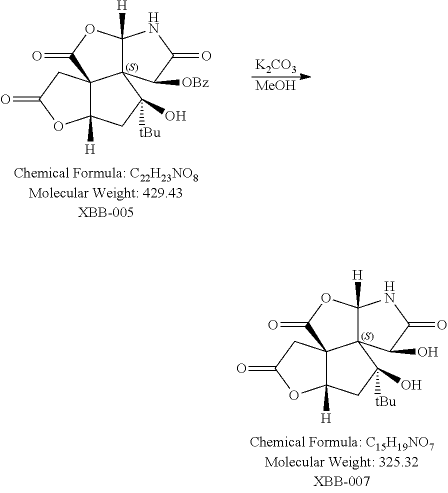

To a solution of XBB-005 (100 mg, 0.23 mmol, 1.0 equiv) in methanol was added potassium carbonate (64 mg, 0.46 mmol, 2.0 equiv). The resulting mixture was allowed to be stirred at room temperature for 2 h. Once the phenomenon of TLC plate indicated the completion of the reaction, the methanol was removed under reduced pressure. The residue was then diluted with water and the pH value of the solution was adjusted to 7.0. The mixture was extracted with ethyl acetate and the organic layer was washed with brine. The combined organic layers were dried with anhydrous sodium sulfate and concentrate under reduced pressure. The crude product was purified by column chromatography with elution system (hexane:EtOAc=1:1) to give XBB-007 (82% yield) as a white powder.

### Example 1.4: Synthesis of N-Arylated Bilobalide Lactam Analogues According to Scheme 4

Chan-Evans-Lam coupling towards the synthesis of N-arylated bilobalide lactam analogues is discussed herein.

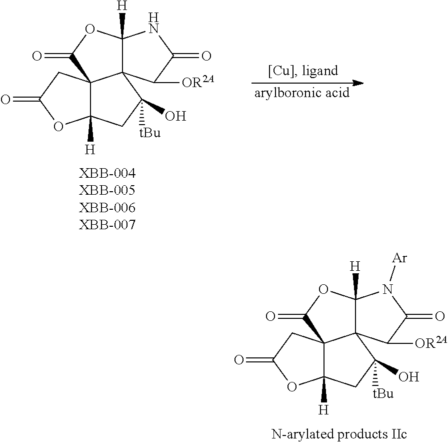

An oven-dried round-bottom flask was charged with XBB-004 (1.00 eq.), arylboronic acid R¹—B(OH)₂ (1.5 eq.), (CuOTf)₂-toluene (20 mol %), ligand, and DMSO (0.1 M). In some examples, 1,10-phenanthroline (in some embodiments, also referred to as ‘1,10-phen’) was used as the ligand. In some examples, no ligand is used. In some examples, XBB-004 was replaced with XBB-005, XBB-006, or XBB-007. The reaction mixture was stirred at room temperature under open air and monitored by TLC. Upon completion of the reaction, the crude reaction mixture was diluted with ice cold water and extracted three times with ethyl acetate. The combined organic layers were dried over Na₂SO₄ and concentrated under reduced pressure. The crude product containing the N-arylated product IIc (i.e., N-arylated bilobalide lactam analogues) according to Table 1f was purified by column chromatography to provide the desired product.

Examples of N-arylated bilobalide lactam analogues N-arylated products IIc are summarized in Table 1f.

TABLE 1f
Example N-arylated bilobalide lactam analogues

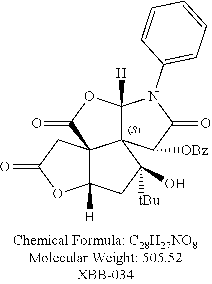

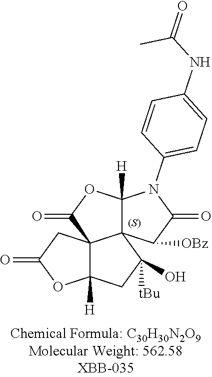

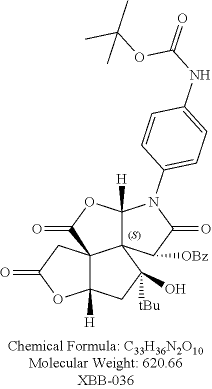

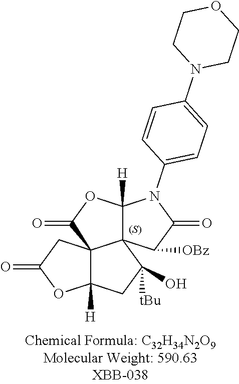

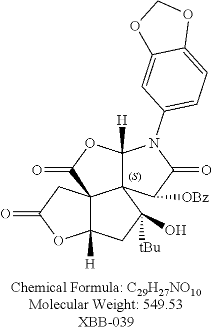

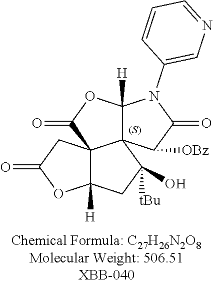

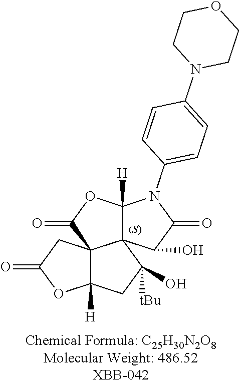

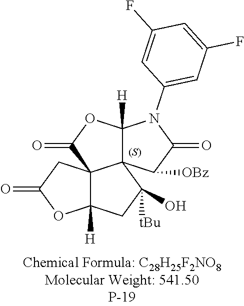

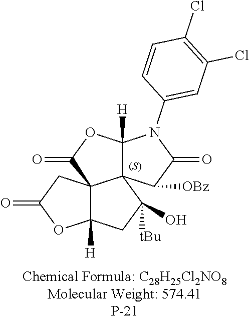

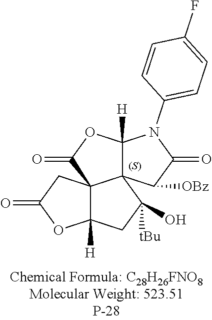

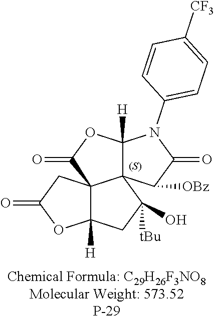

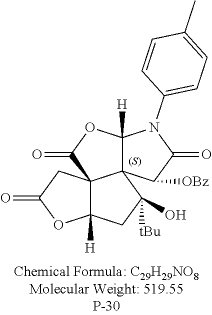

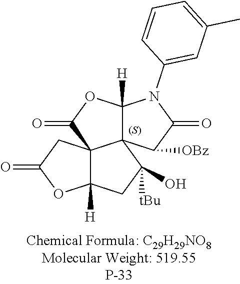

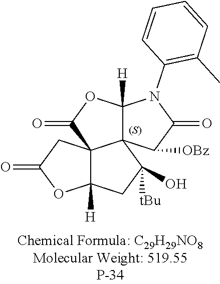

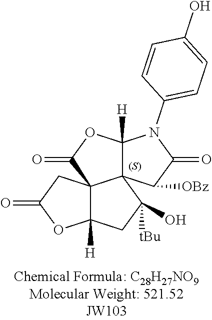

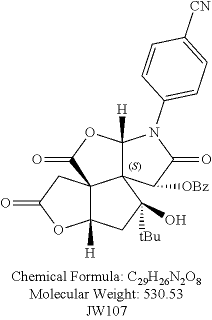

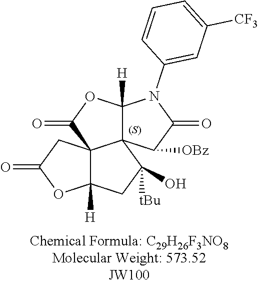

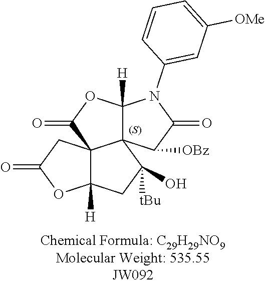

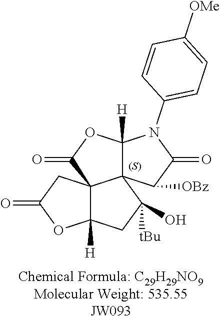

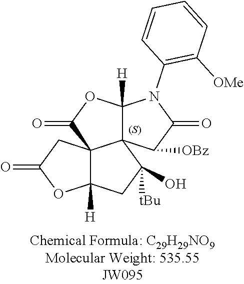

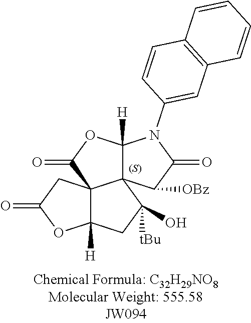

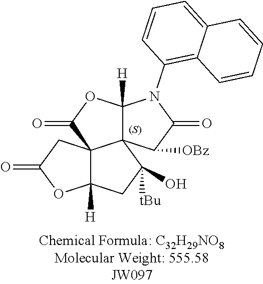

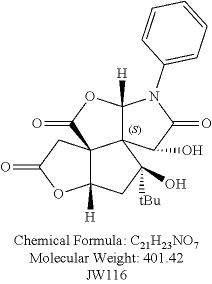

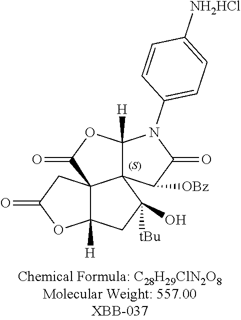

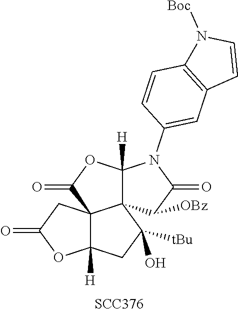

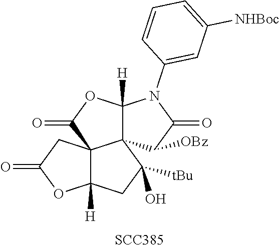

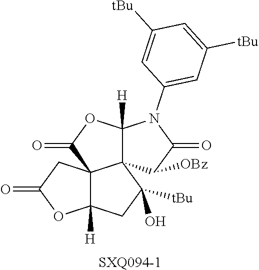

Synthesis of (3aS,5aS,8R,8aS,9R,10aS)-9-(tert-butyl)-9-hydroxy-2,4,7-trioxo-6-phenyloctahydro-4H,9H-furo[3″,2″:2′,3′]cyclopenta [1′,2′:3,4]furo[2,3-b]pyrrol-8-yl benzoate (XBB-034). Using XBB-004 (100 mg, 0.23 mmol), (CuOTf)₂-toluene (20 mol %, 46.6 μmol, 24.1 mg), 1,10-phenanthroline (40 mol %, 93.1 μmol, 16.8 mg), and phenylboronic acid (1.5 equiv.) yielded XBB-034 as a white powder (94 mg, 81%). R_{f}=0.2 (hexane:EtOAc, 1:1); mp=125.7-126.4° C.; ¹H-NMR (500 MHz, CDCl₃): δ [ppm]=8.07-7.90 (m, 2H), 7.71-7.56 (m, 3H), 7.56-7.48 (m, 2H), 7.47-7.38 (m, 2H), 7.34-7.28 (m, 1H), 6.47 (s, 1H), 6.46 (s, 1H), 5.22 (t, J=7.1 Hz, 1H), 3.30 (d, J=18.8 Hz, 1H), 2.95 (d, J=18.9 Hz, 1H), 2.68 (dd, J=14.0, 7.2 Hz, 1H), 2.28 (dd, J=14.0, 7.1 Hz, 1H), 1.13 (s, 9H); ¹³C{¹H}-NMR (100 MHz, CDCl₃): δ [ppm]=177.75, 173.59, 166.34, 165.24, 135.32, 134.35, 133.85, 130.12, 129.46, 129.37, 128.83, 127.73, 127.32, 127.11, 121.82, 88.05, 87.46, 83.40, 70.99, 61.72, 59.36, 44.47, 42.76, 37.43, 36.27, 26.43.; HRMS (ESI) m/z: [M+Na]⁺ Calcd for C₂₈H₂₇NO₈Na⁺ 528.16289, found 528.16338.

Synthesis of (3aS,5aS,8R,8aS,9R,10aS)-6-(4-acetamidophenyl)-9-(tert-butyl)-9-hydroxy-2,4,7-trioxooctahydro-4H,9H-furo [3″,2″:2′,3′]cyclopenta [1′,2′:3,4]furo[2,3-b]pyrrol-8-yl benzoate (XBB-035). Using XBB-004 (100 mg, 0.23 mmol), (CuOTf)₂-toluene (20 mol %, 46.6 μmol, 24.1 mg), 1,10-phenanthroline (40 mol %, 93.1 μmol, 16.8 mg), and 4-acetamidophenylboronic acid (1.5 equiv.) yielded XBB-035 as a white powder (92 mg, 71%). R_{f}=0.3 (hexane:EtOAc, 1:1); mp=185.7-186.2° C.; ¹H-NMR (500 MHz, DMSO-d6): δ [ppm]=10.09 (s, 1H), 8.02-7.90 (m, 2H), 7.78-7.72 (m, 1H), 7.72-7.63 (m, 2H), 7.60-7.51 (m, 4H), 6.49 (s, 1H), 6.47 (s, 1H), 5.12 (t, J=7.1 Hz, 1H), 3.28 (d, J=19.2 Hz, 1H), 2.98 (d, J=19.2 Hz, 1H), 2.73 (dd, J=13.6, 7.2 Hz, 1H), 2.11-2.01 (m, 4H), 1.07 (s, 9H); ¹³C{¹H}-NMR (125 MHz, DMSO-d6): δ [ppm]=177.87, 174.21, 168.90, 166.61, 164.96, 138.63, 134.93, 130.19, 129.95, 129.45, 128.24, 124.89, 119.70, 88.17, 86.66, 83.87, 71.78, 62.89, 59.31, 42.10, 40.85, 37.61, 36.11, 26.82, 24.45; HRMS (ESI) m/z: [M+Na]⁺ Calcd for C₃₀H₃₀N₂O₉Na⁺ 585.18435, found 585.18462.

Synthesis of (3aS,5aS,8R,8aS,9R,10aS)-9-(tert-butyl)-9-hydroxy-2,4,7-trioxo-6-(4-pivalamidophenyl) octahydro-4H,9H-furo [3″,2″:2′,3′]cyclopenta [1′,2′:3,4]furo[2,3-b]pyrrol-8-yl benzoate (XBB-036). Using XBB-004 (100 mg, 0.23 mmol), (CuOTf)₂-toluene (20 mol %, 46.6 μmol, 24.1 mg), 1,10-phenanthroline (40 mol %, 93.1 μmol, 16.8 mg), and a C-(1,1-dimethylethyl)N-(4-boronophenyl)carbamate (2.0 eq, 0.46 mmol) yielded XBB-036 as a white powder (107 mg, 75%). R_{f}=0.4 (hexane:EtOAc, 4:1); mp=131.4-132.8° C.; ¹H-NMR (500 MHz, CDCl₃): δ [ppm]=8.01 (d, J=7.7 Hz, 2H), 7.63 (t, J=7.4 Hz, 1H), 7.56-7.46 (m, 4H), 7.40 (d, J=8.6 Hz, 2H), 6.63 (s, 1H), 6.46 (s, 1H), 6.37 (s, 1H), 5.21 (t, J=7.0 Hz, 1H), 3.29 (d, J=18.8 Hz, 1H), 2.95 (d, J=18.8 Hz, 1H), 2.66 (dd, J=14.0, 7.1 Hz, 1H), 2.27 (dd, J=14.0, 7.1 Hz, 1H), 1.52 (s, 9H), 1.12 (s, 9H); ¹³C{¹H}-NMR (125 MHz, CDCl₃): δ [ppm]=177.60, 173.48, 166.14, 165.24, 152.65, 137.31, 134.30, 130.14, 130.12, 128.80, 127.73, 122.96, 119.17, 88.24, 87.58, 83.27, 70.91, 61.74, 59.33, 42.97, 37.42, 36.31, 28.32, 26.43; HRMS (ESI) m/z: [M+Na]⁺ Calcd for C₃₃H₃₆N₂O₁₀Na⁺ 643.22622, found 643.22659.

Synthesis of (3aS,5aS,8R,8aS,9R,10aS)-9-(tert-butyl)-9-hydroxy-6-(4-morpholinophenyl)-2,4,7-trioxooctahydro-4H,9H-furo [3″,2″:2′,3′]cyclopenta [1′,2′:3,4]furo [2,3-b]pyrrol-8-yl benzoate (XBB-038). Using XBB-004 (100 mg, 0.23 mmol), (CuOTf)₂-toluene (20 mol %, 46.6 μmol, 24.1 mg), 1,10-phenanthroline (40 mol %, 93.1 μmol, 16.8 mg), and a C-(1,1-dimethylethyl)N-(4-boronophenyl)carbamate (2.0 eq, 0.46 mmol) yielded XBB-038 as a white powder (124 mg, 91%). R_{f}=0.15 (hexane:EtOAc, 2:1); mp=160.8-161.2° C.; ¹H-NMR (500 MHz, MeOD): δ [ppm]8.10-7.95 (m, 2H), 7.77-7.64 (m, 1H), 7.61-7.49 (m, 2H), 7.49-7.36 (m, 2H), 7.11-6.90 (m, 2H), 6.59 (s, 1H), 6.42 (s, 1H), 5.22 (t, J=7.1 Hz, 1H), 3.95-3.69 (m, 4H), 3.27-3.07 (m, 6H), 2.78 (dd, J=13.7, 7.1 Hz, 1H), 2.30-2.12 (m, 1H), 1.15 (s, 9H); ¹³C{¹H}-NMR (125 MHz, MeOD): δ [ppm]174.24, 170.85, 164.05, 161.99, 148.37, 132.32, 128.20, 127.17, 126.76, 125.41, 123.80, 114.86, 89.30, 87.06, 84.85, 72.62, 68.08, 64.56, 61.27, 51.26, 44.55, 40.14, 38.98, 29.17; HRMS (ESI) m/z: [M+Na]⁺ Calcd for C₃₂H₃₄N₂O₉H⁺ 591.23371, found 591.23365.

Synthesis of (3aS,5aS,8R,8aS,9R,10aS)-6-(benzo[d][1,3]dioxol-5-yl)-9-(tert-butyl)-9-hydroxy-2,4,7-trioxooctahydro-4H,9H-furo [3″,2″:2′,3′]cyclopenta [1′,2′:3,4]furo [2,3-b]pyrrol-8-yl benzoate (XBB-039). Using XBB-004 (100 mg, 0.23 mmol), (CuOTf)₂-toluene (20 mol %, 46.6 μmol, 24.1 mg), 1,10-phenanthroline (40 mol %, 93.1 μmol, 16.8 mg), and a 3,4-methylenedioxyphenylboronic acid (2.0 eq, 0.46 mmol) yielded XBB-039 as a white powder (128 mg, 85%). R_{f}=0.2 (hexane:EtOAc, 2:1); mp=156.2-156.9° C.; ¹H-NMR (400 MHz, CDCl₃): δ [ppm]=8.08-7.90 (m, 2H), 7.70-7.59 (m, 1H), 7.51 (t, J=7.8 Hz, 2H), 7.09 (d, J=2.2 Hz, 1H), 6.97 (dd, J=8.4, 2.2 Hz, 1H), 6.81 (d, J=8.4 Hz, 1H), 6.45 (s, 1H), 6.31 (s, 1H), 6.00 (s, 2H), 5.21 (t, J=7.1 Hz, 1H), 3.28 (d, J=18.9 Hz, 1H), 2.96 (d, J=18.9 Hz, 1H), 2.67 (dd, J=14.0, 7.2 Hz, 1H), 2.37-2.17 (m, 1H), 1.13 (s, 9H); ¹³C{¹H}-NMR (100 MHz, CDCl₃): δ [ppm]=177.84, 173.64, 166.38, 165.25, 148.23, 146.81, 134.35, 130.12, 128.83, 128.73, 127.74, 116.70, 108.39, 104.90, 101.84, 88.62, 87.42, 83.41, 70.90, 61.96, 59.35, 42.76, 37.41, 36.26, 26.42; HRMS (ESI) m/z: [M+Na]⁺ Calcd for C₂₉H₂₇NO₁₀Na⁺ 572.15272, found 572.15255.

Synthesis of (3aS,5aS,8R,8aS,9R,10aS)-9-(tert-butyl)-9-hydroxy-2,4,7-trioxo-6-(pyridin-3-yl)octahydro-4H,9H-furo [3″,2″:2′,3′]cyclopenta [1′,2′:3,4]furo [2,3-b]pyrrol-8-yl benzoate (XBB-040). Using XBB-004 (100 mg, 0.23 mmol), (CuOTf)₂-toluene (20 mol %, 46.6 μmol, 24.1 mg), 1,10-phenanthroline (40 mol %, 93.1 μmol, 16.8 mg), and pyridin-3-ylboronic acid (2.0 eq, 0.46 mmol) yielded XBB-040 as a white powder (92 mg, 78%). R_{f}=0.2 (hexane:EtOAc, 1:1); mp=269.4-270.1° C.; ¹H-NMR (400 MHz, DMSO-d6): δ [ppm]=8.90 (d, J=2.6 Hz, 1H), 8.53 (dd, J=4.7, 1.5 Hz, 1H), 8.10 (ddd, J=8.4, 2.7, 1.5 Hz, 1H), 8.03-7.84 (m, 2H), 7.85-7.64 (m, 1H), 7.65-7.44 (m, 3H), 6.60 (s, 1H), 6.53 (s, 1H), 5.12 (t, J=7.1 Hz, 1H), 3.32 (d, J=19.4 Hz, 1H), 2.96 (d, J=19.3 Hz, 1H), 2.74 (dd, J=13.6, 7.2 Hz, 1H), 2.08-1.95 (dd, J=13.6, 7.2 Hz, 1H), 1.07 (s, 9H); ¹³C{¹H}-NMR (100 MHz, DMSO-d6): δ [ppm]=177.72, 174.21, 167.00, 164.90, 148.00, 144.92, 134.96, 132.59, 131.06, 129.97, 129.45, 128.17, 124.25, 87.56, 86.75, 83.87, 71.79, 62.90, 59.28, 42.08, 37.61, 36.13, 31.43, 31.12, 26.81, 22.54; HRMS (ESI) m/z: [M+Na]⁺ Calcd for C₂₇H₂₆N₂O₈H⁺ 507.17619, found 507.17663.

Synthesis of (3aS,5aS,8R,8aS,9R,10aS)-9-(tert-butyl)-8,9-dihydroxy-6-(4-morpholinophenyl)tetrahydro-4H,9H-furo [3″,2″:2′,3′]cyclopenta [1′,2′:3,4]furo [2,3-b]pyrrole-2,4,7(3H,8H)-trione (XBB-042). Using XBB-006 (100 mg, 0.23 mmol), (CuOTf)₂-toluene (20 mol %, 46.6 μmol, 24.1 mg), 1,10-phenanthroline (40 mol %, 93.1 μmol, 16.8 mg), and 4-(morpholinophenyl)boronic acid (2.0 eq, 0.46 mmol) yielded XBB-042 as a white powder (66 mg, 80%). R_{f}=0.1 (hexane:EtOAc, 1:2); mp=175.6-176.1° C.; ¹H-NMR (500 MHz, MeOD): δ [ppm]=7.45-7.28 (m, 2H), 7.09-6.97 (m, 2H), 6.21 (s, 1H), 5.88 (s, 1H), 5.51 (s, 1H), 5.09 (t, J=7.0 Hz, 1H), 5.07 (s, 1H), 3.89-3.80 (m, 4H), 3.22-3.16 (m, 4H), 3.14 (d, J=18.0 Hz, 1H), 2.81 (d, J=17.9 Hz, 1H), 2.66 (dd, J=13.5, 7.2 Hz, 1H), 2.40 (dd, J=13.5, 6.8 Hz, 1H), 1.18 (s, 9H); ¹³C{¹H}-NMR (125 MHz, MeOD): δ [ppm]=179.97, 176.27, 174.29, 151.94, 128.85, 126.19, 117.03, 90.22, 87.55, 85.26, 70.90, 67.87, 65.03, 60.87, 50.29, 43.03, 38.58, 38.15, 27.36; HRMS (ESI) m/z: [M+Na]⁺ Calcd for C₂₅H₃₀N₂O₈Na⁺ 509.18944, found 509.18984.

Synthesis of (3aS,5aS,8R,8aS,9R,10aS)-9-(tert-butyl)-9-hydroxy-2,4,7-trioxo-6-(3,5-difluoro-phenyl)octahydro-4H,9H-furo [3″,2″:2′,3′]cyclopenta [1′,2′:3,4]furo [2,3-b]pyrrol-8-yl benzoate (P-19). Using XBB-004 (50 mg, 0.12 mmol), (CuOTf)₂-toluene (50 mol %, 0.06 mmol, 30.1 mg), 1,10-phenanthroline (50 mol %, 0.06 mmol, 10.5 mg), and (2.0 eq, 0.46 mmol) yielded P-19 as a white powder (26.7 mg, 42%). ¹H-NMR (500 MHz, CDCl₃): δ [ppm]=8.01 (d, J=7.9 Hz, 2H), 7.66 (t, J=7.9 Hz, 1H), 7.52 (t, J=7.7 Hz, 2H), 7.40 (d, J=8.2 Hz, 2H), 6.74 (t, J=8.4 Hz, 1H), 6.44 (s, 1H), 6.41 (s, 1H), 5.25 (t, J=7.2 Hz, 1H), 3.24 (d, J=18.9 Hz, 1H), 2.96 (d, J=18.9 Hz, 1H), 2.69 (dd, J=7.3, 14.2 Hz, 1H), 2.32 (dd, J=7.0, 14.2 Hz, 1H), 2.21 (s, 1H), 1.15 (s, 9H).

Synthesis of (3aS,5aS,8R,8aS,9R,10aS)-9-(tert-butyl)-9-hydroxy-2,4,7-trioxo-6-(3,4-dichloro-phenyl)octahydro-4H,9H-furo [3″,2″:2′,3′]cyclopenta [1′,2′:3,4]furo [2,3-b]pyrrol-8-yl benzoate (P-21). Using XBB-004 (150 mg, 0.35 mmol), (CuOTf)₂-toluene (20 mol %, 0.07 mmol, 36.1 mg), 1,10-phenanthroline (20 mol %, 0.07 mmol, 12.6 mg), and (2.0 eq, 0.46 mmol) yielded P-21 as a white powder (57.9 mg, 29%). ¹H-NMR (500 MHz, CDCl₃): δ [ppm]=8.01 (d, J=7.9 Hz, 2H), 7.90 (s, 1H), 7.56-7.66 (m, 2H), 7.45-7.55 (m, 3H), 6.45 (s, 1H), 6.40 (s, 1H), 5.24 (t, J=6.8 Hz, 1H), 3.24 (d, J=19.1 Hz, 1H), 2.96 (d, J=18.9 Hz, 1H), 2.68 (dd, J=7.3, 13.7 Hz, 1H), 2.31 (dd, J=6.9, 13.7 Hz, 1H), 2.28 (s, 1H), 1.15 (s, 9H).

Synthesis of (3aS,5aS,8R,9R,10aS)-9-(tert-butyl)-9-hydroxy-2,4,7-trioxo-6-(4-fluorophenyl)octahydro-4H,9H-furo [3″,2″:2′,3′]cyclopenta [1′,2′:3,4]furo [2,3-b]pyrrol-8-yl benzoate (P-28). Using XBB-004 (42.9 mg, 0.10 mmol), (CuOTf)₂-toluene (20 mol %, 0.02 mmol, 10.3 mg), 1,10-phenanthroline (20 mol %, 0.02 mmol, 3.6 mg), and (4-fluorophenyl)boronic acid (2.0 eq, 0.20 mmol) yielded P-28 as a white powder (48.9 mg, 38%).

Synthesis of (3aS,5aS,8R,9R,10aS)-9-(tert-butyl)-9-hydroxy-2,4,7-trioxo-6-(4-(trifluoromethyl)phenyl)octahydro-4H,9H-furo [3″,2″:2′,3′]cyclopenta [1′,2′:3,4]furo [2,3-b]pyrrol-8-yl benzoate (P-29). Using XBB-004 (42.9 mg, 0.10 mmol), (CuOTf)₂-toluene (20 mol %, 0.02 mmol, 10.3 mg), and (4-(trifluoromethyl)phenyl)boronic acid (2.0 eq, 0.20 mmol) yielded P-29 as a white powder.

Synthesis of (3aS,5aS,8R,9R,10aS)-9-(tert-butyl)-9-hydroxy-2,4,7-trioxo-6-(toluyl)octahydro-4H,9H-furo [3″,2″:2′,3′]cyclopenta [1′,2′:3,4]furo [2,3-b]pyrrol-8-yl benzoate (P-30). Using XBB-004 (150.0 mg, 0.35 mmol), (CuOTf)₂-toluene (20 mol %, 0.07 mmol, 36.1 mg), and toluylboronic acid (2.0 eq, 0.70 mmol) yielded P-30 as a white powder (139.2 mg, 77%).

Synthesis of (3aS,5aS,8R,9R,10aS)-9-(tert-butyl)-9-hydroxy-2,4,7-trioxo-6-(3-methyl phenyl)octahydro-4H,9H-furo [3″,2″:2′,3′]cyclopenta [1′,2′:3,4]furo [2,3-b]pyrrol-8-yl benzoate (P-33). Using XBB-004 (150.0 mg, 0.35 mmol), (CuOTf)₂-toluene (20 mol %, 0.07 mmol, 36.1 mg), and toluylboronic acid (2.0 eq, 0.70 mmol) yielded P-33 as a white powder (144.5 mg, 80%).

Synthesis of (3aS,5aS,8R,9R,10aS)-9-(tert-butyl)-9-hydroxy-2,4,7-trioxo-6-(2-methyl phenyl)octahydro-4H,9H-furo [3″,2″:2′,3′]cyclopenta [1′,2′:3,4]furo [2,3-b]pyrrol-8-yl benzoate (P-34). Using XBB-004 (150.0 mg, 0.35 mmol), (CuOTf)₂-toluene (20 mol %, 0.07 mmol, 36.1 mg), and (2-methyl phenyl)boronic acid (2.0 eq, 0.70 mmol) yielded P-34 as a white powder (71.2 mg, 40%).

Synthesis of (3aS,5aS,8R,9R,10aS)-9-(tert-butyl)-6-(4-hydroxyphenyl)-9-hydroxy-2,4,7-trioxodecahydrofuro [3″,2″:2′,3′]cyclopenta [1′,2′:3,4]furo [2,3-b]pyrrol-8-yl benzoate (JW103). Using XBB-004 (153 mg, 0.35 mmol), (CuOTf)₂-toluene (20 mol %, 0.07 mmol, 36.2 mg), and 4-hydroxyphenyl phenylboronic acid (2.0 eq, 0.70 mmol) yielded JW103 as a white powder (0.16 g, 88%).

Synthesis of (3aS,5aS,8R,9R,10aS)-9-(tert-butyl)-6-(4-cyanophenyl)-9-hydroxy-2,4,7-trioxodecahydrofuro [3″,2″:2′,3′]cyclopenta [1′,2′:3,4]furo [2,3-b]pyrrol-8-yl benzoate (JW107). Using XBB-004 (153 mg, 0.35 mmol), (CuOTf)₂-toluene (20 mol %, 0.07 mmol, 36.2 mg), and 4-hydroxyphenyl phenylboronic acid (2.0 eq, 0.70 mmol) yielded JW107 as a white powder (0.12 g, 63%). R_{f}=0.20 (hexane:EtOAc:DCM, 3:1:1); ¹H-NMR (400 MHz, DMSO-d6): δ [ppm]=7.96-8.03 (m, 2H), 7.89-7.95 (m, 2H), 7.77-7.84 (m, 2H), 7.68-7.75 (m, 1H), 7.50-7.57 (m, 2H), 6.58 (s, 1H), 6.50 (s, 1H), 5.16 (t, J=7.13 Hz, 1H), 3.68 (br. s, 1H), 3.06 (d, J=19 Hz, 1H), 3.00 (d, J=19 Hz, 1H), 2.76 (dd, J=14.0, 7.2 Hz, 1H), 2.21 (dd, J=14.0, 7.2 Hz, 1H), 1.10 (s, 9H).

Synthesis of (3aS,5aS,8R,9R,10aS)-9-(tert-butyl)-9-hydroxy-2,4,7-trioxo-6-(3-(trifluoromethyl)phenyl)octahydro-4H,9H-furo [3″,2″:2′,3′]cyclopenta [1′,2′:3,4]furo [2,3-b]pyrrol-8-yl benzoate (JW100). Using XBB-004 (153 mg, 0.35 mmol), (CuOTf)₂-toluene (20 mol %, 0.07 mmol, 36.2 mg), and (3-(trifluoromethyl)phenyl)boronic acid (2.0 eq, 0.70 mmol) yielded JW100 as a white powder (0.20 g, 69%). R_{f}=0.29 (hexane:EtOAc:DCM, 3:1:1); ¹H-NMR (500 MHz, CDCl₃): δ [ppm]=7.99-8.06 (m, 3H), 7.88-7.94 (m, 1H), 7.62-7.68 (m, 1H), 7.49-7.59 (m, 4H), 6.49 (s, 1H), 6.48 (s, 1H), 5.25 (t, J=7.1 Hz, 1H), 3.29 (d, J=18.9 Hz, 1H), 2.97 (d, J=18.9 Hz, 1H), 2.69 (dd, J=14.2, 7.2 Hz, 1H), 2.33 (dd, J=14.2, 7.2 Hz, 1H), 2.27 (s, 1H), 1.16 (s, 9H).

Synthesis of (3aS,5aS,8R,9R,10aS)-9-(tert-butyl)-9-hydroxy-6-(3-methoxyphenyl)-2,4,7-trioxodecahydrofuro [3″,2″:2′,3′]cyclopenta [1′,2′:3,4]furo [2,3-b]pyrrol-8-yl benzoate (JW092). Using XBB-004 (150.3 mg, 0.35 mmol), (CuOTf)₂-toluene (20 mol %, 0.07 mmol, 36.2 mg), 1,10-phenanthroline (20 mol %, 0.07 mmol, 12.6 mg) and (3-methoxyphenyl)boronic acid (2.0 eq, 0.70 mmol) yielding in JW092 as a white powder (47.4 mg, 25%). R_{f}=0.26 (hexane:EtOAc:DCM, 3:1:1); ¹H-NMR (500 MHz, CDCl₃): δ [ppm]=8.00-8.05 (m, 2H), 7.61-7.67 (m, 1H), 7.48-7.54 (m, 2H), 7.28-7.37 (m, 2H), 7.14-7.21 (m, 1H), 6.83 (dd, J=8.2, 1.7 Hz, 1H), 6.45 (s, 1H), 6.44 (s, 1H), 5.22 (t, J=7.11 Hz, 1H), 3.81 (s, 3H), 3.29 (d, J=18.8 Hz, 1H), 2.95 (d, J=18.8 Hz, 1H), 2.67 (dd, J=14.1, 7.2 Hz, 1H), 2.47 (s, 1H), 2.28 (dd, J=14.1, 7.2 Hz, 1H), 1.13 (s, 9H).

Synthesis of (3aS,5aS,8R,9R,10aS)-9-(tert-butyl)-9-hydroxy-6-(4-methoxyphenyl)-2,4,7-trioxodecahydrofuro [3″,2″:2′,3′]cyclopenta [1′,2′:3,4]furo [2,3-b]pyrrol-8-yl benzoate (JW093). Using XBB-004 (153 mg, 0.35 mmol), (CuOTf)₂-toluene (20 mol %, 0.07 mmol, 36.2 mg), 1,10-phenanthroline (20 mol %, 0.02 mmol, 3.6 mg), and (4-methoxyphenyl)boronic acid (2.0 eq, 0.70 mmol) (1.5 equiv.) yielded JW093 as a white powder (0.13 g, 68%). R_{f}=0.23 (hexane:EtOAc:DCM, 3:1:1); ¹H-NMR (400 MHz, CDCl₃): δ [ppm]=7.99-8.04 (m, 2H), 7.60-7.66 (m, 1H), 7.44-7.55 (m, 4H), 6.90-6.96 (m, 2H), 6.47 (s, 1H), 6.34 (s, 1H), 5.23 (t, J=7.1 Hz, 1H), 3.81 (s, 3H), 3.31 (d, J=18.9 Hz, 1H), 2.97 (d, J=18.9 Hz, 1H), 2.66 (dd, J=14.1, 7.3 Hz, 1H), 2.29 (dd, J=14.1, 7.3 Hz, 1H), 2.27 (s, 1H), 1.14 (s, 9H).

Synthesis of (3aS,5aS,8R,9R,10aS)-9-(tert-butyl)-9-hydroxy-6-(2-methoxyphenyl)-2,4,7-trioxodecahydrofuro [3″,2″:2′,3′]cyclopenta [1′,2′:3,4]furo [2,3-b]pyrrol-8-yl benzoate (JW095). Using XBB-004 (153 mg, 0.35 mmol), (CuOTf)₂-toluene (20 mol %, 0.07 mmol, 36.2 mg), 1,10-phenanthroline (20 mol %, 0.02 mmol, 3.6 mg), and (2-methoxyphenyl)boronic acid (2.0 eq, 0.70 mmol) (1.5 equiv.) yielded JW095 as a white powder (24.4 mg, 13%). ¹H-NMR (400 MHz, CDCl₃): δ [ppm]=8.01 (d, J=7.8 Hz, 2H), 7.62 (t, J=7.5 Hz, 1H), 7.50 (t, J=8.1 Hz, 2H), 7.38 (t, J=7.6 Hz, 1H), 7.19 (d, J=8.0 Hz, 1H), 6.96-7.04 (m, 2H), 6.54 (s, 1H), 6.31 (s, 1H), 5.23 (t, J=7.2 Hz, 1H), 3.83 (s, 3H), 3.43 (d, J=18.8 Hz, 1H), 3.02 (d, J=18.9 Hz, 1H), 2.64 (dd, J=14.2, 7.5 Hz, 1H), 2.29 (dd, J=13.9, 7.0 Hz, 1H), 2.00 (s, 1H), 1.17 (s, 9H).

Synthesis of (3aS,5aS,8R,9R,10aS)-9-(tert-butyl)-9-hydroxy-6-(naphthalen-2-yl)-2,4,7-trioxodecahydrofuro [3″,2″:2′,3′]cyclopenta [1′,2′:3,4]furo [2,3-b]pyrrol-8-yl benzoate (JW094). Using XBB-004 (153 mg, 0.35 mmol), (CuOTf)₂-toluene (20 mol %, 0.07 mmol, 36.2 mg), 1,10-phenanthroline (20 mol %, 0.02 mmol, 3.6 mg), and (naphthalen-2-yl)boronic acid (2.0 eq, 0.70 mmol) (1.5 equiv.) yielded JW094 as a white powder (0.14 g, 74%). R_{f}=0.23 (hexane:EtOAc:DCM, 3:1:1); ¹H-NMR (400 MHz, CDCl₃): δ [ppm]=8.11 (d, J=1.9 Hz, 1H), 8.00-8.07 (m, 2H), 7.86 (d, J=9.1 Hz, 1H), 7.78-7.84 (m, 2H), 7.76 (dd, J=9.0, 2.1 Hz, 1H), 7.61-7.68 (m, 1H), 7.46-7.55 (m, 4H), 6.56 (s, 1H), 6.52 (s 1H), 5.23 (t, J=7.1 Hz, 1H), 3.35 (d, J=19.0 Hz, 1H), 2.99 (d, J=19.0 Hz, 1H), 2.66 (dd, J=14.1, 7.2 Hz, 1H), 2.56 (s, 1H), 2.29 (dd, J=14.1, 7.2 Hz, 1H), 1.12 (s, 9H).

Synthesis of (3aS,5aS,8R,9R,10aS)-9-(tert-butyl)-9-hydroxy-6-(naphthalen-1-yl)-2,4,7-trioxodecahydrofuro [3″,2″:2′,3′]cyclopenta [1′,2′:3,4]furo [2,3-b]pyrrol-8-yl benzoate (JW097). Using XBB-004 (153 mg, 0.35 mmol), (CuOTf)₂-toluene (20 mol %, 0.07 mmol, 36.2 mg), 1,10-phenanthroline (20 mol %, 0.02 mmol, 3.6 mg), and (naphthalen-1-yl)boronic acid (2.0 eq, 0.70 mmol) (1.5 equiv.) yielded JW097 as a white powder (53.1 mg, 27%).

Synthesis of tert-butyl 5-((3aS,5aS,8R,8aS,9R,10aS)-8-(benzoyloxy)-9-(tert-butyl)-9-hydroxy-2,4,7-trioxohexahydro-4H,9H-furo[3″,2″:2′,3′]cyclopenta[1′,2′:3,4]furo[2,3-b]pyrrol-6(5aH)-yl)-1H-indole-1-carboxylate (SCC376). Using XBB-004 (1.00 equiv., 0.16 mmol, 66.8 mg), (CuOTf)₂-toluene (15 mol %, 23.3 μmol, 12.1 mg), and (1-(tert-butoxycarbonyl)-1H-indol-5-yl)boronic acid (1.7 equiv., 0.26 mmol, 69.0 mg) yielded SCC376 as a white powder (60.3 mg, 60%). R_{f}=0.31 (hexane:EtOAc, 2:1); ¹H-NMR (600 MHz, CDCl₃): δ [ppm]=8.16 (brs, 1H), 8.02 (d, J=7.6 Hz, 2H), 7.75 (s, 1H), 7.65-7.60 (m, 2H), 7.50 (t, J=7.1 Hz, 2H), 7.44 (d, J=8.9 Hz, 1H), 6.54 (d, J=3.5 Hz, 1H, 6.50 (s, 1H), 6.42 (s, 1H), 5.20 (t, J=7.1 Hz, 1H), 3.34 (d, J=18.5 Hz, 1H), 2.99 (d, J=18.5 Hz, 1H), 2.65 (dd, J=7.1, 14.1 Hz, 1H), 2.51 (s, 1H), 2.27 (dd, J=7.3, 14.1 Hz, 1H), 1.67 (s, 9H), 1.12 (s, 9H).

Synthesis of (3aS,5aS,8R,8aS,9R,10aS)-6-(3-((tert-butoxycarbonyl)amino)phenyl)-9-(tert-butyl)-9-hydroxy-2,4,7-trioxooctahydro-4H,9H-furo[3″,2″:2′,3′]cyclopenta [1′,2′:3,4]furo [2,3-b]pyrrol-8-ylbenzoate (SCC385). Using XBB-004 (1.00 equiv., 89.9 μmol, 38.6 mg), (CuOTf)₂-toluene (15 mol %, 13.5 μmol, 7.0 mg), and (1-(tert-butoxycarbonyl)-1H-indol-5-yl)boronic acid (2.0 equiv., 0.18 mmol, 42.6 mg) yielded SCC385 as a white powder (32.1 mg, 57%). R_{f}=0.51 (hexane:EtOAc, 1:1); ¹H-NMR (600 MHz, CDCl₃): δ [ppm]=8.00 (d, J=8.4 Hz, 1H), 7.66-7.61 (m, 2H), 7.50 (t, J=8.1 Hz, 2H), 7.37 (brs, 1H), 7.33-7.27 (m, 2H), 6.75 (s, 1H), 6.44 (s, 1H), 6.43 (s, 1H), 5.20 (t, J=7.1 Hz, 1H), 3.27 (d, J=18.7 Hz, 1H), 2.94 (d, J=19.0 Hz, 1H), 2.71 (brs, 1H), 2.66 (dd, J=7.3, 14.2 Hz, 1H), 2.24 (dd, J=7.0, 13.8 Hz, 1H), 1.48 (s, 9H), 1.10 (s, 9H).

### Synthesis of SXQ094-1

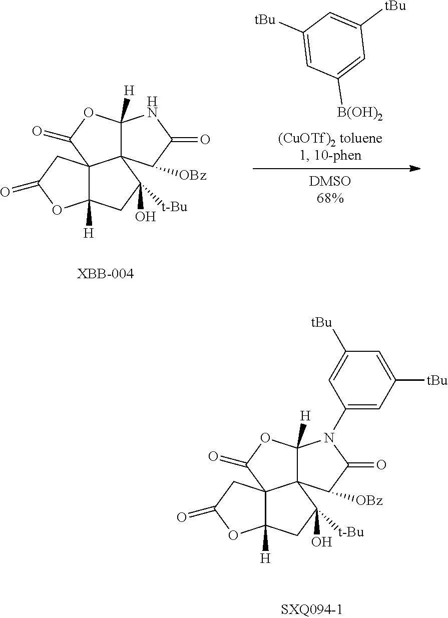

To an oven-dried flask containing a magnetic stir bar was added XBB-004 (20 mg, 0.0465 mmol, 1.0 equiv.) and 0.5 mL of DMSO, followed by the addition of 1,10-phen (4 mg, 0.0232 mmol, 0.5 equiv.), (CuOTf)₂·toluene (12 mg, 0.0232 mmol, 0.5 equiv.) and (3,5-Di-tert-butylphenyl)boronic acid (22 mg, 0.093 mmol, 2 equiv.). The resulting solution was allowed to be stirred at room temperature for 24 h. Once completion indicated by TLC, the resulting solution was diluted with ethyl acetate and was quenched by the addition of saturated aqueous NH₄Cl (6 mL). This mixture was extracted with EtOAc (3×10 mL), the organic layers were combined, washed with saturated aqueous NaCl, and dried over anhydrous Na₂SO₄. The solution was concentrated in vacuo and this crude product was purified by column chromatography (Hexane:EtOAc=2:1, v/v) to give SXQ094-1 (18 mg, 68%) as yellow powder. TLC: R_{f}=0.5 (Hexane/EtOAc=2/1; strongly UV active, stains yellow upon KMnO₄ staining). ¹H NMR (700 MHz, CDCl₃) δ 8.04-8.01 (m, 2H), 7.64 (t, J=7.7 Hz, 1H), 7.51 (t, J=7.7 Hz, 2H), 7.45 (d, J=1.7 Hz, 2H), 7.36 (d, J=1.7 Hz, 1H), 6.47 (m, 2H), 5.23 (m, 1H), 3.33 (d, J=18.9 Hz, 1H), 2.96 (d, J=18.9 Hz, 1H), 2.69 (dd, J=14.0, 7.2 Hz, 1H), 2.48 (s, 1H), 2.29 (dd, J=14.0, 7.2 Hz, 1H), 1.33 (s, 18H), 1.15 (s, 9H). ¹³C NMR (126 MHz, CDCl₃) δ 177.9, 173.6, 166.4, 165.4, 152.3, 135.0, 134.4, 130.3, 128.9, 127.8, 121.4, 116.2, 88.4, 87.8, 83.3, 71.1, 61.8, 59.5, 43.1, 37.6, 36.4, 35.2, 35.2, 31.5, 26.6.

### Example 1.5a: Further Modification of Bilobalide Analogues Via Debenzylation According to Scheme 3

Synthesis of (3aS,5aS,8R,8aS,9R,10aS)-9-(tert-butyl)-8,9-dihydroxy-6-phenyltetrahydro-4H,9H-furo[3″,2″:2′,3′]cyclopenta[1′,2′:3,4]furo[2,3-b]pyrrole-2,4,7(3H,8H)-trione (JW116). Using XBB-034, K₂CO₃ yielding in JW116 as a white powder. ¹H-NMR (500 MHz, MeOD): δ [ppm]=7.57 (d, J=7.6 Hz, 2H), 7.45 (t, J=8.1 Hz, 2H), 7.32 (t, J=7.6 Hz, 1H), 6.32 (s, 1H), 5.08 (t, J=7.1 Hz, 1H), 5.07 (s, 1H), 3.13 (d, J=18.3 Hz, 1H), 2.80 (d, J=17.8 Hz, 1H), 2.66 (dd, J=7.1, 13.4 Hz, 1H), 2.39 (dd, J=7.1, 13.5 Hz, 1H), 1.17 (s, 9H).

### Synthesis of (3aS,5aS,8R,8aS,9R,10aS)-6-benzyl-9-(tert-butyl)-8,9-dihydroxytetrahydro-4H,9H-furo[3″,2″:2′,3′]cyclopenta[1′,2′:3,4]furo[2,3-b]pyrrole-2,4,7(3H,8H)-trione (XBB-041)

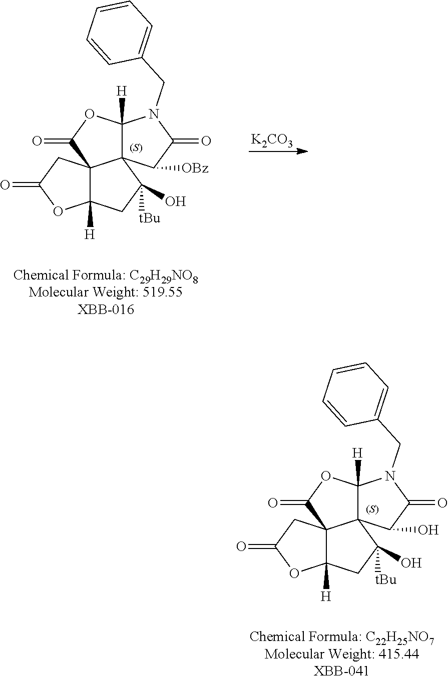

To a solution of N-phenyl analogue XBB-016 (50 mg, 1 eq., 98.9 μmol) in methanol (10 mL) was added potassium carbonate (2.0 equiv, 27.3 mg, 0.20 mmol). The resulting solution was allowed to be stirred at room temperature for 4 h. Once the starting material was fully consumed, the reaction solution was concentrated under reduced pressure and then the residue was resuspended with water. The pH value of the resuspension was adjusted to 7.0 using 3 N HCl aqueous solution. The mixture was extracted with ethyl acetate and washed with brine. The combined organic layers were dried over anhydrous sodium sulfate and concentrated under reduced pressure. The crude product was purified by column chromatography with elution system (hexane:ethyl acetate=1:2) to give XBB-041 as white powder. R_{f}=0.15 (hexane:EtOAc, 1:2); mp=191.2-191.8° C.; ¹H-NMR (500 MHz, MeOD): δ [ppm]=7.39-7.34 (m, 2H), 7.33-7.28 (m, 3H), 5.59 (s, 1H), 5.02 (t, J=6.9 Hz, 1H), 4.94-4.87 (m, 2H), 4.21 (d, J=14.7 Hz, 1H), 2.99 (dd, J=17.9, 1.3 Hz, 1H), 2.65 (dd, J=18.0, 1.4 Hz, 1H), 2.58 (dd, J=13.5, 7.2 Hz, 1H), 2.29 (dd, J=13.5, 6.8 Hz, 1H), 0.99 (s, 9H); ¹³C{¹H}-NMR (125 MHz, MeOD): δ [ppm]=180.17, 176.43, 174.65, 136.72, 130.10, 129.67, 129.34, 88.52, 87.43, 85.41, 71.07, 65.32, 61.01, 45.38, 43.15, 38.48, 38.37, 27.3.

### Example 1.5b: Further Modification of Bilobalide Analogues Via Boc Deprotection According to Scheme 12

According to the method described in Scheme 12, MeOH (20 mL/mmol) was cooled to 0° C. and acetyl chloride (10-15 eq.) was added. After 5 min Boc protected amine (1.00 eq) was added. The reaction is stirred at room temperature until complete conversion. The volatiles are removed, and the product can be crystallized as HCl salt.

Synthesis of 4-(((3aS,5aS,8R,8aS,9R,10aS)-8-(benzoyloxy)-9-(tert-butyl)-9-hydroxy-2,4,7-trioxohexahydro-4H,9H-furo [3″,2″:2′,3′]cyclopenta [1′,2′:3,4]furo [2,3-b]pyrrol-6(5aH)-yl)methyl)piperidin-1-ium chloride (XBB-021). Using XBB-020 (0.68 g, 1.07 mmol, 1.00 eq.) yielding in XBB-021 as a white powder (0.59 g, 84%). R_{f}=0.2 (DCM/MeOH, 10:1); mp=74.6-75.1° C.; ¹H-NMR (400 MHz, MeOD): δ [ppm]=8.05-7.92 (m, 2H), 7.79-7.65 (m, 1H), 7.53 (t, J=7.8 Hz, 2H), 6.43 (s, 1H), 6.11 (s, 1H), 5.20 (t, J=7.1 Hz, 1H), 3.54-3.35 (m, 4H), 3.15-2.94 (m, 4H), 2.77 (dd, J=13.8, 7.2 Hz, 1H), 2.16 (dd, J=13.6, 7.2 Hz, 2H), 1.97-1.81 (m, 2H), 1.60-1.36 (m, 2H), 1.10 (s, 9H); ¹³C{¹H}-NMR (125 MHz, MeOD): δ [ppm]=178.05, 174.33, 168.28, 165.13, 133.96, 129.63, 128.54, 128.05, 88.58, 86.18, 84.09, 71.01, 63.02, 59.22, 46.75, 44.86, 44.82, 41.80, 37.07, 34.09, 29.97, 29.87, 25.64; HRMS (ESI) m/z: [M+Na]⁺ Calcd for C₂₈H₃₄N₂O₈H⁺ 527.23879, found 527.23860.

Synthesis of 4-(((3aS,5aS,8S,8aS,9R,10aS)-8-(benzoyloxy)-9-(tert-butyl)-9-hydroxy-2,4,7-trioxohexahydro-4H,9H-furo [3″,2″:2′,3′]cyclopenta [1′,2′:3,4]furo [2,3-b]pyrrol-6(5aH)-yl)methyl)piperidin-1-ium chloride (XBB-022). R_{f}=0.15 (DCM/MeOH, 10:1); mp=78.4-79.2° C.; ¹H-NMR (400 MHz, MeOD): δ [ppm]=8.03-7.95 (m, 2H), 7.70 (t, J=7.5 Hz, 1H), 7.53 (t, J=7.7 Hz, 2H), 6.43 (s, 1H), 6.12 (s, 1H), 5.22 (t, J=7.1 Hz, 1H), 3.54-3.27 (m, 4H), 3.16-2.93 (m, 4H), 2.84-2.69 (m, 1H), 2.26-2.09 (m, 2H), 1.95-1.82 (m, 2H), 1.64-1.41 (m, 2H), 1.09 (s, 9H); ¹³C{¹H}-NMR (100 MHz, CDCl₃): δ [ppm]=178.14, 174.47, 168.59, 165.21, 134.14, 129.63, 128.64, 127.94, 88.75, 86.32, 84.16, 71.09, 63.12, 59.33, 43.29, 41.79, 37.09, 36.12, 32.24, 26.52, 26.41, 25.72; HRMS (ESI) m/z: [M+Na]⁺ Calcd for C₂₈H₃₄N₂O₈H⁺ 527.23879, found 527.23859.

Synthesis of 4-(2-((3aS,5aS,8S,8aS,9R,10aS)-8-(benzoyloxy)-9-(tert-butyl)-9-hydroxy-2,4,7-trioxohexahydro-4H,9H-furo [3″,2″:2′,3′]cyclopenta [1′,2′:3,4]furo[2,3-b]pyrrol-6(5aH)-yl)ethyl)piperidin-1-ium chloride (XBB-024). R_{f}=0.15 (DCM/MeOH, 20:1); mp=220.3-221.2° C.; ¹H-NMR (400 MHz, DMSO-d6): δ [ppm]=9.08 (s, 1H), 7.95-7.81 (m, 2H), 7.78-7.67 (m, 1H), 7.54 (t, J=7.7 Hz, 2H), 6.23 (s, 1H), 6.04 (s, 1H), 5.69 (s, 1H), 5.09 (t, J=7.0 Hz, 1H), 3.34-3.05 (m, 5H), 2.87-2.62 (m, 4H), 1.96 (dd, J=13.6, 7.1 Hz, 1H), 1.80 (t, J=15.5 Hz, 2H), 1.61-1.28 (m, 5H), 0.99 (s, 9H); ¹³C{¹H}-NMR (100 MHz, DMSO-d6): δ [ppm]=178.12, 174.16, 167.38, 165.03, 134.90, 129.90, 129.42, 128.23, 88.42, 86.30, 83.89, 71.55, 63.17, 59.35, 43.33, 42.07, 37.49, 36.20, 33.47, 31.33, 31.17, 28.41, 26.81.; HRMS (ESI) m/z: [M+H]⁺ Calcd for C₂₉H₃₆N₂O₈H⁺ 541.25444, found 541.25399.

Synthesis of 4-(3-((3aS,5aS,8S,8aS,9R,10aS)-8-(benzoyloxy)-9-(tert-butyl)-9-hydroxy-2,4,7-trioxohexahydro-4H,9H-furo[3″,2″:2′,3′]cyclopenta [1′,2′:3,4]furo [2,3-b]pyrrol-6(5aH)-yl)propyl)piperidin-1-ium chloride (XBB-0026). R_{f}=0.15 (DCM/MeOH, 20:1); mp=146.8-147.2° C.; ¹H-NMR (400 MHz, MeOD): δ [ppm]=8.11 (s, 1H), 8.05-7.92 (m, 2H), 7.76-7.61 (m, 1H), 7.53 (t, J=7.8 Hz, 2H), 7.25 (s, 1H), 6.39 (s, 1H), 6.11 (s, 1H), 5.21 (t, J=7.1 Hz, 1H), 3.84-3.57 (m, 1H), 3.54-3.44 (m, 1H), 3.43-3.35 (m, 3H), 3.11-2.90 (m, 4H), 2.84-2.69 (m, 1H), 2.22-2.08 (m, 1H), 1.99-1.86 (m, 2H), 1.81-1.56 (m, 3H), 1.50-1.24 (m, 3H), 1.09 (s, 9H); ¹³C{¹H}-NMR (100 MHz, MeOD): δ [ppm]=178.17, 174.38, 168.04, 165.17, 134.63, 134.03, 129.61, 128.58, 128.02, 120.56, 88.35, 86.23, 84.13, 71.15, 63.15, 59.35, 43.87, 41.79, 41.16, 37.07, 36.13, 32.93, 32.75, 28.54, 25.67, 24.06; HRMS (ESI) m/z: [M+H]⁺ Calcd for C₃₀H₃₈N₂O₈H⁺ 555.27009, found 555.26993.

Synthesis of 4-(2-((3aS,5aS,8S,8aS,9R,10aS)-8-(benzoyloxy)-9-(tert-butyl)-9-hydroxy-2,4,7-trioxohexahydro-4H,9H-furo [3″,2″:2′,3′]cyclopenta [1′,2′:3,4]furo [2,3-b]pyrrol-6(5aH)-yl)ethyl)piperazin-1-ium chloride (XBB-028). R_{f}=0.20 (DCM/MeOH, 20:1); mp=136.5-137.2° C.; ¹H-NMR (400 MHz, CDCl₃): δ [ppm]=8.05-7.83 (m, 2H), 7.68-7.54 (m, 1H), 7.48 (t, J=7.7 Hz, 2H), 7.03 (s, 1H), 6.35 (s, 1H), 6.19 (s, 1H), 5.20 (t, J=7.1 Hz, 1H), 3.82-3.65 (m, 1H), 3.44 (s, 1H), 3.40-3.28 (m, 1H), 3.14 (d, J=19.0 Hz, 1H), 2.98-2.78 (m, 5H), 2.72-2.38 (m, 7H), 2.17 (dd, J=13.8, 7.1 Hz, 1H), 1.09 (s, 9H); ¹³C{¹H}-NMR (100 MHz, CDCl₃): δ [ppm]=178.53, 173.97, 167.34, 165.35, 134.21, 130.07, 128.77, 127.90, 88.56, 86.74, 83.76, 71.01, 62.79, 59.37, 55.65, 53.43, 42.47, 37.42, 36.95, 36.54, 26.65; HRMS (ESI) m/z: [M+H]⁺ Calcd for C₂₈H₃₅N₃O₈H⁺ 542.24969, found 542.24990.

Synthesis of (3aS,5aS,8R,8aS,9R,10aS)-9-(tert-butyl)-9-hydroxy-2,4,7-trioxo-6-(((S)-pyrrolidin-3-yl)methyl)octahydro-4H,9H-furo [3″,2″:2′,3′]cyclopenta [1′,2′:3,4]furo [2,3-b]pyrrol-8-yl benzoate hydrochloride (XBB-032). R_{f}=0.20 (DCM/MeOH, 20:1); mp=106.2-106.8° C.; ¹H-NMR (400 MHz, MeOD): δ [ppm]=7.88 (d, J=7.8 Hz, 3H), 7.58 (t, J=7.4 Hz, 1H), 7.48-7.20 (m, 2H), 6.31 (s, 1H), 6.02 (s, 1H), 5.09 (t, J=7.0 Hz, 1H), 3.64 (t, J=5.5 Hz, 1H), 3.51-3.36 (m, 3H), 3.36-3.11 (m, 3H), 3.07-2.84 (m, 3H), 2.79-2.52 (m, 2H), 2.23-1.87 (m, 3H), 0.98 (s, 9H); ¹³C{¹H}-NMR (100 MHz, MeOD): δ [ppm]=177.98, 174.33, 168.57, 165.20, 134.21, 129.70, 128.72, 127.96, 88.82, 86.40, 84.10, 72.22, 71.17, 71.09, 66.82, 63.17, 60.87, 59.35, 48.76, 45.14, 44.01, 42.68, 41.83, 37.33, 37.11, 36.17, 28.30, 25.85; HRMS (ESI) m/z: [M+H]⁺ Calcd for C₂₇H₃₂N₂O₈H⁺ 513.22314, found 513.22286.

Synthesis of (3aS,5aS,8R,8aS,9R,10aS)-6-(((1S,3S)-adamantan-2-yl)methyl)-9-(tert-butyl)-9-hydroxy-2,4,7-trioxooctahydro-4H,9H-furo[3″,2″:2′,3′]cyclopenta[1′,2′:3,4]furo[2,3-b]pyrrol-8-yl 4-(aminomethyl)benzoate hydrochloride (SCC506). From SCC505 (90.1 μmol, 63.7 mg) in 3M HCl in MeOH yielded SCC506 (100%). ¹H-NMR (600 MHz, MeOD-d4): δ [ppm]=8.05 (d, J=8.4 Hz, 1H), 7.61 (d, J=8.2 Hz, 2H), 6.42 (s, 1H), 6.13 (s, 1H), 5.17 (t, J=6.9 Hz, 1H), 4.22 (s, 2H), 3.39 (d, J=14.9 Hz, 1H), 3.00 (d, J=19.0 Hz, 1H), 2.95 (d, J=19.0 Hz, 1H), 2.86 (d, J=14.5 Hz, 1H), 2.75 (dd, J=7.1, 13.6 Hz, 1H), 2.10 (dd, J=7.0, 13.3 Hz, 1H), 2.01 (brs, 3H), 1.79 (m, 3H), 1.69 (m, 3H), 1.62 (m, 6H), 1.11 (s, 9H).

### Synthesis of 4-(3aS,5aS,8R,9R,10aS)-8-(benzoyloxy)-9-(tert-butyl)-9-hydroxy-2,4,7-trioxohexahydro-4H,9H-furo[3″,2″:2′,3′]cyclopenta[1′,2′:3,4]furo[2,3-b]pyrrol-6(5aH)-yl)benzenaminium chloride (XBB-037)

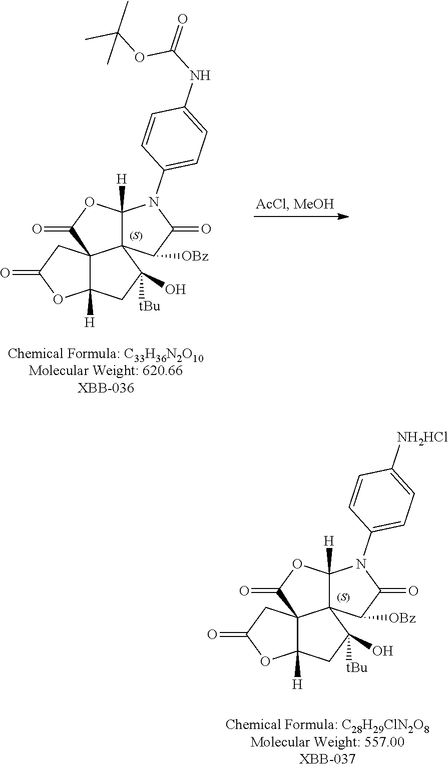

Acetyl chloride (0.35 mL, 4.95 mmol, 15 equiv.) was added dropwise to methanol (10 mL) while cooled in an ice bath. The solution is stirred at room temperature for 15 minutes to generate a 0.5 M HCl solution. XBB-036 (207 mg, 0.33 mmol, 1 equiv.) was added to the HCl solution and allowed to stir for 18 hours at room temperature. On completion, the reaction mixture is concentrated under reduced pressure to give a yellow residue. The residue is then suspended in a mixture of hexane/ethyl acetate (1:1, 10 mL) and sonicated to reach homogeneity, before being filtered through a sintered funnel. The resultant residue is washed several times with hexane/ethyl acetate (1:1) to produce the desired XBB-037 as HCl salt (155 mg, 85%) as a white solid. mp=115.4-116.3° C.; ¹H-NMR (400 MHz, MeOD): δ [ppm]=8.04-7.98 (m, 2H), 7.94-7.84 (m, 2H), 7.72-7.65 (m, 1H), 7.59-7.48 (m, 4H), 6.65 (s, 1H), 6.60 (s, 1H), 5.24 (t, J=7.1 Hz, 1H), 5.04 (s, 2H), 3.23-3.00 (m, 2H), 2.82 (dd, J=13.8, 7.2 Hz, 1H), 2.22 (dd, J=13.8, 7.2 Hz, 1H), 1.13 (s, 9H); ¹³C{¹H}-NMR (100 MHz, MeOD): δ [ppm]=176.35, 173.07, 165.86, 163.80, 134.82, 132.87, 128.41, 127.73, 127.37, 126.97, 126.65, 126.07, 122.54, 86.56, 85.34, 82.87, 65.49, 61.15, 59.54, 57.99, 41.27, 40.55, 35.91, 34.67, 24.47; HRMS (ESI) m/z: [M+Na]⁺ Calcd for C₂₈H₂₈N₂O₈Na⁺ 543.17379, found 543.17396.

### Synthesis of (3aS,5aS,8R,8aS,9R,10aS)-9-(tert-butyl)-9-hydroxy-6-(1H-indol-5-yl)-2,4,7-trioxooctahydro-4H,9H-furo[3″,2″:2′,3′]cyclopenta[1′,2′:3,4]furo[2,3-b]pyrrol-8-yl benzoate (SCC382)

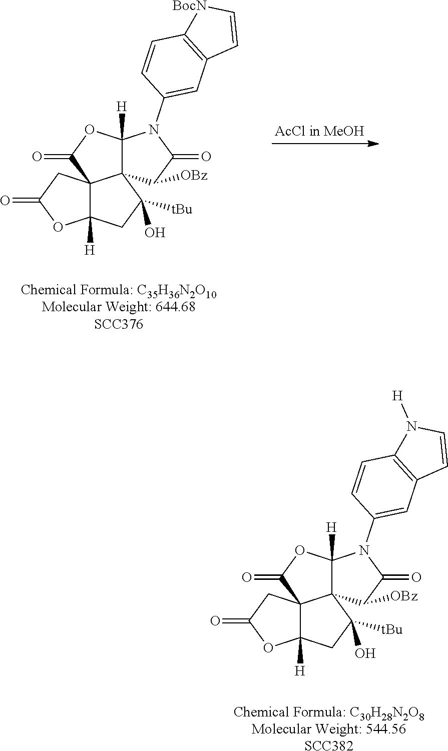

Acetyl chloride (0.43 mL, 6 mmol) was added dropwise to methanol (1.6 mL) while cooled in an ice bath resulting in a 3M HCl solution in Methanol/MeOAc. This solution is added to SCC376 (28.3 mg, 43.9 μmol) and allowed to stir for 22 hours at room temperature. On completion, sat. NaHCO₃ solution (10 mL) was added followed by EtOAc (10 mL). After phase separation, the aqueous phase was extracted with EtOAc (10 mL). The combined organic phases were dried over MgSO₄ and concentrated under reduced pressure. Column chromatography (SiO2, EtOAc:hexane 1:1) yielded the desired SCC382 (14.3 mg, 60%) as a white solid. ¹H-NMR (600 MHz, CDCl₃): δ [ppm]=8.41 (s, 1H), 8.05 (d, J=7.2 Hz, 2H), 7.73 (s, 1H), 7.65 (t, J=7.2 Hz, 1H), 7.52 (t, J=8.1 Hz, 2H), 7.36 (d, J=8.6 Hz, 1H), 7.25 (t, J=2.7 Hz, 1H), 7.21 (dd, J=2.2, 9.0 Hz, 1H), 6.56 (s, 1H), 6.54 (s, 1H), 6.39 (s, 1H), 5.24 (t, J=7.1 Hz, 1H), 3.40 (d, J=19.0 Hz, 1H), 3.03 (d, J=18.6 Hz, 1H), 2.65 (dd, J=7.1, 13.8 Hz, 1H), 2.30 (dd, J=7.2, 14.0 Hz, 1H), 2.29 (s, 1H), 1.15 (s, 9H).

### Synthesis of (3aS,5aS,8R,8aS,9R,10aS)-6-(((1S,5S)-3-(aminomethyl)adamantan-1-yl)methyl)-9-(tert-butyl)-9-hydroxy-2,4,7-trioxooctahydro-4H,9H-furo[3″,2″:2′,3′]cyclopenta[1′,2′:3,4]furo[2,3-b]pyrrol-8-yl benzoate (SCC567)

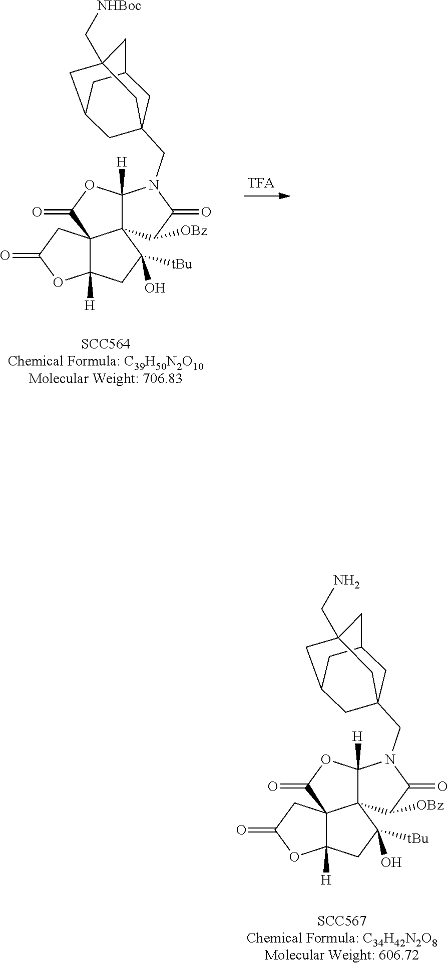

SCC564 (0.10 mmol, 70.1 mg) was dissolved in DCM/TFA (2.5 mL, 5:1) and stirred at room temperature. After 5 h, the solvent was removed and the residue was re-dissolved in DCM (5 mL) and sat. NaHCO₃ (10 mL) was added. After phase separation, the aqueous phase was extracted with DCM (4×10 mL). The combined organic layers were washed with brine (15 mL), dried over MgSO₄. The removal of the volatiles yielded in SCC567 (41.2 mg, 68%).

### Example 1.6: Synthesis of N-Alkylated Bilobalide Lactam Analogues According to Scheme 2 or Scheme 5

Synthesis of N-alkylated bilobalide lactam analogues according to the method described in Scheme 2 or Scheme 5 is described herein.

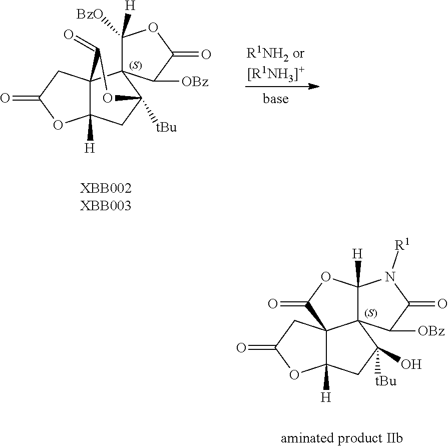

To a solution of XBB-002 or XBB-003 in anhydrous tetrahydrofuran or CH₃Cl (1% EtOH) was added R₁NH₂ or [R₁NH₃]⁺ at 0° C. In some embodiments, the reaction mixture further included Et₃N or DIPEA. The resulting solution was warmed to room temperature and stirred for 30 min up to 24 h. Upon completion indicated by TLC, the reaction solution was concentrated in vacuo. The residue was dissolved in saturated NH₄Cl or saturated NaHCO₃ solution and DCM or EtOAc. After phase separation, the aqueous phase was extracted with DCM or EtOAc. The combined organic layers were washed with brine, dried over Na₂SO₄, filtered and concentrated in vacuo. The crude product was purified via column chromatography to provide the corresponding aminated product IIb (i.e., N-alkylated bilobalide analogues according to Table 1g) as a white powder.

Examples of N-alkylated bilobalide analogues are summarized in Table 1g.

TABLE 1g
Example N-alkylated bilobalide lactam analogues

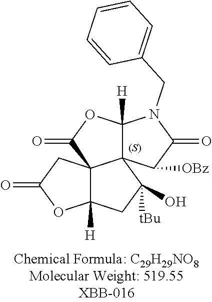

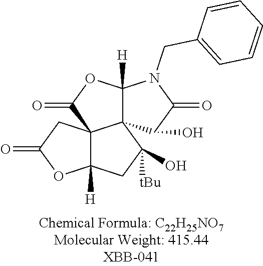 [SMILES: CC(C)(C)[C@]1(O)C[C@@H]2OC(=O)C[C@@]23C(=O)O[C@@H]2N(Cc4ccccc4)C(=O)[C@H](O)[C@]213]

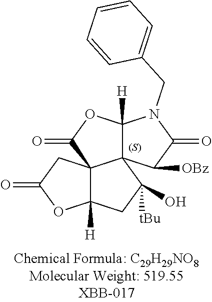

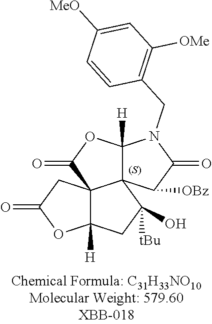

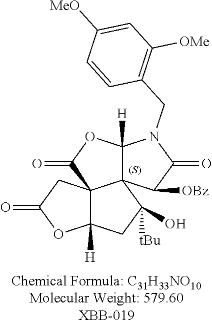

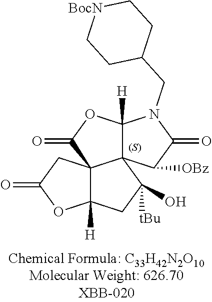

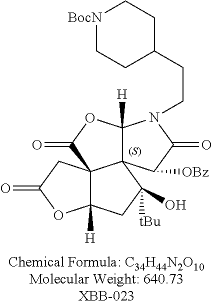

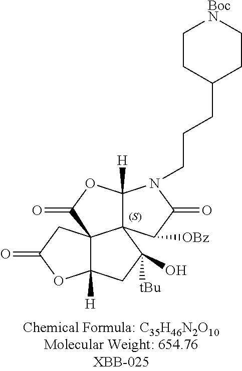

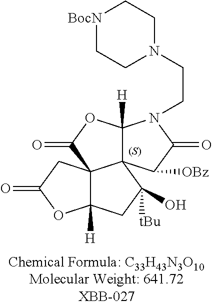

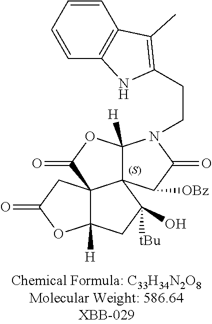

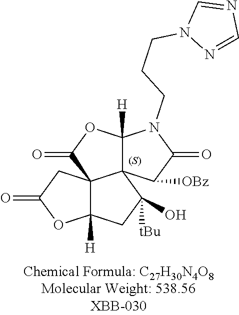

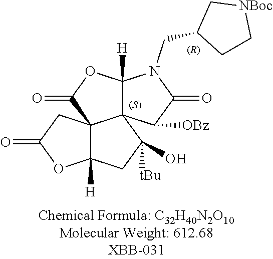

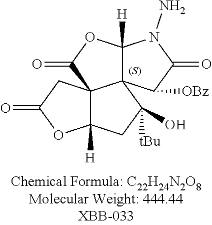

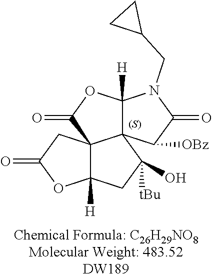

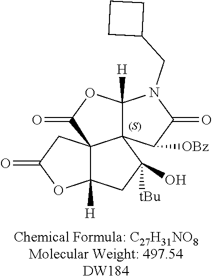

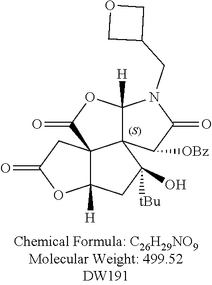

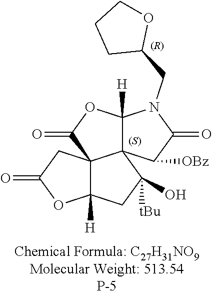

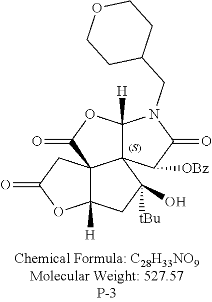

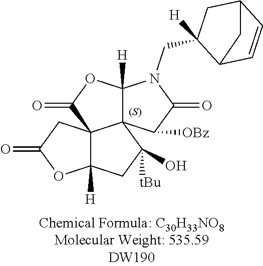

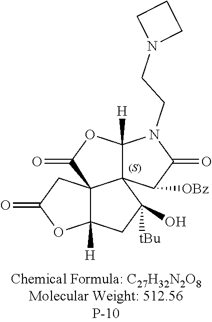

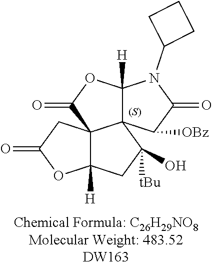

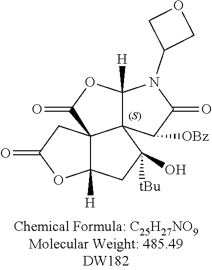

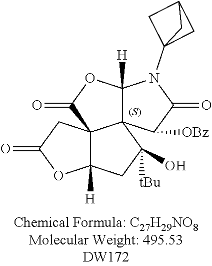

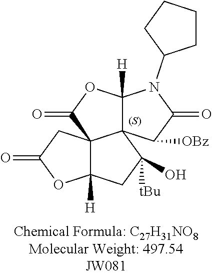

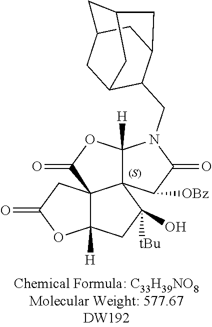

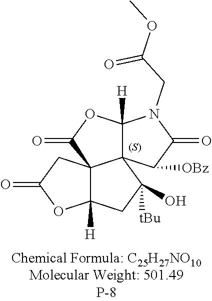

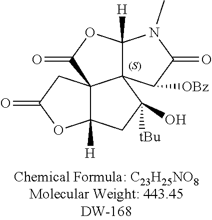

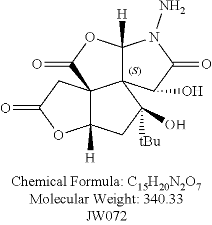

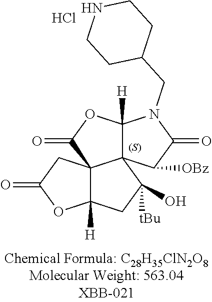

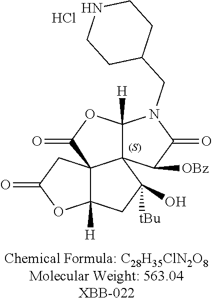

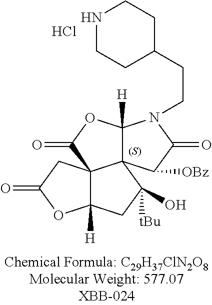

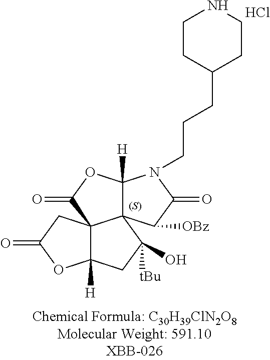

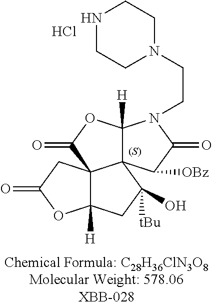

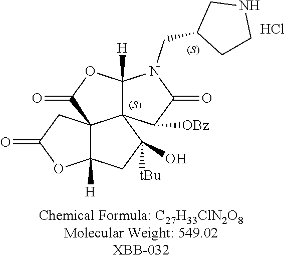

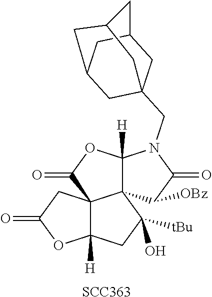

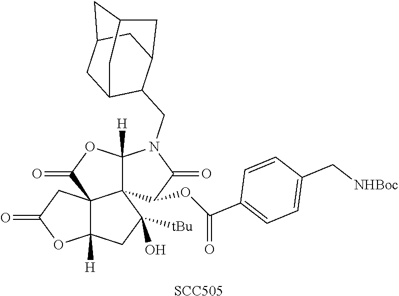

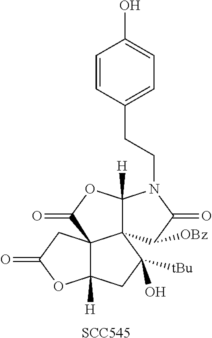

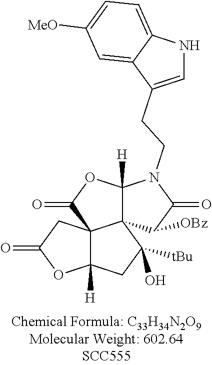

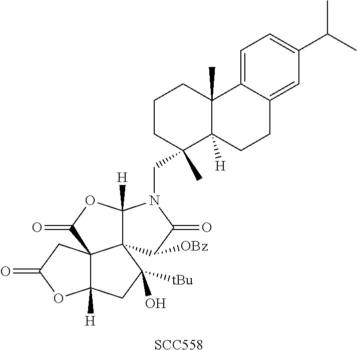

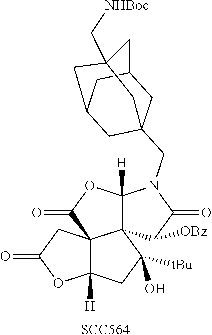

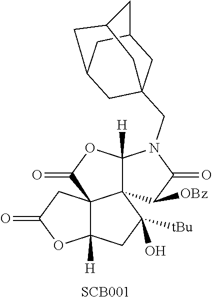

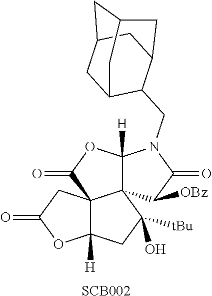

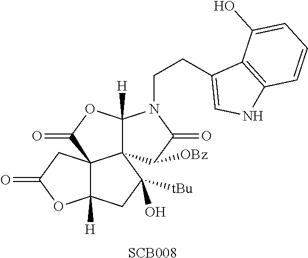

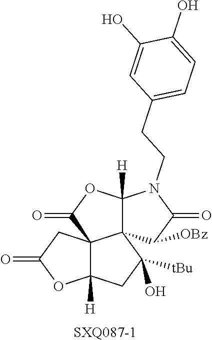

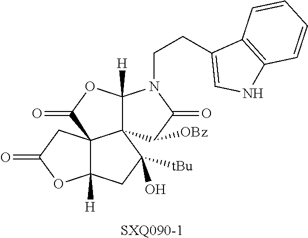

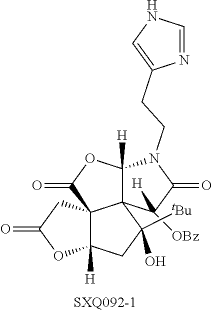

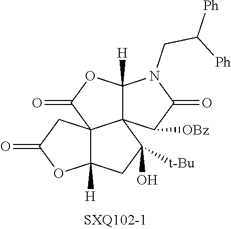

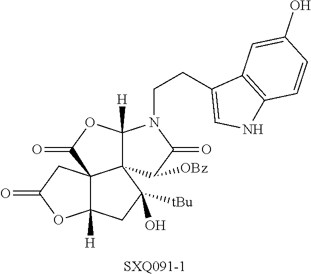

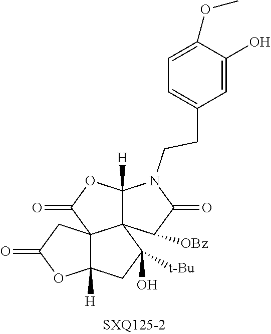

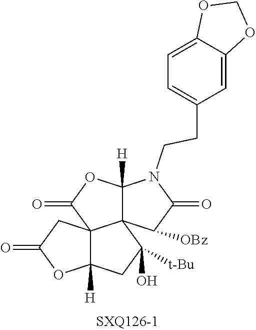

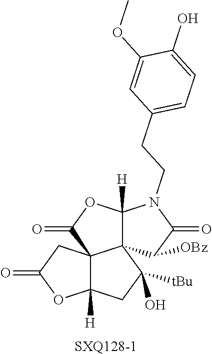

Synthesis of (3aS,5aS,8S,8aS,9R,10aS)-6-benzyl-9-(tert-butyl)-9-hydroxy-2,4,7-trioxooctahydro-4H,9H-furo [3″,2″:2′,3′]cyclopenta [1′,2′:3,4]furo [2,3-b]pyrrol-8-yl benzoate (XBB-017). Using XBB-003 (50.0 mg, 93.6 μmol, 1.00 eq.), benzylamine (2.00 eq.) in THF (2 mL) yielding in XBB-017 as a white powder (39 mg, 80%). R_{f}=0.35 (hexane:EtOAc, 3:1); mp=181.4-182.3° C.; ¹H-NMR (500 MHz, CDCl₃): δ [ppm]=7.96-7.90 (m, 2H), 7.58 (t, J=7.5 Hz, 1H), 7.43 (t, J=7.6 Hz, 2H), 7.37-7.31 (m, 2H), 7.26-7.21 (m, 3H), 6.46 (s, 1H), 5.43 (s, 1H), 4.57 (d, J=14.4 Hz, 1H), 4.41 (d, J=14.2 Hz, 1H), 4.07-3.99 (m, 1H), 3.34 (d, J=14.1 Hz, 1H), 2.76 (d, J=14.0 Hz, 1H), 2.39-2.30 (m, 1H), 2.27-2.15 (m, 1H), 0.89 (s, 9H); ¹³C{¹H}-NMR (125 MHz, CDCl₃): δ [ppm]=176.90, 175.00, 170.03, 165.99, 135.13, 133.34, 129.72, 129.64, 129.55, 128.71, 128.64, 128.15, 100.47, 83.07, 77.29, 77.03, 76.78, 70.07, 67.23, 66.11, 60.84, 45.22, 38.53, 35.11, 32.90, 27.00; HRMS (ESI) m/z: [M+Na]⁺ Calcd for C₂₉H₂₉NO₈Na⁺ 542.17854, found 542.17850.

Synthesis of (3aS,5aS,8R,8aS,9R,10aS)-6-(2,4-dimethoxy-benzyl-9-(tert-butyl)-9-hydroxy-2,4,7-trioxooctahydro-4H,9H-furo [3″,2″:2′,3′]cyclopenta [1′,2′:3,4]furo [2,3-b]pyrrol-8-yl benzoate (XBB-018). Using XBB-002 (50.0 mg, 93.6 μmol, 1.00 eq.), 2,4-dimethoxybenzylamine (2.00 eq.) in THF (2 mL) yielding in XBB-018 as a white powder (49 mg, 90%). R_{f}=0.2 (hexane:EtOAc, 3:1); mp=102.3-102.9° C.; ¹H-NMR (500 MHz, CDCl₃): δ [ppm]=7.99 (d, J=7.7 Hz, 2H), 7.62 (t, J=7.3 Hz, 1H), 7.48 (t, J=7.7 Hz, 2H), 7.21 (d, J=8.8 Hz, 1H), 6.51-6.43 (m, 2H), 6.24 (s, 1H), 5.72 (s, 1H), 5.17 (t, J=7.1 Hz, 1H), 4.97 (d, J=14.3 Hz, 1H), 4.08 (d, J=14.4 Hz, 1H), 3.85 (s, 3H), 3.82 (s, 3H), 3.21 (d, J=19.0 Hz, 1H), 2.90 (d, J=19.0 Hz, 1H), 2.62 (dd, J=13.9, 7.1 Hz, 1H), 2.15 (dd, J=13.9, 7.2 Hz, 1H), 0.95 (s, 9H); ¹³C{¹H}-NMR (125 MHz, CDCl₃): δ [ppm]=178.45, 173.79, 166.90, 165.26, 161.38, 158.68, 134.14, 132.23, 130.06, 128.72, 127.92, 114.19, 104.10, 98.69, 87.25, 87.12, 83.44, 70.90, 61.84, 60.52, 59.46, 55.45, 55.37, 42.77, 41.11, 37.14, 36.45; HRMS (ESI) m/z: [M+Na]⁺ Calcd for C₃₁H₃₃NO₁₀Na⁺ 602.19967, found 602.19967. The X-ray crystal structure of XBB-018 is shown in FIG. 1E.

Synthesis of (3aS,5aS,8S,8aS,9R,10aS)-6-(2,4-dimethoxy-benzyl-9-(tert-butyl)-9-hydroxy-2,4,7-trioxooctahydro-4H,9H-furo [3″,2″:2′,3′]cyclopenta [1′,2′:3,4]furo [2,3-b]pyrrol-8-yl benzoate (XBB-019). Using XBB-003 (50.0 mg, 93.6 μmol, 1.00 eq.), 2,4-dimethoxybenzylamine (2.00 eq.) in THF (2 mL) yielding in XBB-019 as a white powder (47 mg, 86%). R_{f}=0.2 (hexane:EtOAc, 3:1); mp=106.2-107.1° C.; ¹H-NMR (500 MHz, CDCl₃): δ [ppm]=7.80 (d, J=7.6 Hz, 2H), 7.60 (t, J=7.5 Hz, 1H), 7.42 (t, J=7.7 Hz, 2H), 7.24 (d, J=8.2 Hz, 1H), 6.50 (s, 1H), 6.42 (dd, J=8.3, 2.3 Hz, 1H), 6.03 (s, 1H), 5.80 (s, 1H), 5.16 (t, J=7.1 Hz, 1H), 4.87 (d, J=14.2 Hz, 1H), 4.19 (s, 1H), 3.82 (s, 3H), 3.76 (s, 3H), 2.99 (s, 2H), 2.61 (dd, J=14.3, 7.1 Hz, 1H), 2.13 (dd, J=14.3, 7.2 Hz, 1H), 0.95 (s, 9H); ¹³C{¹H}-NMR (125 MHz, CDCl₃): δ [ppm]=177.85, 172.26, 167.15, 164.99, 161.26, 158.70, 133.99, 132.25, 130.15, 128.67, 128.57, 114.73, 104.17, 98.68, 90.98, 90.49, 83.54, 71.21, 61.09, 60.40, 55.58, 55.43, 42.62, 39.98, 37.26, 35.18, 26.69; HRMS (ESI) m/z: [M+Na]⁺ Calcd for C₃₁H₃₃NO₁₀Na⁺ 602.19967, found 602.19961.

Synthesis of tert-butyl 4-(((3aS,5aS,8R,8aS,9R,10aS)-8-(benzoyloxy)-9-(tert-butyl)-9-hydroxy-2,4,7-trioxohexahydro-4H,9H-furo [3″,2″:2′,3′]cyclopenta[1′,2′:3,4]furo [2,3-b]pyrrol-6(5aH)-yl)methyl)piperidine-1-carboxylate (XBB-020). Using XBB-002 (50.0 mg, 93.6 μmol, 1.00 eq.), 1-Boc-4-(aminomethyl)piperidine (2.00 eq.) in THF (2 mL) yielding in XBB-020 as a white powder (42 mg, 72%). R_{f}=0.2 (hexane:EtOAc, 2:1); mp=156.7-157.4° C.; ¹H-NMR (500 MHz, CDCl₃): δ [ppm]=7.98 (d, J=7.7 Hz, 2H), 7.63 (t, J=7.5 Hz, 1H), 7.49 (t, J=7.7 Hz, 2H), 6.36 (s, 1H), 5.95 (s, 1H), 5.19 (t, J=7.2 Hz, 1H), 4.21-3.92 (m, 3H), 3.45 (d, J=12.5 Hz, 1H), 3.23-2.95 (m, 3H), 2.87 (d, J=18.8 Hz, 1H), 2.76-2.61 (m, 3H), 2.26-2.16 (m, 1H), 1.95 (q, J=10.5, 9.9 Hz, 1H), 1.85-1.71 (m, 1H), 1.57 (m, 1H), 1.45 (d, J=2.0 Hz, 9H), 1.10 (s, 9H); ¹³C{¹H}-NMR (125 MHz, CDCl₃): δ [ppm]=177.89, 173.49, 173.46, 167.45, 165.26, 154.74, 134.27, 130.07, 128.77, 127.75, 87.21, 87.19, 87.17, 83.36, 83.34, 79.75, 70.67, 62.73, 62.71, 59.27, 42.87, 42.85, 42.83, 37.42, 36.46, 34.37, 29.81, 28.42, 26.46; HRMS (ESI) m/z: [M+Na]⁺ Calcd for C₃₃H₄₂N₂ONa⁺ 649.27317, found 649.27258.

Synthesis of tert-butyl 4-(2-((3aS,5aS,8R,8aS,9R,10aS)-8-(benzoyloxy)-9-(tert-butyl)-9-hydroxy-2,4,7-trioxohexahydro-4H,9H-furo[3″,2″:2′,3′]cyclopenta[1′,2′:3,4]furo[2,3-b]pyrrol-6(5aH)-yl)ethyl)piperidine-1-carboxylate (XBB-023). Using XBB-002 (50.0 mg, 93.6 μmol, 1.00 eq.), 1-Boc-4-(aminoethyl)piperidine (2.00 eq.) in THF (2 mL) yielding in XBB-023 as a white powder (51 mg, 86%). R_{f}=0.2 (hexane:EtOAc, 2:1); mp=136.7-137.4° C.; ¹H-NMR (400 MHz, CDCl₃): δ [ppm]=8.04-7.91 (m, 2H), 7.67-7.56 (m, 1H), 7.55-7.41 (m, 2H), 6.32 (s, 1H), 5.95 (s, 1H), 5.20 (t, J=7.1 Hz, 1H), 4.20-3.98 (m, 2H), 3.60 (ddd, J=13.8, 9.5, 6.2 Hz, 1H), 3.32 (ddd, J=14.3, 9.4, 5.7 Hz, 1H), 3.16 (d, J=18.8 Hz, 1H), 2.87 (d, J=18.8 Hz, 1H), 2.76-2.57 (m, 4H), 2.27-2.19 (m, 1H), 2.18 (s, 1H), 1.68 (d, J=8.2 Hz, 3H), 1.65-1.49 (m, 2H), 1.45 (s, 9H), 1.10 (s, 9H); ¹³C{¹H}-NMR (100 MHz, CDCl₃): δ [ppm]=178.01, 173.70, 167.15, 165.29, 154.91, 134.25, 130.06, 128.77, 127.81, 87.81, 86.96, 83.56, 79.61, 70.93, 62.87, 59.31, 42.60, 38.89, 37.41, 36.44, 33.81, 33.71, 31.72, 28.45, 26.49; HRMS (ESI) m/z: [M+Na]⁺ Calcd for C₃₄H₄₄N₂O₁₀Na⁺ 663.28882, found 663.28809.

Synthesis of (tert-butyl 4-(3-((3aS,5aS,8R,8aS,9R,10aS)-8-(benzoyloxy)-9-(tert-butyl)-9-hydroxy-2,4,7-trioxohexahydro-4H,9H-furo[3″,2″:2′,3′]cyclopenta[1′,2′:3,4]furo[2,3-b]pyrrol-6(5aH)-yl)propyl) piperidine-1-carboxylate (XBB-025). Using XBB-002 (50.0 mg, 93.6 μmol, 1.00 eq.), 1-Boc-4-(3-aminopropyl)piperidine (2.00 eq.) in THF (2 mL) yielding in XBB-025 as a white powder (48 mg, 78%). R_{f}=0.2 (hexane:EtOAc, 2:1); mp=127.8-128.6° C.; ¹H-NMR (400 MHz, CDCl₃): δ [ppm]=8.04-7.91 (m, 2H), 7.67-7.58 (m, 1H), 7.49 (t, J=7.8 Hz, 2H), 6.33 (s, 1H), 5.97 (s, 1H), 5.20 (t, J=7.1 Hz, 1H), 4.05 (d, J=13.1 Hz, 2H), 3.60-3.47 (m, 1H), 3.33-3.21 (m, 1H), 3.16 (d, J=18.9 Hz, 1H), 3.03-2.95 (m, 1H), 2.87 (d, J=18.7 Hz, 1H), 2.75-2.54 (m, 2H), 2.22 (dd, J=13.9, 7.1 Hz, 1H), 1.83-1.77 (m, 1H), 1.73-1.53 (m, 4H), 1.45 (s, 9H), 1.42-1.35 (m, 1H), 1.26-1.20 (m, 2H), 1.10 (s, 9H); ¹³C{¹H}-NMR (100 MHz, CDCl₃): δ [ppm]=178.08, 173.80, 167.22, 165.35, 155.00, 134.26, 130.07, 128.77, 127.81, 87.88, 86.95, 83.60, 79.58, 70.97, 62.85, 60.57, 59.34, 42.62, 41.31, 37.41, 36.45, 35.49, 33.51, 32.02, 28.46, 26.50, 24.33; HRMS (ESI) m/z: [M+Na]⁺ Calcd for C₃₅H₄₆N₂O₁₀Na⁺ 677.30447, found 677.30410.

Synthesis of tert-butyl 4-(2-((3aS,5aS,8R,8aS,9R,10aS)-8-(benzoyloxy)-9-(tert-butyl)-9-hydroxy-2,4,7-trioxohexahydro-4H,9H-furo[3″,2″:2′,3′]cyclopenta[1′,2′:3,4]furo[2,3-b]pyrrol-6(5aH)-yl)ethyl) piperazine-1-carboxylate (XBB-027). Using XBB-002 (50.0 mg, 93.6 μmol, 1.00 eq.), 1-boc-4-(2-aminoethyl)piperazine (2.00 eq.) in THF (2 mL) yielding in XBB-027 as a white powder (51 mg, 85%). R_{f}=0.1 (hexane:EtOAc, 2:1); mp=134.4-135.1° C.; ¹H-NMR (400 MHz, CDCl₃): δ [ppm]=8.03-7.95 (m, 2H), 7.62 (t, J=7.4 Hz, 1H), 7.49 (t, J=7.8 Hz, 2H), 6.36 (s, 1H), 6.20 (s, 1H), 5.21 (t, J=7.1 Hz, 1H), 3.80 (dt, J=14.7, 5.5 Hz, 1H), 3.60-3.21 (m, 5H), 3.17 (d, J=18.9 Hz, 1H), 2.90 (d, J=18.9 Hz, 1H), 2.81 (s, 1H), 2.71-2.58 (m, 3H), 2.55-2.30 (m, 5H), 2.23 (dd, J=13.9, 7.1 Hz, 1H), 1.46 (s, 9H), 1.11 (s, 9H); ¹³C{¹H}-NMR (100 MHz, CDCl₃): δ [ppm]=178.28, 173.68, 167.09, 165.30, 154.79, 134.20, 130.08, 128.75, 127.85, 88.37, 87.13, 83.45, 79.97, 70.91, 62.48, 59.31, 55.33, 52.68, 42.84, 37.41, 36.75, 36.57, 28.40, 26.51; HRMS (ESI) m/z: [M+H]⁺ Calcd for C₃₃H₄₃N₃O₁₀H⁺ 642.30212, found 642.30203.

Synthesis of (3aS,5aS,8R,8aS,9R,10aS)-9-(tert-butyl)-9-hydroxy-6-(2-(3-methyl-1H-indol-2-yl)ethyl)-2,4,7-trioxooctahydro-4H,9H-furo [3″,2″:2′,3′]cyclopenta[1′,2′:3,4]furo[2,3-b]pyrrol-8-yl benzoate (XBB-029). Using XBB-002 (50.0 mg, 93.6 μmol, 1.00 eq.), 2-(3-methyl-1H-indol-2-yl)ethylamine (2.00 eq.) in THF (2 mL) yielding in XBB-029 as a white powder (45 mg, 82%). R_{f}=0.15 (hexane:EtOAc, 2:1); mp=143.2-144.1° C.; ¹H-NMR (500 MHz, MeOD): δ [ppm]=7.97 (d, J=7.7 Hz, 2H), 7.71-7.59 (m, 1H), 7.49 (t, J=7.6 Hz, 3H), 7.27-7.17 (m, 1H), 7.10-6.92 (m, 2H), 6.05 (s, 1H), 5.19 (s, 1H), 5.05 (t, J=7.1 Hz, 1H), 3.81 (ddd, J=14.2, 6.8, 3.2 Hz, 1H), 3.55 (ddd, J=14.1, 10.5, 6.1 Hz, 1H), 3.28 (ddd, J=14.5, 8.3, 4.6 Hz, 1H), 3.01-2.74 (m, 3H), 2.56 (dd, J=13.7, 7.2 Hz, 1H), 2.42 (s, 3H), 2.00-1.90 (m, 1H), 0.62 (s, 9H); ¹³C{H}-NMR (125 MHz, MeOD): δ [ppm]=179.48, 175.81, 169.40, 166.43, 137.22, 135.24, 133.86, 130.92, 129.90, 129.46, 121.83, 120.19, 118.25, 111.70, 107.24, 90.32, 87.35, 85.36, 72.45, 64.50, 60.48, 42.95, 42.45, 37.97, 37.17, 26.54, 22.22, 11.44; HRMS (ESI) m/z: [M+Na]⁺ Calcd for C₃₃H₃₄N₂O₈Na⁺ 609.22074, found 609.22060.

Synthesis of (3aS,5aS,8R,8aS,9R,10aS)-6-(3-(1H-1,2,4-triazol-1-yl)propyl)-9-(tert-butyl)-9-hydroxy-2,4,7-trioxooctahydro-4H,9H-furo [3″,2″:2′,3′]cyclopenta[1′,2′:3,4]furo[2,3-b]pyrrol-8-yl benzoate (XBB-030). Using XBB-002 (50.0 mg, 93.6 μmol, 1.00 eq.), 3-(1H-1,2,4-triazol-1-yl)propylamine (2.00 eq.) in THF (2 mL) yielding in XBB-030 as a white powder (38 mg, 75%). R_{f}=0.2 (hexane:EtOAc, 3:1); mp=163.2-163.5° C.; ¹H-NMR (400 MHz, DMSO-d6): δ [ppm]=8.02-7.87 (m, 2H), 7.78-7.68 (m, 1H), 7.64 (t, J=1.1 Hz, 1H), 7.55 (t, J=7.8 Hz, 2H), 7.20 (d, J=1.3 Hz, 1H), 6.90 (t, J=1.0 Hz, 1H), 6.26 (s, 1H), 6.03 (s, 1H), 5.61 (s, 1H), 5.07 (t, J=7.1 Hz, 1H), 4.00 (t, J=6.9 Hz, 2H), 3.25-3.07 (m, 3H), 2.79 (d, J=19.2 Hz, 1H), 2.69 (d, J=6.5 Hz, 1H), 2.20-1.69 (m, 3H), 1.00 (s, 9H); HRMS (ESI) m/z: [M+H]⁺ Calcd for C₂₈H₃₀N₃O₈H⁺ 538.21839, found 538.21835.

Synthesis of tert-butyl (R)-3-(((3aS,5aS,8R,8aS,9R,10aS)-8-(benzoyloxy)-9-(tert-butyl)-9-hydroxy-2,4,7-trioxohexahydro-4H,9H-furo[3″,2″:2′,3′]cyclopenta[1′,2′:3,4]furo[2,3-b]pyrrol-6(5aH)-yl)methyl) pyrrolidine-1-carboxylate (XBB-031). Using XBB-002 (50.0 mg, 93.6 μmol, 1.00 eq.), (R)-2-(Aminomethyl)pyrrolidine (2.00 eq.) in THF (2 mL) yielding in XBB-031 as a white powder (48 mg, 82%). R_{f}=0.2 (hexane:EtOAc, 3:1); mp=126.7-127.2° C.; ¹H-NMR (500 MHz, CDCl₃): δ [ppm]=8.11-7.88 (m, 2H), 7.61 (t, J=7.5 Hz, 2H), 7.48 (t, J=7.7 Hz, 2H), 6.30 (s, 1H), 5.96 (s, 1H), 5.03 (t, J=7.0 Hz, 1H), 3.87 (dd, J=14.2, 4.8 Hz, 1H), 3.59-3.34 (m, 3H), 3.29-3.01 (m, 3H), 2.96-2.46 (m, 3H), 2.24-2.09 (m, 1H), 2.01-1.90 (m, 1H), 1.79-1.58 (m, 1H), 1.47 (s, 9H), 1.12 (s, 9H); ¹³C{¹H}-NMR (125 MHz, CDCl₃): δ [ppm]=178.33, 173.77, 167.25, 165.28, 155.44, 134.20, 130.06, 128.76, 127.86, 87.96, 86.23, 83.76, 80.14, 70.79, 63.11, 59.24, 49.73, 45.77, 43.17, 42.24, 37.57, 37.32, 36.44, 29.17, 28.58, 28.50; HRMS (ESI) m/z: [M+Na]⁺ Calcd for C₃₂H₄₀N₂O₁₀Na⁺ 635.25752, found 635.25714.

Synthesis of (3aS,5aS,8R,8aS,9R,10aS)-6-amino-9-(tert-butyl)-9-hydroxy-2,4,7-trioxooctahydro-4H,9H-furo[3″,2″:2′,3′]cyclopenta [1′,2′:3,4]furo [2,3-b]pyrrol-8-yl benzoate (XBB-033). Using XBB-002 (50.0 mg, 93.6 μmol, 1.00 eq.), hydrazine (2.00 eq.) in THF (2 mL) yielding in XBB-033 as a white powder (32 mg, 78%). R_{f}=0.1 (hexane:EtOAc, 2:1); mp=108.4° C.; ¹H-NMR (400 MHz, MeOD): δ [ppm]=8.09-7.93 (m, 2H), 7.74-7.61 (m, 1H), 7.52 (t, J=7.8 Hz, 2H), 6.40 (s, 1H), 5.97 (s, 1H), 5.19 (t, J=7.2 Hz, 1H), 3.35 (d, J=17.7 Hz, 1H), 3.03 (s, 2H), 2.74 (dd, J=13.7, 7.2 Hz, 1H), 2.14 (dd, J=13.7, 7.2 Hz, 1H), 1.09 (s, 9H); ¹³C{¹H}-NMR (100 MHz, MeOD): δ [ppm]=178.44, 174.63, 166.93, 165.13, 133.92, 129.59, 128.51, 128.05, 89.70, 86.22, 84.13, 70.04, 62.50, 59.42, 41.79, 37.02, 36.19, 25.59; HRMS (ESI) m/z: [M+H]⁺ Calcd for C₂₂H₂₄N₂O₈H⁺ 445.16122, found 445.16126.

Synthesis of (3aS,5aS,8R,8aS,9R,10aS)-9-(tert-butyl)-6-(cyclopropylmethyl)-9-hydroxy-2,4,7-trioxooctahydro-4H,9H-furo [3″,2″:2′,3′]cyclopenta [1′,2′:3,4]furo[2,3-b]pyrrol-8-yl benzoate (DW189). Using XBB-002 (150.0 mg, 0.28 mmol, 1.00 eq.), cyclopropylmethane amine (2.50 eq.) in THF (3 mL) yielding in DW189 as a white powder (78 mg, 57%). ¹H-NMR (500 MHz, CDCl₃): δ [ppm]=8.02-7.97 (m, 2H), 7.66-7.59 (m, 1H), 7.49 (t, J=7.8 Hz, 2H), 6.36 (s, 1H), 6.14 (s, 1H), 5.22 (t, J=7.1 Hz, 1H), 3.65 (dd, J=14.3, 6.4 Hz, 1H), 3.19 (d, J=18.9 Hz, 1H), 2.99-2.89 (m, 2H), 2.87 (s, 1H), 2.62 (dd, J=14.0, 7.2 Hz, 1H), 2.26 (dd, J=14.0, 7.2 Hz, 1H), 1.57 (s, 6H), 1.13 (s, 9H), 1.10-1.00 (m, 1H), 0.67-0.59 (m, 1H), 0.59-0.50 (m, 1H), 0.43 (dq, J=9.8, 4.9 Hz, 1H), 0.26 (dq, J=9.9, 5.0 Hz, 1H).

Synthesis of (3aS,5aS,8R,8aS,9R,10aS)-9-(tert-butyl)-6-(cyclobutyl methyl)-9-hydroxy-2,4,7-trioxooctahydro-4H,9H-furo [3″,2″:2′,3′]cyclopenta [1′,2′:3,4]furo [2,3-b]pyrrol-8-yl benzoate (DW184). Using XBB-002 (150.0 mg, 0.28 mmol, 1.00 eq.), cyclobutylmethane amine hydrochloride (3.00 eq.), Et₃N (6.00 eq.) in CHCl₃ (3 mL, 1% EtOH) yielding in DW184 as a white powder (62 mg, 44%). ¹H-NMR (500 MHz, CDCl₃): δ [ppm]=8.01-7.96 (m, 2H), 7.65-7.58 (m, 1H), 7.53-7.46 (m, 2H), 6.32 (s, 1H), 5.90 (s, 1H), 5.20 (t, J=7.1 Hz, 1H), 3.61 (dd, J=13.8, 8.0 Hz, 1H), 3.31 (dd, J=13.8, 7.7 Hz, 1H), 3.15 (s, 1H), 2.89 (s, 1H), 2.70 (hept, J=8.0 Hz, 1H), 2.61 (dd, J=14.0, 7.1 Hz, 1H), 2.25 (dd, J=14.0, 7.2 Hz, 1H), 2.13-1.69 (m, 7H), 1.10 (s, 9H).

Synthesis of (3aS,5aS,8R,8aS,9R,10aS)-9-(tert-butyl)-6-(1-oxetan-3-yl methyl)-9-hydroxy-2,4,7-trioxooctahydro-4H,9H-furo [3″,2″:2′,3′]cyclopenta [1′,2′:3,4]furo [2,3-b]pyrrol-8-yl benzoate (DW191). Using XBB-002 (150.0 mg, 0.28 mmol, 1.00 eq.), 1-(oxetan-3-yl)methanamine (2.50 eq.) in THF (3 mL) yielding in DW191 as a white powder.

Synthesis of (3aS,5aS,8R,8aS,9R,10aS)-9-(tert-butyl)-9-hydroxy-2,4,7-trioxo-6-(((R)-tetrahydrofuran-2-yl) methyl) octahydro-4H,9H-furo [3″,2″:2′,3′]cyclopenta [1′,2′:3,4]furo[2,3-b]pyrrol-8-yl benzoate (P-5). Using XBB-002 (75.0 mg, 0.14 mmol, 1.00 eq.), ((R)-tetrahydrofuran-2-yl)methane amine (2.50 eq.) in THF (3 mL) yielding in P-5 as a white powder (46.7 mg, 65%).

Synthesis of (3aS,5aS,8R,8aS,9R,10aS)-9-(tert-butyl)-9-hydroxy-2,4,7-trioxo-6-((tetrahydro-2H-pyran-4-yl) methyl) octahydro-4H,9H-furo [3″,2″:2′,3′]cyclopenta [1′,2′:3,4]furo [2,3-b]pyrrol-8-yl benzoate (P-3). Using XBB-002 (75.0 mg, 0.14 mmol, 1.00 eq.), tetrahydro-2H-pyran-4-ylmethane amine (2.50 eq.) in THF (3 mL) yielding in P-3 as a white powder (43.7 mg, 59%).

Synthesis of (3aS,5aS,8R,8aS,9R,10aS)-6-(((2R)-bicyclo[2.2.1]hept-5-en-2-yl)methyl)-9-(tert-butyl)-9-hydroxy-2,4,7-trioxooctahydro-4H,9H-furo[3″,2″:2′,3′]cyclopenta [1′,2′:3,4]furo [2,3-b]pyrrol-8-yl benzoate (DW190). Using XBB-002 (150.0 mg, 0.28 mmol, 1.00 eq.), ((2R)-bicyclo[2.2.1]hept-5-en-2-yl)methane amine (2.50 eq.) in THF (3 mL) yielding in DW190 as a white powder (42 mg, 28%). ¹H-NMR (500 MHz, CDCl₃): δ [ppm]=7.98 (dt, J=8.4, 1.6 Hz, 2H), 7.66-7.59 (m, 1H), 7.49 (t, J=7.8 Hz, 2H), 6.33 (s, 1H), 5.90 (s, 1H), 5.20 (t, J=7.1 Hz, 1H), 4.80 (dd, J=7.8, 6.5 Hz, 2H), 4.49 (dt, J=12.8, 6.3 Hz, 2H), 3.75 (d, J=7.1 Hz, 2H), 3.31 (hept, J=7.1 Hz, 1H), 3.11 (s, 1H), 2.88 (s, 1H), 2.62 (dd, J=14.1, 7.2 Hz, 1H), 2.25 (dd, J=14.1, 7.2 Hz, 1H), 2.01 (s, 1H), 1.33-1.23 (m, 2H), 1.10 (s, 6H), 0.88 (t, J=6.8 Hz, 2H).

Synthesis of (3aS,5aS,8R,8aS,9R,10aS)-6-(2-(azetidin-1-yl)ethyl)-9-(tert-butyl)-9-hydroxy-2,4,7-trioxooctahydro-4H,9H-furo[3″,2″:2′,3′]cyclopenta [1′,2′:3,4]furo[2,3-b]pyrrol-8-yl benzoate (P-10). Using XBB-002 (0.10 g, 0.19 mmol, 1.00 eq.), 2-(azetidin-1-yl)ethylamine (2.50 eq.) in THF (2 mL) yielding in P-10 as a white powder (10.8 mg, 23%).

Synthesis of (3aS,5aS,8R,8aS,9R,10aS)-9-(tert-butyl)-6-cyclobutyl-9-hydroxy-2,4,7-trioxooctahydro-4H,9H-furo [3″,2″:2′,3′]cyclopenta [1′,2′:3,4]furo [2,3-b]pyrrol-8-yl benzoate (DW163). Using XBB-002 (80.0 mg, 0.15 mmol, 1.00 eq.), cyclobutylamine (1.50 eq.) in THF (2 mL) yielding in DW163 (54%). ¹H-NMR (400 MHz, CDCl₃): δ [ppm]=7.98 (d, J=7.1 Hz, 2H), 7.62 (t, J=7.3 Hz, 1H), 7.49 (t, J=8.1 Hz, 2H), 6.28 (s, 1H), 6.05 (s, 1H), 5.21 (t, J=7.0 Hz, 1H), 4.35 (p, J=8.7 Hz, 1H), 3.19 (d, J=18.8 Hz, 1H), 2.88 (d, J=18.2 Hz, 1H), 2.61 (dd, J=7.1, 13.9 Hz, 1H), 2.34-2.49 (m, 2H), 2.20-2.31 (m, 3H), 1.99 (s, 1H), 1.70-1.89 (m, 2H), 1.11 (s, 9H).

Synthesis of (3aS,5aS,8R,8aS,9R,10aS)-9-(tert-butyl)-9-hydroxy-6-(oxetan-3-yl)-2,4,7-trioxooctahydro-4H,9H-furo [3″,2″:2′,3′]cyclopenta [1′,2′:3,4]furo [2,3-b]pyrrol-8-yl benzoate (DW182). Using XBB-002 (0.10 g, 0.19 mmol, 1.00 eq.), oxetan-3-ylamine (2.50 eq.) in THF (2 mL) yielding in DW182. ¹H-NMR (400 MHz, CDCl₃): δ [ppm]=7.98 (d, J=7.4 Hz, 2H), 7.63 (t, J=7.3 Hz, 1H), 7.50 (t, J=7.9 Hz, 2H), 6.32 (s, 1H), 6.20 (s, 1H), 5.21 (t, J=7.0 Hz, 1H), 4.99-5.08 (m, 2H), 4.82-4.93 (m, 3H), 3.16 (d, J=19.0 Hz, 1H), 2.89 (d, J=18.7 Hz, 1H), 2.65 (dd, J=7.0, 14.0 Hz, 1H), 2.26 (dd, J=6.9, 13.7 Hz, 1H), 2.24 (s, 1H), 1.12 (s, 9H).

Synthesis of (3aS,5aS,8R,8aS,9R,10aS)-6-(bicyclo[1.1.1]pentan-1-yl)-9-(tert-butyl)-9-hydroxy-2,4,7-trioxooctahydro-4H,9H-furo [3″,2″:2′,3′]cyclopenta [1′,2′:3,4]furo[2,3-b]pyrrol-8-yl benzoate (DW172). Using XBB-002 (0.10 g, 0.19 mmol, 1.00 eq.), bicyclo[1.1.1]pentylamine (2.00 eq.), Et₃N (3.00 eq) in CHCl₃ (2 mL, 1% EtOH) yielding in DW172 as a white powder (53 mg, 56%). ¹H-NMR (400 MHz, CDCl₃): δ [ppm]=7.99-7.96 (m, 2H), 7.61 (t, J=7.4 Hz, 1H), 7.48 (t, J=7.7 Hz, 2H), 6.28 (s, 1H), 5.93 (s, 1H), 5.20 (t, J=7.1 Hz, 1H), 3.20 (d, J=18.9 Hz, 1H), 2.90 (d, J=18.9 Hz, 1H), 2.61 (dd, J=14.0, 7.2 Hz, 1H), 2.56 (s, 1H), 2.23 (s, 7H), 1.11 (s, 9H).

Synthesis of (3aS,5aS,8R,8aS,9R,10aS)-9-(tert-butyl)-6-cyclopentyl-9-hydroxy-2,4,7-trioxooctahydro-4H,9H-furo[3″,2″:2′,3′]cyclopenta [1′,2′:3,4]furo[2,3-b]pyrrol-8-yl benzoate (JW081). Using XBB-002 (0.16 g, 0.30 mmol, 1.00 eq.), cyclopropylamine (2.50 eq.) in THF (2.5 mL) yielding in JW081 as a white powder (110 mg, 74%). ¹H-NMR (500 MHz, CDCl₃): δ [ppm]=7.97 (d, J=7.1 Hz, 2H), 7.61 (t, J=7.2 Hz, 1H), 7.48 (t, J=7.6 Hz, 2H), 6.28 (s, 1H), 6.03 (s, 1H), 5.18 (t, J=6.4 Hz, 1H), 4.07-4.16 (m, 1H), 3.18 (d, J=18.8 Hz, 1H), 2.86 (d, J=19.1 Hz, 1H), 2.62 (dd, J=8.2, 14.1 Hz, 1H), 2.38 (s, 1H), 2.22 (dd, J=7.5, 14.2 Hz, 1H), 1.96-2.07 (m, 1H), 1.75-1.95 (m, 3H), 1.64-1.74 (m, 2H), 1.54-1.65 (m, 2H), 1.08 (s, 9H).

Synthesis of (3aS,5aS,8R,8aS,9R,10aS)-6-(((1S,5R,7S)-adamantan-2-yl)methyl)-9-(tert-butyl)-9-hydroxy-2,4,7-trioxooctahydro-4H,9H-furo [3″,2″:2′,3′]cyclopenta[1′,2′:3,4]furo[2,3-b]pyrrol-8-yl benzoate (DW192). Using XBB-002 (0.18 g, 0.33 mmol, 1.00 eq.), adamantan-2-yl methylamine hydrochloride (3.00 eq.), Et₃N (6.00 eq.) in CHCl₃ (4 mL, 1% EtOH) yielding in DW192 as a white powder (32 mg, 17%). ¹H-NMR (500 MHz, CDCl₃): δ [ppm]=8.02-7.96 (m, 2H), 7.67-7.58 (m, 1H), 7.49 (t, J=7.9 Hz, 2H), 6.39 (s, 1H), 6.08 (s, 1H), 5.20 (t, J=7.1 Hz, 1H), 3.45 (d, J=14.3 Hz, 1H), 3.15 (s, 1H), 2.87 (s, 1H), 2.86-2.79 (m, 1H), 2.62 (dd, J=14.0, 7.2 Hz, 1H), 2.27 (dd, J=14.0, 7.1 Hz, 1H), 2.00 (s, 2H), 1.88 (s, 1H), 1.73 (d, J=12.5 Hz, 2H), 1.61 (d, J=12.4 Hz, 3H), 1.14 (s, 9H).

Synthesis of (3aS,5aS,8R,8aS,9R,10aS)-9-(tert-butyl)-9-hydroxy-6-(2-(methylamino)-2-oxoethyl)-2,4,7-trioxooctahydro-4H,9H-furo [3″,2″:2′,3′]cyclopenta[1′,2′:3,4]furo[2,3-b]pyrrol-8-yl benzoate (P-8). Using XBB-002 (0.15 g, 0.28 mmol, 1.00 eq.), glycine methyl ester hydrochloride hydrochloride (3.00 eq.), Et₃N (5.40 eq.) in CHCl₃ (3 mL, 1% EtOH) yielding in P-8 as a white powder (108.1 mg, 77%).

Synthesis of (3aS,5aS,8R,8aS,9R,10aS)-9-(tert-butyl)-9-hydroxy-6-methyl-2,4,7-trioxooctahydro-4H,9H-furo [3″,2″:2′,3′]cyclopenta [1′,2′:3,4]furo[2,3-b]pyrrol-8-ylbenzoate (DW168). Using XBB-002 (0.10 g, 0.19 mmol, 1.00 eq.), MeNH₂ (2M in THF, 1.50 eq.) THF (2 mL) yielding in DW168 as a white powder (53%). ¹H-NMR (400 MHz, CDCl₃): δ [ppm]=7.98 (m, 2H), 7.62 (m, 1H), 7.49 (m, 2H), 6.32 (s, 1H), 5.92 (s, 1H), 5.19 (t, J=6.3 Hz, 1H), 3.13 (d, J=19.9 Hz, 1H), 2.99 (s, 3H), 2.88 (d, J=19.1 Hz, 1H), 2.64 (m, 1H), 2.53 (s, 1H), 2.21 (m, 1H), 1.08 (s, 9H).

Synthesis of (3aS,5aS,8R,8aS,9R,10aS)-6-amino-9-(tert-butyl)-8,9-dihydroxytetrahydro-4H,9H-furo [3″,2″:2′,3′]cyclopenta [1′,2′:3,4]furo [2,3-b]pyrrole-2,4,7(3H,8H)-trione (JW072). Using XBB-002 and hydrazine yielding in JW072 as a white powder. ¹H-NMR (400 MHz, CDCl₃): δ [ppm]=7.92-7.97 (m, 2H), 7.39-7.43 (m, 1H), 7.33-7.39 (m, 2H), 5.76 (s, 1H), 5.05 (t, J=7.0 Hz, 1H), 4.88 (s, 1H), 2.96 (d, J=18.0 Hz, 1H), 2.70 (d, J=18.0 Hz, 1H), 2.61 (dd, J=13.5, 7.0 Hz, 1H), 2.30 (dd, J=13.5, 7.0 Hz, 1H), 1.10 (s, 9H). The X-ray crystal structure of JW072 is shown in FIG. 1F.

Synthesis of (3aS,5aS,8R,8aS,9R,10aS)-6-(adamantan-1-ylmethyl)-9-(tert-butyl)-9-hydroxy-2,4,7-trioxooctahydro-4H,9H-furo[3″,2″:2′,3′]cyclopenta[1′,2′:3,4]furo[2,3-b]pyrrol-8-yl benzoate (SCC363). Using XBB-002 (1.0 equiv., 0.10 mmol, 53.5 mg), 1-adamantanemethylamine (2.0 equiv., 0.20 mmol., 33.1 mg, 35 μL) in THF (1 mL) yielding SCC363 (46.9 mg, 81%). ¹H-NMR (600 MHz, CDCl₃): δ [ppm]=7.98 (d, J=7.9 Hz, 2H), 7.61 (t, J=7.9 Hz, 1H), 7.48 (t, J=8.1 Hz, 1H), 6.38 (s, 1H), 6.07 (s, 1H), 5.17 (t, J=7.2 Hz, 1H), 3.44 (d, J=14.4 Hz, 1H), 3.14 (d, J=18.3 Hz, 1H), 2.84 (d, J=18.6 Hz, 1H), 2.81 (d, J=13.4 Hz, 1H), 2.64 (dd, J=7.0, 13.8 Hz, 1H), 2.47 (s, 1H), 2.22 (dd, J=7.3, 14.1 Hz, 1H), 2.00 (s, 3H), 1.72 (d, J=11.2 Hz, 3H), 1.61 (d, J=11.4 Hz, 3H), 1.56 (brs, 6H), 1.11 (s, 9H).

Synthesis of (3aS,5aS,8R,8aS,9R,10aS)-6-(((1S,3S)-adamantan-2-yl)methyl)-9-(tert-butyl)-9-hydroxy-2,4,7-trioxooctahydro-4H,9H-furo[3″,2″:2′,3′]cyclopenta[1′,2′:3,4]furo[2,3-b]pyrrol-8-yl 4-(((tert-butoxycarbonyl)amino)methyl)benzoate (SCC505). Using SCC501 (1.0 equiv., 0.13 mmol, 100 mg), adamantan-2-ylmethanamine hydrochloride (1.5 equiv., 0.19 mmol, 38.2 mg), Et₃N (4.0 equiv., 0.50 mmol, 51.1 mg, 70 μL) in CHCl₃ (1% EtOH, 2 mL) yielding SCC505 (63.7 mg, 73%). MS (ESI): m/z=729.4 [M+Na]⁺.

Synthesis of (3aS,5aS,8R,8aS,9R,10aS)-9-(tert-butyl)-9-hydroxy-6-(4-hydroxyphenethyl)-2,4,7-trioxooctahydro-4H,9H-furo[3″,2″:2′,3′]cyclopenta[1′,2′:3,4]furo[2,3-b]pyrrol-8-yl benzoate (SCC545). Using XBB-002 (1.0 equiv., 50 μmol, 26.7 mg), tyramine (1.3 equiv., 64.9 μmol., 8.9 mg), Et₃N (2.0 equiv., 0.10 mmol, 10.1 mg, 14 μL) in THF (1 mL) yielding SCC545 (14.0 mg, 51%). ¹H-NMR (700 MHz, MeOD-d4): δ [ppm]=7.96 (d, J=8.2 Hz, 2H), 7.67 (t, J=7.9 Hz, 1H), 7.50 (t, J=7.8 Hz, 1H), 7.10 (d, J=8.5 Hz, 2H), 6.75 (d, J=8.2 Hz, 2H), 6.16 (s, 1H), 5.69 (s, 1H), 5.10 (t, J=7.2 Hz, 1H), 4.65 (s, 1H), 3.83 (dd, J=5.9, 13.3 Hz, 1H), 3.57 (dd, J=7.4, 14.0 Hz, 1H), 2.96 (dt, J=7.0, 13.9 Hz, 1H), 2.89 (dt, J=5.0, 12.6 Hz, 1H), 2.84 (d, J=19.0 Hz, 1H), 2.80 (d, J=19.0 Hz, 1H), 2.67 (dd, J=7.0, 13.8 Hz, 1H), 2.05 (dd, J=7.3, 14.1 Hz, 1H), 0.92 (s, 9H).

Synthesis of (3aS,5aS,8R,8aS,9R,10aS)-6-(2-(5-methoxy-1H-indol-3-yl)ethyl)-9-(tert-butyl)-9-hydroxy-2,4,7-trioxooctahydro-4H,9H-furo[3″,2″:2′,3′]cyclopenta[1′,2′:3,4]furo[2,3-b]pyrrol-8-yl benzoate (SCC555). Using XBB-002 (1.0 equiv., 50 μmol, 26.7 mg), 5-methoxytryptamine (1.3 equiv., 64.9 μmol., 12.4 mg), Et₃N (2.0 equiv., 0.10 mmol, 10.1 mg, 14 μL) in THF (1 mL) yielding SCC555 (16.7 mg, 55%). ¹H-NMR (700 MHz, CDCl₃): δ [ppm]=8.09 (s, 1H), 7.98 (d, J=7.8 Hz, 2H), 7.62 (t, J=8.0 Hz, 1H), 7.49 (t, J=7.7 Hz, 1H), 7.25 (d, J=7.8 Hz, 1H), 7.07 (d, J=9.8 Hz, 2H), 6.88 (dd, J=2.3, 8.7 Hz, 1H), 6.11 (s, 1H), 5.54 (s, 1H), 5.12 (t, J=7.1 Hz, 1H), 3.96 (dt, J=6.5, 14.0 Hz, 1H), 3.91 (s, 3H), 3.64 (dt, J=7.0, 14.2 Hz, 1H), 3.22 (dt, J=7.0, 14.9 Hz, 1H), 3.08-30.3 (m, 1H), 3.05 (d, J=19.2 Hz, 1H), 2.71 (d, J=18.9 Hz, 1H), 2.52 (dd, J=7.2, 14.1 Hz, 1H), 2.13 (dd, J=7.3, 14.0 Hz, 1H), 2.10 (brs, 1H), 0.78 (s, 9H).

Synthesis of (3aS,5aS,8R,8aS,9R,10aS)-9-(tert-butyl)-9-hydroxy-6-(((1R,4aS,10aR)-7-isopropyl-1,4a-dimethyl-1,2,3,4,4a,9,10,10a-octahydrophenanthren-1-yl)methyl)-2,4,7-trioxooctahydro-4H,9H-furo[3″,2″:2′,3′]cyclopenta[1′,2′:3,4]furo[2,3-b]pyrrol-8-yl benzoate (SCC558). Using XBB002 (1.0 equiv., 75.0 μmol, 40.1 mg), leelamine (1.3 equiv., 97.5 μmol, 26.1 mg), Et₃N (2.0 equiv., 0.15 mmol, 15.2 mg, 21 μL) in THF (2 mL) yielding in SCC558 (29.7 mg, 57%). ¹H-NMR (700 MHz, CDCl₃): δ [ppm]=7.97 (d, J=7.2 Hz, 2H), 7.61 (t, J=7.0 Hz, 1H), 7.48 (t, J=7.1 Hz, 2H), 7.14 (d, J=8.2 Hz, 1H), 6.98 (d, J=8.2 Hz, 1H), 6.89 (s, 1H), 6.30 (s, 1H), 6.05 (s, 1H), 5.18 (t, J=7.0 Hz, 1H), 3.71 (d, J=14.1 Hz, 1H), 3.14 (d, J=19.3 Hz, 1H), 3.03-2.97 (m, 2H), 2.89-2.79 (m, 3H), 2.60 (dd, J=7.1, 14.1 Hz, 1H), 2.32 (d, J=12.5 Hz, 1H), 2.20 (dd, J=7.0, 13.5 Hz, 1H), 1.96-1.91 (m, 1H), 1.88-1.82 (m, 1H), 1.76-1.65 (m, 2H), 1.54 (d, J=12.5 Hz, 1H), 1.39-1.28 (m, 3H), 1.22 (s, 3H), 1.21 (s, 3H), 1.20 (d, J=7.1 Hz, 6H), 1.00 (s, 3H), 0.95 (s, 9H).

Synthesis of (3aS,5aS,8R,8aS,9R,10aS)-6-(((1S,5S)-3-(((tert-butoxycarbonyl)amino)methyl)adamantan-1-yl)methyl)-9-(tert-butyl)-9-hydroxy-2,4,7-trioxo octahydro-4H,9H-furo[3″,2″:2′,3′]cyclopenta[1′,2′:3,4]furo[2,3-b]pyrrol-8-yl benzoate (SCC564). Using XBB002 (1.0 equiv., 0.16 mmol, 84 mg), tert-butyl (((3S,5R)-3-(aminomethyl)adamantan-1-yl)methyl)carbamate (1.2 equiv., 0.19 mmol, 55 mg), Et3N (3.0 equiv., 0.47 mmol, 47.7 mg, 66 μL) in CHCl₃ (4 mL, 1% EtOH) yielding SCC564 (70.1 mg, 63%). MS(ESI): m/z=729.2 (100%, [M+Na]⁺).

### Synthesis of SXQ087-1

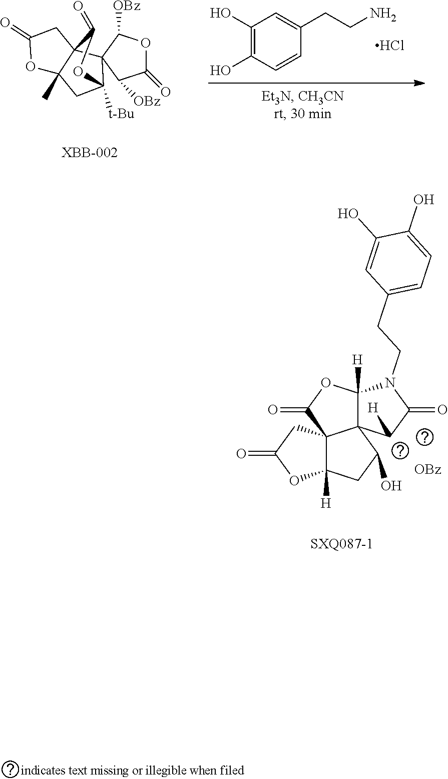

To an oven-dried flask containing a magnetic stir bar was added XBB-002 (20 mg, 0.037 mmol, 1.0 equiv.) and 0.3 mL of CH₃CN, followed by the addition of Et₃N (70 g L, 0.502 mmol, 14 equiv.) and dopamine hydrochloride (11 mg, 0.056 mmol, 1.5 equiv.). The resulting solution was allowed to be stirred at room temperature for 30 min. Once completion indicated by TLC, the resulting solution was diluted with ethyl acetate and was quenched by the addition of saturated aqueous NH₄Cl (6 mL). This mixture was extracted with EtOAc (3×10 mL), the organic layers were combined, washed with saturated aqueous NaCl, and dried over anhydrous Na₂SO₄. The solution was concentrated in vacuo and this crude product was purified by column chromatography (hexane:EtOAc=2:1, v/v) to give SXQ087-1 (16 mg, 76%) as white powder. TLC: R_{f}=0.3 (Hexane/EtOAc=1/1; strongly UV active, stains yellow upon KMnO₄ staining). ¹H NMR (700 MHz, MeOD) δ 7.96 (d, J=7.6 Hz, 2H), 7.67 (t, J=7.5 Hz, 1H), 7.50 (t, J=7.7 Hz, 2H), 6.72 (d, J=8.0 Hz, 1H), 6.70 (d, J=2.3 Hz, 1H), 6.59 (dd, J=8.0, 2.2 Hz, 1H), 6.18 (s, 1H), 5.65 (s, 1H), 5.11 (t, J=7.2 Hz, 1H), 3.82 (m, 1H), 3.55-3.49 (m, 1H), 2.87 (d, J=3.1 Hz, 2H), 2.82 (m, 1H), 2.69-2.63 (m, 1H), 2.07-2.03 (m, 1H), 0.91 (s, 9H). ¹³C NMR (126 MHz, MeOD) δ 179.4, 175.8, 169.3, 166.5, 146.7, 145.3, 135.3, 130.9, 130.5, 129.9, 129.5, 121.0, 116.9, 116.7, 89.9, 89.9, 87.5, 85.4, 72.4, 64.5, 60.6, 43.1, 42.9, 38.3, 37.2, 33.3, 26.9, 14.5.

### Synthesis of SXQ090-1

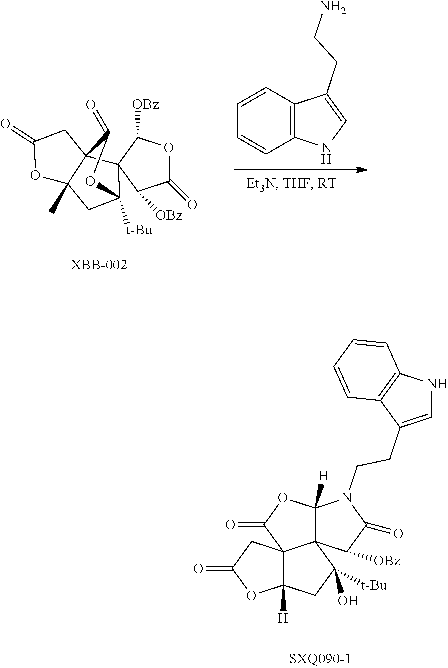

To an oven-dried flask containing a magnetic stir bar was added XBB-002 (20 mg, 0.037 mmol, 1.0 equiv.) and 0.3 mL of THF, followed by the addition of Et₃N (11 g L, 0.075 mmol, 2 equiv.) and tryptamine (9 mg, 0.056 mmol, 1.5 equiv.). The resulting solution was allowed to be stirred at room temperature for 3 h. Once completion indicated by TLC, the resulting solution was diluted with ethyl acetate and was quenched by the addition of saturated aqueous NH₄Cl (6 mL). This mixture was extracted with EtOAc (3×10 mL), the organic layers were combined, washed with saturated aqueous NaCl, and dried over anhydrous Na₂SO₄. The solution was concentrated in vacuo and this crude product was purified by column chromatography (hexane:EtOAc=1:1, v/v) to give SXQ090-1 (18 mg, 85%) as white powder. TLC: R_{f}=0.3 (Hexane/EtOAc=2/1; strongly UV active, stains yellow upon KMnO₄ staining). ¹H NMR (700 MHz, MeOD) δ 7.99-7.95 (m, 2H), 7.66 (tt, J=7.3, 1.3 Hz, 1H), 7.62 (dt, J=7.6, 1.0 Hz, 1H), 7.50 (dd, J=8.3, 7.5 Hz, 2H), 7.35 (dt, J=8.0, 1.0 Hz, 1H), 7.14 (s, 1H), 7.11 (dddd, J=21.8, 8.0, 7.0, 1.2 Hz, 3H), 6.09 (s, 1H), 5.42 (s, 1H), 5.07 (t, J=7.1 Hz, 1H), 3.92 (ddd, J=14.1, 6.6, 4.9 Hz, 1H), 3.62 (ddd, J=14.1, 9.3, 6.2 Hz, 1H), 3.26 (ddd, J=15.4, 9.3, 6.6 Hz, 1H), 3.08-3.03 (m, 1H), 2.93-2.81 (m, 2H), 2.59 (dd, J=13.8, 7.2 Hz, 1H), 2.01-1.98 (m, 1H), 0.68 (s, 9H). ¹³C NMR (176 MHz, MeOD) δ 179.4, 175.8, 169.4, 166.5, 138.2, 135.2, 130.9, 129.9, 129.5, 128.7, 124.0, 122.7, 120.2, 119.2, 112.6, 111.8, 90.0, 87.4, 85.4, 72.5, 64.4, 60.5, 43.0, 42.8, 38.0, 37.2, 26.6, 23.4, 14.5.

### Synthesis of SXQ092-1

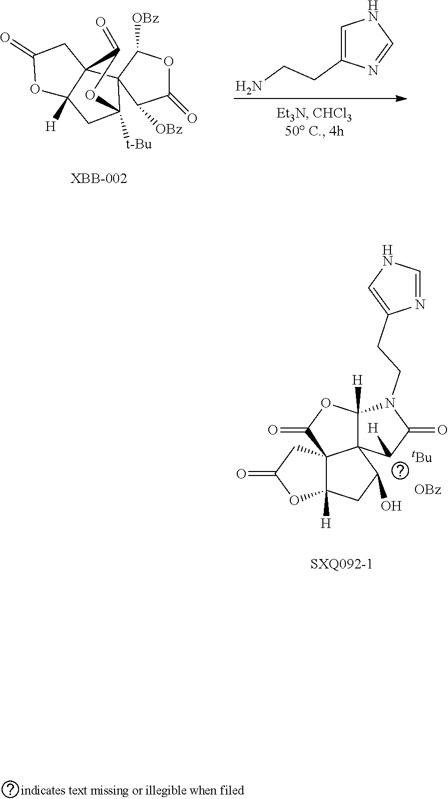

To an oven-dried flask containing a magnetic stir bar was added XBB-002 (20 mg, 0.037 mmol, 1.0 equiv.) and 0.3 mL of CHCl₃, followed by the addition of Et₃N (50 g L, 0.37 mmol, 10 equiv.) and histamine (7 mg, 0.056 mmol, 1.5 equiv.). The resulting solution was allowed to be stirred at 50° C. for 4 h. Once completion indicated by TLC, the resulting solution was diluted with ethyl acetate and was quenched by the addition of saturated aqueous NH₄Cl (6 mL). This mixture was extracted with EtOAc (3×10 mL), the organic layers were combined, washed with saturated aqueous NaCl, and dried over anhydrous Na₂SO₄. The solution was concentrated in vacuo and this crude product was purified by column chromatography (DCM:MeOH=30:1, v/v) to give SXQ092-1 (5 mg, 26%) as white powder. TLC: R_{f}=0.3 (DCM/MeOH=30/1; strongly UV active, stains yellow upon KMnO₄ staining). ¹H NMR (700 MHz, MeOD) δ 7.95 (d, J=7.8 Hz, 2H), 7.65 (t, J=7.5 Hz, 1H), 7.62 (s, 1H), 7.49 (t, J=7.7 Hz, 2H), 6.89 (s, 1H), 6.21 (s, 1H), 5.74 (s, 1H), 5.13 (t, J=7.1 Hz, 1H), 3.85 (m, 1H), 3.63 (m, 1H), 3.02 (m, 1H), 2.95-2.89 (m, 2H), 2.85 (d, J=19.1 Hz, 1H), 2.68 (dd, J=13.7, 7.2 Hz, 1H), 2.08 (dd, J=13.7, 7.2 Hz, 1H), 0.96 (s, 9H).

### Synthesis of SXQ102-1

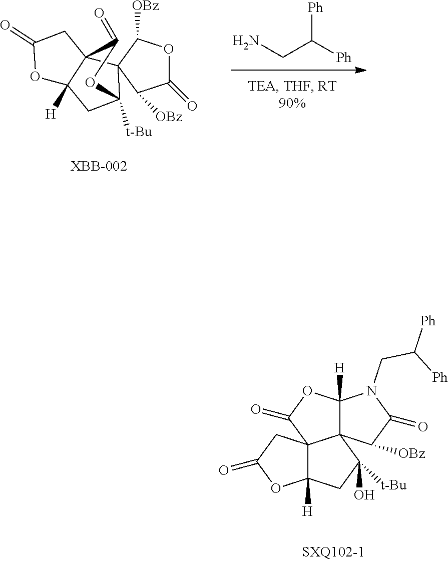

To an oven-dried flask containing a magnetic stir bar was added XBB-002 (20 mg, 0.037 mmol, 1.0 equiv.) and 0.4 mL of THF, followed by the addition of Et₃N (16 μL, 0.112 mmol, 3 equiv.) and 2,2-diphenylethan-1-amine (11 mg, 0.056 mmol, 1.5 equiv.). The resulting solution was allowed to be stirred at room temperature for 15 h. Once completion indicated by TLC, the resulting solution was diluted with ethyl acetate and was quenched by the addition of saturated aqueous NH₄Cl (6 mL). This mixture was extracted with EtOAc (3×10 mL), the organic layers were combined, washed with saturated aqueous NaCl, and dried over anhydrous Na₂SO₄. The solution was concentrated in vacuo and this crude product was purified by column chromatography (hexane:EtOAc=2:1, v/v) to give SXQ102-1 (20 mg, 90%) as white powder. TLC: R_{f}=0.3 (Hexane/EtOAc=2/1; strongly UV active, stains yellow upon KMnO₄ staining). ¹H NMR (700 MHz, CDCl₃) δ 8.13-8.10 (m, 1H), 7.98-7.95 (m, 2H), 7.62 (m, 1H), 7.49 (t, J=7.7 Hz, 3H), 7.33 (m, 8H), 7.27-7.22 (m, 3H), 6.02 (s, 1H), 5.62 (s, 1H), 5.12 (t, J=7.1 Hz, 1H), 4.60 (t, J=8.9 Hz, 1H), 4.19 (m, 1H), 3.97 (m, 1H), 2.86 (m, 1H), 2.55 (dd, J=14.1, 7.2 Hz, 1H), 2.48 (m, 1H), 2.13 (dd, J=14.1, 7.1 Hz, 1H), 0.82 (s, 9H). ¹³C NMR (126 MHz, CDCl₃) δ 178.0, 173.7, 167.2, 165.3, 140.8, 140.3, 134.4, 133.8, 130.3, 130.2, 129.1, 128.9, 128.6, 128.3, 127.9, 127.8, 127.5, 127.4, 88.2, 87.5, 83.2, 70.7, 62.4, 59.3, 48.7, 45.3, 43.0, 37.2, 35.9, 26.2.

### Synthesis of SXQ091-1

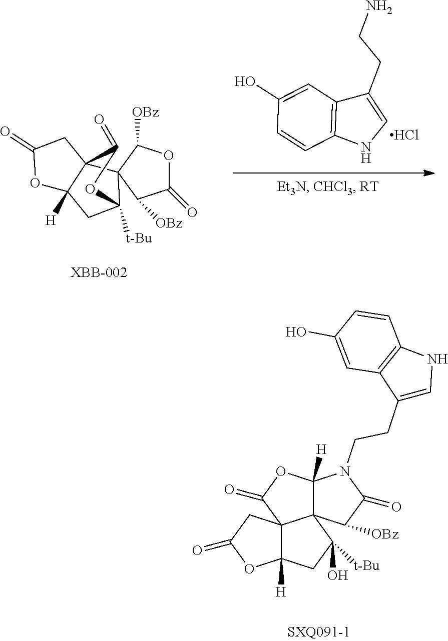

To an oven-dried flask containing a magnetic stir bar was added XBB-002 (20 mg, 0.037 mmol, 1.0 equiv.) and 0.5 mL of CHCl₃, followed by the addition of Et₃N (32 μL, 0.224 mmol, 6 equiv.) and serotonin hydrochloride (12 mg, 0.056 mmol, 1.5 equiv.). The resulting solution was allowed to be stirred at room temperature for 19 h. Once completion indicated by TLC, the resulting solution was diluted with ethyl acetate and was quenched by the addition of saturated aqueous NH₄Cl (6 mL). This mixture was extracted with EtOAc (3×10 mL), the organic layers were combined, washed with saturated aqueous NaCl, and dried over anhydrous Na₂SO₄. The solution was concentrated in vacuo and this crude product was purified by column chromatography (hexane:EtOAc=1:1, v/v) to give SXQ091-1 (6 mg, 28%) as white powder. TLC: R_{f}=0.3 (Hexane/EtOAc=1/1; strongly UV active, stains yellow upon KMnO₄ staining). ¹H NMR (700 MHz, CDCl₃) δ 8.48 (t, J=6.5 Hz, 2H), 8.15 (t, J=7.5 Hz, 1H), 8.01 (t, J=7.7 Hz, 2H), 7.75-7.70 (m, 1H), 7.59-7.50 (m, 2H), 7.28 (m, 1H), 6.61 (s, 1H), 6.03 (d, J=5.3 Hz, 1H), 5.64 (t, J=7.1 Hz, 1H), 4.42 (m, 1H), 4.07 (m, 1H), 3.71 (m, 1H), 3.59-3.47 (m, 2H), 3.30 (m, 1H), 3.13 (m, 1H), 2.59-2.55 (m, 1H), 1.24 (s, 9H). ¹³C NMR (176 MHz, CDCl₃) δ 178.3, 174.3, 167.7, 165.4, 150.0, 134.1, 131.3, 129.8, 129.7, 128.6, 128.6, 127.7, 127.6, 123.3, 123.2, 111.9, 109.6, 102.0, 88.5, 86.1, 83.9, 71.0, 62.8, 59.1, 41.7, 41.5, 36.8, 36.0, 25.7, 22.2.

### Synthesis of SXQ125-2

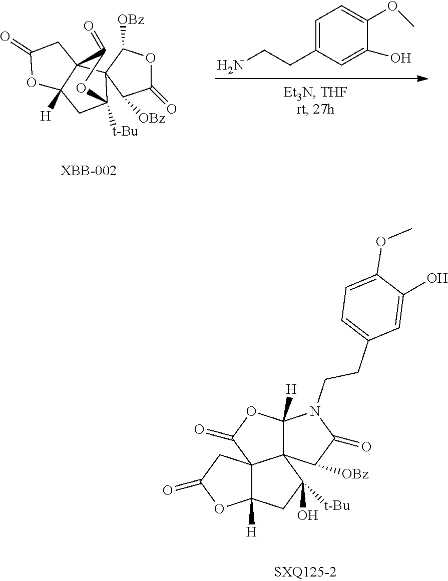

To an oven-dried flask containing a magnetic stir bar was added XBB-002 (30 mg, 0.056 mmol, 1.0 equiv.) and 0.5 mL of THF, followed by the addition of Et₃N (24 μL, 0.168 mmol, 3 equiv.) and 3-hydroxy-4-methoxyphenethylamine (14 mg, 0.084 mmol, 1.5 equiv.). The resulting solution was allowed to be stirred at room temperature for 27 h. Once completion indicated by TLC, the resulting solution was diluted with ethyl acetate and was quenched by the addition of saturated aqueous NH₄Cl (6 mL). This mixture was extracted with EtOAc (3×10 mL), the organic layers were combined, washed with saturated aqueous NaCl, and dried over anhydrous Na₂SO₄. The solution was concentrated in vacuo and this crude product was purified by column chromatography (Hexane:EtOAc=1:1, v/v) to give SXQ125-2 (19 mg, 60%) as white powder. TLC: R_{f}=0.3 (Hexane/EtOAc=1/1; strongly UV active, stains yellow upon KMnO₄ staining). ¹H NMR (700 MHz, MeOD) δ 7.98-7.95 (m, 2H), 7.67 (m, 1H), 7.50 (t, J=7.7 Hz, 2H), 6.87 (d, J=8.2 Hz, 1H), 6.74 (d, J=2.1 Hz, 1H), 6.71 (m, 1H), 6.17 (s, 1H), 5.66 (s, 1H), 5.10 (t, J=7.1 Hz, 1H), 3.83 (s, 3H), 3.81 (q, J=6.1, 5.2 Hz, 1H), 3.56 (ddd, J=14.4, 8.5, 6.3 Hz, 1H), 2.92 (ddd, J=14.8, 8.4, 6.5 Hz, 1H), 2.84 (d, J=4.9 Hz, 3H), 2.66 (dd, J=13.8, 7.2 Hz, 1H), 2.08-2.03 (m, 1H), 0.91 (s, 9H). ¹³C NMR (176 MHz, MeOD) δ 179.4, 175.8, 169.3, 166.5, 148.1, 147.9, 135.3, 132.0, 130.9, 129.9, 129.4, 120.9, 116.9, 113.2, 89.9, 87.4, 85.4, 72.3, 64.5, 60.6, 56.5, 43.1, 43.0, 38.3, 37.2, 33.3, 26.9, 14.5.

### Synthesis of SXQ126-1

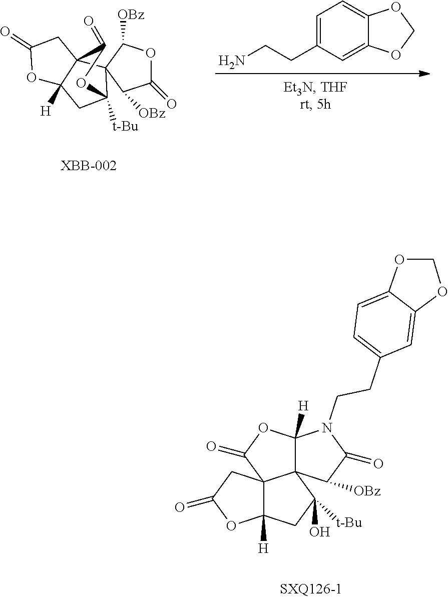

To an oven-dried flask containing a magnetic stir bar was added XBB-002 (30 mg, 0.056 mmol, 1.0 equiv.) and 0.5 mL of THF, followed by the addition of Et₃N (24 μL, 0.168 mmol, 3 equiv.) and 3,4-Methylenedioxyphenethylamine (12 μL, 0.084 mmol, 1.5 equiv.). The resulting solution was allowed to be stirred at room temperature for 5 h. Once completion indicated by TLC, the resulting solution was diluted with ethyl acetate and was quenched by the addition of saturated aqueous NH₄Cl (6 mL). This mixture was extracted with EtOAc (3×10 mL), the organic layers were combined, washed with saturated aqueous NaCl, and dried over anhydrous Na₂SO₄. The solution was concentrated in vacuo and this crude product was purified by column chromatography (Hexane:EtOAc=1:1, v/v) to give SXQ126-1 (22 mg, 68%) as white powder. TLC: R_{f}=0.5 (Hexane/EtOAc=1/1; strongly UV active, stains yellow upon KMnO₄ staining). ¹H NMR (700 MHz, MeOD) δ 7.98-7.95 (m, 2H), 7.66 (tt, J=7.5, 1.3 Hz, 1H), 7.50 (dd, J=8.4, 7.5 Hz, 2H), 6.80 (d, J=1.7 Hz, 1H), 6.76 (d, J=7.9 Hz, 1H), 6.72 (dd, J=7.9, 1.7 Hz, 1H), 6.19 (s, 1H), 5.92 (s, 2H), 5.73 (s, 1H), 5.11 (t, J=7.2 Hz, 1H), 3.81 (dt, J=14.2, 6.3 Hz, 1H), 3.57 (ddd, J=14.4, 8.1, 6.5 Hz, 1H), 2.97-2.92 (m, 1H), 2.91-2.86 (m, 1H), 2.85 (d, J=9.4 Hz, 2H), 2.67 (dd, J=13.8, 7.2 Hz, 1H), 2.08-2.04 (m, 1H), 0.94 (s, 9H). ¹³C NMR (176 MHz, MeOD) δ 179.4, 175.8, 169.3, 166.5, 149.5, 148.0, 135.3, 133.0, 131.0, 129.9, 129.4, 123.0, 110.1, 109.4, 102.3, 89.8, 87.5, 85.4, 72.3, 64.4, 60.6, 43.2, 43.1, 38.3, 37.2, 33.7, 26.9, 14.5.

### Synthesis of SXQ128-1

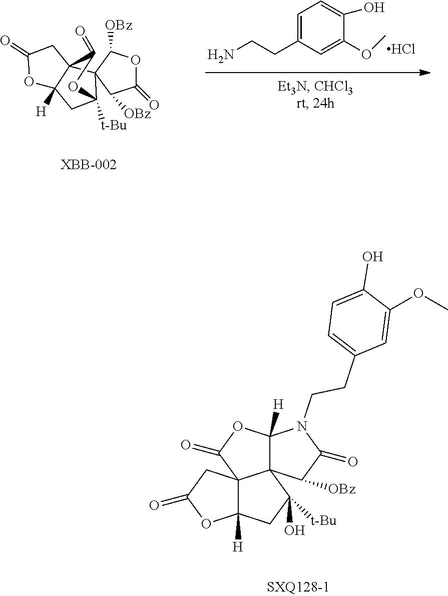

To an oven-dried flask containing a magnetic stir bar was added XBB-002 (30 mg, 0.056 mmol, 1.0 equiv.) and 0.5 mL of THF, followed by the addition of Et₃N (24 μL, 0.168 mmol, 3 equiv.) and 3-O-Methyldopamine hydrochloride (18 mg, 0.084 mmol, 1.5 equiv.). The resulting solution was allowed to be stirred at room temperature for 24 h. Once completion indicated by TLC, the resulting solution was diluted with ethyl acetate and was quenched by the addition of saturated aqueous NH₄Cl (6 mL). This mixture was extracted with EtOAc (3×10 mL), the organic layers were combined, washed with saturated aqueous NaCl, and dried over anhydrous Na₂SO₄. The solution was concentrated in vacuo and this crude product was purified by column chromatography (Hexane:EtOAc=1:1, v/v) to give SXQ128-1 (15 mg, 47%) as white powder. TLC: R_{f}=0.4 (Hexane/EtOAc=1/1; strongly UV active, stains yellow upon KMnO₄ staining). ¹H NMR (700 MHz, MeOD) δ 7.98-7.94 (m, 2H), 7.68-7.65 (m, 1H), 7.50 (dd, J=8.4, 7.4 Hz, 2H), 6.84 (d, J=1.9 Hz, 1H), 6.75 (d, J=8.0 Hz, 1H), 6.71 (dd, J=7.9, 1.9 Hz, 1H), 6.13 (s, 1H), 5.63 (s, 1H), 5.10 (t, J=7.2 Hz, 1H), 3.89 (s, 3H), 3.60 (ddd, J=14.4, 8.4, 6.2 Hz, 1H), 2.99 (ddd, J=14.7, 8.5, 6.4 Hz, 1H), 2.91 (dt, J=14.5, 6.1 Hz, 1H), 2.81 (q, J=19.2 Hz, 2H), 2.65 (dd, J=13.8, 7.2 Hz, 1H), 2.06-2.02 (m, 1H), 0.88 (s, 9H).

¹³C NMR (176 MHz, MeOD) δ 179.4, 175.8, 169.3, 166.5, 149.3, 146.5, 135.3, 130.9, 130.3, 129.9, 129.4, 122.3, 116.5, 113.1, 89.9, 87.4, 85.4, 72.4, 64.4, 60.6, 56.3, 43.1, 42.6, 38.2, 37.1, 33.2, 26.9, 14.5.

### Example 1.7: Synthesis of Piperazine Substituted-Bilobalide Lactam Analogues, According to Schemes 5, 10 and 11

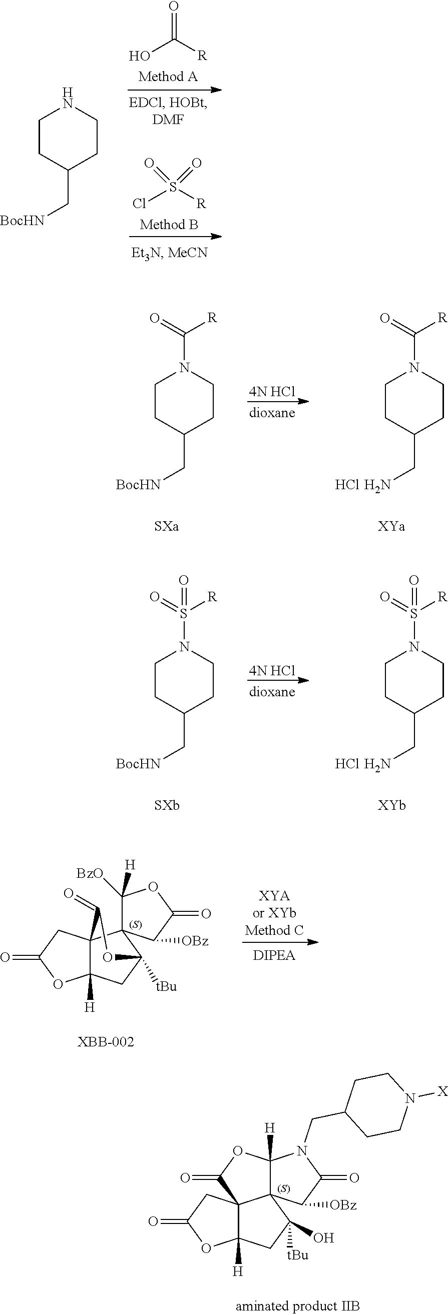

Method A (Scheme 10): To a solution of substituted carboxylic acid (2 mmol, 1.0 equiv.) in DMF (20 mL), 1-hydroxybenzotrizole (HOBt) (2.2 mmol, 1.1 equiv.) and N-(3-dimethylaminopropyl)-N′-ethylcarbodiimide hydrochloride (EDCI) (2.2 mmol, equiv.) were added. This mixture was stirred for 30 minutes at room temperature, then tert-butyl (piperidin-4-ylmethyl)carbamate (2 mmol, 1.0 equiv.) was added. Upon completion monitored by TLC, the crude reaction mixture was diluted with EtOAc (20 mL) and washed with water (20 mL×3) and brine (20 mL×3). The combined organic layers were dried over Na₂SO₄. The solvents were then removed under reduced pressure. The crude residue was purified by silica gel column chromatography (hexane/EtOAc=3:1) to afford the desired Boc-protected amines (SXa1 through SXa4 according to Table 1 h). To an oven-dried flask was added compound and 4 N HCl in dioxane, respectively. The resulting solution was stirred at room temperature for 1 h. Once completed, the reaction solution was concentrated under reduced pressure to provide the respective alkyl amines XYa as a white powder, which was directly used for the next step without purification.

Method B(Scheme 11): To a solution of tert-butyl (piperidin-4-ylmethyl)carbamate (2 mmol, 1.0 equiv.) in acetonitrile was added substituted sulfonyl chloride (2.2 mmol, 1.1 equiv.) and triethylamine, respectively. The resulting mixture was stirred at room temperature for 4 h. Upon completion monitored by TLC, the reaction solution was concentrated under reduced pressure. The residue was purified by silica gel column chromatography (hexane/EtOAc=3:1) to afford the desired Boc-protected amines (SXb1 according to Table 1 h). To an oven-dried flask was added compound and 4 N HCl in dioxane, respectively. The resulting solution was stirred at room temperature for 1 h. Once completed, the reaction solution was concentrated under reduced pressure to provide the respective alkyl amines XYb white powder, which was directly used for the next step without purification.

Method C (Scheme 5): To a solution of XBB-002 (100 mg, 1.0 equiv) in anhydrous tetrahydrofuran (2 mL) was added substituted alkyl amines XYa or XYb (1.1 equiv.) and DIPEA. The resulting solution was stirred at room temperature for 1 h. Upon completion indicated by TLC, the reaction solution was concentrated in vacuo. The residue was dissolved with dichloromethane and the organic layer was washed with saturated NaCl solution, dried over Na₂SO₄, filtered and concentrated in vacuo. The crude product was purified via column chromatography with elution system (hexane:EtOAc=3:1) to provide the corresponding aminated product IIb according to Table 1i as a white powder.

Examples of Boc-protected amines are summarized in Table 1 h.

TABLE 1h
Example Boc-protected amines
Method A (Scheme 10)

| | | | |
|---|---|---|---|
| 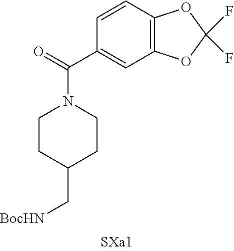 [SMILES: CC(C)(C)OC(=O)NCC1CCN(C(=O)c2ccc3c(c2)OC(F)(F)O3)CC1] | 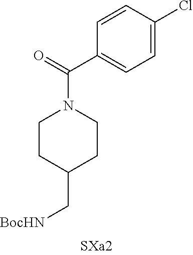 | 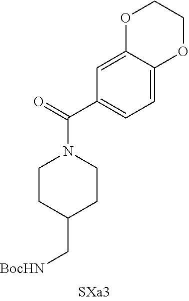 | 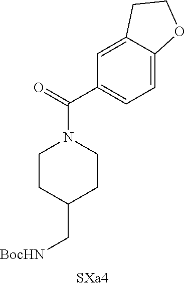 |

Method B (Scheme 11)

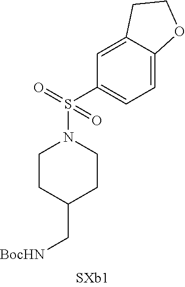 [SMILES: CC(C)(C)OC(=O)NCC1CCN(S(=O)(=O)c2ccc3c(c2)CCO3)CC1]

Synthesis of tert-butyl ((1-(2,2-difluorobenzo[d][1,3]dioxole-5-carbonyl)piperidin-4-yl)methyl)carbamate (SXa1). Method A (Scheme 10). Colourless oil (0.76 g, 95%). R_{f}=0.30 (hexane/EtOAc, 5:1); ¹H-NMR (400 MHz, CDCl₃): δ [ppm]=7.21-7.12 (m, 2H), 7.11-7.02 (m, 1H), 4.95-4.26 (m, 2H), 3.76 (s, 1H), 3.22-2.62 (m, 4H), 1.75 (s, 2H), 1.43 (s, 9H), 1.34-0.96 (m, 2H); ¹³C{¹H}-NMR (100 MHz, CDCl₃): δ [ppm]=168.61, 156.09, 144.43, 143.63, 134.14, 132.14, 131.59, 129.04, 122.86, 109.39, 108.88, 79.39, 47.90, 45.75, 42.52, 36.94, 30.29, 28.38; HRMS (ESI) m/z: [M+Na]⁺ Calcd for C₁₉H₂₄F₂N₂O₅Na⁺ 421.15455, found 421.15424.

Synthesis of tert-butyl ((1-(4-chlorobenzoyl)piperidin-4-yl)methyl)carbamate (SXa2). Method A (Scheme 10). Colourless oil (0.64 g, 90%). R_{f}=0.40 (hexane/EtOAc, 6:1); mp=138.5-139.0° C.; ¹H-NMR (400 MHz, CDCl₃): δ [ppm]=7.49-7.31 (m, 4H), 4.66 (s, 2H), 3.76 (s, 1H), 3.23-2.63 (m, 4H), 1.94-1.64 (s, 2H), 1.46 (s, 9H), 1.35-0.95 (m, 2H); ¹³C{¹H}-NMR (100 MHz, CDCl₃): δ [ppm]=169.29, 156.11, 135.57, 134.53, 128.72, 128.41, 79.36, 47.71, 45.77, 42.27, 36.95, 30.37, 29.45, 28.40; HRMS (ESI) m/z: [M+Na]⁺ Calcd for C₁₈H₂₅ClN₂O₃Na⁺ 375.14459, found 375.14422.

Synthesis of tert-butyl ((1-(2,3-dihydrobenzo[b][1,4]dioxine-6-carbonyl)piperidin-4-yl)methyl)carbamate (SXa3). Method A (Scheme 10). Colourless oil (0.66 g, 87%). R_{f}=0.30 (hexane/EtOAc, 5:1); ¹H-NMR (400 MHz, CDCl₃): δ [ppm]=7.00-6.92 (m, 1H), 6.93-6.83 (m, 2H), 4.67 (s, 1H), 4.29 (s, 4H), 3.90 (s, 1H), 3.23-2.62 (m, 4H), 1.75 (s, 2H), 1.46 (s, 9H), 1.20 (s, 2H); ¹³C{¹H}-NMR (100 MHz, CDCl₃): δ [ppm]=169.89, 156.05, 144.78, 143.31, 129.34, 120.51, 117.19, 116.54, 79.39, 64.46, 64.31, 45.91, 37.02, 28.41; HRMS (ESI) m/z: [M+Na]⁺ Calcd for C₂₀H₂₈N₂O₅Na⁺ 399.20014, found 399.20010.

Synthesis of tert-butyl ((1-(2,3-dihydrobenzofuran-5-carbonyl)piperidin-4-yl)methyl)carbamate (SXa4). Method A (Scheme 10). White powder (0.61 g, 84%). R_{f}=0.30 (hexane/EtOAc, 5:1); mp=138.7-139.5° C.; ¹H-NMR (400 MHz, CDCl₃): δ [ppm]=7.30 (d, J=1.7 Hz, 1H), 7.18 (dd, J=8.2, 1.8 Hz, 1H), 6.78 (d, J=8.2 Hz, 1H), 4.68 (s, 1H), 4.62 (t, J=8.7 Hz, 2H), 4.17-3.64 (m, 1H), 3.24 (t, J=8.7 Hz, 2H), 3.12-2.70 (m, 4H), 1.86-1.61 (m, 3H), 1.46 (s, 9H), 1.32-1.05 (m, 2H); ¹³C{¹H}-NMR (100 MHz, CDCl₃): δ [ppm]=170.56, 161.21, 156.15, 128.22, 127.57, 127.25, 124.38, 108.75, 78.99, 71.52, 45.78, 36.96, 36.43, 29.34, 28.36; HRMS (ESI) m/z: [M+Na]⁺ Calcd for C₁₅H₁₉NO₈Na⁺ 364.10029, found 364.09999.

Synthesis of tert-butyl ((1-((2,3-dihydrobenzofuran-5-yl) sulfonyl) piperidin-4-yl)methyl)carbamate (SXb2). Method B(Scheme 11). White powder (0.70 g, 88%). R_{f}=0.20 (hexane/EtOAc, 5:1); mp=158.7-159.4° C.; ¹H-NMR (400 MHz, CDCl₃): δ [ppm]=7.65-7.49 (m, 2H), 6.87 (d, J=8.4 Hz, 1H), 4.71 (t, J=8.8 Hz, 2H), 4.62 (s, 1H), 3.78 (dt, J=12.2, 3.4 Hz, 2H), 3.30 (t, J=8.8 Hz, 2H), 3.00 (t, J=6.4 Hz, 2H), 2.26 (td, J=11.7, 2.5 Hz, 2H), 1.76 (dd, J=12.7, 3.1 Hz, 2H), 1.44 (s, 9H), 1.39-1.24 (m, 2H); ¹³C{¹H}-NMR (100 MHz, CDCl₃): δ [ppm]=163.42, 155.56, 128.78, 127.82, 127.05, 124.42, 108.97, 78.86, 71.83, 45.65, 45.18, 35.35, 28.67, 28.59, 27.93; HRMS (ESI) m/z: [M+Na]⁺ Calcd for C₁₉H₂₈N₂O₅SNa⁺ 419.16111, found 419.16088.

Examples of piperazine substituted-bilobalide lactam analogues according to Method C as described in this example (Scheme 5) are summarized in Table 1i.

TABLE li
Example piperazine substituted-bilobalide lactam analogues

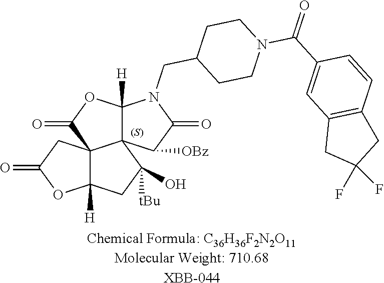

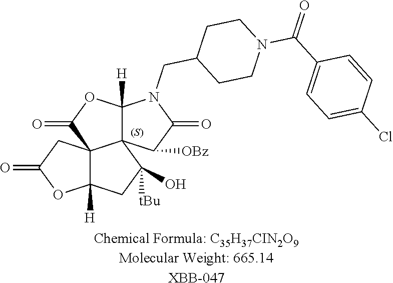

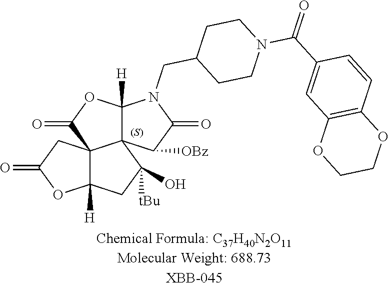

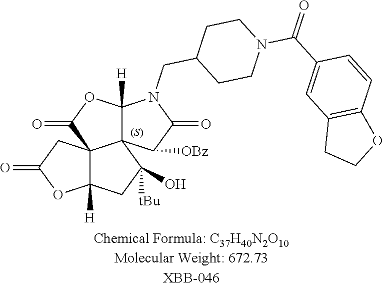

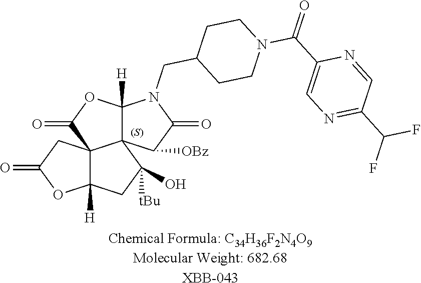

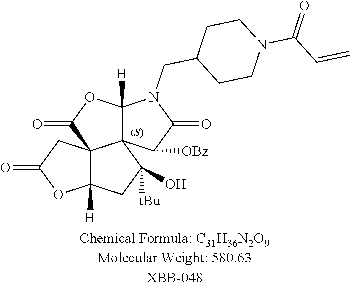

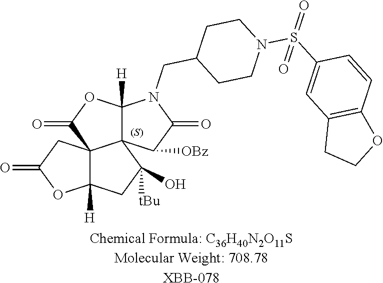

Synthesis of (3aS,5aS,8R,8aS,9R,10aS)-9-(tert-butyl)-6-((1-(2,2-difluorobenzo[d][1,3]dioxole-5-carbonyl)piperidin-4-yl)methyl)-9-hydroxy-2,4,7-trioxooctahydro-4H,9H-furo [3″,2″:2′,3′]cyclopenta [1′,2′:3,4]furo [2,3-b]pyrrol-8-yl benzoate (XBB-044). Using method C (Scheme 5) and SXa1 yielding in XBB-044 as a white powder (93 mg, 70%). R_{f}=0.20 (DCM/MeOH, 10:1); mp=103.6-104.5° C.; ¹H-NMR (400 MHz, CDCl₃): δ [ppm]=8.05-7.94 (m, 2H), 7.71-7.63 (m, 1H), 7.51 (t, J=7.8 Hz, 2H), 7.22-7.08 (m, 3H), 6.36 (s, 1H), 5.95 (s, 1H), 5.18 (t, J=7.1 Hz, 1H), 4.61 (m, 1H), 3.79 (m, 1H), 3.62-2.77 (m, 9H), 2.70 (dd, J=13.9, 7.2 Hz, 1H), 2.23 (dd, J=13.9, 7.1 Hz, 1H), 2.13 (m, 2H), 1.72 (m, 2H), 1.11 (s, 9H); ¹³C{¹H}-NMR (100 MHz, CDCl₃): δ [ppm]=177.90, 173.51, 168.93, 167.69, 165.27, 144.64, 143.71, 131.52, 130.06, 128.80, 127.71, 122.91, 109.60, 108.84, 86.93, 83.51, 70.69, 62.90, 59.25, 42.56, 37.46, 36.45, 31.59, 26.48, 22.66.

Synthesis of (3aS,5aS,8R,8aS,9R,10aS)-9-(tert-butyl)-6-((1-(2,2-difluorobenzo[d][1,3]dioxole-5-carbonyl)piperidin-4-yl)methyl)-9-hydroxy-2,4,7-trioxooctahydro-4H,9H-furo [3″,2″:2′,3′]cyclopenta [1′,2′:3,4]furo [2,3-b]pyrrol-8-yl benzoate (XBB-047). Using method C (Scheme 5) and SXa2 yielding in XBB-047 as a white powder (97 mg, 78%). R_{f}=0.30 (DCM/MeOH, 10:1); mp=137.3-137.9° C.; ¹H-NMR (400 MHz, CDCl₃): δ [ppm]=8.03-7.87 (m, 2H), 7.62 (t, J=7.4 Hz, 1H), 7.48 (t, J=7.7 Hz, 2H), 7.40 (d, J=8.1 Hz, 2H), 7.32 (d, J=8.2 Hz, 2H), 6.33 (s, 1H), 5.93 (s, 1H), 5.13 (t, J=7.1 Hz, 1H), 4.59 (s, 1H), 4.06-3.63 (m, 2H), 3.58-3.19 (m, 2H), 3.18-3.08 (m, 2H), 3.08-2.75 (m, 2H), 2.69 (dd, J=14.0, 7.1 Hz, 1H), 2.19-2.06 (m, 2H), 1.80-1.48 (m, 2H), 1.43-1.18 (m, 2H), 1.05 (s, 9H); ¹³C{H}-NMR (100 MHz, CDCl₃): δ [ppm]=177.90, 173.59, 169.62, 167.71, 165.27, 135.97, 134.31, 133.92, 130.06, 128.93, 128.80, 128.38, 127.73, 86.84, 83.58, 70.71, 62.93, 59.24, 47.48, 46.04, 42.49, 42.07, 37.46, 36.47, 34.46, 30.29, 29.51, 26.50. HRMS (ESI) m/z: [M+Na]⁺ Calcd for C₃₅H₃₇ClN₂O₉Na⁺ 687.20798, found 687.20744.

Synthesis of (3aS,5aS,8R,8aS,9R,10aS)-9-(tert-butyl)-6-((1-(2,3-dihydrobenzo[b][1,4]dioxine-6-carbonyl)piperidin-4-yl)methyl)-9-hydroxy-2,4,7-trioxooctahydro-4H,9H-furo [3″,2″:2′,3′]cyclopenta [1′,2′:3,4]furo[2,3-b]pyrrol-8-yl benzoate (XBB-045). Using method C (Scheme 5) and SXa3 yielding in XBB-045 as a white powder (77 mg, 66%). R_{f}=0.2 (DCM/MeOH, 10:1); mp=156.7-157.4° C.; ¹H-NMR (400 MHz, MeOD): δ [ppm]=8.11-7.87 (m, 2H), 7.76-7.59 (m, 1H), 7.51 (t, J=7.7 Hz, 2H), 6.89 (d, J=10.8 Hz, 3H), 6.41 (s, 1H), 6.08 (s, 1H), 5.19 (t, J=7.1 Hz, 1H), 4.55 (s, 1H), 4.26 (s, 4H), 3.82 (s, 1H), 3.42 (dd, J=14.1, 7.5 Hz, 1H), 3.27 (dd, J=14.2, 7.3 Hz, 1H), 3.05 (s, 1H), 2.84 (s, 1H), 2.74 (dd, J=13.7, 7.1 Hz, 1H), 2.13 (dd, J=13.8, 7.2 Hz, 2H), 1.86-1.45 (m, 2H), 1.37-1.15 (m, 1H), 1.08 (s, 9H); ¹³C{¹H}-NMR (100 MHz, MeOD): δ [ppm]=178.09, 174.35, 170.57, 168.29, 165.15, 145.26, 143.55, 133.99, 129.66, 128.56, 128.43, 128.03, 120.00, 116.98, 116.03, 86.20, 84.05, 71.04, 64.38, 64.24, 63.04, 60.18, 59.31, 53.47, 41.80, 37.08, 36.14, 34.31, 30.35, 29.26, 25.67.

Synthesis of (3aS,5aS,8R,8aS,9R,10aS)-9-(tert-butyl)-6-((1-(2,3-dihydrobenzofuran-5-carbonyl)piperidin-4-yl)methyl)-9-hydroxy-2,4,7-trioxooctahydro-4H,9H-furo[3″,2″:2′,3′]cyclopenta[1′,2′:3,4]furo[2,3-b]pyrrol-8-yl benzoate (XBB-046). Using method C (Scheme 5) and SXa4 yielding in XBB-046 as a white powder (85 mg, 68%). R_{f}=0.20 (DCM/MeOH, 10:1); mp=142.5-143.3° C.; ¹H-NMR (400 MHz, CDCl₃): δ [ppm]=8.04-7.83 (m, 2H), 7.70-7.56 (m, 1H), 7.49 (t, J=7.8 Hz, 2H), 7.26 (s, 1H), 7.16 (dd, J=8.2, 1.8 Hz, 1H), 6.78 (d, J=8.2 Hz, 1H), 6.35 (s, 1H), 5.94 (s, 1H), 5.16 (t, J=7.1 Hz, 1H), 4.61 (t, J=8.7 Hz, 2H), 3.60-3.37 (m, 1H), 3.31-3.04 (m, 5H), 3.00-2.79 (m, 3H), 2.69 (dd, J=13.8, 7.1 Hz, 1H), 2.20 (dd, J=14.0, 7.1 Hz, 1H), 2.15-2.02 (m, 2H), 1.72.-1.50 (s, 2H), 1.44-1.21 (m, 2H), 1.10 (s, 9H); ¹³C{¹H}-NMR (100 MHz, CDCl₃): δ [ppm]=177.90, 173.67, 171.02, 167.71, 165.28, 161.64, 134.25, 130.05, 128.77, 127.80, 127.75, 127.65, 127.47, 124.41, 109.07, 88.46, 86.68, 83.70, 71.73, 70.78, 62.99, 59.22, 53.53, 42.38, 37.48, 36.49, 34.55, 30.10, 29.40, 26.56; HRMS (ESI) m/z: [M+K]⁺ Calcd for C₃₇H₄₀N₂O₁₀K⁺ 711.25243, found: 711.25198.

Synthesis of (3aS,5aS,8R,8aS,9R,10aS)-9-(tert-butyl)-6-((1-(5-(difluoromethyl)pyrazine-2-carbonyl) piperidin-4-yl)methyl)-9-hydroxy-2,4,7-trioxooctahydro-4H,9H-furo [3″,2″:2′,3′]cyclopenta [1′,2′:3,4]furo[2,3-b]pyrrol-8-yl benzoate (XBB-043). Using method C (Scheme 5) yielding in XBB-043 as a white powder (79 mg, 62%). R_{f}=0.20 (DCM/MeOH, 30:1); mp=84.5-85.2° C.; ¹H-NMR (400 MHz, CDCl₃): δ [ppm]=8.97-8.81 (m, 2H), 7.95 (d, J=7.8 Hz, 2H), 7.61 (t, J=7.5 Hz, 1H), 7.53-7.43 (m, 2H), 6.99-6.54 (m, 1H), 6.33 (d, J=5.2 Hz, 1H), 5.94 (s, 1H), 5.15 (q, J=6.5 Hz, 1H), 4.79-4.56 (m, 1H), 3.92 (d, J=13.4 Hz, 1H), 3.73 (d, J=17.1 Hz, 1H), 3.54-3.36 (m, 1H), 3.32-3.18 (m, 1H), 3.17-3.05 (m, 2H), 2.94-2.80 (m, 2H), 2.73-2.62 (m, 1H), 2.15 (dd, J=13.9, 7.2 Hz, 2H), 1.78 (d, J=13.2 Hz, 1H), 1.61 (d, J=13.1 Hz, 1H), 1.49-1.25 (m, 2H), 1.05 (s, 9H); ¹³C{¹H}-NMR (100 MHz, CDCl₃): δ [ppm]=177.87, 173.54, 167.72, 167.64, 165.23, 164.53, 164.46, 150.91, 144.29, 140.03, 134.28, 130.03, 128.78, 127.72, 112.83 (t, J=241.2 Hz), 88.65, 88.30, 87.03, 86.98, 83.50, 70.69, 62.87, 59.24, 46.89, 46.68, 46.14, 42.57, 42.32, 37.42, 36.43, 34.48, 34.18, 30.41, 30.29, 29.50, 26.44; HRMS (ESI) m/z: [M+Na]⁺ Calcd for C₃₄H₃₆F₂N₆O₉Na⁺ 705.23426, found: 705.23463.

Synthesis of (3aS,5aS,8R,8aS,9R,10aS)-6-((1-acryloylpiperidin-4-yl)methyl)-9-(tert-butyl)-9-hydroxy-2,4,7-trioxooctahydro-4H,9H-furo [3″,2″:2′,3′]cyclopenta[1′,2′:3,4]furo[2,3-b]pyrrol-8-yl benzoate (XBB-048). Using method C (Scheme 5) yielding in XBB-048 as a white powder (54 mg, 50%). R_{f}=0.20 (DCM/MeOH, 5:1); mp=159.2-160.1° C.; ¹H-NMR (400 MHz, CDCl₃): δ [ppm]=8.05-7.91 (m, 2H), 7.72-7.59 (m, 1H), 7.51 (t, J=7.7 Hz, 2H), 6.70-6.50 (m, 1H), 6.37 (s, 1H), 6.36-6.20 (m, 1H), 5.94 (s, 1H), 5.84-5.66 (m, 1H), 5.24-5.02 (m, 1H), 4.59 (d, J=13.1 Hz, 1H), 4.19-3.98 (m, 1H), 3.64-3.19 (m, 1H), 3.17-3.04 (m, 3H), 2.95-2.59 (m, 3H), 2.29-2.11 (m, 2H), 1.77-1.63 (m, 2H), 1.49-1.30 (m, 2H), 1.13 (s, 9H); ¹³C{¹H}-NMR (100 MHz, CDCl₃): δ [ppm]=177.40, 173.07, 167.06, 165.36, 164.93, 133.84, 129.65, 128.35, 127.36, 126.71, 88.44, 86.12, 83.00, 70.27, 62.70, 58.76, 45.56, 41.86, 37.15, 35.90, 34.03, 29.53, 29.27, 28.77, 26.10; HRMS (ESI) m/z: [M+Na]⁺ Calcd for C₃₁H₃₆N₂O₉Na⁺ 603.23130, found: 603.23116.

Synthesis of (3aS,5aS,8R,8aS,9R,10aS)-9-(tert-butyl)-6-((1-((2,3-dihydrobenzofuran-5-yl) sulfonyl) piperidin-4-yl) methyl)-9-hydroxy-2,4,7-trioxooctahydro-4H,9H-furo [3″,2″:2′,3′]cyclopenta [1′,2′:3,4]furo[2,3-b]pyrrol-8-yl benzoate (XBB-078). Using method C (Scheme 5) yielding in XBB-078 as a white powder (99 mg, 75%). R_{f}=0.1 (DCM/MeOH, 10:1); mp=116.5-117.2° C.; ¹H-NMR (400 MHz, CDCl₃): δ [ppm]=8.13-7.88 (m, 2H), 7.64 (t, J=7.5 Hz, 1H), 7.60-7.38 (m, 5H), 6.95-6.81 (m, 1H), 6.33 (s, 1H), 5.91 (s, 1H), 5.21 (t, J=7.0 Hz, 1H), 4.71 (t, J=8.8 Hz, 2H), 3.74 (d, J=11.8 Hz, 2H), 3.46-3.18 (m, 4H), 3.15 (d, J=18.6 Hz, 1H), 2.86 (d, J=18.8 Hz, 1H), 2.74-2.65 (m, 1H), 2.62 (s, 1H), 2.30-2.08 (m, 2H), 1.89-1.73 (m, 2H), 1.52-1.29 (m, 2H), 1.10 (s, 9H); ¹³C{¹H}-NMR (100 MHz, CDCl₃): δ [ppm]=177.93, 173.60, 167.64, 165.24, 164.09, 134.31, 130.04, 129.22, 128.80, 128.66, 128.58, 127.72, 127.41, 127.01, 126.89, 124.83, 109.56, 88.58, 87.08, 83.54, 72.36, 70.69, 62.83, 59.25, 46.58, 45.76, 42.59, 37.40, 36.44, 33.56, 29.22, 29.09, 26.48; HRMS (ESI) m/z: [M+K]⁺ Calcd for C₃₇H₄₀N₂O₁₀K⁺ 711.25243, found: 711.25198.

### Example 1.8: Synthesis of Other BB Derivatives (XBB-008 and XBB-009) According to

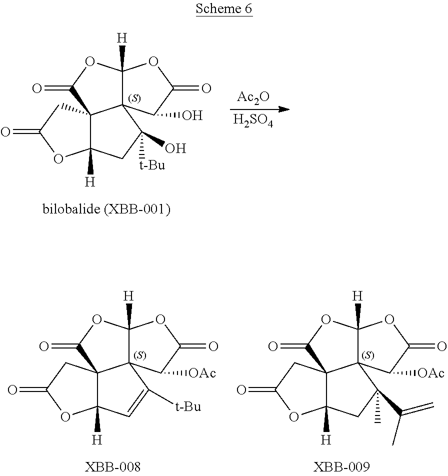

To a solution of bilobalide (1.0 g) in acetic anhydride (20 mL) was added a trace of concentrated sulfuric acid (20 L). The resulting solution was allowed to be stirred at 50° C. for 3 h. Once the starting material was fully consumed, the reaction solution was quenched with saturated sodium bicarbonate solution. The mixture was extracted with ethyl acetate and the organic layer was washed with brine. The combined organic layers were dried over anhydrous sodium sulfate and then concentrated under reduced pressure. The crude product was purified by column chromatography with elution system (hexane:EtOAc=8:1) to yield XBB-008 (54%) and XBB-009 (42%).

XBB-008: R_{f}=0.40 (hexane:EtOAc, 2:1); mp=127.1-128.0° C.; ¹H-NMR (500 MHz, CDCl₃): δ [ppm]=6.57 (s, 1H), 6.42 (s, 1H), 6.16 (d, J=2.8 Hz, 1H), 5.24 (d, J=2.8 Hz, 1H), 3.10 (d, J=17.9 Hz, 1H), 2.97 (d, J=17.9 Hz, 1H), 2.17 (s, 3H), 1.32 (s, 9H); ¹³C{¹H}-NMR (125 MHz, CDCl₃): δ [ppm]=174.64, 171.95, 168.84, 166.64, 156.21, 129.16, 99.29, 86.09, 68.91, 67.09, 58.35, 37.14, 35.05, 31.36, 19.90; HRMS (ESI) m/z: [M+Na]⁺ Calcd for C₁₇H₁₈O₈Na⁺ 373.08939, found 373.08930.

XBB-009: R_{f}=0.35 (hexane:EtOAc, 2:1); mp=170.5-171.2° C.; ¹H-NMR (500 MHz, CDCl₃): δ [ppm]=6.06 (s, 1H), 6.02 (s, 1H), 5.16 (q, J=1.4 Hz, 1H), 5.00 (s, 1H), 4.96 (dd, J=5.6, 1.4 Hz, 1H), 3.21 (d, J=17.7 Hz, 1H), 3.07 (d, J=17.7 Hz, 1H), 2.42 (dd, J=15.4, 5.6 Hz, 1H), 2.26-2.17 (m, 4H), 1.88 (d, J=1.4 Hz, 3H), 1.34 (s, 3H); ¹³C{H}-NMR (125 MHz, CDCl₃): δ [ppm]=174.22, 172.60, 168.48, 167.19, 145.20, 115.93, 115.75, 101.10, 86.90, 69.23, 63.53, 61.57, 52.82, 41.21, 37.63, 24.49, 21.63, 20.44; HRMS (ESI) m/z: [M+Na]⁺ Calcd for C₁₇H₁₈O₈Na⁺ 373.08939, found 373.08945. The X-ray crystal structure of XBB-009 is shown in FIG. 1G.

### Synthesis of (3aS,5aS,8R,8aS,10aS)-9-(tert-butyl)-2,4,7-trioxo-2,3,5a,6,7,8-hexahydro-4H,10aH-furo[3″,2″:2′,3′]cyclopenta[1′,2′:3,4]furo[2,3-b]pyrrol-8-yl acetate (XBB-010)

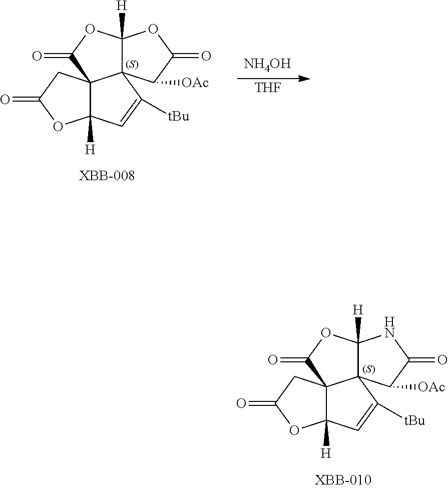

To a solution of XBB-008 (50 mg, 93.607 mmol, 1.0 equiv) in anhydrous tetrahydrofuran (2 mL) was added 25%-28% ammonia solution (13 mg, 0.187 mmol, 2.0 equiv) at 0° C. The resulting solution was then allowed to be stirred for 30 min at room temperature. The reaction was monitored by TLC and upon completion the reaction solution was diluted ethyl acetate. The organic layer was washed with brine and the combined organic layers were dried over Na₂SO₄, filtered and concentrated in vacuo. The crude product was purified via column chromatography (hexane:EtOAc=1:1) to provide XBB-010 as a white powder (86% yield, 43 mg). The X-ray crystal structure of XBB-010 is shown in FIG. 1H. R_{f}=0.15 (hexane:EtOAc, 1:1); mp=216.2-217.1° C.; ¹H-NMR (500 MHz, CDCl₃): δ [ppm]=7.52 (s, 1H), 6.13 (d, J=2.7 Hz, 1H), 6.08 (s, 1H), 5.24 (d, J=2.7 Hz, 1H), 5.01 (s, 1H), 3.09-2.90 (m, 2H), 1.74 (s, 3H), 1.31 (s, 9H); ¹³C{¹H}-NMR (125 MHz, CDCl₃): δ [ppm]=176.89, 174.61, 174.57, 157.97, 127.58, 87.03, 85.60, 70.87, 67.83, 59.52, 37.82, 34.90, 31.51, 30.01; HRMS (ESI) m/z: [M+Na]⁺ Calcd for C₁₇H₁₉NO₇Na⁺ 372.10537, found 372.10513.

### Synthesis of (3aS,5aS,8R,10aS)-9-(tert-butyl)-8-hydroxy-5a,6-dihydro-4H,10aH-furo[3″,2″:2′,3′]cyclopenta[1′,2′:3,4]furo[2,3-b]pyrrole-2,4,7(3H,8H)-trione (XBB-012)

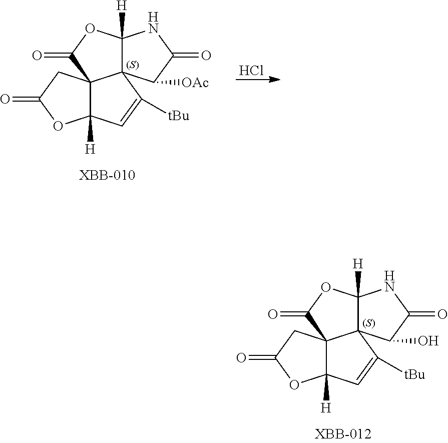

To a round-bottom flask was added XBB-010 (100 mg) and 3 N HCl in H₂O (10 mL), respectively. The resulting solution was allowed to be stirred under reflux condition for 12 h. Once the starting material was fully consumed, the reaction solution was cooled down to room temperature and then the pH value was adjusted to 7.0 using saturated sodium bicarbonate solution. The mixture was extracted with ethyl acetate and washed with brine. The combined organic layers were dried over anhydrous sodium sulfate and concentrated under reduced pressure. The crude product was purified by column chromatography with elution system (hexane:EtOAc=1:2) to give XBB-012 as a white powder (70% yield, 62 mg). R_{f}=0.15 (hexane:EtOAc, 1:2); mp=230.4-231.0° C.; ¹H-NMR (500 MHz, MeOD): δ [ppm]=6.19 (s, 1H), 6.18 (d, J=2.8 Hz, 1H), 5.23 (s, 1H), 5.14 (d, J=2.8 Hz, 1H), 2.86 (d, J=17.5 Hz, 1H), 2.77 (d, J=17.5 Hz, 1H), 1.31 (s, 9H); ¹³C{¹H}-NMR (125 MHz, MeOD): δ [ppm]=179.54, 176.67, 175.39, 159.30, 128.89, 88.00, 87.59, 71.41, 69.09, 60.94, 37.61, 36.10, 31.87; HRMS (ESI) m/z: [M+Na]⁺ Calcd for C₁₅H₁₇NO₆Na⁺ 330.09445, found 330.09451.

### Synthesis of (3aS,5aS,8R,8aS,9R,10S,10aR)-9-(tert-butyl)-9,10-dihydroxy-2,4,7-trioxooctahydro-4H,9H-furo[3″,2″:2′,3′]cyclopenta[1′,2′:3,4]furo[2,3-b]pyrrol-8-yl acetate (XBB-014)

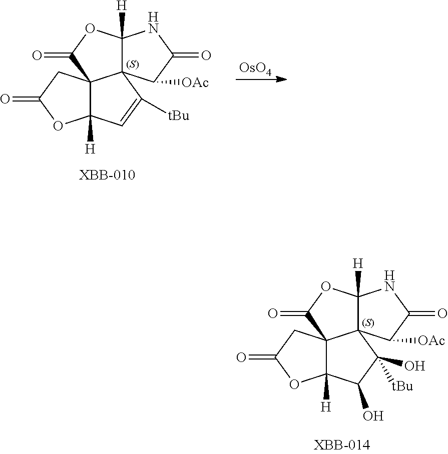

To a round-bottom flask was added XBB-010 (1.0 equiv, 200 mg) and 14 mL of acetone/H₂O (v/v=6:1), followed by the addition of pyridine (1 mL) and osmium (VIII) oxide. The resulting solution was allowed to be stirred at room temperature for 18 h. Once completion indicated by TLC, acetone was removed under reduced pressure and the resultant was diluted with ethyl acetate and washed with 10% aqueous sodium sulfite solution and brine, respectively. The combined organic layers was dried over anhydrous sodium sulfate and concentrated under reduced pressure, and the residue was purified by column chromatography with elution system (hexane:EtOAc=1:2, v/v) to give XBB-014 as white powder (86% yield, 189 mg). R_{f}=0.1 (hexane:EtOAc, 1:2); mp=201.2-201.9° C.; ¹H-NMR (500 MHz, MeOD): δ [ppm]=6.11 (s, 1H), 6.07 (s, 1H), 4.83 (d, J=5.0 Hz, 1H), 4.23 (d, J=5.0 Hz, 1H), 2.90 (q, J=18.2 Hz, 2H), 2.14 (s, 3H), 1.16 (s, 9H); ¹³C{¹H}-NMR (125 MHz, MeOD): δ [ppm]=177.57, 174.17, 171.62, 169.85, 89.84, 84.64, 84.21, 81.34, 70.33, 62.52, 55.61, 53.42, 37.13, 36.73, 25.76, 19.47; HRMS (ESI) m/z: [M+Na]⁺ Calcd for C₁₇H₂₁NO₉Na⁺ 406.11085, found 406.11079.

### Synthesis of (3aS,5aS,8R,8aS,9R,10S,10aR)-9-(tert-butyl)-9,10-dihydroxy-2,4,7-trioxooctahydro-4H,9H-furo[3″,2″:2′,3′]cyclopenta[1′,2′:3,4]furo[2,3-b]pyrrol-8-yl acetate

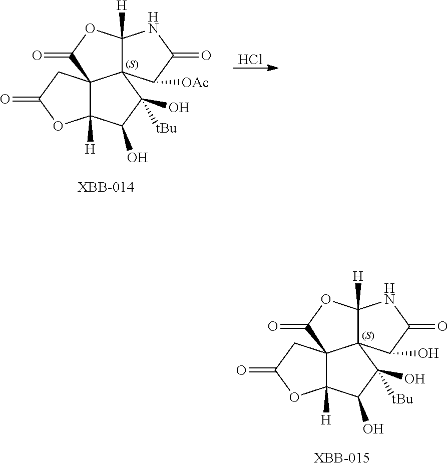

To a round-bottom flask was added XBB-014 (100 mg) and 3 N HCl in H₂O (10 mL), respectively. The resulting solution was allowed to be stirred at 85° C. for 12 h. Once he starting material was fully consumed, the reaction solution was cooled down to room temperature and then the pH value was adjusted to 7.0 using saturated sodium bicarbonate solution. The mixture was extracted with ethyl acetate and washed with brine. The combined organic layers were dried over anhydrous sodium sulfate and concentrated under reduced pressure. The crude product was purified by column chromatography with elution system (dichloromethane:MeOH=30:1) to give XBB-015 as white powder (86% yield, 71 mg). R_{f}=0.2 (DCM/MeOH=30:1); mp=2061.7-207.5° C.; ¹H-NMR (400 MHz, acetone-d6): δ [ppm]=8.29 (s, 1H), 6.02 (s, 1H), 5.90 (d, J=3.6 Hz, 1H), 5.30 (d, J=5.9 Hz, 1H), 4.97 (d, J=3.7 Hz, 1H), 4.71 (d, J=5.1 Hz, 1H), 4.62 (t, J=5.5 Hz, 1H), 4.31 (s, 1H), 3.05 (d, J=17.9 Hz, 1H), 2.63 (d, J=17.9 Hz, 1H), 1.25 (s, 9H); ¹³C{¹H}-NMR (100 MHz, acetone-d6): δ [ppm]=178.21, 173.67, 173.31, 89.65, 84.40, 81.18, 69.75, 63.66, 55.06, 37.21, 36.86, 26.35; HRMS (ESI) m/z: [M+Na]⁺ Calcd for C₁₅H₁₉NO₈Na⁺ 364.10029, found 364.09999.

### Example 1.9: Synthesis of (3aS,5aS,8R,8aS,10aS)-9-(tert-butyl)-2,4,7-trioxo-2,3,5a,6,7,8-hexahydro-4H,10aH-furo[3″,2″:2′,3′]cyclopenta[1′,2′:3,4]furo[2,3-b]pyrrol-8-yl acetate (XBB-010) According to Scheme 7 (or Scheme 2)

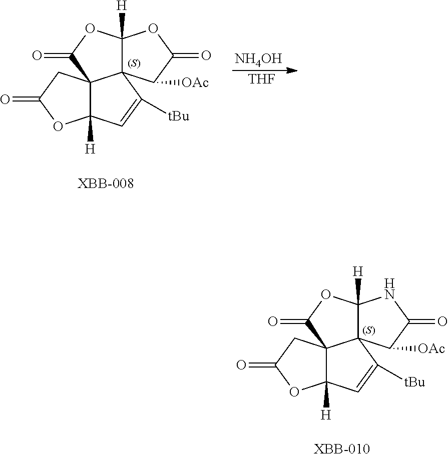

To a solution of XBB-008 (50 mg, 93.607 mmol, 1.0 equiv) in anhydrous tetrahydrofuran (2 mL) was added 25%-28% ammonia solution (13 mg, 0.187 mmol, 2.0 equiv) at 0° C. The resulting solution was then allowed to be stirred for 30 min at room temperature. The reaction was monitored by TLC and upon completion the reaction solution was diluted ethyl acetate. The organic layer was washed with brine and the combined organic layers were dried over Na₂SO₄, filtered and concentrated in vacuo. The crude product was purified via column chromatography (hexane:EtOAc=1:1) to provide XBB-010 as a white powder (86% yield, 43 mg). R_{f}=0.15 (hexane:EtOAc, 1:1); mp=216.2-217.1° C.; ¹H-NMR (500 MHz, CDCl₃): δ [ppm]=7.52 (s, 1H), 6.13 (d, J=2.7 Hz, 1H), 6.08 (s, 1H), 5.24 (d, J=2.7 Hz, 1H), 5.01 (s, 1H), 3.09-2.90 (m, 2H), 1.74 (s, 3H), 1.31 (s, 9H); ¹³C{¹H}-NMR (125 MHz, CDCl₃): δ [ppm]=176.89, 174.61, 174.57, 157.97, 127.58, 87.03, 85.60, 70.87, 67.83, 59.52, 37.82, 34.90, 31.51, 30.01; HRMS (ESI) m/z: [M+Na]⁺ Calcd for C₁₇H₁₉NO₇Na⁺ 372.10537, found 372.10513.

### Example 1.10: Synthesis of (3aS,5aS,8R,10aS)-9-(tert-butyl)-8-hydroxy-5a,6-dihydro-4H,10aH-furo[3″,2″:2′,3′]cyclopenta[1′,2′:3,4]furo[2,3-b]pyrrole-2,4,7(3H,8H)-trione (XBB-012) According to Scheme 8

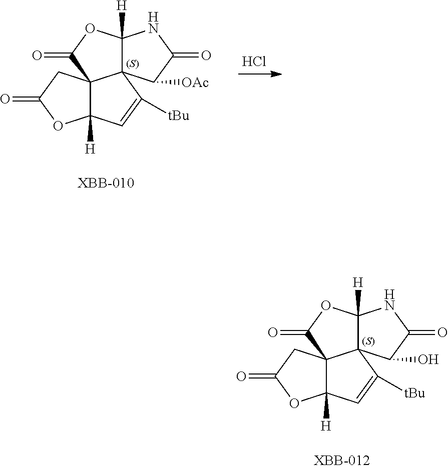

To a round-bottom flask was added XBB-010 (100 mg) and 3 N HCl in H₂O (10 mL), respectively. The resulting solution was allowed to be stirred under reflux condition for 12 h. Once the starting material was fully consumed, the reaction solution was cooled down to room temperature and then the pH value was adjusted to 7.0 using saturated sodium bicarbonate solution. The mixture was extracted with ethyl acetate and washed with brine. The combined organic layers were dried over anhydrous sodium sulfate and concentrated under reduced pressure. The crude product was purified by column chromatography with elution system (hexane:EtOAc=1:2) to give XBB-012 as a white powder (70% yield, 62 mg). R_{f}=0.15 (hexane:EtOAc, 1:2); mp=230.4-231.0° C.; ¹H-NMR (500 MHz, MeOD): δ [ppm]=6.19 (s, 1H), 6.18 (d, J=2.8 Hz, 1H), 5.23 (s, 1H), 5.14 (d, J=2.8 Hz, 1H), 2.86 (d, J=17.5 Hz, 1H), 2.77 (d, J=17.5 Hz, 1H), 1.31 (s, 9H); ¹³C{¹H}-NMR (125 MHz, MeOD): δ [ppm]=179.54, 176.67, 175.39, 159.30, 128.89, 88.00, 87.59, 71.41, 69.09, 60.94, 37.61, 36.10, 31.87; HRMS (ESI) m/z: [M+Na]⁺ Calcd for C₁₅H₁₇NO₆Na⁺ 330.09445, found 330.09451.

### Example 1.11a: Synthesis of (3aS,5aS,8R,8aS,9R,10S,10aR)-9-(tert-butyl)-9,10-dihydroxy-2,4,7-trioxooctahydro-4H,9H-furo[3″,2″:2′,3′]cyclopenta[1′,2′:3,4]furo[2,3-b]pyrrol-8-yl acetate (XBB-014) According to Scheme 9

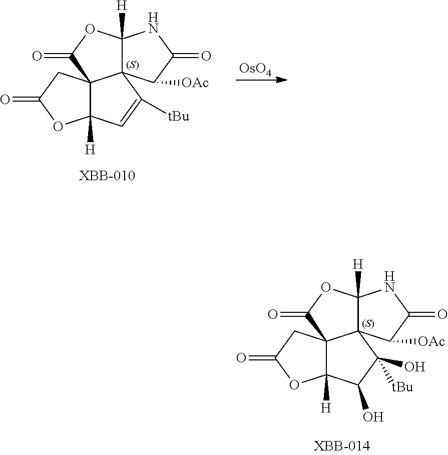

To a round-bottom flask was added XBB-010 (1.0 equiv, 200 mg) and 14 mL of acetone/H₂O (v/v=6:1), followed by the addition of pyridine (1 mL) and osmium (VIII) oxide. The resulting solution was allowed to be stirred at room temperature for 18 h. Once completion indicated by TLC, acetone was removed under reduced pressure and the resultant was diluted with ethyl acetate and washed with 10% aqueous sodium sulfite solution and brine, respectively. The combined organic layers was dried over anhydrous sodium sulfate and concentrated under reduced pressure, and the residue was purified by column chromatography with elution system (hexane:EtOAc=1:2, v/v) to give XBB-014 as white powder (86% yield, 189 mg). R_{f}=0.1 (hexane:EtOAc, 1:2); mp=201.2-201.9° C.; ¹H-NMR (500 MHz, MeOD): δ [ppm]=6.11 (s, 1H), 6.07 (s, 1H), 4.83 (d, J=5.0 Hz, 1H), 4.23 (d, J=5.0 Hz, 1H), 2.90 (q, J=18.2 Hz, 2H), 2.14 (s, 3H), 1.16 (s, 9H); ¹³C{¹H}-NMR (125 MHz, MeOD): δ [ppm]=177.57, 174.17, 171.62, 169.85, 89.84, 84.64, 84.21, 81.34, 70.33, 62.52, 55.61, 53.42, 37.13, 36.73, 25.76, 19.47; HRMS (ESI) m/z: [M+Na]⁺ Calcd for C₁₇H₂₁N₉Na⁺ 406.11085, found 406.11079.

### Example 1.11b: Synthesis of (3aS,5aS,8R,8aS,9R,10S,10aR)-9-(tert-butyl)-9,10-dihydroxy-2,4,7-trioxooctahydro-4H,9H-furo[3″,2″:2′,3′]cyclopenta[1′,2′:3,4]furo[2,3-b]pyrrol-8-yl acetate (XBB-015) According to Scheme 8

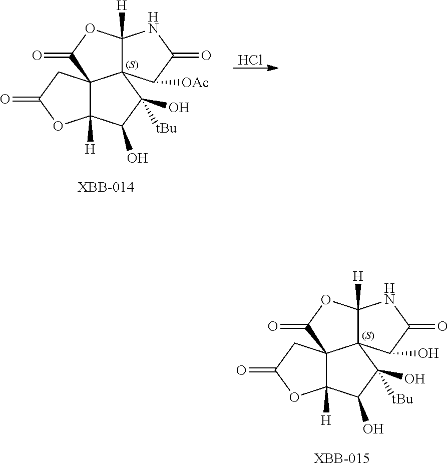

To a round-bottom flask was added XBB-014 (100 mg) and 3 N HCl in H₂O (10 mL), respectively. The resulting solution was allowed to be stirred at 85° C. for 12 h. Once he starting material was fully consumed, the reaction solution was cooled down to room temperature and then the pH value was adjusted to 7.0 using saturated sodium bicarbonate solution. The mixture was extracted with ethyl acetate and washed with brine. The combined organic layers were dried over anhydrous sodium sulfate and concentrated under reduced pressure. The crude product was purified by column chromatography with elution system (dichloromethane:MeOH=30:1) to give XBB-015 as white powder (86% yield, 71 mg). R_{f}=0.2 (DCM/MeOH=30:1); mp=2061.7-207.5° C.; ¹H-NMR (400 MHz, acetone-d6): δ [ppm]=8.29 (s, 1H), 6.02 (s, 1H), 5.90 (d, J=3.6 Hz, 1H), 5.30 (d, J=5.9 Hz, 1H), 4.97 (d, J=3.7 Hz, 1H), 4.71 (d, J=5.1 Hz, 1H), 4.62 (t, J=5.5 Hz, 1H), 4.31 (s, 1H), 3.05 (d, J=17.9 Hz, 1H), 2.63 (d, J=17.9 Hz, 1H), 1.25 (s, 9H); ¹³C{¹H}-NMR (100 MHz, acetone-d6): δ [ppm]=178.21, 173.67, 173.31, 89.65, 84.40, 81.18, 69.75, 63.66, 55.06, 37.21, 36.86, 26.35; HRMS (ESI) m/z: [M+Na]⁺ Calcd for C₁₅H₁₉NO₈Na⁺ 364.10029, found 364.09999.

### Example 2: Initial Screening Results of Bilobalide Analogues

In this example, the anti-cancer properties of the BB analogues described in Table 1d were evaluated. Single-dose screening approach is used for initial testing to assess the cytotoxic effect of the BB analogues (also referred to as ‘BB compounds’) on Jurkat cells (human T cell leukaemia cells) and A549 cells (human lung cancer cells). Cell viability was measured using cell counting kit-8 (CCK-8) (MCE).

### Method for Adherent Cells (A549 Cells)

In this example, 5000 cells/100 uL were seeded in 96-well plates and incubate at 37° C.+5% CO₂ overnight. Media were replaced with 100 uL of 50 uM compound per well in 6 replicates. The following controls were prepared.

Blank control: 100 uL 0.1% DMSO in media with no cells per well.

Positive control: 100 uL of 10 uM 5-flurouracil per well.

Negative control: 100 uL of 0.1% DMSO in media per well.

Each 96-well plate included 7 compounds and the complete set of controls (in 6 replicates).

Cells were incubated at 37° C.+5% CO₂ for 46 hours. 10 uL of CCK8 solution was added to each well. Cells were then incubated at 37° C.+5% CO₂ for 2 hours. Absorbance at 450 nm was measured using a multiplate reader.

Viability was calculated using the equation:

$$\% \text{Viability} = \frac{Test - Blank}{Negative - Blank} \times 100 \%$$

### Method for Suspension Cells (Jurkat Cells)

5000 cells/90 uL were seeded in 96-well plates. 10 uL of 500 uM compound (10×) was added to each well in 6 replicates. The following controls were prepared.

Blank control: 10 uL 1% DMSO in media with no cells per well.

Positive control: 10 uL of 100 uM 5-flurouracil (10×) per well.

Negative control: 10 uL of 1% DMSO in media (10×) per well.

Each 96-well plate included 7 BB analogues and the complete set of controls (in 6 replicates).

Cells were incubated at 37° C.+5% CO₂ for 46 hours. 10 uL of CCK8 solution was added to each well. Cells were then incubated at 37° C.+5% CO₂ for 2 hours. Absorbance at 450 nm was measured using a multiplate reader.

Viability was calculated using the equation:

$$\% \text{Viability} = \frac{Test - Blank}{Negative - Blank} \times 100 \%$$

The benchmark in Table 2 was used to define the activities of each compound.

TABLE 2a
Benchmark for determining activities

| % Viability | Interpretation |
|---|---|
| <50 | Excellent activity |
| 50-70 | Good activity |
| 70-90 | Moderate activity |
| >90 | No detectable activity at 50 uM in two cell lines |

### Results

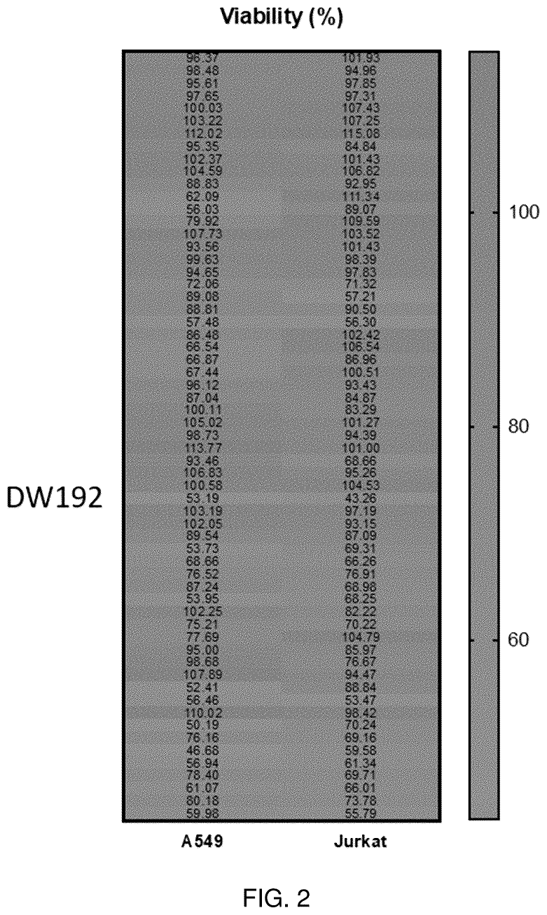

Now referring to FIG. 2, a heat map of viability after DW192 treatment (5 uM, 48 h) in A549 and Jurkat cells. In which, 59 uM DW192 treatment for 48 hours could achieve 53% and 43% viability on A549 and Jurkat cells respectively. Out of all the compounds according to Table 1d, DW192 and several other compounds exhibited significant cytotoxic effect against both cell lines, and the results are shown in Table 2b. The compounds showing sensitivities towards the A549 and Jurkat cell lines with S.D.<10 were chosen for further studies.

TABLE 2b
Initial screening of BB analogues

| | |
|---|---|
| % Viability (50 uM, 48 h) | Average |

| Compound | A549 | Jurkat | (%) | S.D. |
|---|---|---|---|---|
| DW192 | 53.19 | 43.26 | 48.23 | 7.02 |
| P-29 | 46.68 | 59.58 | 53.13 | 9.12 |
| P-21 | 56.46 | 53.47 | 54.97 | 2.11 |
| P-30 | 56.94 | 61.34 | 59.14 | 3.11 |
| P-19 | 50.19 | 70.24 | 60.22 | 14.18 |
| JW100 | 53.95 | 68.25 | 61.10 | 10.11 |
| JW092 | 53.73 | 69.31 | 61.52 | 11.02 |
| P-33 | 61.07 | 66.01 | 63.54 | 3.49 |
| JW093 | 68.66 | 66.26 | 67.46 | 1.70 |
| XBB-036 | 52.41 | 88.84 | 70.63 | 25.76 |
| XBB-023 | 72.06 | 71.32 | 71.69 | 0.52 |
| XBB-034 | 56.03 | 89.07 | 72.55 | 23.36 |
| P-28 | 76.16 | 69.16 | 72.66 | 4.95 |
| JW107 | 75.21 | 70.22 | 72.72 | 3.53 |
| XBB-039 | 78.4 | 69.71 | 74.06 | 6.14 |
| JW094 | 76.52 | 76.91 | 76.72 | 0.28 |
| XBB-035 | 66.87 | 86.96 | 76.92 | 14.21 |
| P-34 | 80.18 | 73.78 | 76.98 | 4.53 |
| JW095 | 87.24 | 68.98 | 78.11 | 12.91 |
| DW184 | 93.46 | 68.66 | 81.06 | 17.54 |
| XBB-075 | 67.44 | 100.51 | 83.98 | 23.38 |
| XBB-045 | 87.04 | 84.83 | 85.94 | 1.56 |
| XBB-073 | 66.54 | 106.54 | 86.54 | 28.28 |
| P-5 | 98.68 | 75.67 | 87.18 | 16.27 |
| JW081 | 89.54 | 87.09 | 88.32 | 1.73 |
| XBB-028 | 88.81 | 90.5 | 89.66 | 1.20 |
| XBB-038 | 95.35 | 84.84 | 90.10 | 7.43 |
| XBB-037 | 95 | 85.97 | 90.49 | 6.39 |
| XBB-054 | 88.83 | 92.95 | 90.89 | 2.91 |
| JW116 | 77.69 | 104.79 | 91.24 | 19.16 |
| XBB-025 | 100.11 | 83.29 | 91.70 | 11.89 |
| JW103 | 102.25 | 82.22 | 92.24 | 14.16 |
| XBB-018 | 86.48 | 102.42 | 94.45 | 11.27 |
| XBB-058 | 79.92 | 109.59 | 94.76 | 20.98 |
| XBB-029 | 96.12 | 93.43 | 94.78 | 1.90 |
| XBB-024 | 94.65 | 97.83 | 96.24 | 2.25 |
| DW172 | 98.73 | 94.39 | 96.56 | 3.07 |
| XBB-004 | 98.48 | 94.96 | 96.72 | 2.49 |
| XBB-042 | 95.61 | 97.85 | 96.73 | 1.58 |
| XBB-068 | 93.56 | 101.43 | 97.50 | 5.56 |
| XBB-040 | 99.63 | 98.39 | 99.01 | 0.88 |
| XBB-006 | 96.37 | 101.93 | 99.15 | 3.93 |
| JW072 | 103.19 | 97.19 | 100.19 | 4.24 |
| DW189 | 106.83 | 95.26 | 101.05 | 8.18 |
| P-8 | 107.89 | 94.47 | 101.18 | 9.49 |
| DW191 | 100.58 | 104.53 | 102.56 | 2.79 |
| DW168 | 105.02 | 101.27 | 103.15 | 2.65 |
| XBB-013 | 100.03 | 107.43 | 103.73 | 5.23 |
| XBB-037′ | 110.02 | 98.42 | 104.22 | 8.20 |
| XBB-009 | 103.22 | 107.25 | 105.24 | 2.85 |
| XBB-060 | 107.73 | 103.52 | 105.63 | 2.98 |
| XBB-016 | 104.59 | 106.82 | 105.71 | 1.58 |
| DW182 | 113.77 | 101 | 107.39 | 9.03 |
| XBB-010 | 112.02 | 115.08 | 113.55 | 2.16 |

### Example 3: Dose-Dependent Screening

In this example, dose-dependent CCK8 Viability assay was performed to determine the IC50 (the concentration of compound to achieve 50% viability) of several of the hit compounds identified from Example 2. A variety of cell lines were used:
Jurkat cells: Human leukemia
A549 cells: Human NSCLC
KP-1 cells: Mouse NSCLC with KRAS and P53 mutations
MCF-7 cells: Human breast cancer

### Method for Adherent Cells (A549, MCF-7 and KP-1 Cells)

5000 cells/100 uL were seeded in 96-well plates and incubate at 37° C.+5% CO₂ overnight. Media were replaced with 100 uL of compound per well in 6 replicates in a series of 2-fold dilutions. The following controls were prepared.

Blank control: 100 uL 0.1% DMSO in media with no cells per well.

Positive control: 100 uL of 10 uM 5-flurouracil per well.

Negative control: 100 uL of 0.1% DMSO in media per well.

Each 96-well plate included 1 compound in 7 concentrations and a complete set of controls (in 6 replicates).

Cells were incubated at 37° C.+5% CO₂ for 46 hours. 10 uL of CCK8 solution was added to each well. Cells were incubated at 37° C.+5% CO₂ for 2 hours. Absorbance at 450 nm was measured using a multiplate reader.

Viability was calculated using the equation:

$$\% \text{Viability} = \frac{Test - Blank}{Negative - Blank} \times 100 \%$$

### Method for Suspension Cells (Jurkat Cells)

5000 cells/90 uL were seeded in 96-well plates. 10 uL of compound (10×) in various concentrations was added to each well in 6 replicates. The following controls were prepared.

Blank control: 10 uL 1% DMSO in media with no cells per well.

Positive control: 10 uL of 100 uM 5-flurouracil (10×) per well.

Negative control: 10 uL of 1% DMSO in media (10×) per well.

Note: Each 96-well plate can test 1 compound in 7 concentrations with a complete set of controls (in 6 replicates)

Cells were incubated at 37° C.+5% CO₂ for 46 hours. 10 uL of CCK8 solution was added to each well. Cells were incubated at 37° C.+5% CO₂ for 2 hours. Absorbance at 450 nm was measured using a multiplate reader.

Viability was calculated using this equation:

$$\% \text{Viability} = \frac{Test - Blank}{Negative - Blank} \times 100 \%$$

### Results

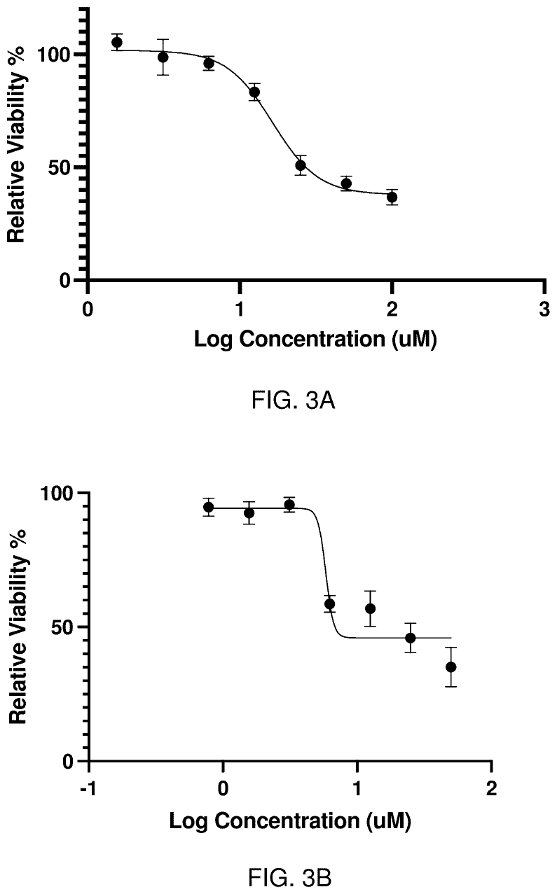

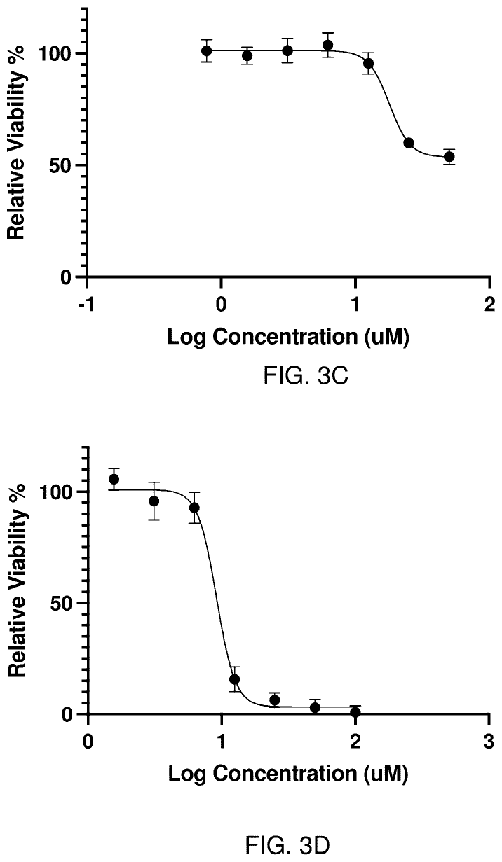

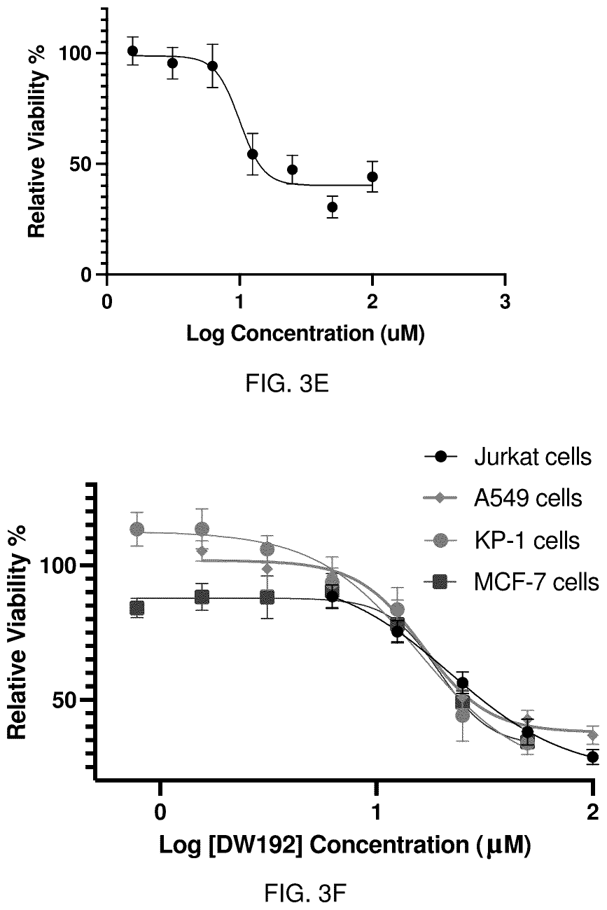

Now referring to FIGS. 3A-3E, plots showing the dose-response curve on A549 and KP-1 cells treated with the compounds DW192, P-29, P-21, SCC506, and SCC363 for 48 hours, respectively. BB analogue DW192 exhibited cytotoxic effect towards A549 cells with an IC₅₀ value of 21.92 μM, as shown in FIG. 3A. BB analogue P-29 exhibited cytotoxic effect towards A549 cells with an IC₅₀ value of 5.84 μM, as shown in FIG. 3B. BB analogue P-21 exhibited cytotoxic effect towards A549 cells with an IC₅₀ value of 17.92 μM, as shown in FIG. 3C. BB analogue SCC506 exhibited cytotoxic effect towards KP-1 cells with an IC₅₀ value of 17.15 μM, as shown in FIG. 3D. BB analogue SCC363 exhibited cytotoxic effect towards A549 cells with an IC₅₀ value of 9.987 μM, as shown in FIG. 3E. The IC₅₀ values in μM are summarized in Table 3. These results indicate that the compounds DW192, P-29, P-21, SCC506, and SCC363 are therapeutically effective against human NSCLC.

TABLE 3
Calculated IC₅₀ values towards A549 and KP-1 cells

| Compound | Cell lines | IC₅₀ (μM) |
|---|---|---|
| DW192 | A549 | 21.92 μM |
| P-29 | A549 | 5.84 μM |
| P-21 | A549 | 17.92 μM |
| SCC506 | KP-1 | 17.15 μM |
| SCC363 | A549 | 9.987 μM |

Now referring to FIG. 3F, a plot showing overlaid dose-dependent curves on Jurkat cells, A549 cells, KP-1 cells, and MCF-7 cells treated with DW192 for 48 hours. The IC₅₀ is 21.92 μM, 16.40 μM, 15.83 μM and 19.56 μM respectively. These results indicate that the compound DW192 is therapeutically effective against several cancer cell lines, including human leukemia, NSCLC, breast cancer, as well as mouse NSCLC cell lines with KRAS and P53 mutations.

### Example 4: NCI-60 Human Tumor Cell Line Screen

In this example, the cytotoxic effect of DW192 was assess via the NCI-60 Human Tumor Cell Lines Screen, provided by the Developmental Therapeutics Program (DTP) of the National Cancer Institute (NCI) in the United States. This screening helps to identify and characterize the cytotoxic effect of compounds on 60 different human cancer cell lines, including leukemia, melanoma, and cancers of the lung, colon, brain, ovary, breast, prostate and kidneys. The screening comprised of two assays: one-dose screen and five-dose screen. In one-dose screen, 10 μM of DW192 was tested for 48 hours. In five-dose screen, four, 10-fold of ½ log serial dilutions were tested for 48 hours.

Method (Extracted from the NCI-60 Website)

Cells were seeded into 96-well plates in 100 uL at plating densities ranging from 5,000 to 40,000 cells/well depending on the doubling time of individual cell lines. Plates were incubated at 37° C.+5% CO₂₊₉₅% air+100% relative humidity for 24 hours before adding compounds. Two plates of each cell line were fixed in situ with TCA, to represent a measurement of the cell population for each cell line at the time of drug addition (Tz). 100 uL of compounds in four, 10-fold of ½ log serial dilutions were added to the wells already containing 100 uL of medium. Plates were incubated at 37° C.+5% CO₂₊₉₅% air+100% relative humidity for an additional 48 hours.

To terminate the assay, adherent cells were fixed in situ by the gentle addition of 50 uL of cold 50% (w/v) TCA (final concentration, 10% TCA) and incubated for 60 min at 4° C. For suspension cells, 50 uL of 80% TCA (final concentration, 16% TCA) was used.

Supernatant was discarded, and the plates were wash five times with tap water and air dried.

Sulforhodamine B(SRB) solution (100 uL) at 0.4% (w/v) in 1% acetic acid was added to each well, and the plates were incubated for 10 min at room temp. The plates were washed five times with 1% acetic acid and air dried.

Remaining stained cells were solubilized with 10 mM trizma base. Absorbance at 515 nm was measured.

Percentage growth was calculated using the following equations:

$$\frac{T i − T z}{C − T z} \times 100 for which Ti \geq Tz ; and$$

$$\frac{T i − T z}{T z} \times 100 for which Ti < Tz ,$$

where Tz=absorbance at time zero, C=absorbance of control growth, Ti=absorbance of test growth in the presence of drug at the five concentration levels.

In five-dose screen, three dose response parameters were calculated with the steps below (extracted from NCI-60 website):

Growth inhibition of 50% (GI50):

$$\frac{T i − T z}{C − T z} \times 100 = 50 ,$$

which is the drug C-Tz concentration resulting in a 50% reduction in the net protein increase (as measured by SRB staining) in control cells during drug incubation.

Total growth inhibition (TGI): Ti=Tz

Lethality (LC50):

$$\frac{T i − T z}{T z} \times 100 = - 50 ,$$

concentration of drug resulting in a 50% reduction in the measured protein at the end of drug treatment as compared to that at the beginning, indicating a net loss of cells after treatment.

### Results

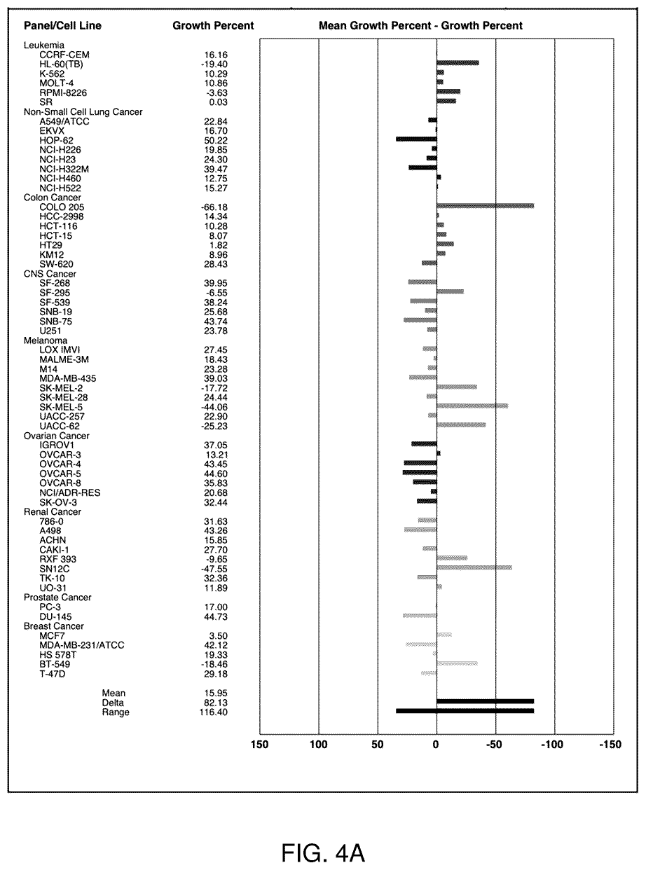

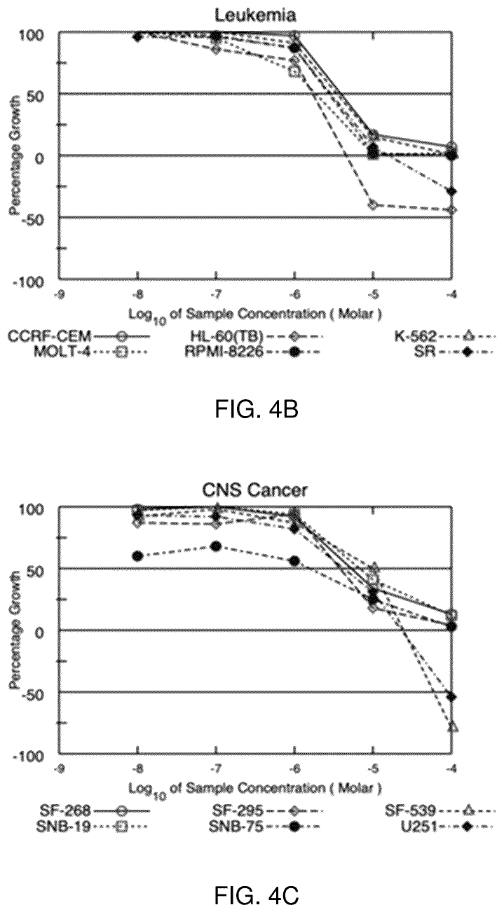

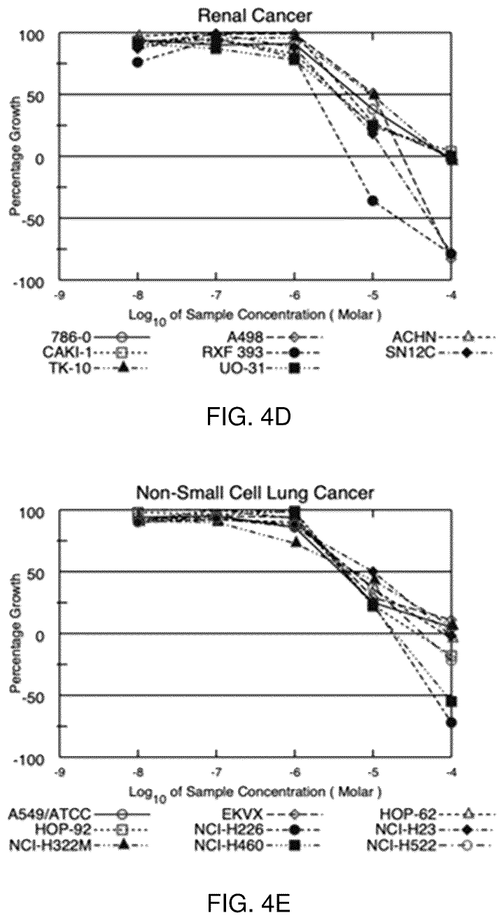

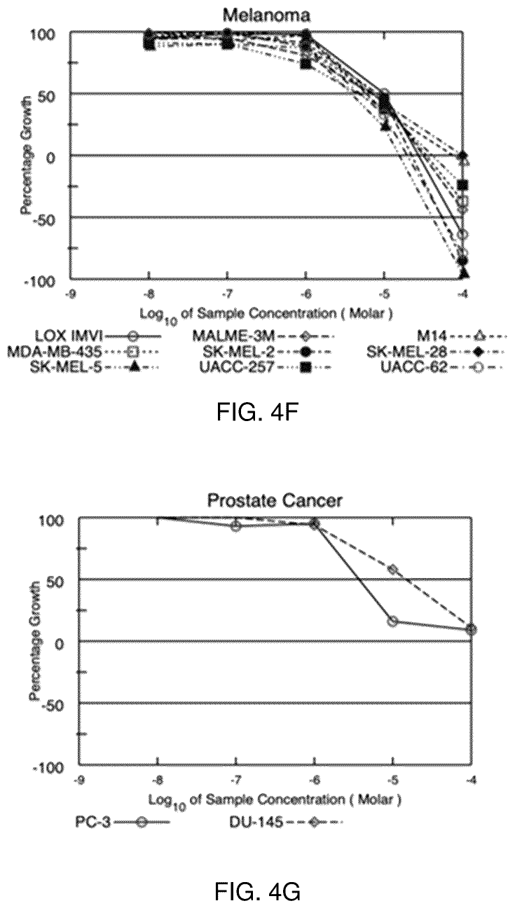

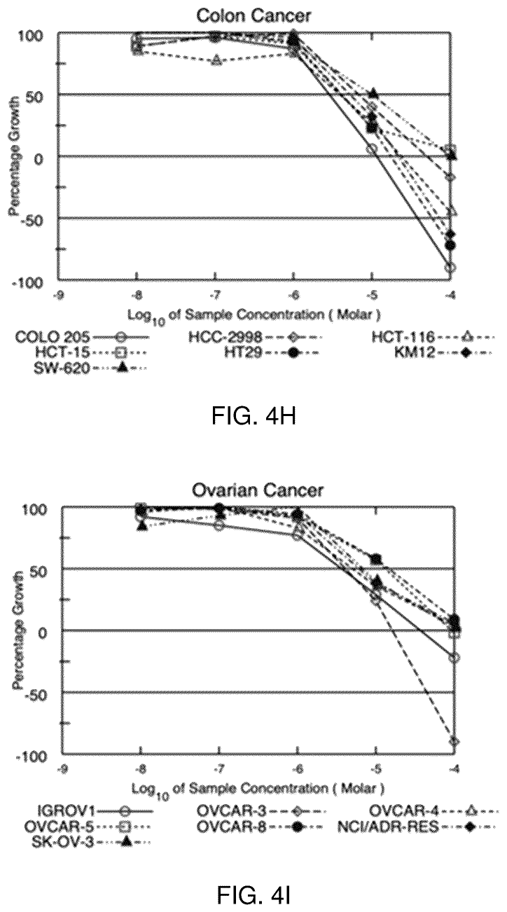

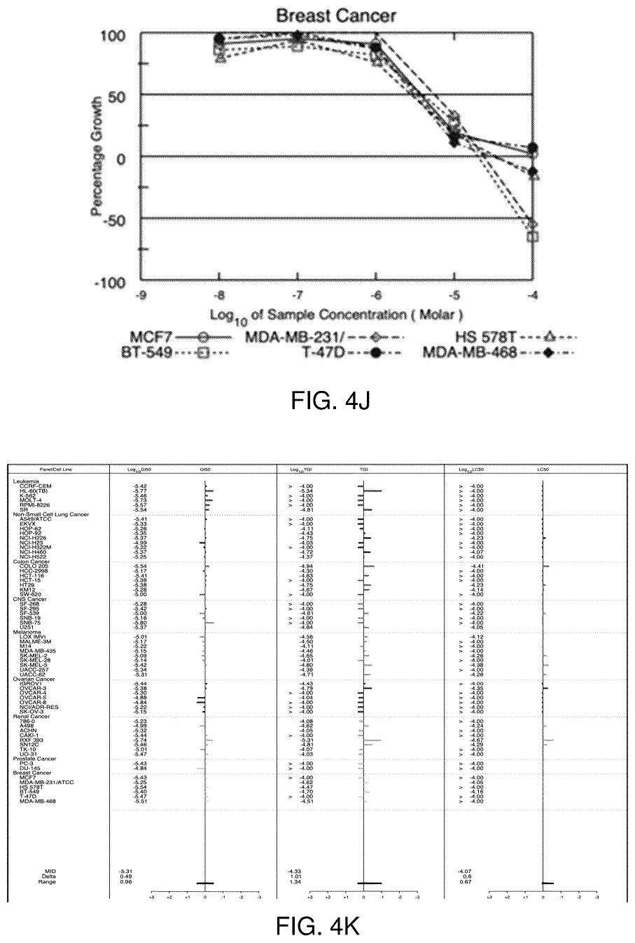

Now referring to FIG. 4A, showing a one-dose mean graph of percentage growth of cell lines across the NCI-60 cell line panel when treated with 10 μM DW192 for 48 hours. The one-dose data was reported as a mean graph of the percent growth of treated cells, detecting both growth inhibition (values between 0 and 100) and lethality (values less than 0). As shown in FIG. 4A, DW192 exhibits cytotoxic effects towards multiple cell lines of different cancer type, including leukemia (such as HL-60(TB)), NSCLC (such as HCl—H460), colon cancer (such as COL0205), CNS cancer (such as SF-295), melanoma (such as SK-MEL-2, SK-MEL-5, UACC-62), ovarian cancer (such as OVCAR-3), renal cancer (such as RXF 393, SN12C), and breast cancer (such as BT-529). The most sensitive cell line against DW192 is COL0205 (colon cancer), achieving 66.18% lethality. The comparatively less sensitive cell line is HOP62 (NSCLC), achieving 50.22% growth inhibition. The dose-response curves of cell lines across the NCI-60 cell line panel when treated with DW192 for 48 hours are shown in FIGS. 4B-4J for leukemia, CNS cancer, renal cancer, NSCLC, melanoma, prostate cancer, colon cancer, ovarian cancer and breast cancer, respectively. FIG. 4K shows the mean graphs of GI50, TGI and LC50 calculated from five-dose screen results (Unit: Molar). These results have demonstrated the pan-anti-cancer effect of DW192 against most of the human cell lines with various mutation profiles.

### Example 5: Stability of Bilobalide Analogues

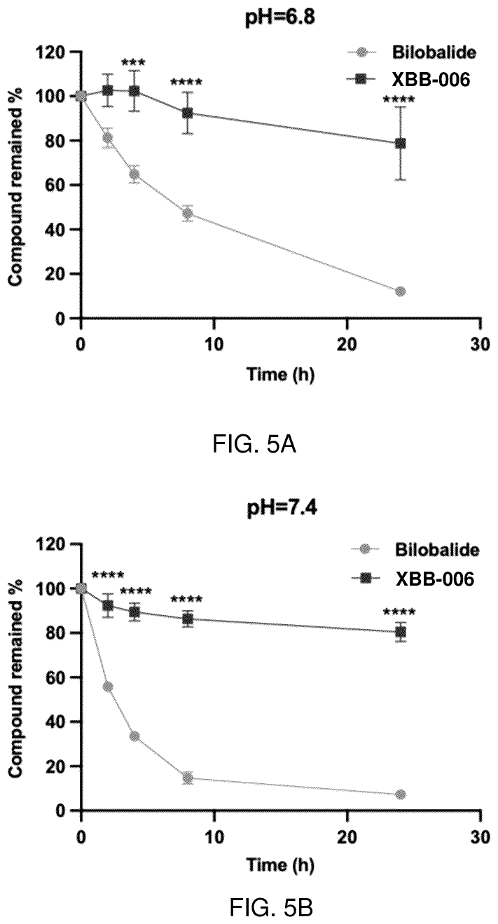

Now referring to FIGS. 5A-5B, the hydrolytic stabilities of bilobalide and BB analogue (XBB-006) monitored using LC-MS/MS in buffer with pH=6.8 and 7.4 are shown, respectively. As shown in FIG. 5A, under the pH of 6.8, the concentration of bilobalide has dropped to below 20% after 20 hours whereas the concentration of XBB-006 has remained at about 80%. As shown in FIG. 5B, under the pH of 7.4, the concentration of bilobalide has dropped to below 20% within the first 10 hours whereas the concentration of XBB-006 has remained above 80%. These results showed a much higher hydrolytic stability of bilobalide analogue with respect to bilobalide at physiological pH values.

### Example 6: Activities as a Ferroptosis Inhibitor

In this example, the anti-ferroptotic properties of the BB analogues described in preceding examples were evaluated by a RSL3-induced ferroptosis model. RSL3 is an allosteric covalent inhibitor of GSH-dependent enzyme GSH peroxidase 4 (GPX4), which is responsible for removing ROS from cells. By covalently binding to GPX4 protein, RSL3 induces the degradation of this antioxidant enzyme, leading to an accumulation of ROS and causing oxidative damage to cellular proteins. Cell lines derived from the central nervous system, such as the hippocampal cell line HT22, and the microglial cell lines HMC3 and BV-2, are highly sensitive to RSL3-induced lethality. To evaluate the anti-ferroptosis activity of BB analogues, we developed a phenotypic screening based on RSL3-induced ferroptosis model on HT22 mouse hippocampal cell line, HMC3 microglial cell line and BV-2 murine microglial cell line which were co-treated or pre-treated with RSL3 and BB analogues for 2-24 h, and the cell viability was subsequently measured. Compounds which showed significant rescue effects against ferroptosis were selected as hits. Dose-response studies were carried out for the hits, which will be discussed in the next example.

### General Methods

HMC3 cell line was maintained in Minimum Essential Medium (MEM, Gibco) supplemented with 1× non-essential amino acid (Gibco), 1 mM sodium pyruvate (Gibco), 10% fetal bovine serum (FBS) (Gibco) and 1% penicillin/streptomycin (Gibco). BV-2 cell line was maintained in Roswell Park Memorial Institute (RPMI) 1640 medium (Gibco) supplemented with 10% FBS and 1% penicillin/streptomycin, and HT22 cell line was maintained in Dulbecco's Modified Eagle's Medium (DMEM) (Gibco) supplemented with 10% FBS and 1% penicillin/streptomycin. All cells were incubated at 37° C. in a humidified atmosphere under 5% CO2.

For phenotypic screening of bilobalide (BB) analogues, 5,000 cells per well (100 uL volume) of HT22, HMC3 or BV-2 cells were placed in 96-well plate and allowed to adhere for 22 h. After which, cells were pre-treated with 200 nM (on HT22, HMC3) or 500 nM (on BV-2) of RSL3 (Bidepharm) for 2 h respectively. Medium was then replaced by 50 μM BB analogues and followed by 22 h incubation subsequently. Cell viability was measured using cell counting kit-8 (CCK-8) (MCE). For dose-response of BB analogues, RSL3, HT22 and HMC3 cells (seeded on 96-well plates at 5,000 cells per well) were treated with multiple doses of RSL3 for 2 h. Medium was then replaced by DMSO (0.1%) only, XBB-037 (50 PM) or bilobalide (50 μM). BV-2 cells (seeded on 96-well plates at 5,000 cells per well) were co-treated with multiple doses of RSL3 and DMSO (0.1%) only, XBB-037 (50 PM), bilobalide (50 PM) for 24 h. Cell viability was measured after 22 h treatment by CCK-8. For evaluation of ferroptosis inducers, HMC3 cells (seeded on 96-well plates at 5,000 cells per well for 24 h) was pre-treated with multiple concentrations of erastin (MCE), ML210 (MCE), ML162 (MCE) or FIN56 (MCE), for 2 h prior the 22 h treatment with DMSO (0.1%) only, XBB-037 (50 μM), bilobalide (50 μM). Cell viability was measured by CCK-8 using CLARIOstar monochromator multimode plate reader under 450 nm wavelength.

For flow cytometry, HMC3 cells (seeded 50,000 cells per well on 24-well plate one day prior to the experiment) were pretreated with 50 nM RSL3 for 2 h. After RSL3 treatment, medium was replaced by DMSO (0.1%) only, XBB-037 (50, 25 or 12.5 μM), bilobalide (50, 25 or 12.5 μM) or ferrostatin-1 (1 μM, MCE) following by 3 h treatment. After which medium was replaced with BODIPY™ 581/591C₁₁ (10 μM, Invitrogen) or CellROX™ Green (5 μM, Invitrogen) for 30 min staining. Cells were then washed 3 times with PBS and collected. Fluorescence was measured by BD FACSympgony A5.2 SORP Flow Cell Analyzer (BD Biosciences).

For fluorescent imaging, 10,000 cells per well of HMC3 were laid on cover slip and incubated overnight. After the same compound treatment as described above in this section, cells were subsequently incubated with CellROX™ Green and Hoechst 34580 (MCE) for 30 min. After incubation, cells were washed 3 times gently with PBS. Cover slips were then attached to glass slides by Anti-Fade Fluorescence Mounting Medium (Abcam) and imaged via ECLIPSE Ti fluorescence microscope (Nikon). Fluorescent intensity was quantified using imageJ software.

All data were plotted as mean±s.d., ns, no significance, **p<0.01, ***p<0.001, and ****p<0.0001. Statistical analyses were performed by one-way ANOVA with multiple comparisons using Tukey's multiple comparisons test. The statistical analysis of all test groups was compared to the NR group respectively.

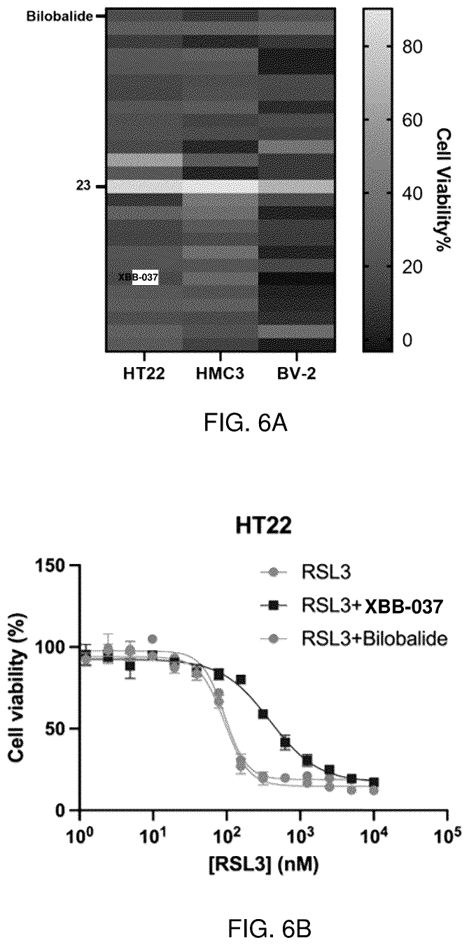

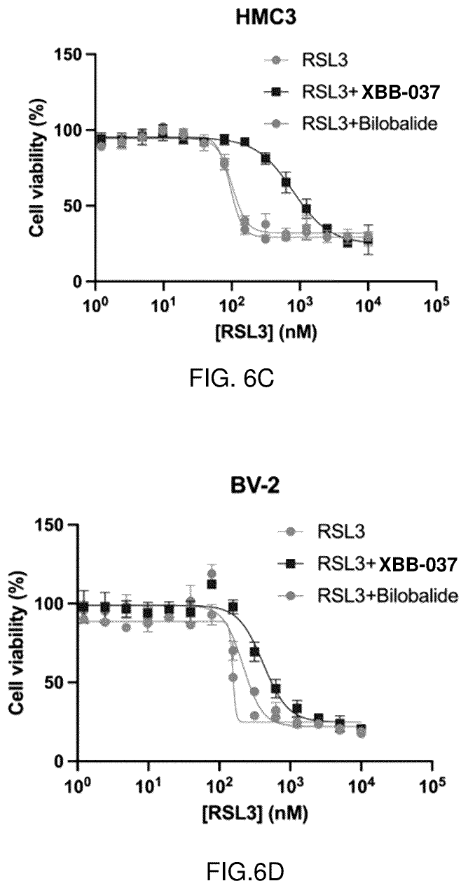

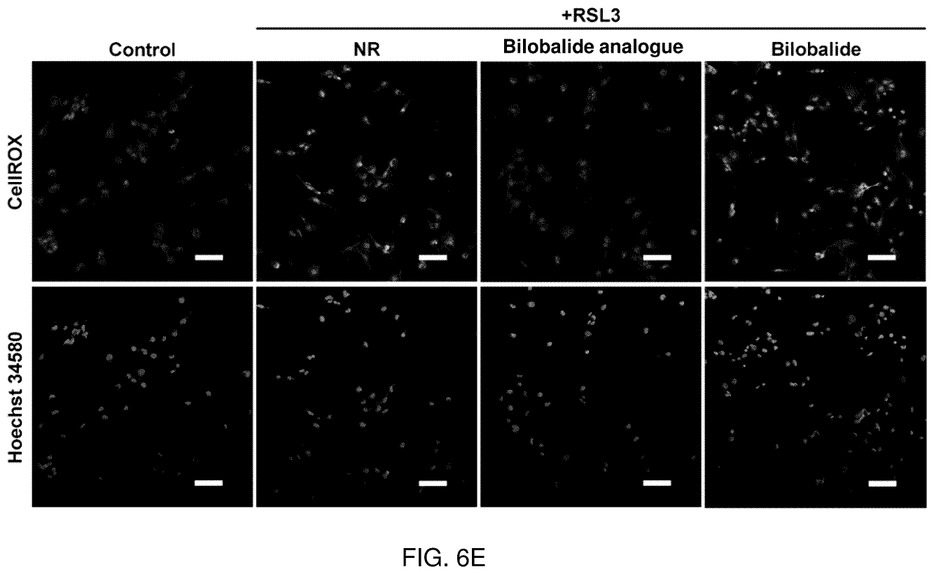

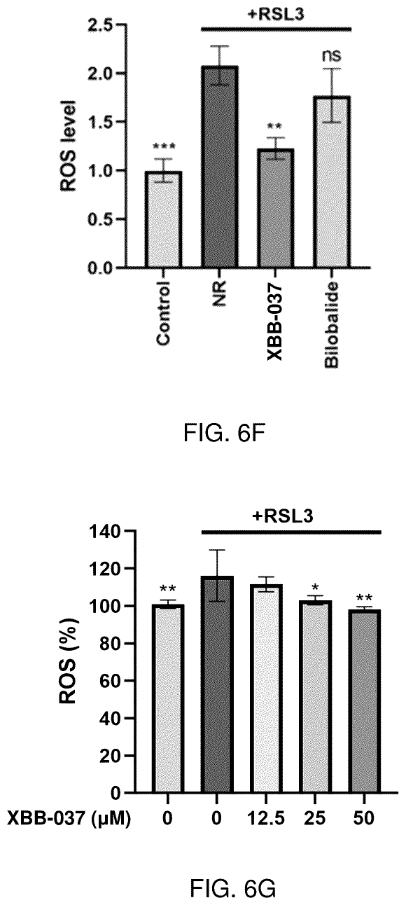

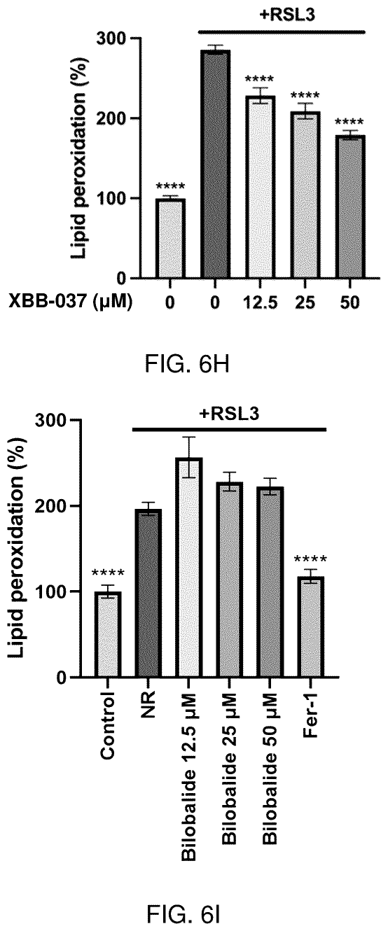

Now referring to FIG. 6A, a chart comparing the phenotypic screening of unmodified bilobalide and a bilobalide analogue against RSL3-induced ferroptosis through 3 cell lines is shown. In this example, the cells were pre-treated with RSL3 (200 nM for HT22 and HMC3, 500 nM for BV-2) for two hours, followed by bilobalide analogues treatment (50 PM) for 22 hours. Cell viability was measured using cell counting kit-8 (CCK-8); n=5 technical replicates. In this example, the bilobalide analogue was XBB-037 described in preceding examples, which was found to effectively inhibit ferroptosis across 3 cell lines, as shown in FIG. 6A. The result suggested that N-arylated analogue such as XBB-037 effectively inhibited RSL3-induced ferroptosis. Such effect was further investigated by treating the cell lines with various doses of RSL3.

Now referring to FIGS. 6B-6D, the dose-dependent curves of RSL3 with or without treatment of bilobalide (50 μM) or XBB-037 (50 μM) on HT22, HMC3 and BV-2 cell lines, respectively, are shown (n=3 technical replicates). As shown in FIGS. 6B-6D, treatments of 50 μM bilobalide analogue (RSL3+XBB-037) displayed anti-ferroptotic activity in a dose-dependent manner, compared to the control group which was not treated with XBB-037 or bilobalide (RSL3) and the sample group treated with unmodified bilobalide (RSL3+Bilobalide). Consistent with the phenotypic screening result shown in FIG. 6A, treatment with bilobalide analogue (RSL3+XBB-037) diminished RSL3 lethality in 3 cell lines compared to the treatment with no bilobalide analogue (RSL3) and the treatment with unmodified bilobalide (RSL3+Bilobalide). Intriguingly, the curve shifts post BB treatment were relatively marginal, which indicated the modification brought new pharmacological effects (such as anti-ferroptotic effects) to bilobalide analogues.

To further evaluate the anti-ferroptotic properties of bilobalide analogues, we tested its effect on the total level of intracellular reactive oxygen species (ROS), we performed fluorescent staining on the HMC3 cell line using CellROX, which is a probe which can emit green fluorescence after reacting with ROS. In this experiment, HMC3 cells were pre-treated with 50 nM RSL3 for two hours, and subsequently treated with 50 μM bilobalide analogue (XBB-037) or bilobalide for three hours. A control of untreated HMC3 cells, as well as a comparative condition NR containing HMC3 cells treated with RSL3 where RSL3 doubled the ROS level but not treated with any bilobalide or bilobalide analogues, were also prepared. The conditions are summarized in Table 4. Now referring to FIG. 6E, a chart comparing the fluorescent staining on HMC3 cell lines under the conditions according to Table 4 is shown (sale bars=100 m). ROS was stained with CellROX and the cell nucleus was stained with Hoechst 34580. FIG. 6F shows the normalization of ROS level against the Control based on the fluorescent intensity of CellROX (n=3 technical replicates). Based on the data shown in FIGS. 6E-6F, the treatment with 50 μM XBB-037 significantly reduced intracellular ROS level. By contrast, treatment with 50 μM unmodified bilobalide showed no reduction in ROS level.

TABLE 4
Example conditions for the evaluation of ROS level

| Condition | [RSL3] (nM) | Rescuer/inhibitor |
|---|---|---|
| Control | — | — |
| NR | 50 | — |
| Bilobalide analogue | 50 | XBB-037 |
| Bilobalide | 50 | Bilobalide |

Now referring to FIG. 6G, a plot showing the normalization of ROS level (%) in cells pre-treated with 50 nM of RSL3 for 2 hours, and then treated with various concentrations of XBB-037 for 3 hours, is presented. ROS level was measured by flow cytometry using CellROX.

The results further indicated a dose-dependent reduction in ROS levels following treatment with bilobalide analogue, as shown in FIG. 6G, where disruptions of ROS level were observed.

Disequilibrium ROS level acts as a direct initiator of lipid peroxidation, which is one of the major hallmarks of ferroptosis. In this example, HMC3 cells according to the conditions in Table 4 were subjected to measurement of lipid peroxidation level using flow cytometry and the lipid peroxidation sensor C11-BODIPY.

Now referring to FIG. 6H, a chart showing the lipid peroxidation level (%) measured by flow cytometry using C11-BODIPY is presented (n=3 technical replicates). Compared to the control and NR groups, the groups treated with 50, 25 and 12.5 μM bilobalide analogue XBB-037 showed significant down-regulation of RSL3-induced lipid peroxidation accumulation in a dose-dependent manner. However, no significant alteration was found after treatment with the same concentrations of bilobalide (12.5, 25 and 50 μM) as lipid peroxidation in samples treated with bilobalide were higher than the Control, NR and Fer-1, as shown in FIG. 6I.

### Example 7: Mechanistic Studies

To further explore how bilobalide analogues regulated ferroptosis, we tested their protection against various ferroptosis inducers (Class I-III) and their mechanisms in the glutathione peroxidase 4 (GPX4) pathway, which is crucial for reducing hydroperoxides during ferroptosis.

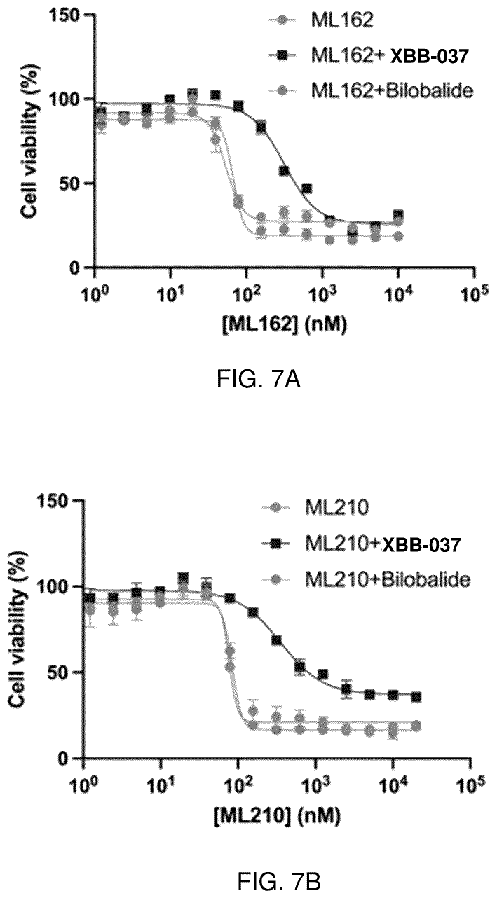

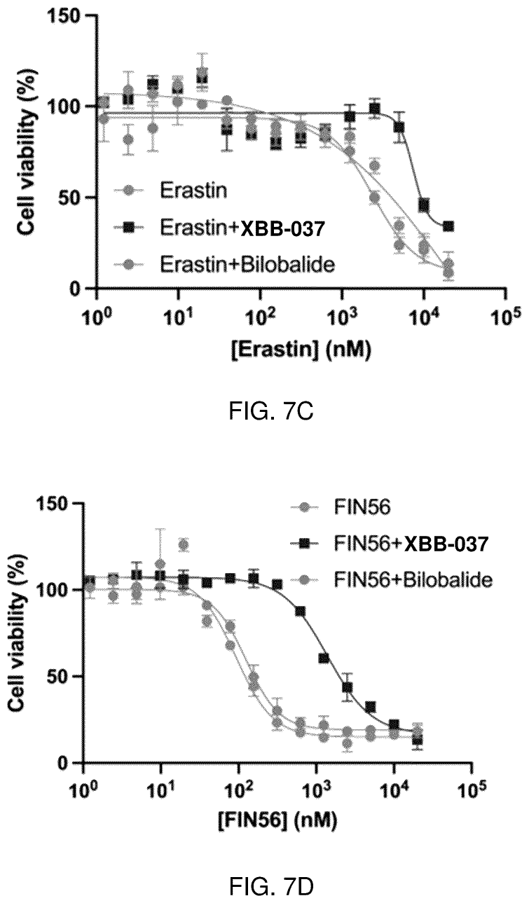

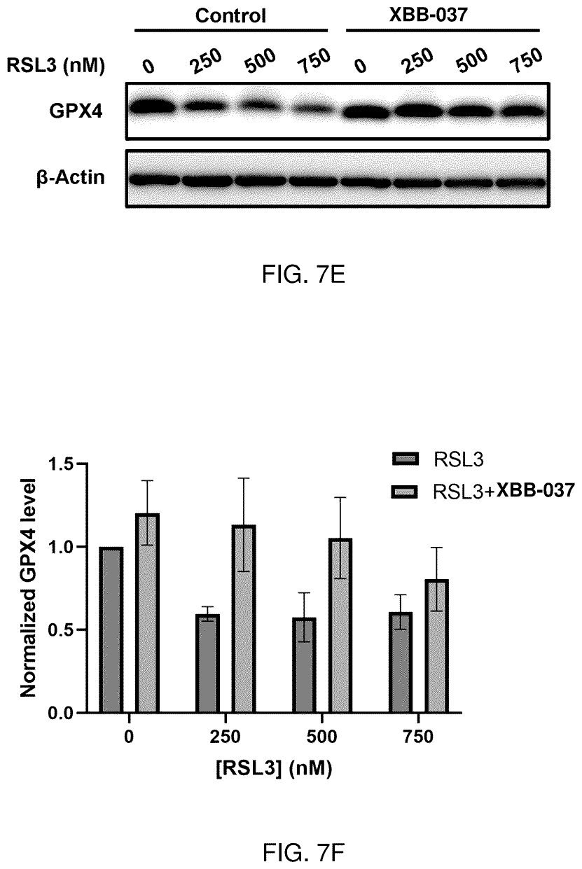

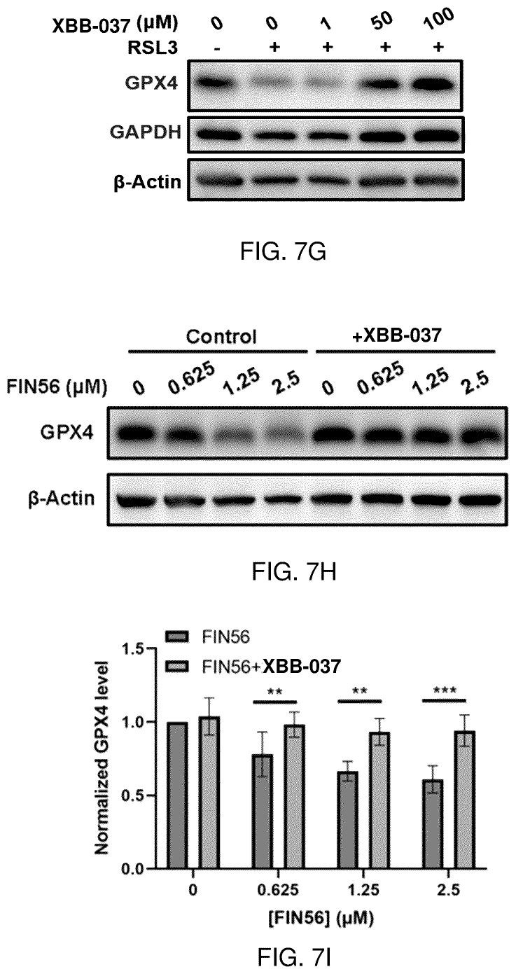

Class II ferroptosis inducers ML162 and ML210 are GPX4 covalent inhibitors. As shown in FIG. 7A, the treatment of 50 μM bilobalide analogue (ML162+XBB-037) alleviated the lethality of ML162, compared to the control group which was not treated with XBB-037 or bilobalide (ML162) and the sample group treated with unmodified bilobalide (ML162+Bilobalide). Consistently, as shown in FIG. 7B, the treatment of 50 μM bilobalide analogue (ML210+XBB-037) alleviated the lethality of ML210, compared to the control group (ML210) and the sample group treated with 50 μM unmodified bilobalide (ML210+Bilobalide). These results demonstrated the ability of bilobalide analogues to alleviate the lethality of Class II ferroptosis inducers such as ML162 and ML210.

As shown in FIG. 7C, albeit the drift in cell viability within the range of 101-10² nM erastin, the treatment of 50 μM bilobalide analogue (Erastin+XBB-037) suppressed erastin-induced lethality, compared to the control group (Erastin) and the sample group treated with 50 μM unmodified bilobalide (Erastin+Bilobalide). Collectively, our observation on system Xc-inhibitors reflected that the mechanism of bilobalide analogue XBB-037 may directly interact with ferroptosis.

Lastly, FIN56 is a class III ferroptosis inducer, which induces ferroptosis via the depletion of GPX4 instead of acting as a GPX4 binder. As shown in FIG. 7D, the treatment of 50 μM bilobalide analogue (FIN56+XBB-037) effectively suppressed FIN56-induced cell death, compared to the control group (FIN56) and the sample group treated with 50 μM unmodified bilobalide (FIN56+Bilobalide). Intriguingly, the results showed that bilobalide analogue effectively repressed FIN56-induced cell death, suggesting its utility effect on the regulation of GPX4 level.

Aside from the GPX4 inhibition, RSL3 has also been reported to degrade GPX4 through inhibiting the mammalian target of rapamycin (mTOR) and enhancing chaperone-mediated autophagy (CMA). As mentioned above, RSL3 treatment results in an oxidized environment in cells, which promotes the CMA to scavenge damaged proteins including GPX4 or glyceraldehyde-3-phosphate dehydrogenase (GAPDH), a well-known CMA substrate. To gain insights into the mechanism of action of bilobalide analogue XBB-037 against the RSL3-induced ferroptosis, HMC3 cells were pre-treated with RSL3, followed by treatment with or without bilobalide analogue XBB-037.

In FIN56 assay, HMC3 (300,000 cells per well on 6-well plate) was pre-treated with FIN56 (2.5, 1.25, 0.625 μM) for 2 h, followed by 8 h treatment of DMSO only (0.1%) or XBB-037 (50 μM). In RSL3 assay, HMC3 (300,000 cells per well on 6-well plate) was pre-treated with RSL3 (750, 500, 250 nM) for 2 h, followed by 4 h or 8 h treatment of DMSO only (0.1%) or XBB-037 (50 μM). After incubation, cells were washed 3 times with ice-cold PBS and lysed in RIPA buffer containing 1× protease inhibitor cocktail (MCE) and nuclease (Biyotime) on ice for 30 min. All samples were centrifuged and then quantified by bicinchoninic acid (BCA) assay (Pierce). Cell lysates were diluted with Laemmli Sample Buffer (Bio-Rad) and heated at 95° C. for 10 min. Samples were separated by 12% SDS-PAGE and transferred to a polyvinylidene difluoride membrane. Membrane was incubated with 3% bovine serum albumin (BSA, Sigma-Aldrich) for 1 h, and subsequently incubated with indicated primary antibody overnight at 4° C. After incubation, membrane was washed by wash buffer (containing 20 mM Tris, 150 mM NaCl and 0.1% Tween 20), and incubated with peroxidase-conjugated secondary antibody at room temperature for 1 h. Antibodies for GPX4 (52455, CST) was used at 1:500 dilution, β-Actin (AC026, ABclonal) was used at 1:400,000 dilution, GAPDH (AC035, ABclonal) was used at 1:3000 dilution. Anti-mouse IgG HRP-linked antibody (7076, CST) and anti-rabbit IgG HRP-linked antibody (7074, CST) was used at 1:6000. Blot was washed and visualized using Clarity Western ECL Substrate (Bio-Rad) with ChemiDoc MP Imaging System (Bio-Rad). Band intensity was normalized by image lab software.

Now referring to FIGS. 7E-7F, GPX4 levels in HMC3 cells pre-treated with RSL3 are shown. Protein levels of GPX4 and 3-actin at 0, 250, 500 or 750 nM RSL3 were measured by Western-blot. HMC3 cells were pretreated with RSL3 for 2 hours and then treated with or without 50 μM bilobalide analogue XBB-037 for 4 hours. We found that RSL3 diminished the GPX4 level, and RSL3-induced GPX4 degradation was reduced after the treatment of bilobalide analogue XBB-037. As shown in FIG. 7E, higher RSL3 concentrations clearly diminished the GPX4 in the control without bilobalide analogue treatment. On the other hand, the GPX4 remained constant at all four RSL3 concentrations (0, 250, 500 and 750 nM) in the sample group treated with 50 μM bilobalide analogue XBB-037, as seen in FIG. 7F, where the normalized GPX4 levels with or without bilobalide analogue XBB-037 treatment is shown. These data revealed that RSL3-induced GPX4 degradation was blocked after bilobalide analogue treatment.

RSL3 also induced the degradation of glycer-aldehyde-3-phosphate dehydrogenase (GAPDH), a ferroptosis-related marker. Interestingly, as shown in FIG. 7G, when HMC3 cells were pre-treated with 0 (−) or 500 (+) nM RSL3 for 2 hours, then treated with various concentrations of XBB-037 (0, 0, 1, 50 and 100 μM) for 8 hours, it was observed that XBB-037 restored the level of GAPDH accordingly. This observation reflected the governing of GPX4 by bilobalide analogue XBB-037, which played an important role in the mechanism against RSL3-induced ferroptosis.

In addition to GPX4, FIN56 was further used to investigate the GPX4 regulation of bilobalide analogue XBB-037. As mentioned above, FIN56 is a Class III ferroptosis inducer, which causes the depletion of cellular GPX4 levels without affecting GPX4 activity. In this experiment, HMC3 cells were pretreated with FIN56 for 2 hours and then treated with or without 50 μM XBB-037 for 8 hours. Protein levels were measured by Western-blotting using indicated antibodies. Data were plotted as mean±s.d., n=3 technical replicates. **p<0.01. Statistical analyses were performed by two-way ANOVA with multiple comparisons using Two-stage linear step-up procedure of Benjamini, Krieger and Yekutieli multiple comparisons test.

Now referring to FIGS. 7H-7I, GPX4 levels in HMC3 cells pre-treated with FIN56 were shown. Protein levels of GPX4 and 3-actin at 0, 0.625, 1.25 and 2.5 μM FIN56 were measured by Western-blot, as shown in FIG. 7H. The normalized plot of GPX4 levels are shown in FIG. 7I.

The results showed the treatment of bilobalide analogue XBB-037 significantly restored GPX4 level against depletion by different concentrations of FIN56, supporting the role of bilobalide analogue XBB-037 in regulating the GPX4 level. Our results suggest that bilobalide analogue could selectively inhibit ferroptosis induced by class I, II, and/or III ferroptosis inducers. By restoring GPX4 level under ferroptotic environment, cell viability against ferroptosis was maintained.

In conclusion, the results have shown that bilobalide analogues such as XBB-037 significantly reduce key ferroptosis markers, such as ROS levels and lipid peroxidation, and notably counteracted GPX4 degradation induced by RSL3 and FIN56, highlighting the therapeutic potential of bilobalide analogues against neurological diseases (e.g., neurodegenerative diseases) by inhibiting ferroptosis.

### Example 8: Activities as a Ferroptosis Inhibitor

In this example, the anti-ferroptotic properties of the BB analogues described in preceding examples were evaluated by a RSL3-induced ferroptosis model similar to the conditions as discussed in Example 6 herein. For the sake of brevity and simplicity of the present disclosure, the full discussion is not reproduced here.

For phenotypic screening of bilobalide (BB) analogues, 5,000 cells per well (100 μL volume) of HT22, HMC3 or BV-2 cells were placed in 96-well plate and allowed to adhere for 22 h. After which, cells were pre-treated with 200 nM (on HT22, HMC3) or 500 nM (on BV-2) of RSL3 (Bidepharm) for 2 h respectively. Medium was then replaced by 50 μM BB analogues and followed by 22 h incubation subsequently. Cell viability was measured using cell counting kit-8 (CCK-8) (MCE). For dose-response of SXQ087-1 and XBB-037 (HMC3 cells were seeded on 96-well plates at 5,000 cells per well) were treated with 200 nM of RSL3 for 2 h. Medium was then replaced by multiple concentrations of SXQ087-1 or XBB-037. Cell viability was measured after 22 h treatment by CCK-8. For evaluation of ferroptosis inducers, HMC3 cells (seeded on 96-well plates at 5,000 cells per well for 24 h) was pre-treated with multiple concentrations of ML210 (MCE), ML162 (MCE) or FIN56 (MCE), for 2 h prior the 22 h treatment with DMSO (0.1%) only or SXQ087-1 (10 μM). For erastin, HMC3 cells were co-treated with multiple concentration of erastin with DMSO (0.1%) only or SXQ087-1 (10 μM). Cell viability was measured by CCK-8 using CLARIOstar monochromator multimode plate reader under 450 nm wavelength.

For flow cytometry, HMC3 cells (seeded 50,000 cells per well on 24-well plate one day prior to the experiment) were pretreated with 50 nM RSL3 for 2 h. After RSL3 treatment, medium was replaced by DMSO (0.1%) only, SXQ087-1 (10, 5 and 2.5 μM), following by 3 h treatment. After which medium was replaced with BODIPY™ 581/591C₁₁ (10 μM, Invitrogen) for 30 min staining. Cells were then washed 3 times with PBS and collected. Fluorescence was measured by BD FACSympgony A5.2 SORP Flow Cell Analyzer (BD Biosciences).

All data were plotted as mean±s.d., ns, no significance; *<0.05, **p<0.01, ***p<0.001, and ****p<0.0001. Statistical analyses were performed by one-way ANOVA with multiple comparisons.

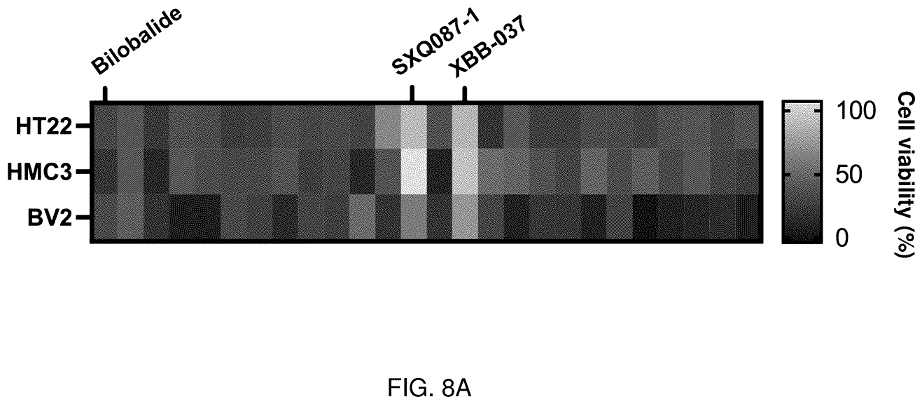

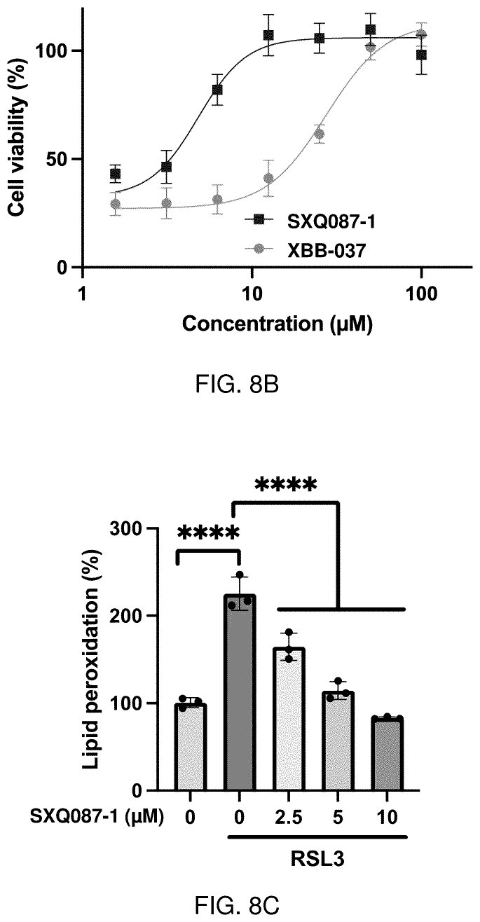

Now referring to FIG. 8A, a chart comparing the phenotypic screening of unmodified bilobalide, SXQ087-1 and XBB-037 against RSL3-induced ferroptosis through 3 cell lines is shown. In this example, the cells were pre-treated with RSL3 (200 nM for HT22 and HMC3, 500 nM for BV-2) for two hours, followed by bilobalide analogues treatment (50 PM) for 22 hours. Cell viability was measured using cell counting kit-8 (CCK-8); n=5 technical replicates. In this example, the bilobalide analogues were SXQ087-1 and XBB-037 described in preceding examples, which was found to effectively inhibit ferroptosis across 3 cell lines, as shown in FIG. 8A. The result suggested that N-alkylated and N-arylated BB analogues such as SXQ087-1 and XBB-037, respectively, inhibited RSL3-induced ferroptosis effectively.

Now referring to FIG. 8B, a plot showing the dose-dependent cell viability (%) curves of RSL3 on HMC3 cell line treated with SXQ087-1 or XBB-037.

Lipid peroxidation is one of the major hallmarks of ferroptosis. In this example, HMC3 cells according to the conditions in Table 5 were subjected to measurement of lipid peroxidation level using flow cytometry and the lipid peroxidation sensor C11-BODIPY.

TABLE 5
Example conditions for the evaluation of lipid peroxidation level

| Condition | [RSL3] (nM) | Rescuer/inhibitor |
|---|---|---|
| Control | — | — |
| NR | 50 | — |
| Bilobalide analogue | 50 | SXQ087-1 |

Now referring to FIG. 8C, a chart showing the lipid peroxidation level (%) measured by flow cytometry using C11-BODIPY is presented (n=3 technical replicates). Compared to the control and NR groups, the groups treated with 10, 5 and 2.5 μM bilobalide analogue SXQ087-1 showed significant down-regulation of RSL3-induced lipid peroxidation accumulation in a dose-dependent manner.

### Example 9: Mechanistic Studies

Aside from the GPX4 inhibition, RSL3 has also been reported to degrade GPX4 through inhibiting the mammalian target of rapamycin (mTOR) and enhancing chaperone-mediated autophagy (CMA). In this example, HMC3 cells (100,000 cells per well on 12-well plate) were co-treated with RSL3 (1 μM) and SXQ087-1 (10, 5, 2.5, 0 μM) for 3 h. After incubation, cells were washed 3 times with ice-cold PBS and lysed in RIPA buffer containing 1× protease inhibitor cocktail (MCE) and nuclease (Biyotime) on ice for 30 min. All samples were centrifuged and then quantified by bicinchoninic acid (BCA) assay (Pierce). Cell lysates were diluted with Laemmli Sample Buffer (Bio-Rad) and heated at 95° C. for 5 min. Samples were separated by 12% SDS-PAGE and transferred to a polyvinylidene difluoride membrane. Membrane was incubated with 3% bovine serum albumin (BSA, Sigma-Aldrich) for 1 h, and subsequently incubated with indicated primary antibody overnight at 4° C. After incubation, membrane was washed by wash buffer (containing 20 mM Tris, 150 mM NaCl and 0.1% Tween 20), and incubated with peroxidase-conjugated secondary antibody at room temperature for 1 h. Antibodies for GPX4 (52455, Cell Signaling Technology) was used at 1:500 dilution, GAPDH (sc-32233, Santacruz) was used at 1:3000 dilution, LC3B (A19665, ABclonal) was used at 1:1000 dilution. Goat anti-rabbit IgG HRP-linked antibody (7074, CST) was used at 1:2500 dilution, goat anti-mouse IgG (H+L) secondary antibody DyLight™ 488 (35502, Invitrogen) was used at 1:5000 dilution. Blot was washed and visualized using Clarity Western ECL Substrate (Bio-Rad) with ChemiDoc MP Imaging System (Bio-Rad). Band intensity was normalized by image J software.

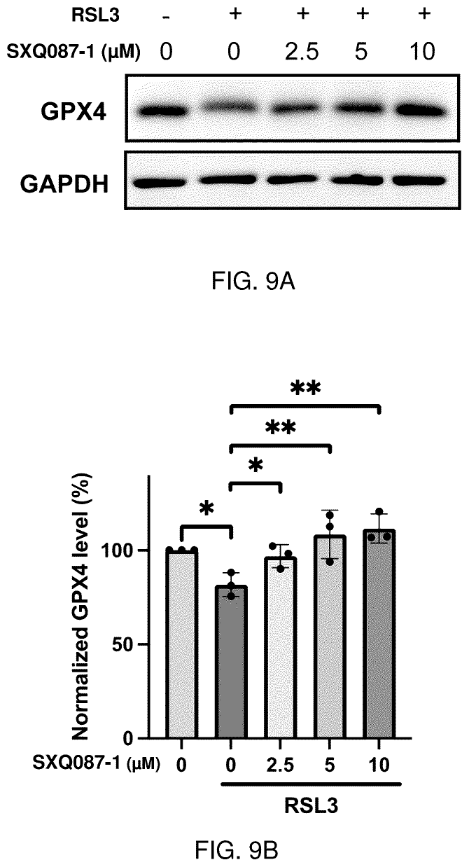

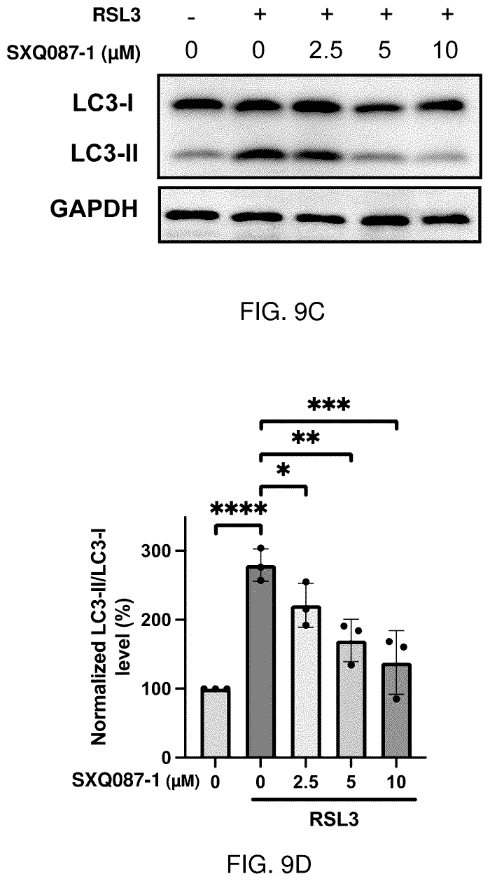

Now referring to FIGS. 9A-9B, Western-blot images showing the levels of GPX4 and GAPDH at various concentrations of SXQ087-1 (10, 5, 2.5, 0 μM) against RSL3 (1 μM) and a normalized GPX4 level (%) plot of are shown. In this example, GAPDH was a reference protein. The results showed that RSL3 diminished the GPX4 level, and RSL3-induced GPX4 degradation was reduced after the treatment of bilobalide analogue SXQ087-1 compared to GAPDH.

Now referring to FIGS. 9C-9D, Western-blot images showing the levels of LC3-II/LC3-I and GAPDH at various concentrations of SXQ087-1 (10, 5, 2.5, 0 μM) against RSL3 (1 μM) and a normalized LC3-II/LC3-I level (%) plot of are shown. The results showed that RSL3 significantly increased the autophagy related protein LC3-II/LC3-I ratio, which indicated the up regulation of autophagy, as seen in FIGS. 9C-9D. The increasing LC3-II/LC3-I ratio was also reduced after the treatment of bilobalide analogue SXQ087-1, indicating the potential inhibition activity of SXQ087-1 on autophagy to protect GPX4 from degradation.

### Example 10: Further Mechanistic Studies

In this example, the protection effect of bilobalide analogues against various ferroptosis inducers (Class I-III), their mechanisms in the glutathione peroxidase 4 (GPX4) pathway, cytotoxicity and antioxidative activity were studied by the procedures similar to those as discussed in Example 8 herein. For the sake of brevity and simplicity of the present disclosure, the full discussion is not reproduced here.

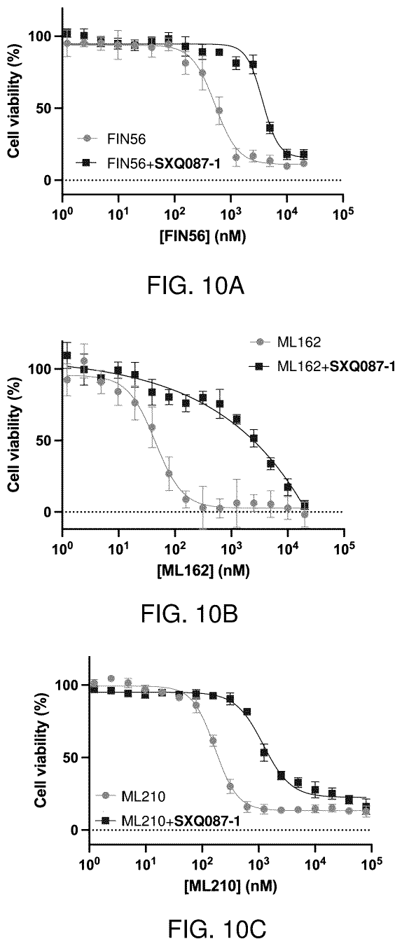

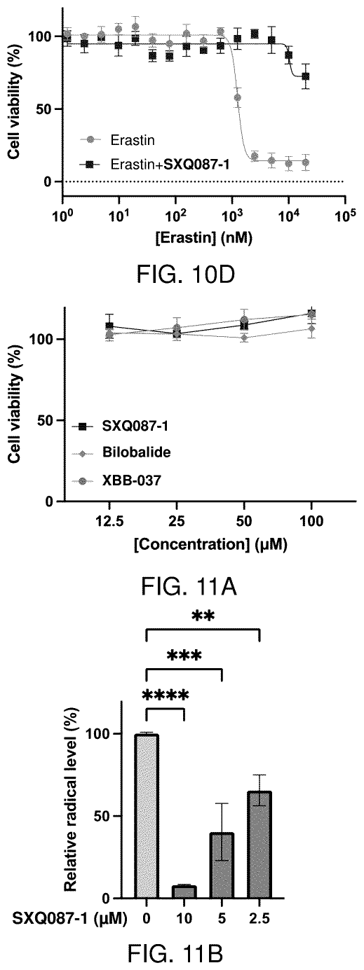

Now referring to FIGS. 10A-10D, charts showing the protection effect of bilobalide analogues against various ferroptosis inducers (Class I-III). FIN56 is a class III ferroptosis inducer, which induces ferroptosis via the depletion of GPX4 instead of acting as a GPX4 binder. As shown in FIG. 10A, the treatment of 10 μM bilobalide analogue (FIN56+SXQ087-1) effectively suppressed FIN56-induced cell death, compared to the control group (FIN56).

Class II ferroptosis inducers ML162 and ML210 are GPX4 covalent inhibitors. As shown in FIG. 10B, the treatment of 10 μM bilobalide analogue (ML162+SXQ087-1) alleviated the lethality of ML162, compared to the control group which was not treated with SXQ087-1 (ML162). Consistently, as shown in FIG. 10C, the treatment of 10 μM bilobalide analogue (ML210+SXQ087-1) alleviated the lethality of ML210, compared to the control group (ML210) These results demonstrated the ability of bilobalide analogues to alleviate the lethality of Class II ferroptosis inducers such as ML162 and ML210.

As shown in FIG. 10D, the treatment of 10 μM bilobalide analogue (Erastin+SXQ087-1) suppressed erastin-induced lethality, compared to the control group (Erastin). Collectively, our observation on system X_{c}⁻ inhibitors reflected that the mechanism of bilobalide analogue SXQ087-1 may directly interact with ferroptosis.

Now referring to FIG. 11A, a chart showing the cell viability (%) of HMC3 cells treated with SXQ087-1, XBB-037 or bilobalide. As shown in FIG. 11A, after 24 h treatment of SXQ087-1, XBB-037 or bilobalide, no significant cytotoxicity was observed in HMC3 cells.

The mechanism of the antioxidative activity of SXQ087-1 is also demonstrated herein by further evaluating the radical scavenging activity of SXQ087-1 using a cell free radical assay. FIG. 11B is a chart showing the relative radical level (%) of SXQ087-1. As shown in FIG. 11B, SXQ087-1 demonstrated a significant dose-dependent radical scavenging activity.

In conclusion, bilobalide analogue SXQ087-1 was shown to significantly reduce key ferroptosis markers lipid peroxidation, and notably counteracted GPX4 degradation and LC3-II/LC3-I ratio induction induced by RSL3. The results in this example highlight the therapeutic utility of bilobalide analogues against neurological diseases (e.g., neurodegenerative diseases) by inhibiting ferroptosis.

### Example 11: Animal Studies

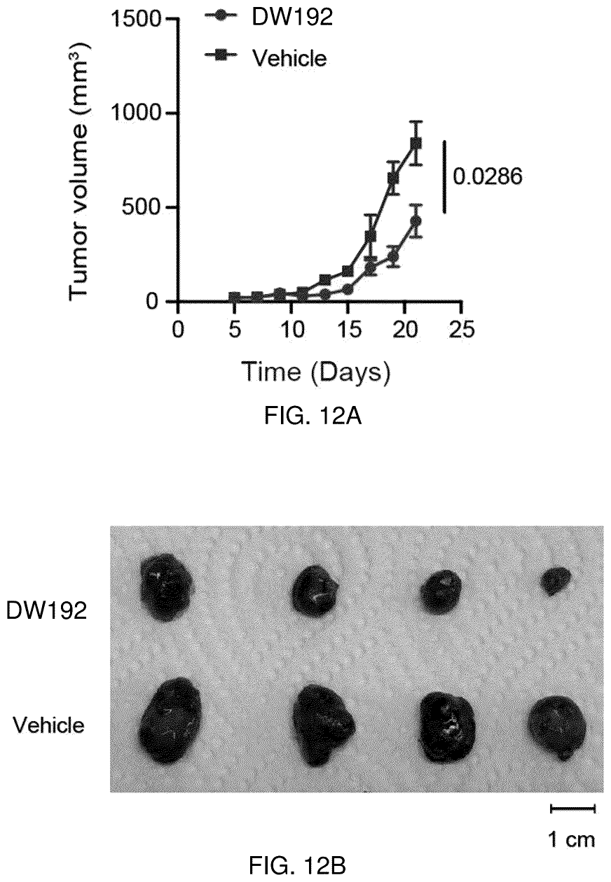

In the example, C57BL/6 mice were subcutaneously injected with B16 melanoma cells. Tumor cells were allowed to develop for one week, after that mice were treated with daily subcutaneous injections of vehicle or DW192 (40 mg/kg) for 21 days. FIG. 12A is a plot showing the tumor growth curves. As shown in FIG. 12A, the tumor volume is lowered in mice treated with DW192 40 mg/kg daily. FIG. 12B is a photograph of the tumors collected from vehicle and DW192 treated mice after the 21-day treatment, where tumors from mice treated with DW192 were significantly smaller compared to those from mice treated with vehicle. From the results, DW192 significantly reduced the growth and size of melanoma tumors.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

1. A compound of Formula I: 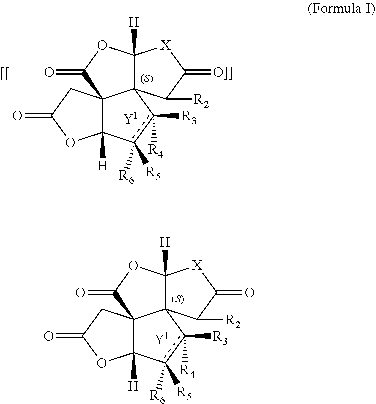
or a stereomer, a tautomer, or a pharmaceutically acceptable salt thereof,
wherein
X is —O—, —NR¹—, —N═CR¹—NH—, or —NR¹—NH—; wherein when X is —O—, R¹ is absent;
bond Y¹ is between R⁴ and R⁵ and is a single bond or a double bond;
R¹ is H, R^{1B}, or -(L¹)ᵤ-(Z¹)ᵥ; wherein
L¹ is C₁-C₁₀ aliphatic wherein up to three carbon atoms of the C₁-C₁₀ aliphatic are optionally replaced by N, O, or S; wherein L¹ is optionally substituted with 1-3 occurrences of halo, CN, R, OR′, or R^{1C};
u is 0 or 1;
v is 0 or 1;
Z¹ is a 5-16 membered aromatic or nonaromatic monocyclic, bicyclic, or tricyclic ring system having 0-7 heteroatoms selected from O, N, or S; wherein Z¹ is optionally substituted with 1-5 occurrences of R^{1A}, R^{1C} or combinations thereof;
R^{1A} is -(L²)ₘ-(Z²)_{w}; wherein
L² is C₁-C₁₀ aliphatic wherein up to three carbon atoms of the C₁-C₁₀ aliphatic are optionally replaced by N, NR, O, S, C═O, SO₂, S═O, (C═O)N, N(C═O)N, (C═O)O, or Si; wherein L² is optionally substituted with 1-3 occurrences of halo, CN, R, OR′ or R^{1C};
m is 0 or 1;
w is 0 or 1;
Z² is a C₁-C₁₀ aliphatic, or 3-16 membered aromatic or nonaromatic monocyclic, bicyclic or tricylic ring system having 0-7 heteroatoms selected from O, N, or S; wherein Z² is optionally substituted with 1-5 occurrences of R^{1B};
R^{1B} is H, halo, CN, R*, OR*, NRR*; or two R^{1B}, taken together with the atom to which they are attached, form a 3-6 membered ring having 0-4 heteroatoms;
R^{1C} is H, halo, CN, a 5-10 membered aromatic or nonaromatic monocyclic or bicyclic ring system having 0-5 heteroatoms selected from O, N, or S; R*, OR*, NRR*; or two R^{1C}, taken together with the atom or atoms to which they are attached, optionally form a 3-16 membered ring having 0-4 heteroatoms; wherein R^{1C} is optionally substituted with 1-3 occurrences of halo, CN, R′ or OR′;
R* is C₁-C₆ aliphatic wherein up to three methylene units of the C₁-C₆ aliphatic are optionally replaced by N, NR, O, S, C═O, SO, SO₂ or Si and wherein the C₁-C₆ aliphatic is optionally substituted with 1-3 occurrences of halo, CN, R′ or OR′;
R² is R^{2A} or OR^{2A}, wherein R^{2A} is H, a C₁-C₁₆ aliphatic, a 5-10 membered aromatic or nonaromatic monocyclic or bicyclic ring system, or —(C₁-C₁₆ aliphatic)-(5-10 membered aromatic or nonaromatic monocyclic or bicyclic ring system); wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{2A} is optionally substituted with 1-5 occurrences of R^{2B}, wherein R^{2B} is halo, R′ or OR′;
R³ is OH, R^{3A}, or OR^{3A}; wherein R^{3A} is C₁-C₁₀ aliphatic optionally substituted with 1-3 occurrences of halo, R or OR′;
R⁴ is OH, R^{4A}, OR^{4A}; or when bond Y¹ between R⁴ and R⁵ is a double bond, R⁴ is absent; wherein R^{4A} is C₁-C₇ aliphatic and R^{4A} is optionally substituted with 1-3 occurrences of halo, R′ or OR′;
R⁵ is H or OH;
R⁶ is H; or when bond Y¹ between R⁴ and R⁵ is a double bond, R⁶ is absent;
R is H or C₁-C₆ aliphatic optionally substituted by 1-3 occurrences of F; or two R, taken together with the atom(s) to which they are attached, form a 3-6 membered ring having 0-4 heteroatoms; and
R′ is H, a C₁-C₆ aliphatic wherein up to three carbon atoms of the C₁-C₆ aliphatic are optionally replaced with O, NH, N(C₁-C₆ alkyl), C(O), or S(O)₂; wherein said C₁-C₆ aliphatic is optionally substituted by 1-3 occurrences of F, OR, NH₂, NHR″, or NR″₂, wherein R″ is C₁-C₆ aliphatic or a 5-10 membered aromatic or nonaromatic monocyclic or bicyclic ring system having 0-5 heteroatoms selected from O, N, or S;
wherein when R² is OH, R³ is tert-butyl, R⁴ is OH, R⁵ is H, and R⁶ is H, X is not —O—.

2. The compound 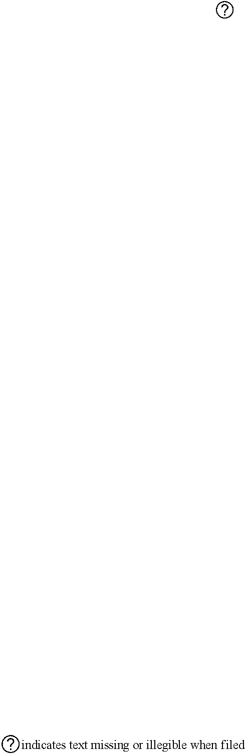
of claim 1, wherein X is —NR¹—, —N═CR¹—NH—, or —NR¹—NH—.

3. The compound of claim 1, having Formula Ia:

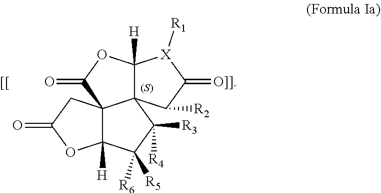

4. The compound of claim 1, having Formula Ib:

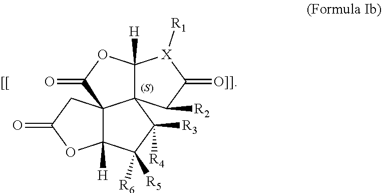

5. The compound of claim 1, wherein R² is R^{2A} or OR^{2A}, wherein R^{2A} is H, C═O(C₁₋₁₀ aliphatic), SO₂(C₁₋₁₀ aliphatic), SO₂(phenyl), phenyl, Si(C₁₋₁₀ aliphatic)₁₋₂, Si(phenyl)₁₋₂, —(C₁₋₁₀ aliphatic)O(C₁₋₁₀ aliphatic)-, (C═O)(phenyl), NH(C═O) (C₁₋₁₀ aliphatic) 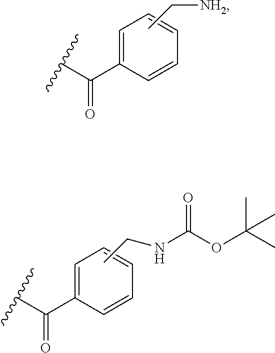
or NH(C═O)O(C₁₋₁₀ aliphatic);
wherein each R^{2A} is independently and optionally substituted with 1-5 occurrences of R^{2B}, wherein R^{2B} is halo, R′ or OR′;
R³ is C₁₋₁₀ aliphatic;
the bond Y¹ between R⁴ and R⁵ is a single bond;
R⁴ is OH or OR^{4A};
and R⁵ is H or OH.

6. The compound of claim 1, wherein R² is R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅, 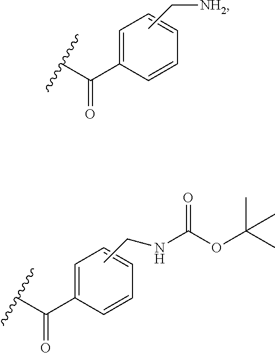
or NH(C═O)OC(CH₃)₃;
wherein phenyl, C₆H₄, and C₆H₅ are each independently and optionally substituted with 1-5 occurrences of R^{2B}, wherein R^{2B} is halo, R′ or OR′;
R³ is methyl, ethyl, propyl, isopropyl, butyl, isobutyl, or tert-butyl;
the bond Y¹ between R⁴ and R⁵ is a single bond;
R⁴ is OH or OR^{4A};
and R⁵ is H or OH.

7. The compound of claim 1, wherein R¹ is H.

8. The compound of claim 1, wherein R¹ is -(L¹)ᵤ-(Z¹)ᵥ;
wherein
L¹ is C₁-C₁₀ aliphatic wherein up to three carbon atoms of the C₁-C₁₀ aliphatic are optionally replaced by N, O, or S;
Z¹ is phenyl, 1-methyl-1,2,3,4-tetrahydronaphthalen-2-y, 1-methyl-2H-isoindol-2-yl, imidazol, indolyl, napthalenyl, adamantanyl, azetidinyl, bicyclo[1.1.1]pentyl, 1-oxa-8-azaspiro[4.5]decan-3-yl, cyclobutanyl, cyclohexanyl, cyclopentanyl, cyclopropanyl, norbornenyl, oxetanyl, piperazinyl, piperidinyl, pyridinyl, pyrrolidinyl, tetrahydrofuranyl, tetrahydropyranyl, tetrahydrothiopyranyl, or C₃-C₁₂ cycloaliphatic having 0-5 heteroatoms selected from O, N, or S;
u is 0 or 1; and
v is 0 or 1; wherein Z¹ is optionally substituted with 1-5 occurrences of R^{1C}, morpholinyl, —OCH₂O—, —(C═O)-(pyrazinyl)-R^{1B}, —(C═O)-(phenyl)-R^{1B}, or —(SO₂)-(phenyl)-R^{1B}; wherein each independent occurrence of R^{1B} is H, halo, R*, OR*, or NRR*; wherein each independent occurrence of R^{1C} is H, halo, R*, OR*, or NRR*; and wherein each independent occurrence of R* is H, ═N, —C≡CH, —N═N—, —CH₃, —CH₂F, —CHF₂, —CF₃, —CN, —CH₂O—, —CF₂O—, —CH₂CH₂O—, or -Boc (—(C═O)OC(CH₃)₃).

9. The compound of claim 8, wherein when Z¹ is phenyl, Z¹ is optionally substituted with 1-5 occurrences of morpholinyl or R^{1C}, wherein R^{1C} is halo, CH₃, —CH₂F, —CHF₂, —CF₃, —CN—OCH₃, —OCH₂O—, —OCF₂O—, —OCH₂CH₂O—, —NH₂, —NH(C═O)CH₃, or —NH(Boc) (NH(C═O)OC(CH₃)₃).

10. The compound of claim 8, wherein when Z¹ is piperidinyl, Z¹ is optionally substituted with 1-2 occurrences of R^{1C}, wherein R^{1C} is tert-butoxylcarbonyl, 5-(difluoromethyl)pyrazine-2-carbonyl, 2,2-difluoro-2H-1,3-benzodioxole-5-carbonyl, 2,3-dihydro-1,4-benzodioxine-6-carbonyl, 2,3-dihydro-1-benzofuran-5-sulfonyl, 4-chlorobenzoyl, 2,3-dihydro-1-benzofuran-5-carbonyl, or prop-2-enoyl.

11. The compound of claim 8, wherein when Z¹ is pyrrolidinyl, Z¹ is optionally substituted with 1-2 occurrences of R^{1C}, wherein R^{1C} is tert-butoxylcarbonyl.

12. The compound of claim 8, wherein R¹ is
H,
2,4-dimethoxybenzyl,
[1-(tert-butoxycarbonyl)piperidin-4-yl]methyl,
piperidin-4-yl methyl,
2-[1-(tert-butoxycarbonyl)piperidin-4-yl]ethyl,
2-(piperidin-4-yl)ethyl,
3-[1-(tert-butoxycarbonyl)piperidin-4-yl]propyl,
3-(piperidin-4-yl)propyl,
2-[4-(tert-butoxycarbonyl)piperazin-1-yl]ethyl,
2-(piperazin-1-yl)ethyl,
2-(3-methyl-1H-indol-2-yl)ethyl,
3-(1H-imidazol-1-yl)propyl,
[1-(tertbutoxycarbonyl)pyrrolidin-3-yl]methyl,
(pyrrolidin-3-yl)methyl,
(bicyclo[2.2.1]hept-5-en-2-yl)methyl,
phenyl,
4-acetamidophenyl,
4-[(tert-butoxycarbonyl)amino]phenyl,
4-aminophenyl,
4-(morpholin-4-yl)phenyl,
benzo[d][1,3]dioxol-5-yl,
pyridin-3-yl,
benzyl,
methyl,
bicyclo[1.1.1]pentyl,
oxetan-3-yl,
cyclobutyl methyl,
cyclopropyl methyl,
(oxetan-3-yl)methyl,
adamantan-2-yl methyl,
NH₂,
cyclopropyl,
3-methoxy phenyl,
4-methoxy phenyl,
naphthalen-2-yl,
3-(trifluoromethyl) phenyl,
4-cyano phenyl,
2-[3-(but-3-yn-1-yl)-3H-diazirin-3-yl]ethyl,
cyclohexyl,
4-fluoro phenyl,
4-(trifluoromethyl) phenyl,
4-toluyl,
3-toluyl,
2-toluyl,
(oxolan-2-yl)methyl,
2-methoxy-2-oxoethyl,
(1-(5-(difluoromethyl)pyrazine-2-carbonyl)piperidin-4-yl)methyl,
[1-(2,3-dihydro-1-benzofuran-5-sulfonyl)piperidin-4-yl]methyl,
(1-(2,2-difluorobenzo[d][1,3]dioxole-5-carbonyl)piperidin-4-yl)methyl,
(1-(2,3-dihydrobenzo[b][1,4]dioxine-6-carbonyl)piperidin-4-yl)methyl,
(1-(4-chlorobenzoyl)piperidin-4-yl)methyl,
(1-(2,3-dihydrobenzofuran-5-carbonyl)piperidin-4-yl)methyl,
(1-acryloylpiperidin-4-yl)methyl,
(1-(quinoxaline-6-carbonyl)piperidin-4-yl)methyl,
(tetrahydro-2H-pyran-4-yl)methyl,
(tetrahydro-2H-thiopyran-4-yl)methyl,
2-(1-methyl-1,2,3,4-tetrahydronaphthalen-2-yl)ethyl,
2-(1-methyl-2H-isoindol-2-yl)ethyl,
2-(azetidin-1-yl)ethyl,
2-(trifluoromethyl) phenyl,
2-fluoro phenyl,
2-methoxy phenyl,
3,4-difluoro phenyl,
3,4-dichloro phenyl,
3,5-difluoro phenyl,
3-fluoro phenyl,
4-hydroxy phenyl,
8-(tert-butoxycarbonyl)-1-oxa-8-azaspiro[4.5]decan-3-yl,
anilinyl,
benzo[d][1,3]dioxol-4-yl,
cyclobutyl,
cyclohexyl methyl,
naphthalen-1-yl,
pyridin-2-yl,
pyridin-4-yl,
adamantan-1-yl methyl,
1-(tert-butoxycarbonyl)-1H-indol-5-yl,
1H-indol-5-yl,
3-[(tert-butoxycarbonyl)amino]phenyl,
4-Hydroxyphenyl ethyl,
1H-indole-3-ethyl,
((1R,4aS,10aR)-7-isopropyl-1,4a-dimethyl-1,2,3,4,4a,9,10,10a-octahydrophenanthren-1-yl) methyl,
[((tert-butoxycarbonyl)aminomethyl) adamantan-1-yl]methyl,
(aminomethyl)adamantan-1-yl) methyl,
3,5-di-tert butyl phenyl,
3,4-dihydroxyphenyl,
3-methoxy-4-hydroxyphenyl ethyl,
1H-indole-5-hydroxy-3-ethyl,
1H-indole-5-methoxy-3-ethyl,
1H-indole-4-hydroxy-3-ethyl,
piperonyl,
2-(4-Imidazolyl)ethyl (histamine),
2,2-diphenylethyl,
3-hydroxy-4-methoxyphenyl ethyl,
3,4-methylenedioxyphenyl ethyl,
1H-indole-5-hydroxy-3-ethyl (serotonin),
3,4-dihydroxyphenyl ethyl (dopamine),
1H-indole-3-ethyl (tryptamine),
3-methoxy-4-hydroxyphenyl ethyl (3-O-methyldopamine), or
methylenedioxyphenyl.

13. The compound of claim 12, wherein
R² is R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅, 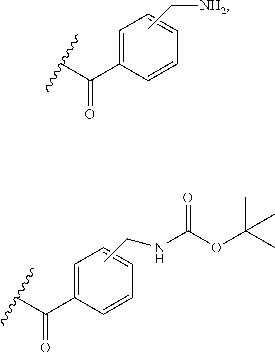
or NH(C═O)OC(CH₃)₃;
wherein phenyl is optionally substituted with 1-5 occurrences of halo, R′ or OR′;
R³ is tert-butyl;
the bond Y¹ between R⁴ and R⁵ is a single bond;
R⁴ is OH;
R⁵ is H;
X is —NR¹—, —N═CR¹—NH—, or —NR¹—NH—; and.
R¹ is selected from 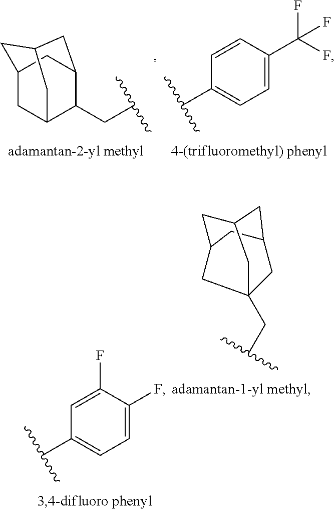 [SMILES: *CC1C2CC3CC(C2)CC1C3 ; *c1ccc(C(F)(F)F)cc1 ; *CC12CC3CC(CC(C3)C1)C2 ; *c1ccc(F)c(F)c1]
2-(4-imidazolyl)ethyl (histamine),
1H-indole-5-hydroxy-3-ethyl (serotonin),
3,4-dihydroxyphenyl ethyl (dopamine),
1H-indole-3-ethyl (tryptamine), or
3-methoxy-4-hydroxyphenyl ethyl (3-O-methyldopamine).

14. The compound of claim 1, selected from

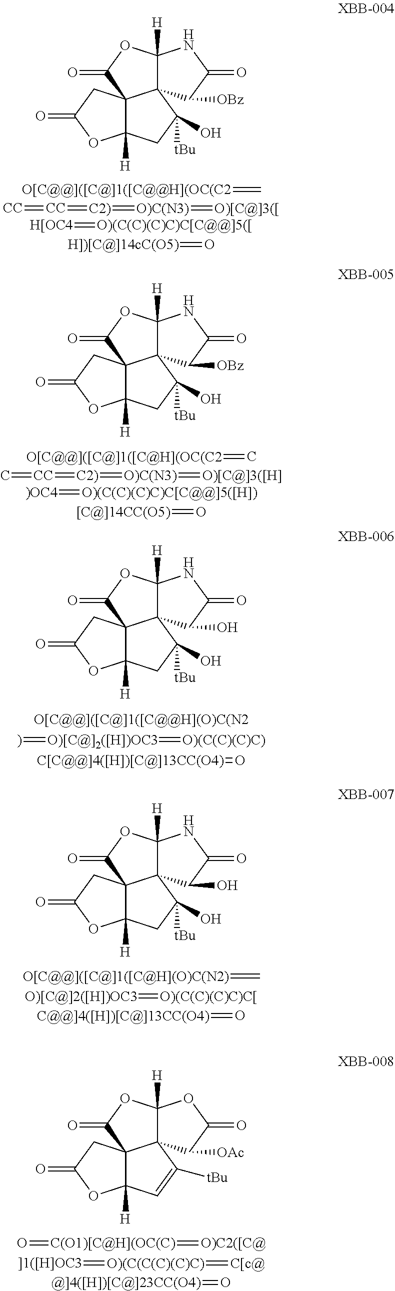

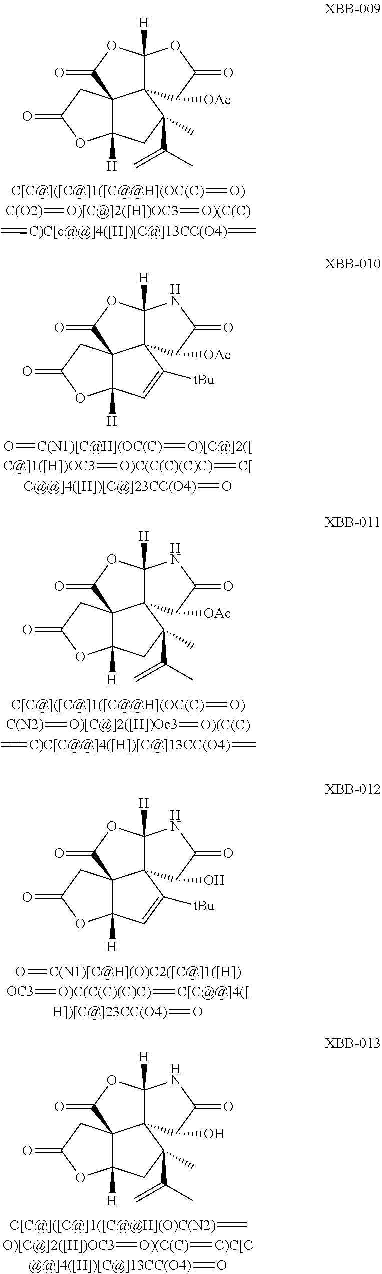

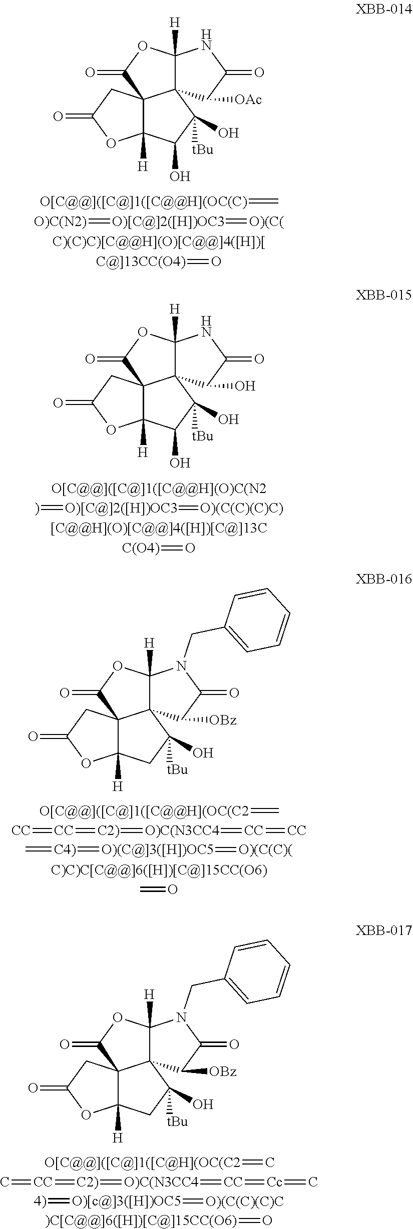

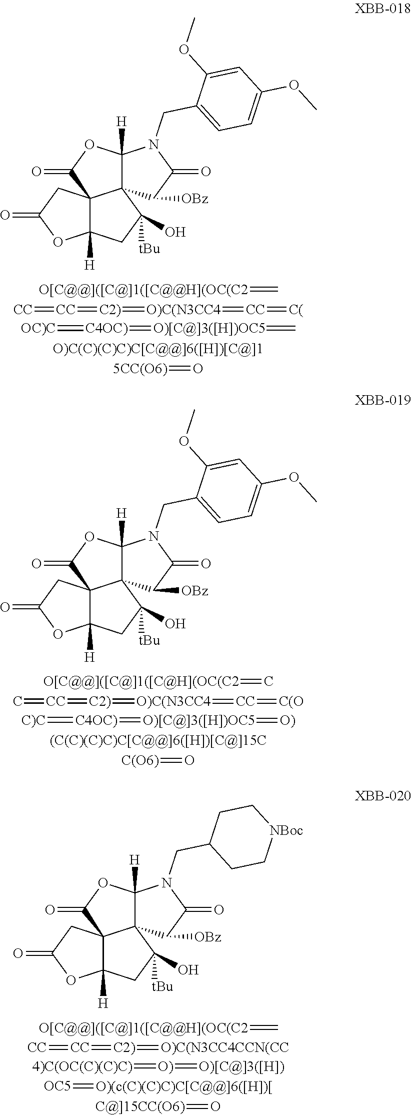

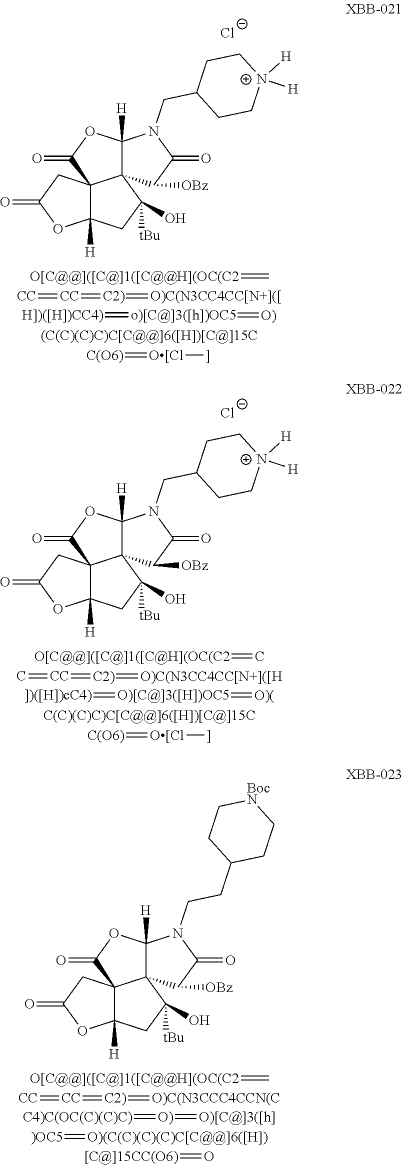

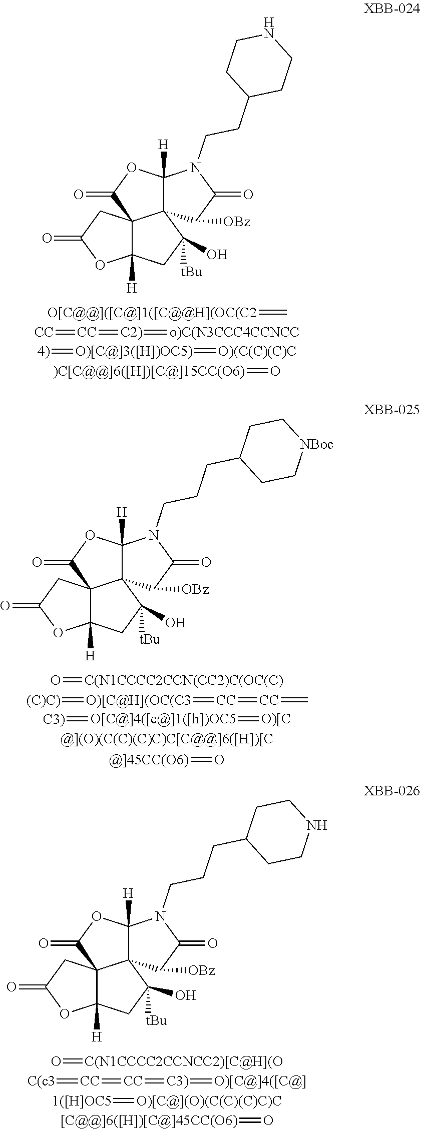

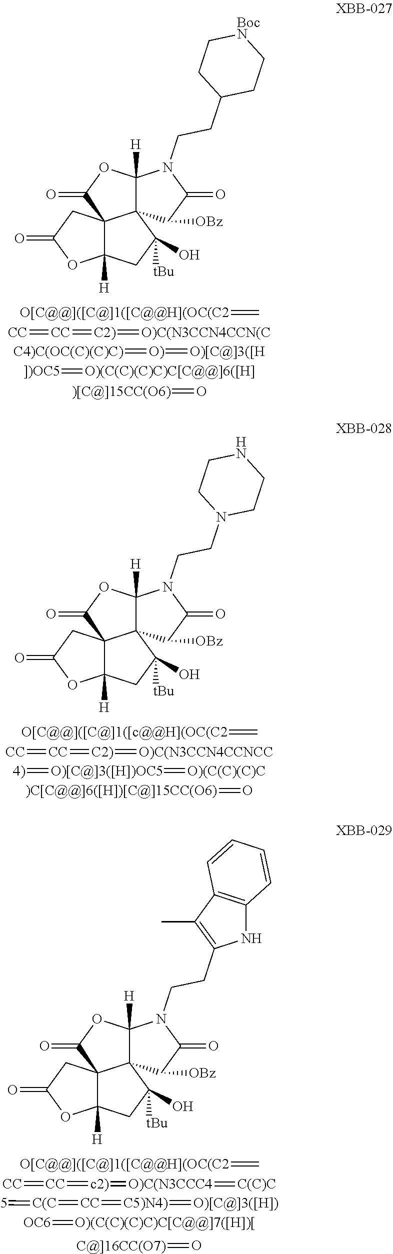

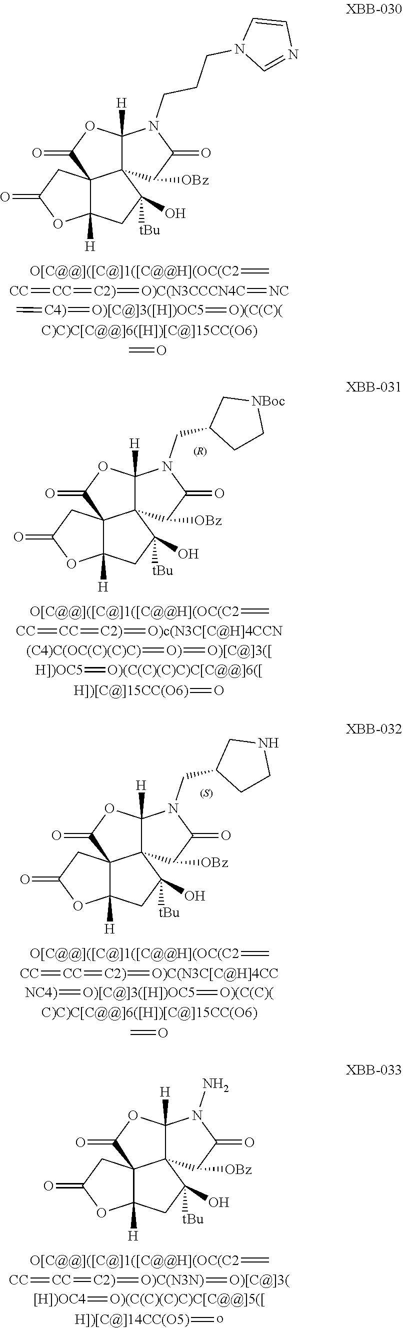

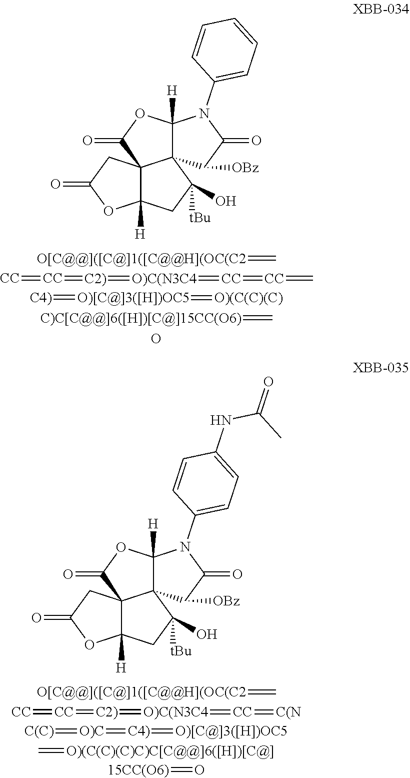

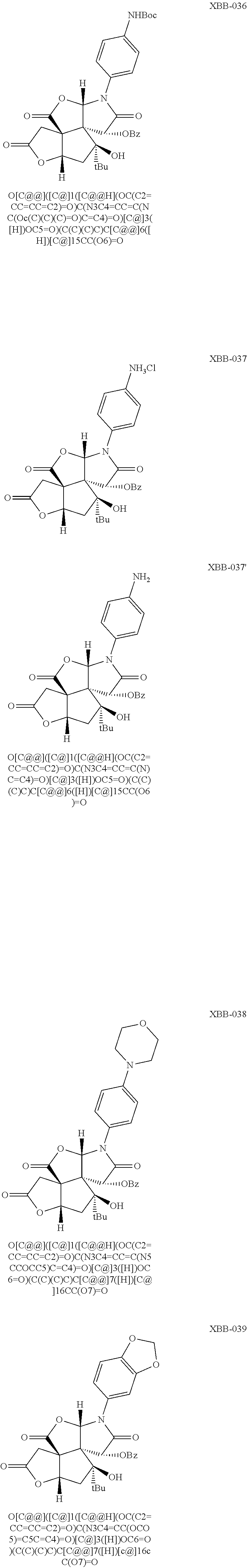

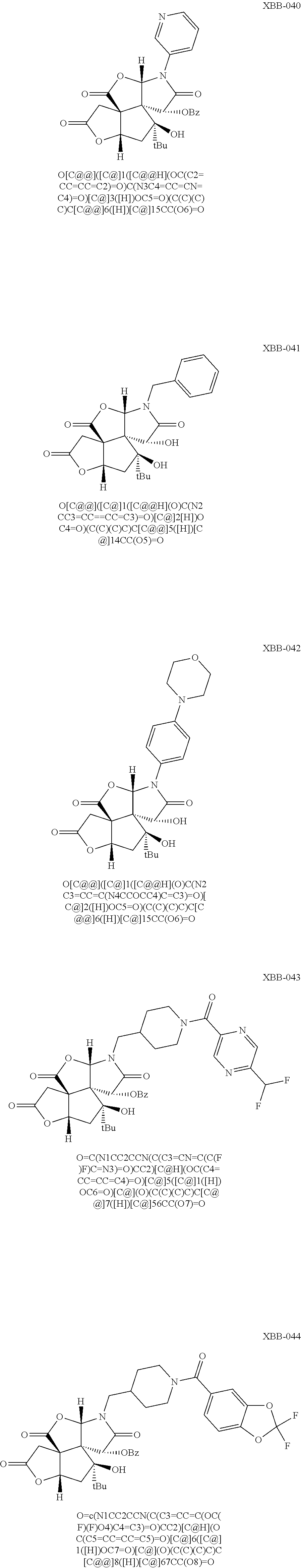

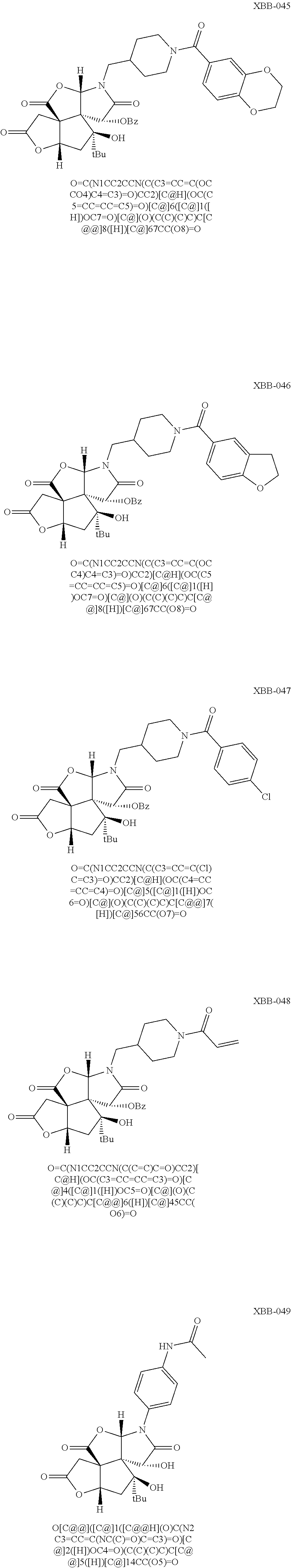

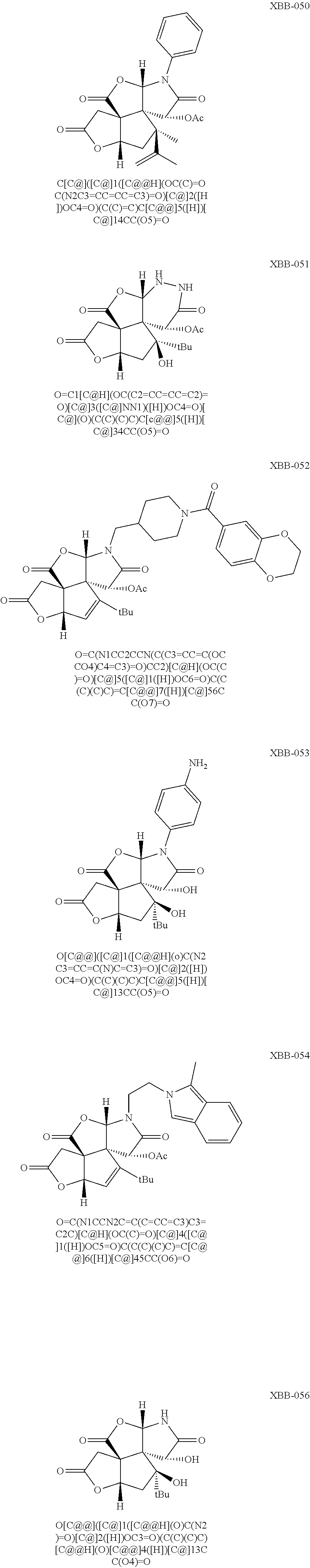

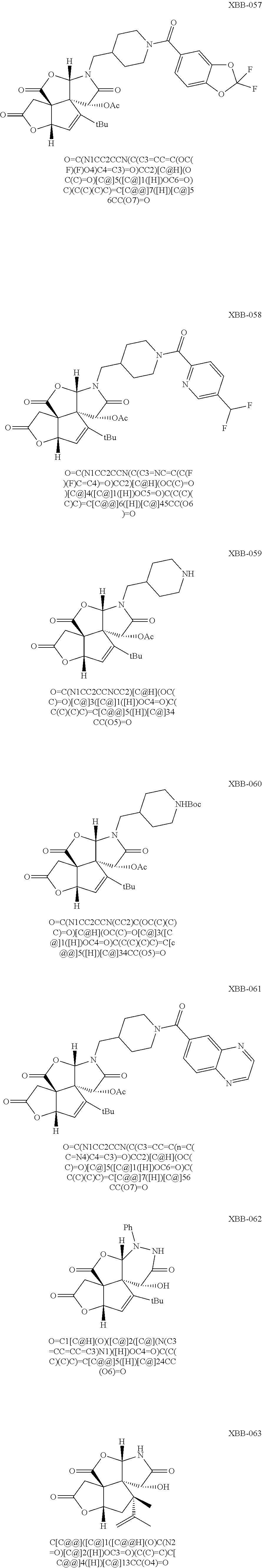

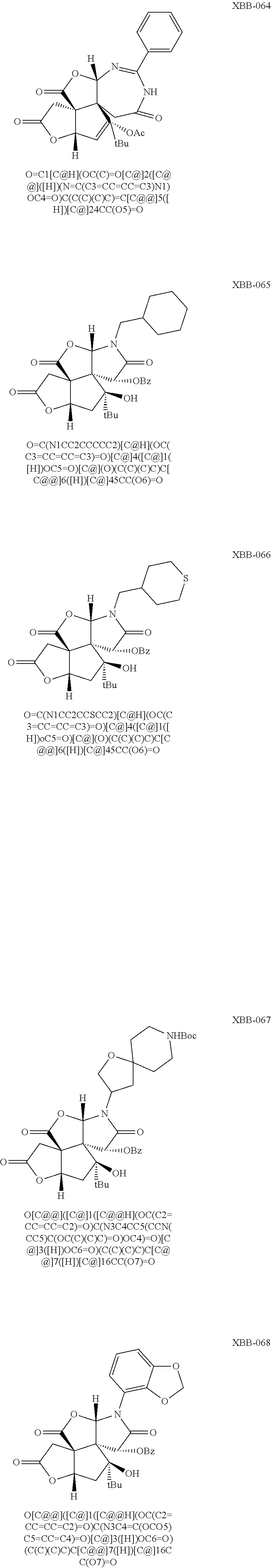

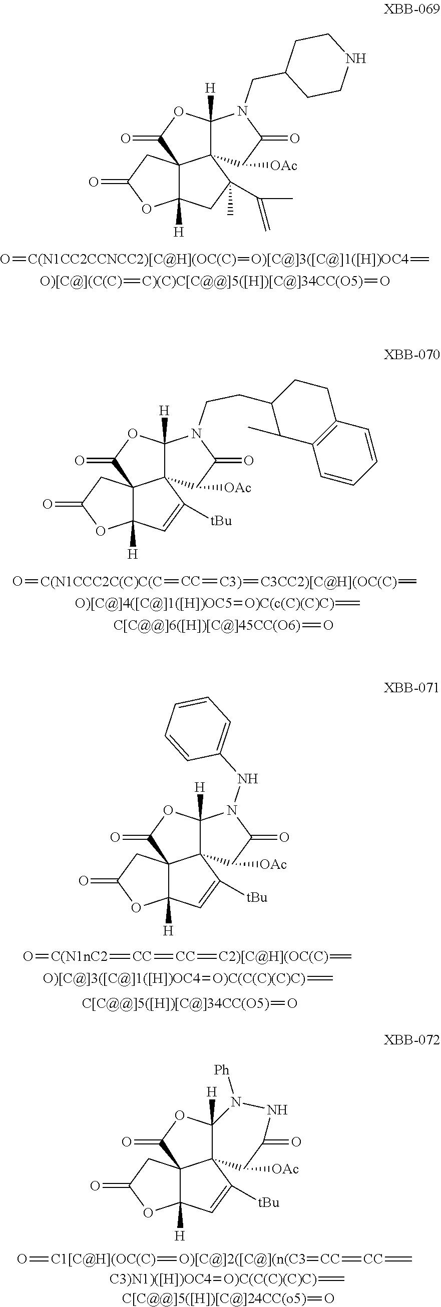

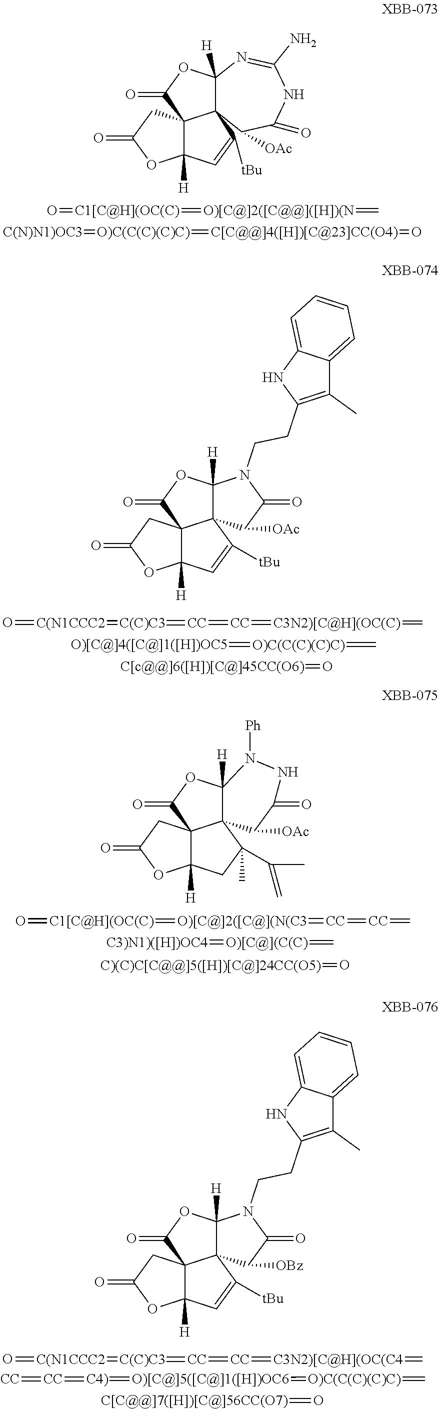

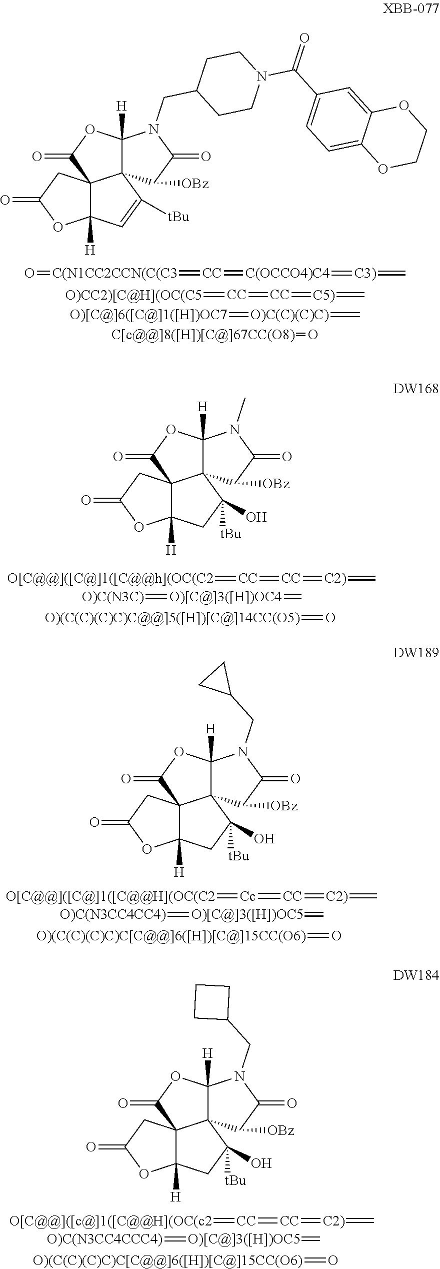

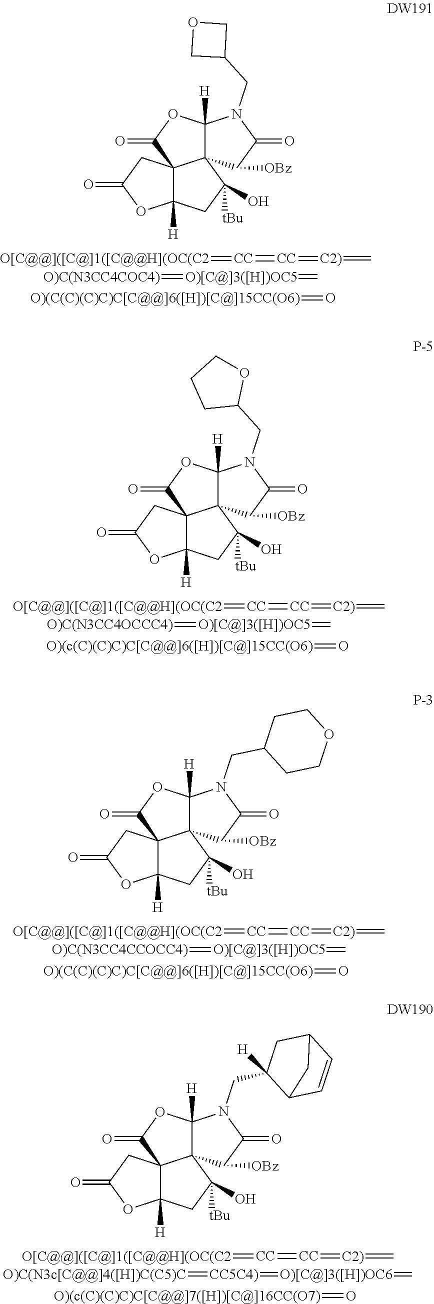

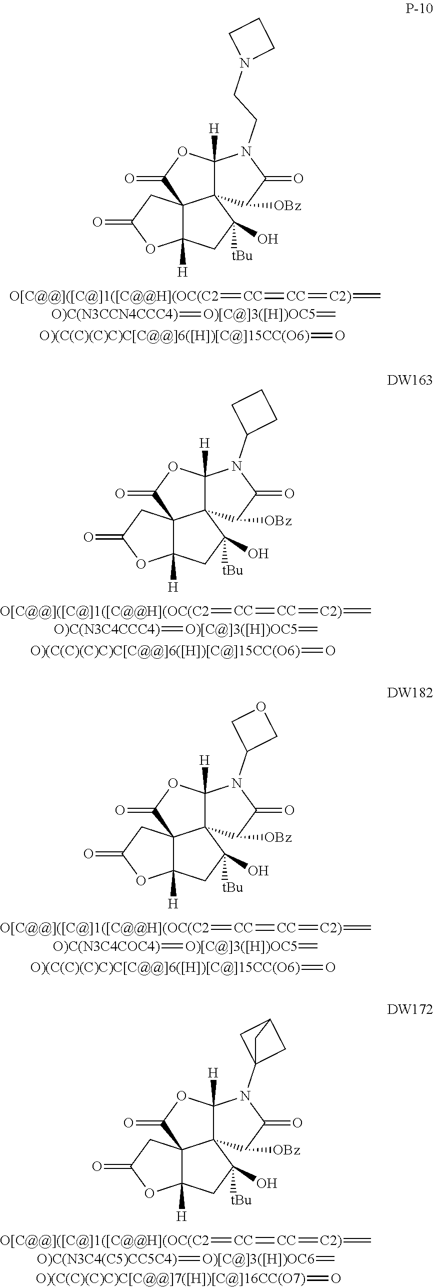

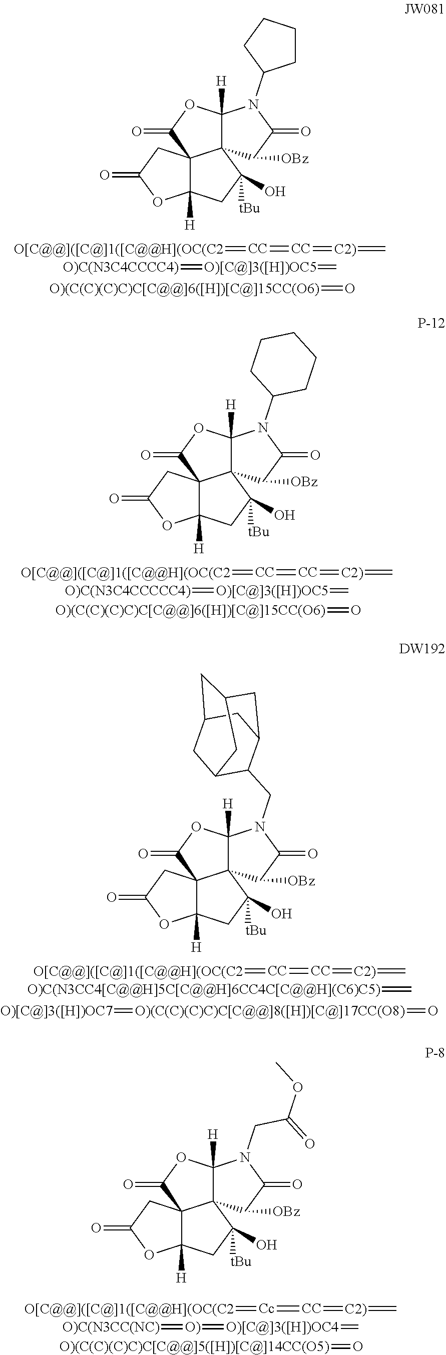

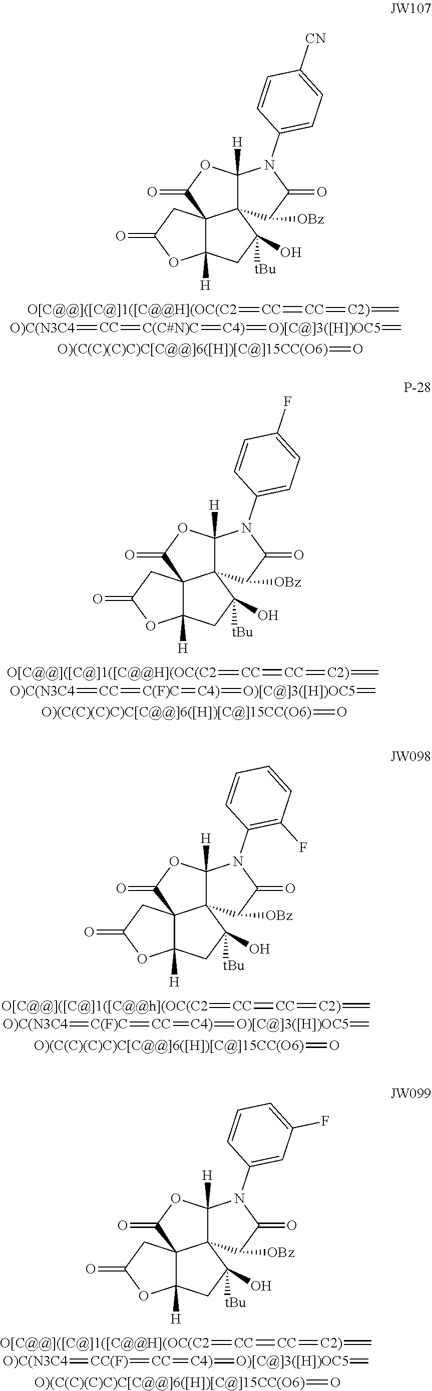

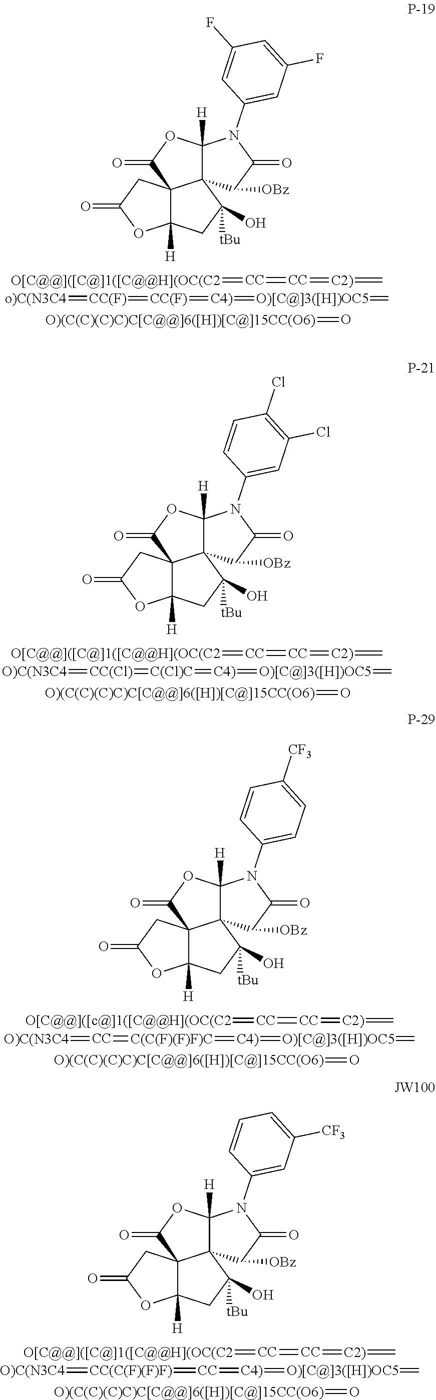

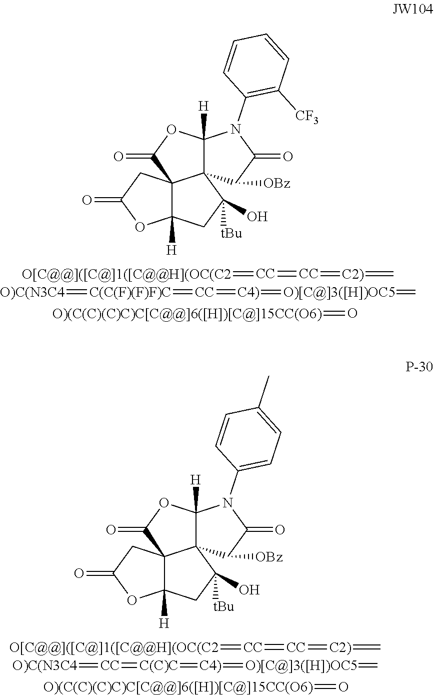

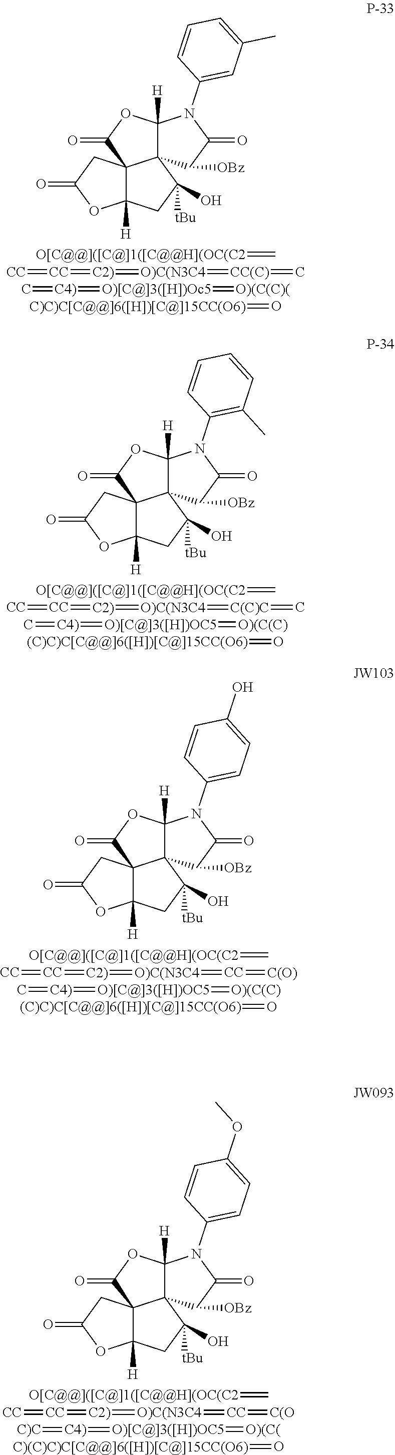

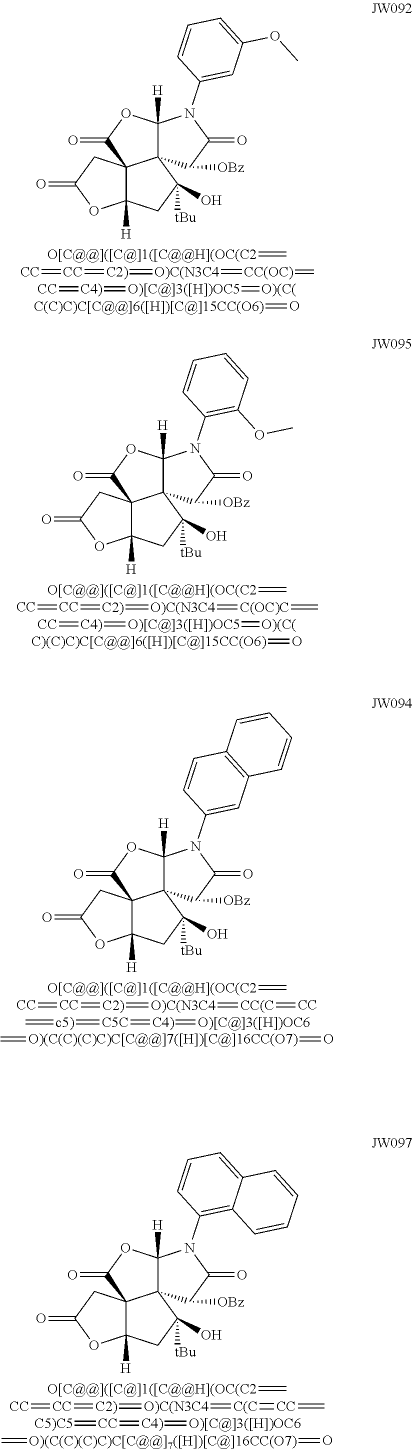

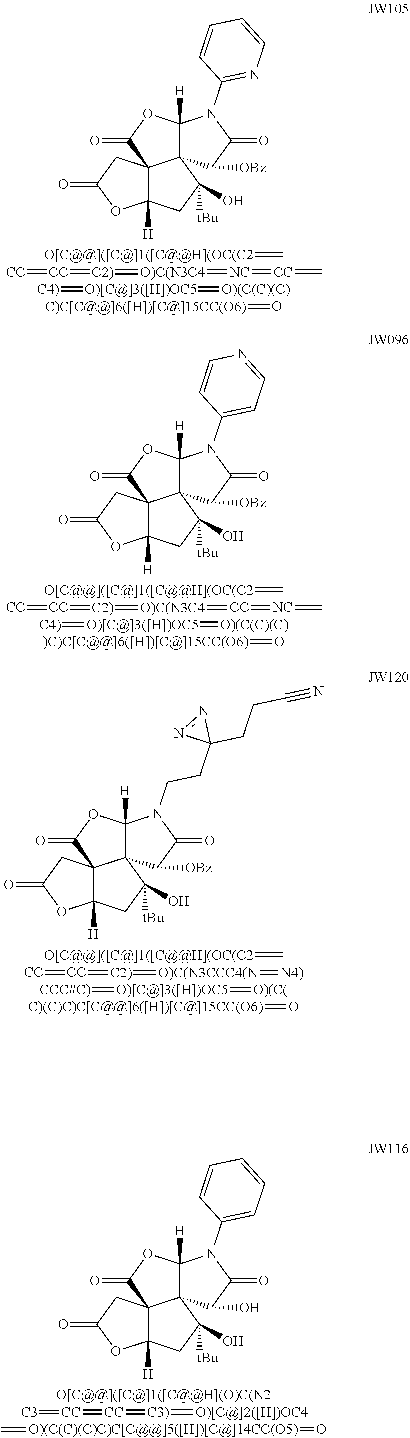

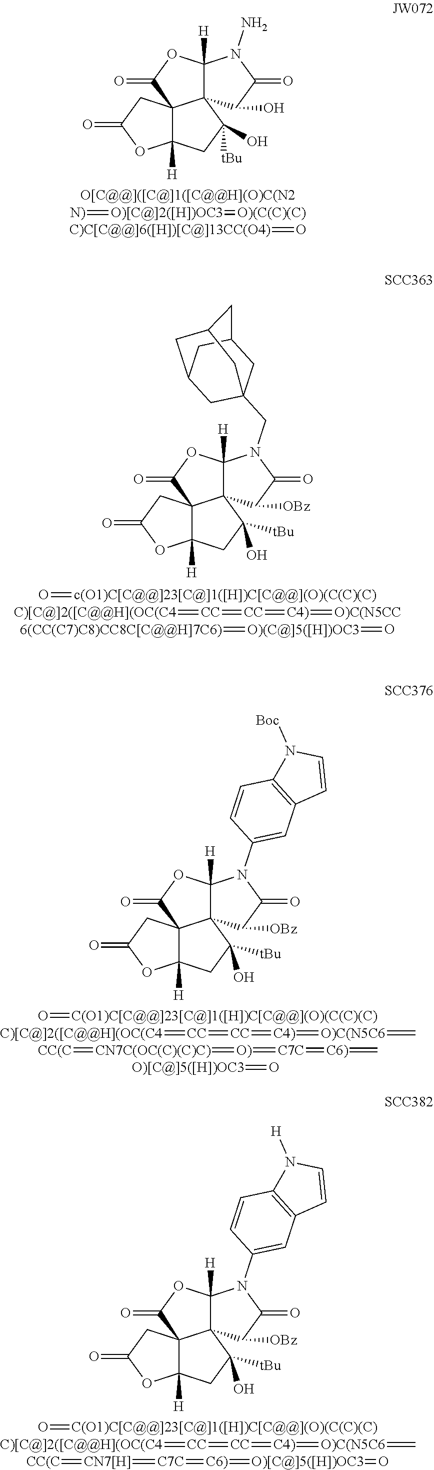

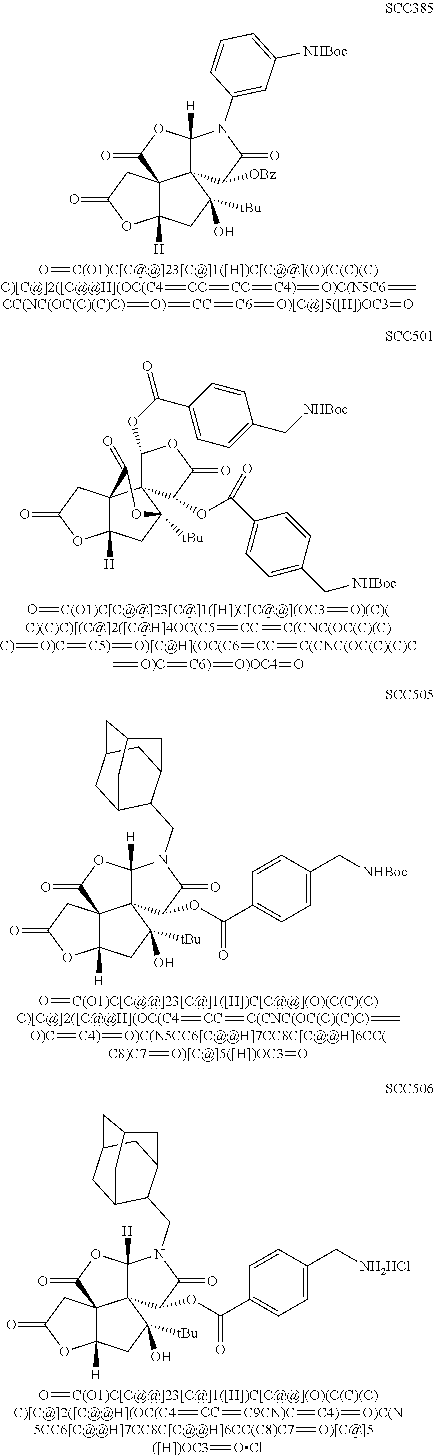

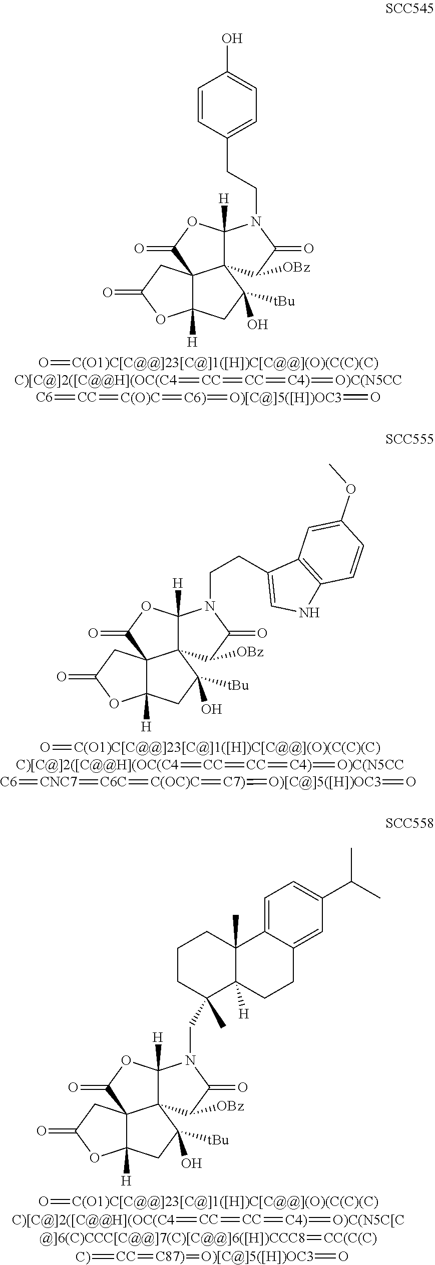

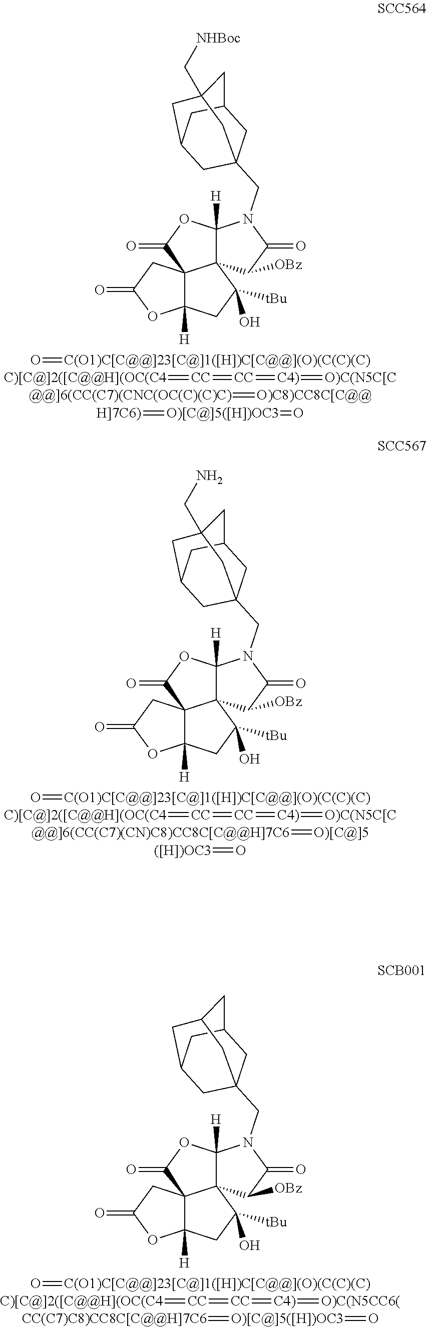

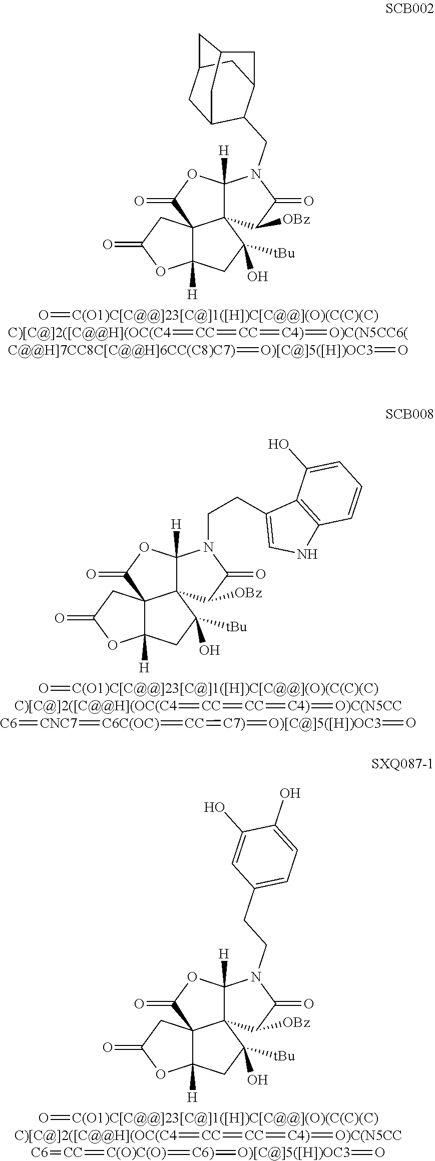

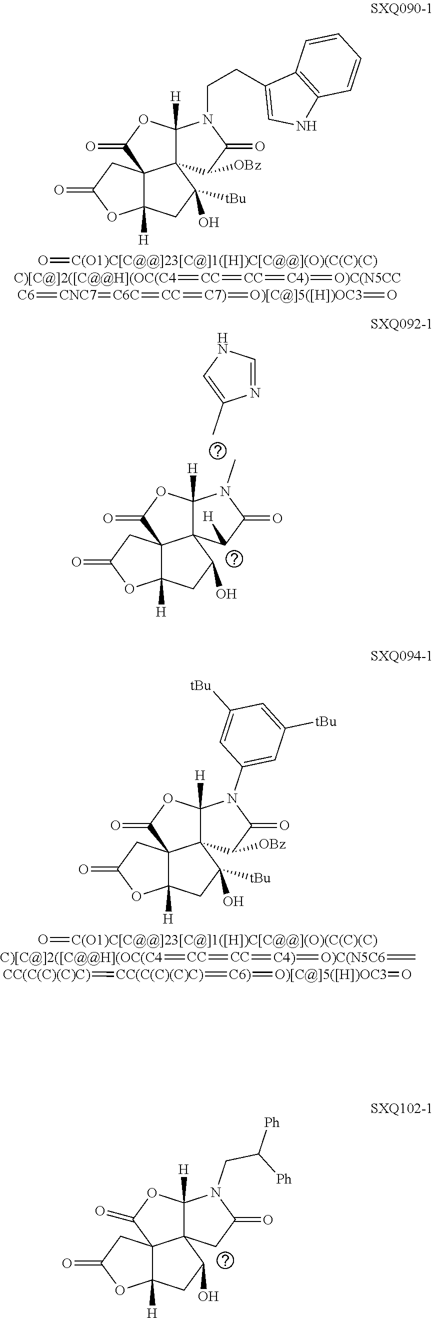

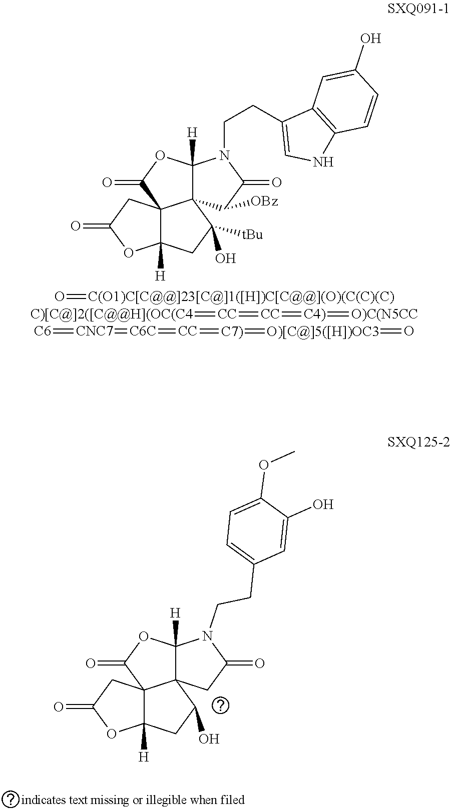

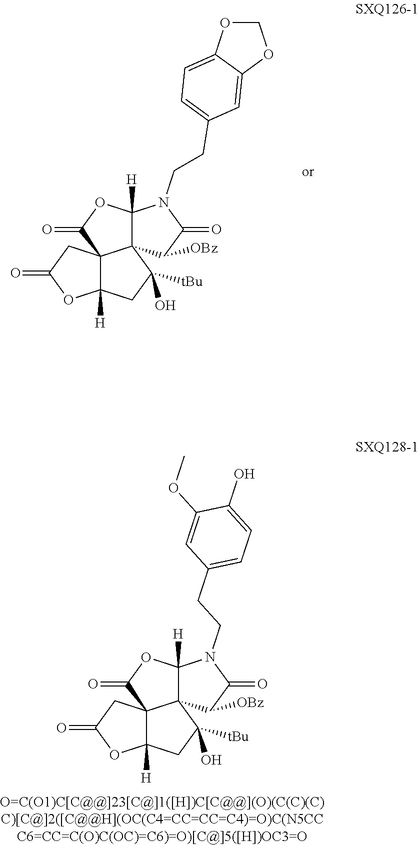

15. The compound of claim 1, wherein the compound is DW192, P-29, P-21, P-30, P-33, JW093, XBB-023, P-28, JW107, XBB-039, JW094, P-34, XBB-045, JW081, XBB-028, XBB-038, XBB-037, XBB-054, XBB-025, XBB-029, XBB-024, DW172, XBB-004, XBB-042, 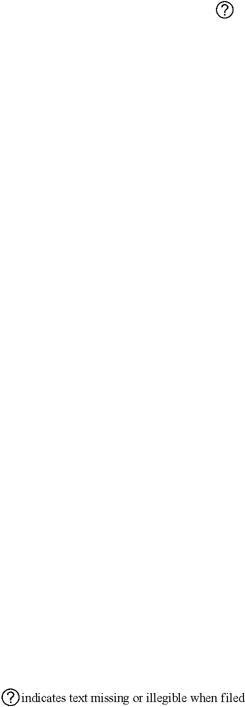
068 XBB-040 XBB-006, JW072, DW189, P-8, DW191, DW168, XBB-013, XBB-037′, XBB-009, XBB-060, XBB-016, DW182, XBB-010, SCC506, SCC363, or SXQ087-1.

16. The compound of claim 1, wherein bond Y¹ is a double bond, having Formula I′:

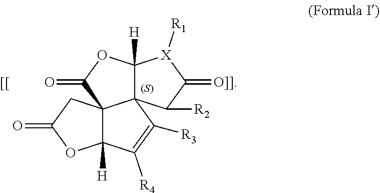

17. The compound of claim 16, wherein
X is —O— and R¹ is absent;
R² is R^{2A} or OR^{2A}, wherein R^{2A} is H, (C═O)CH₃, SO₂CH₃, S^{O}₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅, 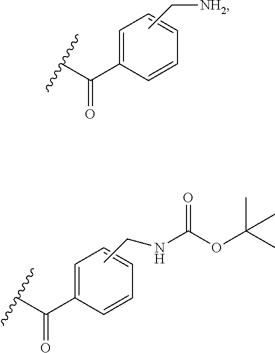
or NH(C═O)OC(CH₃)₃;
wherein phenyl is option 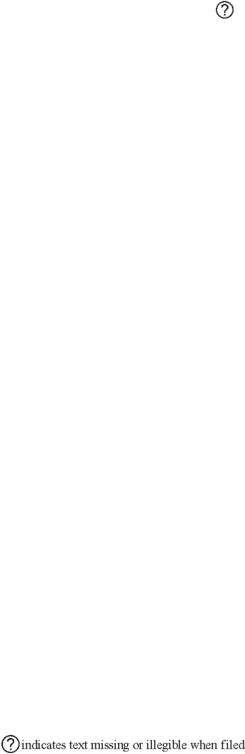
substituted with 1-5 occurrences of halo, R′ or OR′;
R³ is tert-butyl;
R⁴ is absent;
R⁵ is H; and
R⁶ is absent.

18. The compound of claim 1, having Formula I′a:

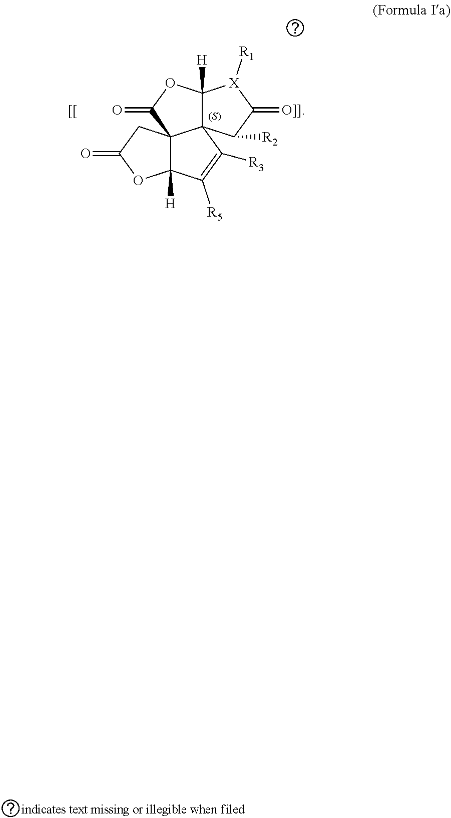

19. The compound of claim 1, having Formula I′b:

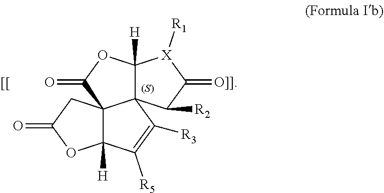

20. The compound of claim 1, wherein
X is —O— and R¹ is absent;
bond Y¹ is a single bond;
R² is OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, (C═O)C₆H₅, 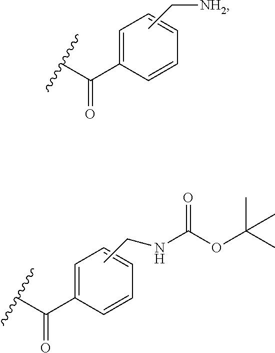
CH₂CH₂OCH₃, or NH(C═O)OC(CH₃)₃;
wherein phenyl is optionally substituted with 1-5 occurrences of halo, R′ or OR′;
R³ is isopropenyl;
R⁴ is CH₃;
R⁵ is H; and
R⁶ is H.

21. 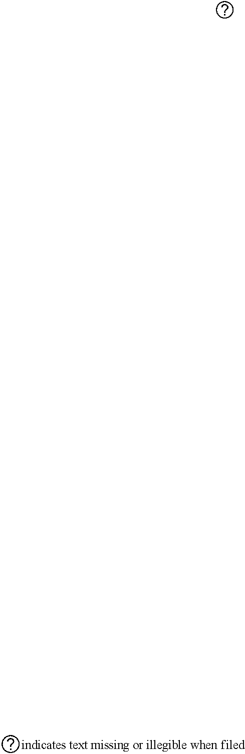
A compound of Formula II: 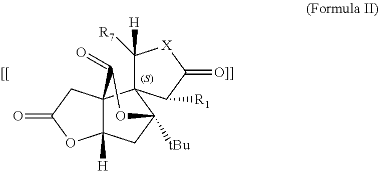
or a stereomer, a tautomer, or a pharmaceutically acceptable salt thereof,
wherein
X is —O—, —NR¹—, —N═CR¹—NH—, or —NR¹—NH—; wherein R¹ is as defined in claim 1;
R² is R^{2A} or OR^{2A}, wherein R^{2A} is H, a C₁-C₁₆ aliphatic or a 5-10 membered aromatic or nonaromatic monocyclic or bicyclic ring system, wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{2A} is optionally substituted with 1-5 occurrences of R^{2B}, wherein R^{2B} is halo, R′ or OR′; and
R⁷ is R^{7A} or OR^{7A}, wherein R^{7A} is H, a C₁-C₁₆ aliphatic or a 5-10 membered aromatic or nonaromatic monocyclic or bicyclic ring system, wherein up to five carbon atoms of the C₁-C₁₆ aliphatic or the 5-10 membered aromatic or nonaromatic monocyclic or bicyclic ring system are optionally replaced by N, NR, O, S, C═O, SO₂, S═O, (C═O)N, N(C═O)N, (C═O)O, or Si; wherein R^{7A} is optionally substituted with 1-5 occurrences of R^{7B}, wherein R^{7B} is halo, R′ or OR′.

22. The compound of claim 21, having Formula IIa:

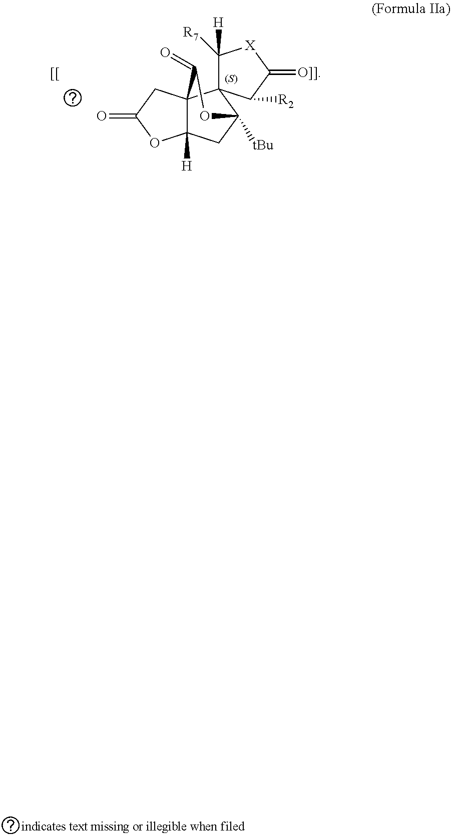

23. The compound of claim 21, having Formula IIb:

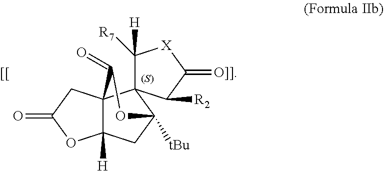

24. The compound of claim 21, wherein
X is —O—;
R² is R^{2A} or OR^{2A}, wherein R^{2A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅, 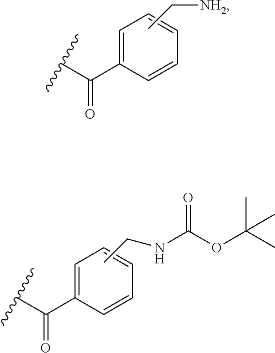
or NH(C═O)OC(CH₃)₃;
wherein phenyl is optionally substituted with 1-5 occurrences of halo, R′ or OR′; and
R⁷ is R^{7A} or OR^{7A}, wherein R^{7A} is H, (C═O)CH₃, SO₂CH₃, SO₂C₆H₄CH₃, SO₂CF₃, phenyl, Si(CH₃)₂C(CH₃)₃, Si(CH₂CH₃)₃, Si(CH₃)₃, Si(C₆H₅)₂C(CH₃)₃, Si(iPr)₃, CH₂OCH₃, CH₂CH₂OCH₃, (C═O)C₆H₅, 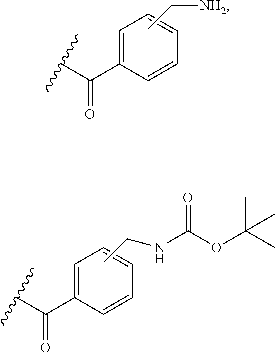
or NH(C═O)OC(CH₃)₃;
wherein phenyl is optionally substituted with 1-5 occurrences of halo, R′ or OR′.

25. The compound of claim 21, wherein the compound is

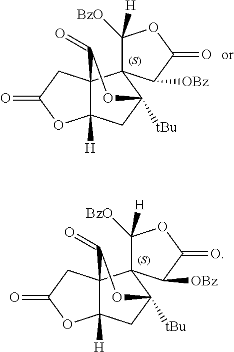

26. A process for preparing a compound of claim 1, comprising at least the following steps:
i) treating bilobalide with R^{2A}—X in a suitable solvent to form protected product IIa 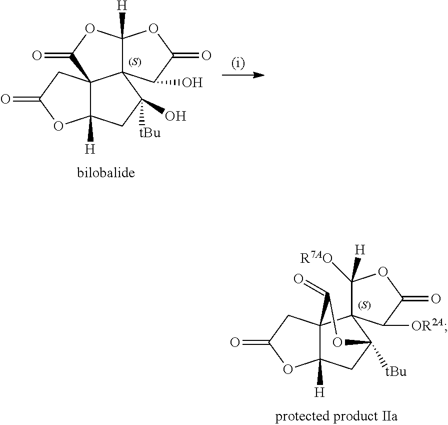
and
ii) treating protected product IIa with at least one base or an acceptable salt thereof to form aminated product IIb 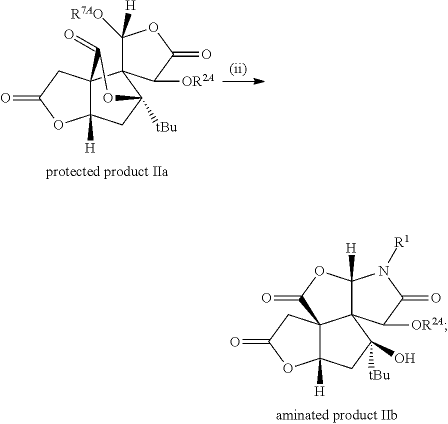
wherein R^{2A} and R^{7A} are as defined in claim 21.

27. The process of claim 26, wherein the aminated product IIb is aminated product IIb′, further comprising the step of:
iii) treating the aminated product IIb′ with R¹—B(OH)₂ in the presence of a catalyst to form a N-arylated product IIc 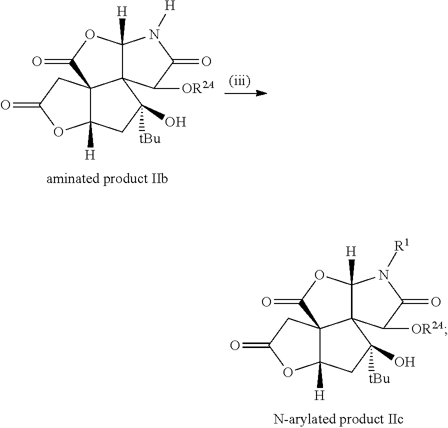
wherein R¹ and R^{2A} are as defined in claim 1.

27. (canceled)

28. The process of claim 26, wherein R^{2A} and R^{7A} of the protected product IIa is as defined in claim 21.

29. The process of claim 26, wherein the R^{2A}—X is benzoyl chloride, or 4-(Boc-aminomethyl) benzoic acid, and the suitable solvent is pyridine or dichloromethane.

30. The process of claim 26, wherein the at least one base is ammonia, and the aminated product IIb′ is:

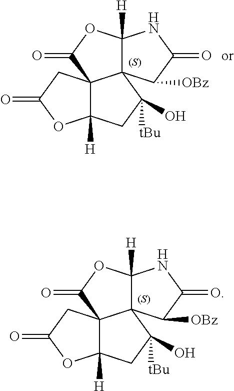

31. The process of claim 26, further comprising the step of:
treating the aminated product IIb of claim 26 in a protic solvent to form a deprotected product, wherein the deprotected product is as defined in claim 1.

32. The process of claim 26, wherein the at least one base is NH₂R¹ or [H₃NR¹]⁺.

33. The process of claim 32, further includes a second base in step (ii), wherein in the second base is a hindered base selected from triethylamine, diisopropylethylamine, tributylamine, or tetramethylethylenediamine.

34. The process of claim 26, wherein the protected product IIa is as defined in claim 21.

35. The process of claim 32, wherein the [H₃NR¹]⁺ is provided as XYa prepared by the steps of:
a) treating R—COOH with 1-hydroxybenzotriazole, N-(3-dimethylaminopropyl)-N′-ethylcarbodiimide hydrochloride, and tert-butyl (piperidin-4-ylmethyl)carbamate, to form a boc-protected product SXa, wherein R═(Z²)_{w}—R^{1B}, wherein Z², w and R^{1B} are as defined in claim 1; and
b) treating the boc-protected product SXa with an acid in a solvent to form XYa

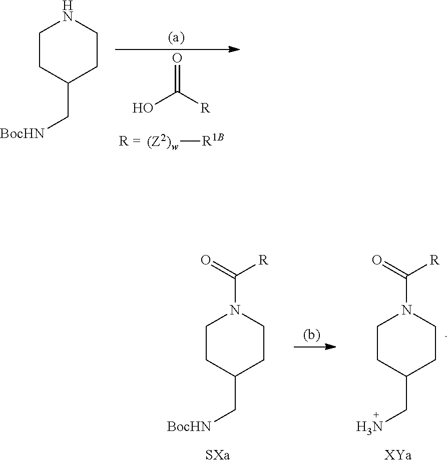

36. The process of claim 33, wherein the [H₃NR¹]⁺ is provided as XYb prepared by the steps of:
a) treating R—SO₂ with tert-butyl (piperidin-4-ylmethyl)carbamate and triethylamine, to form a boc-protected product SXb, wherein R═(Z²)_{w}—R^{1B}, wherein Z², w and R^{1B} are as defined in claim 1; and
b) treating the boc-protected product SXb with an acid in a solvent to form XYb

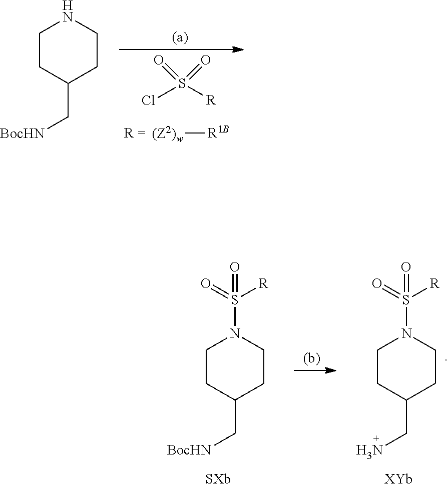

37. A process of preparing a compound of claim 1, comprising at least one of the steps of:
i) treating bilobalide with Ac₂O and an acid to form a protected product IVa and/or protected product Va 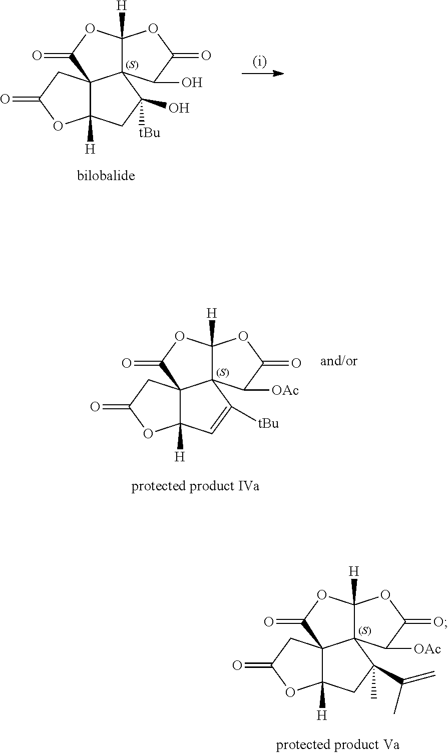
and
ii) treating protected product IVa or protected product Va with at least one base or an acceptable salt thereof to form aminated product IVb or aminated product Vb 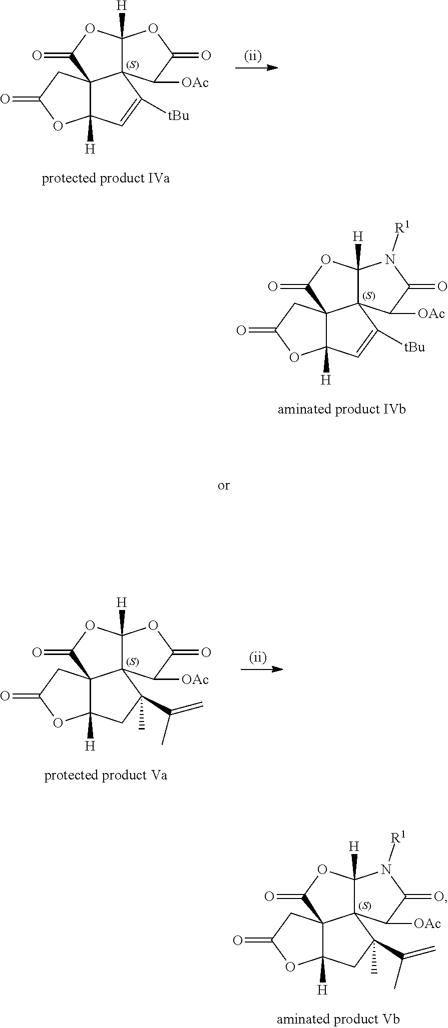
wherein R¹ is as defined in claim 1.

38. The process of claim 37, further comprising the step of:
iii) treating the aminated product VIb with an oxidizing agent and a solvent to form oxidized product VIc 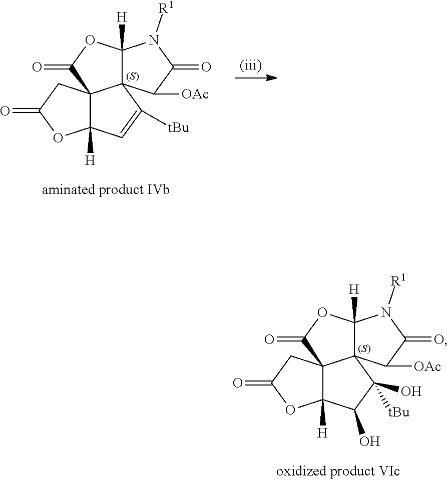
wherein R¹ is as defined in claim 1.

39. The process of claim 37, further comprising at least one of the steps of:
iv) treating the aminated product IVb or the aminated product Vb with an acid to form deprotected product IVd or deprotected product Vd, 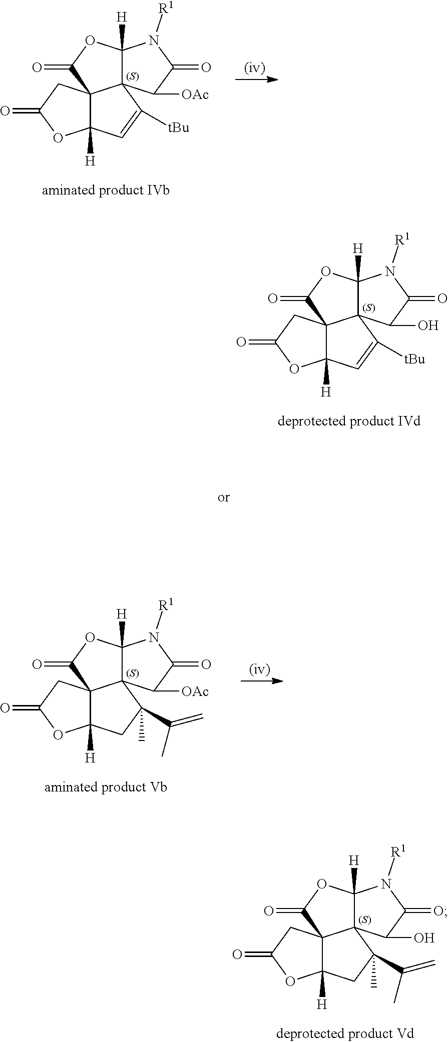
wherein R¹ is as defined in claim 1.

40. The process of claim 38, further comprising the step of:
v) treating the oxidized product VIc with an acid to form deprotected product VId, 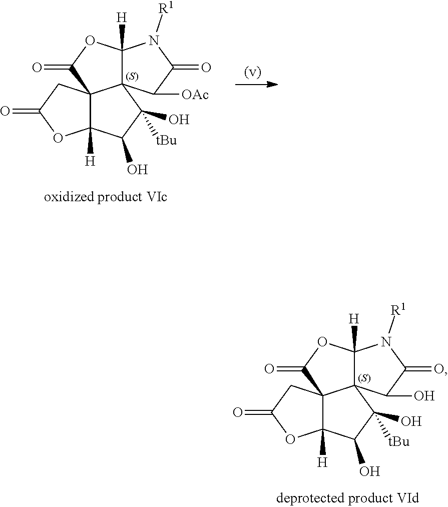
wherein R¹ is as defined in claim 1.

41. A method of treating or preventing cancer in a subject in need thereof, comprising administering to the subject a compound of claim 1.

42. The method of claim 41, wherein the cancer is bladder cancer, brain cancer, breast cancer, CNS cancer, colon cancer, hematopoietic cancer, kidney cancer, leukemia, lung cancer, melanoma, ovarian cancer, pancreatic cancer, prostate cancer, or renal cancer.

43. The method of claim 42, wherein the cancer is leukemia, colon cancer, lung cancer, melanoma or renal cancer.

44. The method of claim 43, wherein the lung cancer is non-small cell lung cancer (NSCLC).

45. The method of claim 44, wherein the leukemia is lymphocytic leukemia.

46-47. (canceled)

48. A method of inducing cell death in a cancer cell, comprising contacting a compound of claim 1 with the cancer cell.

49. A method of inhibiting cell growth in a cancer cell, comprising contacting a compound of claim 1 with the cancer cell.

50. The method of claim 48, wherein the method is an in vitro method.

51. The method of claim 41, wherein the compound is DW192, P-29, P-21, P-30, P-33, JW093, XBB-023, P-28, JW107, XBB-039, JW094, P-34, XBB-045, JW081, XBB-028, XBB-038, XBB-037, XBB-054, XBB-025, XBB-029, XBB-024, DW172, XBB-004, XBB-042, XBB-068, XBB-040, XBB-006, JW072, DW189, P-8, DW191, DW168, XBB-013, XBB-037′, XBB-009, XBB-060, XBB-016, DW182, XBB-010, SCC506, SCC363, or SXQ087-1.

52. The method of claim 51, wherein the compound is DW192, P-29, P-21, SCC506, SCC363, or SXQ087-1.

53. A method of treating or preventing neurological related disease in a subject in need thereof, comprising administering to the subject a compound of claim 1.

54. The method of claim 53, wherein the neurological related disease is caused by ferroptosis.

55. The method of claim 54, wherein the neurological related disease is Alzheimer's disease or Parkinson's disease.

56. (canceled)

57. A method of inhibiting ferroptosis comprising contacting a compound of claim 1 with a cell.

58-61. (canceled)

62. The method of claim 53, wherein the compound is DW192, P-29, P-21, P-30, P-33, JW093, XBB-023, P-28, JW107, XBB-039, JW094, P-34, XBB-045, JW081, XBB-028, XBB-038, XBB-037, XBB-054, XBB-025, XBB-029, XBB-024, DW172, XBB-004, XBB-042, XBB-068, XBB-040, XBB-006, JW072, DW189, P-8, DW191, DW168, XBB-013, XBB-037′, XBB-009, XBB-060, XBB-016, DW182, XBB-010, SCC506, SCC363, or SXQ087-1.

63. The method of claim 62, wherein the compound is DW192, P-29, P-21, SCC506, SCC363, or SXQ087-1.

64. The process of claim 27, further comprising the step of: treating the N-arylated product IIc of claim 27 in a protic solvent to form a deprotected product, wherein the deprotected product is as defined in claim 1.

65. The method of claim 49, wherein the method is an in vitro method.
